(19) United States
(12) Patent Application Publication
(10) Pub. No.: US 2025/0223277 A1
(43) Pub. Date: Jul. 10, 2025

(54) COMPLEMENT FACTOR B INHIBITORS AND USES THEREOF

(71) Applicant: Novartis AG, Basel (CH)

(72) Inventors: Lara C. CZABANIUK, Arlington, MA (US); Michael Christoph HEDIGER, Rheinfelden (CH); Konstanze HURTH, Lörrach (DE); Martin SENDZIK, Belmont, MA (US); Nancy LABBE-GIGUERE, Arlington, MA (US); Anna VULPETTI, Basel (CH)

(21) Appl. No.: 18/851,253

(22) PCT Filed: Mar. 30, 2023
(86) PCT No.: PCT/IB2023/053202

§ 371 Date: Sep. 26, 2024

Related U.S. Application Data
(60) Provisional application No. 63/326,532, filed on Apr. 1, 2022.

(51) Int. Cl. C07D 401/06; A61K 31/404; A61K 31/41; A61K 31/454; A61K 31/55; C07D 401/14; C07D 403/06; C07D 403/14; C07D 405/14

(52) U.S. Cl. CPC C07D 401/06; A61K 31/404; A61K 31/41; A61K 31/454; A61K 31/55; C07D 401/14; C07D 403/06; C07D 403/14; C07D 405/14

(57) ABSTRACT

Provided herein are compounds of formula (I) and pharmaceutical compositions thereof useful for treating diseases or disorders mediated by the complement factor B.

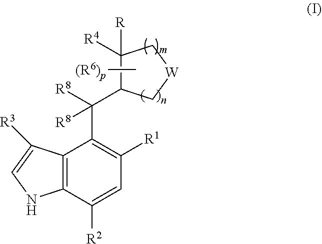

### FIELD

The disclosure relates to the inhibition of the complement alternative pathway and particularly to inhibition of Factor B, in patients suffering from conditions and diseases associated with complement alternative pathway activation such as age-related macular degeneration, diabetic retinopathy and related ophthalmic diseases.

### BACKGROUND

The complement system is a crucial component of the innate immunity system and comprises a group of proteins that are normally present in an inactive state. These proteins are organized in three activation pathways: the classical, the lectin, and the alternative pathways (V. M. Holers, In Clinical Immunology: Principles and Practice, ed. R. R. Rich, Mosby Press; 1996, 363-391). Molecules from microorganisms, antibodies or cellular components can activate these pathways resulting in the formation of protease complexes known as the C3-convertase and the C5-convertase. The classical pathway is a calcium/magnesium-dependent cascade, which is normally activated by the formation of antigen-antibody complexes. It can also be activated in an antibody-independent manner by the binding of C-reactive protein complexed to ligand and by many pathogens including gram-negative bacteria. The alternative pathway is a magnesium-dependent cascade which is activated by deposition and activation of C3 on certain susceptible surfaces (e.g., cell wall polysaccharides of yeast and bacteria, and certain biopolymer materials).

Factor B may be a suitable target for the inhibition of this amplification of the complement pathways because its plasma concentration in humans is typically about 200 μg/mL (or about 2 μM), and it has been shown to be a critical enzyme for activation of the alternative complement pathway (P. H. Lesavre and H. J. Müller-Eberhard. J. Exp. Med., 1978; 148: 1498-1510; J. E. Volanakis et al., New Eng. J. Med., 1985; 312:395-401).

Macular degeneration is a clinical term that is used to describe a family of diseases that are characterized by a progressive loss of central vision associated with abnormalities of Bruch's membrane, the choroid, the neural retina and/or the retinal pigment epithelium. In the center of the retina is the macula lutea, which is about ⅓ to ½ cm in diameter. The macula provides detailed vision, particularly in the center (the fovea), because the cones are higher in density and because of the high ratio of ganglion cells to photoreceptor cells. Blood vessels, ganglion cells, inner nuclear layer and cells, and the plexiform layers are all displaced to the side (rather than resting above the photoreceptor cells), thereby allowing light a more direct path to the cones. Under the retina is the choroid, a part of the uveal tract, and the retinal pigmented epithelium (RPE), which is between the neural retina and the choroid. The choroidal blood vessels provide nutrition to the retina and its visual cells.

Age-related macular degeneration (AMD), the most prevalent form of macular degeneration, is associated with progressive loss of visual acuity in the central portion of the visual field, changes in color vision, and abnormal dark adaptation and sensitivity. Two principal clinical manifestations of AMD have been described as the dry, or atrophic, form and the neovascular, or exudative, form. The dry form is associated with atrophic cell death of the central retina or macula, which is required for fine vision used for activities such as reading, driving or recognizing faces. About 10-20% of these AMD patients progress to the second form of AMD, known as neovascular AMD (also referred to as wet AMD).

Neovascular AMD is characterized by the abnormal growth of blood vessels under the macula and vascular leakage, resulting in displacement of the retina, hemorrhage and scarring. This results in a deterioration of sight over a period of weeks to years. Neovascular AMD cases originate from Intermediate or advanced dry AMD. The neovascular form accounts for 85% of legal blindness due to AMD. In neovascular AMD, as the abnormal blood vessels leak fluid and blood, scar tissue is formed that destroys the central retina.

The new blood vessels in neovascular AMD are usually derived from the choroid and are referred to as choroidal neovascularizaton (CNV). The pathogenesis of new choroidal vessels is poorly understood, but such factors as inflammation, ischemia, and local production of angiogenic factors are thought to be important. A published study suggests that CNV is caused by complement activation in a mouse laser model (Bora P. S., J. Immunol. 2005; 174; 491-497).

Human genetic evidence implicates the involvement of the complement system, particularly the alternative pathway, in the pathogenesis of Age-related Macular Degeneration (AMD). Significant associations have been found between AMD and polymorphisms in complement factor H (CFH) (Edwards A O, et al. Complement factor H polymorphism and age-related macular degeneration. Science. 2005 Apr. 15; 308(5720):421-4; Hageman G S, et al A common haplotype in the complement regulatory gene factor H (HF1/CFH) predisposes individuals to age-related macular degeneration. Proc Natl Acad Sci USA. 2005 May 17; 102(20):7227-32; Haines J L, et al. Complement factor H variant increases the risk of age-related macular degeneration. Science. 2005 Apr. 15; 308(5720):419-21; Klein R J, et al Complement factor H polymorphism in age-related macular degeneration. Science. 2005 Apr. 15; 308(5720):385-9; Lau L I, et al. Association of the Y402H polymorphism in complement factor H gene and neovascular age-related macular degeneration in Chinese patients. Invest Ophthalmol Vis Sci. 2006 August; 47(8):3242-6; Simonelli F, et al. Polymorphism p.402Y>H in the complement factor H protein is a risk factor for age related macular degeneration in an Italian population. Br J Ophthalmol. 2006 September; 90(9):1142-5; and Zareparsi S, et al Strong association of the Y402H variant in complement factor H at 1q32 with susceptibility to age-related macular degeneration. Am J Hum Genet. 2005 July; 77(1):149-53.), complement factor B (CFB) and complement C2 (Gold B, et al. Variation in factor B (BF) and complement component 2 (C2) genes is associated with age-related macular degeneration. Nat Genet. 2006 April; 38(4):458-62 and Jakobsdottir J, et al. C2 and CFB genes inage-related maculopathy and joint action with CFH and LOC387715 genes. PLOS One. 2008 May 21; 3(5):e2199), and most recently in complement C3 (Despriet D D, et al Complement component C3 and risk of age-related macular degeneration. Ophthalmology. 2009 March; 116(3):474-480.e2; Maller J B, et al Variation in complement factor 3 is associated with risk of age-related macular degeneration. Nat Genet. 2007 October; 39(10):1200-1 and Park K H, et al Complement component 3 (C3) haplotypes and risk of advanced age-related macular degeneration. Invest Ophthalmol Vis Sci. 2009 July; 50(7):3386-93. Epub 2009 Feb. 21.). Taken together, the genetic variations in the alternative pathway components CFH, CFB, and C3 can predict clinical outcome in nearly 80% of cases.

Currently there is no proven medical therapy for dry AMD and many patients with neovascular AMD become legally blind despite current therapy with anti-VEGF agents such as Lucentis. Thus, it would be desirable to provide therapeutic agents for the treatment or prevention of complement mediated diseases and particularly for the treatment of AMD.

### SUMMARY

The present disclosure provides compounds that modulate, and/or inhibit, activation of the alternative complement pathway. In certain embodiments, the present disclosure provides compounds that modulate, and/or inhibit, Factor B activity and/or Factor B mediated complement pathway activation. Such Factor B modulators are preferably high affinity Factor B inhibitors that inhibit the catalytic activity of complement Factor B, such as primate Factor B and particularly human Factor B.

The compounds of the present disclosure inhibit or suppress the amplification of the complement system caused by C3 activation irrespective of the initial mechanism of activation (including for example activation of the classical, lectin or alternative pathways).

The disclosure also relates to compounds effective as Factor B modulators, pharmaceutically acceptable salts thereof, compositions thereof, and their use in therapies for the conditions and purposes detailed herein.

The disclosure provides, in a first aspect, a compound of formula (I) or a pharmaceutically acceptable salt thereof,

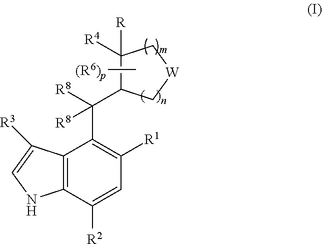

wherein: m is 0, 1, 2, or 3; n is 0, 1, 2, or 3, with the proviso that both m and n are not 0;
R is hydrogen, halogen, C₁-C₄alkyl, haloC₁-C₄alkyl, or hydroxyC₁-C₄alkyl;
R¹ is hydrogen, halogen, hydroxy, C₁-C₆alkyl, C₂-C₆alkenyl, C₃-C₆cycloalkyl, C₁-C₆alkoxy, haloC₁-C₆alkyl, haloC₃-C₆cycloalkyl, haloC₁-C₆alkoxy, hydroxyC₁-C₆alkyl, hydroxyC₃-C₆cycloalkyl aminoC₁-C₆alkyl, C₁-C₆alkoxyC₁-C₆alkyl, C₁-C₆alkoxyC₁-C₆alkoxy, C₃-C₆cycloalkylC₁-C₆alkoxy, haloC₁-C₆alkoxy, —S—C₁-C₆alkyl, —S(O)C₁-C₆alkyl, —S(O)₂C₁-C₆alkyl, CH₂NHC(O)C₁-C₄alkyl or —OCH₂C(O)R^{1a};
   R^{1a} is hydroxy, C₁-C₆alkoxy, amino or mono- and di-C₁-C₆alkylamino;
R² is C₁-C₃alkyl, C₃cycloalkyl, C₁-C₃alkoxy, hydroxyC₁-C₆alkyl or halogen, wherein the C₁-C₃alkyl, C₃cycloalkyl, or C₁-C₃alkoxy are unsubstituted or substituted with 1, 2, or 3 halogen substituents;
p is 0, 1, 2, or 3;
R³ is hydrogen, halogen, or cyano;
R⁴ is phenyl, naphthyl or heteroaryl, where the heteroaryl is a five to 10 member heteroaryl having 1, 2 or 3 ring heteroatoms independently selected from N, O, or S, and where the phenyl or heteroaryl is unsubstituted or substituted with 1 R⁵ and substituted with 0, 1, or 2 R^{5′}; wherein
R⁵ is selected from —CO₂R^{5b}, —CON(R^{5c})₂, C₁-C₆alkyl, C₁-C₆alkoxyC₁-C₆alkyl, —CH₂CO₂R^{5b}, —CH₂CON(R^{5c})₂, —C(O)NHSO₂C₁-C₄alkyl, —SO₂NHC(O)C₁-C₄alkyl, —SO₂N(H)ₘ(C₁-C₄alkyl)₂₋ₘ, —SO₂C₁-C₄alkyl, cyano, hydroxy, halogen, 5- to 6-membered heteroaryl having 1-4 heteroatoms independently selected from N, O, and S, and 4- to 6-membered heterocyclyl having 1-2 heteroatoms independently selected from N, O, and S(O)_{q}, wherein the 5- to 6-membered heteroaryl and 4- to 6-membered heterocyclyl are unsubstituted or substituted with 1, 2, or 3 R^{5a};
each R^{5a} is independently selected from fluoro, hydroxyl and C₁-C₆alkyl that is unsubstituted or substituted with —COOH or 1, 2, or 3 fluoro;
   wherein when R⁵ is a 4- to 6-membered heterocyclyl, two R^{5a} are not fluoro and hydroxyl substituted on the same position;
each R^{5b} is independently selected from hydrogen or C₁-C₆alkyl;
each R^{5c} is independently selected from hydrogen, C₁-C₄alkyl unsubstituted or substituted with halogen, hydroxy or C₁-C₄alkyl;
each R⁵ is independently selected from the group consisting of haloC₁-C₄alkyl, C₁-C₄alkoxy, haloC₁-C₄alkoxy, halogen, cyanomethyl, hydroxyC₁-C₆alkyl, and C₃-C₅cycloalkyl;
W is O, C(R⁶)₂, or NR⁷;
R⁶ is independently selected at each occurrence from the group consisting of hydrogen, halogen, hydroxy, amino, mono- and di-C₁-C₄alkylamino, C₁-C₆alkyl, haloC₁-C₆alkyl, hydroxyC₁-C₆alkyl, cyanoC₁-C₆alkyl, C₁-C₆alkoxy, or haloC₁-C₆alkoxy; or two R⁶ in combination with the carbon atom to which they are attached form a spirocyclic carbocycle having 3 to 6 ring atoms, wherein the spirocyclic carbocycle is unsubstituted or substituted with 1 or 2 substituents selected from halogen, C₁-C₆alkyl, or C₁-C₆alkoxy; or two R⁶ in combination with the carbon atom to which they are attached form a spirocyclic heterocycle having 1 or 2 ring heteroatoms independently selected from N, O, or S(O)_{q}, wherein the spirocyclic heterocycle is unsubstituted or substituted with 1 or 2 halogen substituents, C₁-C₆alkyl, haloC₁-C₆alkyl, C₃-C₆cycloalkyl, haloC₃-C₆cycloalkyl;
q is 0, 1, or 2;
R⁷ is hydrogen, C₁-C₆alkyl, haloC₁-C₆alkyl, hydroxyC₂-C₆alkyl, C₃-C₆cycloalkyl, C₃-C₆cycloalkylC₁-C₃alkyl, haloC₃-C₆cycloalkyl, C₁-C₆alkoxyC₁-C₆alkyl, haloC₁-C₆alkoxyC₁-C₆alkyl, aryl, 4- to 6-membered heterocyclyl having 1-2 heteroatoms independently selected from N, O, and S(O)_{q}, or 5- to 6-membered heteroaryl having 1-4 heteroatoms independently selected from N, O, and S, wherein C₁-C₆alkyl is unsubstituted or substituted with 4- to 6-membered heterocyclyl having 1-2 heteroatoms independently selected from N, O, and S(O)_{q}, wherein the 4- to 6-membered heterocyclyl is unsubstituted or substituted with 1, 2, or 3 halogen, further wherein the aryl or heteroaryl is unsubstituted or substituted with halo, C₁-C₆alkyl, haloC₁-C₆alkyl, C₃-C₅cycloalkyl, haloC₃-C₅cycloalkyl;
each R⁸ is independently hydrogen, halogen, C₁-C₄alkyl, haloC₁-C₄alkyl, or two R⁸ together with the carbon atom to which they are attached form a C₃-C₅cycloalkyl or haloC₃-C₅cycloalkyl.

In a second aspect, there is provided a pharmaceutical composition comprising a therapeutically effective amount of a compound of Formula (I), or a pharmaceutically acceptable salt thereof, and a pharmaceutically acceptable carrier or excipient.

In a third aspect, there is provided a method of treating or preventing a disease or disorder in a subject in need thereof, the method comprising administering to the subject a therapeutically effective amount of a compound of Formula (I), or a pharmaceutically acceptable salt thereof.

In a further aspect, there is provided a method of modulating the complement alternative pathway activity in a subject, the method comprising administering to the subject a therapeutically effective amount of a compound of Formula (I), or a pharmaceutically acceptable salt thereof.

In a further aspect, there is provided a method of treating a disease or disorder mediated by complement activation, in particular mediated by activation of the complement alternative pathway, in a subject in need thereof, the method comprising administering to the subject a therapeutically effective amount of a compound of Formula (I), or a pharmaceutically acceptable salt thereof.

In a further aspect, there is provided a method of treating a disease or disorder that is affected by the modulation of complement alternative pathway comprising administering to the subject a therapeutically effective amount of a compound of Formula (I), or a pharmaceutically acceptable salt thereof.

In a further aspect, there is provided a method of treating a disease or disorder associated with dysregulation of the complement alternative pathway comprising administering to the subject a therapeutically effective amount of a compound of Formula (I), or a pharmaceutically acceptable salt thereof.

In a further aspect, there is provided a method of inhibiting the expression or activity of complement factor B, the method comprising administering to the subject a compound of Formula (I), or a pharmaceutically acceptable salt thereof.

In a further aspect, there is provided a method of treating age-related macular degeneration comprising administering to a subject in need thereof an effective amount of a composition comprising a compound of Formula (I), or a pharmaceutically acceptable salt thereof.

In a further aspect, there is provided a compound of Formula (I), or a pharmaceutically acceptable salt thereof, for use as a medicament.

In a further aspect, there is provided a compound of Formula (I), or a pharmaceutically acceptable salt thereof, for use in inhibiting the expression or activity of complement factor B, in a subject in need thereof.

In a further aspect, there is provided a compound of Formula (I), or a pharmaceutically acceptable salt thereof, for use in treating a disease or disorder associated with dysregulation of the complement alternative pathway.

In a further aspect, there is provided the use of a compound of Formula (I), or a pharmaceutically acceptable salt thereof, in the manufacture of a medicament for the treatment of a disease or disorder mediated by complement activation or activation of the complement alternative pathway.

In a further aspect, there is provided the use of a compound of Formula (I), or a pharmaceutically acceptable salt thereof, for the treatment of a disease or disorder mediated by complement activation or activation of the complement alternative pathway.

In a further aspect, there is provided the use of a compound of Formula (I), or a pharmaceutically acceptable salt thereof, for the treatment of a disease or disorder that is affected by the modulation of complement alternative pathway.

### DETAILED DESCRIPTION

As noted above, the present disclosure provides compounds that modulate Factor B activation and/or Factor B-mediated signal transduction of the complement system. Such compounds may be used in vitro or in vivo to modulate Factor B activity in a variety of contexts. The compounds disclosed herein are effective as Factor B modulators and/or inhibitors. Without wishing to be bound by any theory, it is believed that the disclosed compounds may treat disorders associated with Factor B, including the treatment of age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammation, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), immune complex membranoproliferative glomerulonephritis (IC-MPGN), IgA nephropathy, membranous nephropathy including idiopathic membranous nephropathy, diabetic nephropathy, atypical hemolytic uremic syndrome (aHUS), Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, neuromyelitis optica (NMO), IgA vasculitis (formerly known as Henoch-Schonlein purpura or HSP), CHAPLE syndrome, hematopoietic stem cell transplantation-associated thrombotic microangiopathy (HSCT-TMA), adult respiratory distress syndrome (ARDS,), myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury mesenteric artery reperfusion after aortic reconstruction, infectious disease or sepsis; COVID-19, immune complex disorders and autoimmune diseases, rheumatoid arthritis, osteoarthritis, Spondyloarthropathies, psoriatic arthritis, systemic lupus erythematosus (SLE), lupus nephritis, SLE nephritis, proliferative nephritis, myasthenia gravis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, antineutrophil cytoplasmic antibody (ANCA)-associated vasculitides (AAV), Buerger's vasculitis, cryoglobulinemia, Kawasaki disease, Takayasu arteritis, cryoglobulinemia, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenia purpura (TTP), and abdominal aortic aneurisms, and Grave's disease.

### Definitions

Unless specified otherwise, the terms “compounds of the present disclosure,” “compounds of the disclosure,” or “compound of the disclosure” refer to compounds of formulae (I), (I-A), (I-B), (I-C), exemplified compounds, salts thereof, particularly pharmaceutically acceptable salts thereof, hydrates, solvates, as well as all stereoisomers (including diastereoisomers and enantiomers), rotamers, tautomers, and isotopically labeled compounds (including deuterium substitutions), as well as inherently formed moieties.

In the groups, radicals, or moieties defined below, the number of carbon atoms is often specified preceding the group, for example, C₁-C₁₀alkyl means an alkyl group or radical having 1 to 10 carbon atoms.

Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups. The articles “a” and “an” refer to one or more than one (e.g., to at least one) of the grammatical object of the article. By way of example, “an element” means one element or more than one element.

The term “and/or” means either “and” or “or” unless indicated otherwise.

The term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. The terms C₁-C₃alkyl and C₁-C₄alkyl are to be construed accordingly. Examples of C₁-C₆alkyl include, but are not limited to, methyl, ethyl, n-propyl, 1-methylethyl (iso-propyl), n-butyl, 1-methylpropyl (sec-butyl), 2-methylpropyl (iso-butyl), 1,1-dimethylethyl (tert-butyl), n-pentyl and n-hexyl. As used herein, C₁-C₆alkyl also includes cycloalkyl groups attached to alkyl group, e.g.,

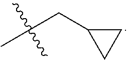 [SMILES: *CC1CC1]

As used herein, the term “C₁-C₆alkoxyl” refers to a radical of the formula —ORₐ where Rₐ is a C₁-C₆alkyl radical as generally defined above. Examples of C₁-C₆alkoxyl include, but are not limited to, methoxy, ethoxy, propoxy, iso-propoxy, butoxy, iso-butoxy, tert-butoxy, sec-butoxy, pentoxy, and hexoxy.

The term “halogen” or “halo” means fluorine, chlorine, bromine or iodine.

As used herein, the term “cycloalkyl” means a monocyclic or polycyclic saturated or partially unsaturated carbon ring containing 3-18 carbon atoms wherein there are no delocalized pi electrons (aromaticity) shared among the ring carbons. The term “C₃-C₅cycloalkyl” is to be construed accordingly. The term polycyclic encompasses bridged (e.g., norbornane), fused (e.g., decalin) and spirocyclic cycloalkyl. Preferably, cycloalkyl, e.g., C₃-C₅cycloalkyl, is a monocyclic hydrocarbon group of 3 to 5 carbon atoms.

Examples of cycloalkyl groups include, without limitations, cyclopropenyl, cyclopropyl cyclobutyl, cyclobutenyl, cyclopentyl, bicyclo[1.1.1]pentanyl and derivatives thereof.

Examples of C₃-C₅cycloalkyl include, but are not limited to, cyclopropyl, cyclobutyl, and cyclopentyl.

“Heterocyclyl” means a saturated or partially saturated monocyclic or polycyclic ring containing carbon and at least one heteroatom selected from oxygen, nitrogen, and sulfur (O, N, and S) and wherein there are no delocalized pi electrons (aromaticity) shared among the ring carbons or heteroatoms. The term “4- to 6-membered heterocyclyl” is to be construed accordingly. The heterocyclyl ring structure may be substituted by one or more substituents. The substituents can themselves be optionally substituted. The heterocyclyl may be bonded via a carbon atom or heteroatom. The term polycyclic encompasses bridged, fused and spirocyclic heterocyclyl.

Examples of heterocyclyl rings include, but are not limited to, oxetanyl, azetidinyl, tetrahydrofuranyl, tetrahydropyranyl, pyrrolidinyl, oxazolinyl, isoxazolinyl, oxazolidinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, 1,4-dioxanyl, dihydrofuranyl, 1,3-dioxolanyl, imidazolidinyl, dihydroisoxazolinyl, pyrrolinyl, pyrazolinyl, oxazepinyl, dithiolanyl, homotropanyl, dihydropyranyl (e.g., 3,6-dihydro-2H-pyranyl), oxaspiroheptanyl (e.g., 2-oxaspiro[3.3]heptan-6-yl) and the like.

Examples of 4- to 6-membered heterocyclyl include, without limitations, azetidinyl, pyrrolidinyl, tetrahydrofuranyl, tetrahydrothienyl, piperidinyl, piperazinyl, dihydroisoxazolinyl, tetrahydropyranyl, morpholinyl, dihydropyranyl (e.g., 3,6-dihydro-2H-pyranyl) and oxaspiroheptanyl (e.g., 2-oxaspiro[3.3]heptan-6-yl).

As used herein, the term “heteroaryl” is intended to include monocyclic heterocyclic aromatic rings containing one or more heteroatoms selected from oxygen, nitrogen, and sulfur (O, N, and S). Representative examples are pyrrolyl, furanyl, thienyl, oxazolyl, thiazolyl, imidazolyl, pyrazolyl, isothiazolyl, isooxazolyl, triazolyl, (e.g., 1,2,4-triazolyl), oxadiazolyl, (e.g., 1,2,3-oxadiazolyl, 1,2,4-oxadiazolyl, 1,2,5-oxadiazolyl, 1,3,4-oxadiazolyl), thiadiazolyl (e.g., 1,2,3-thiadiazolyl, 1,2,4-thiadiazolyl, 1,2,5-thiadiazolyl, 1,3,4-thiadiazolyl), tetrazolyl, pyranyl, pyridinyl, pyridazinyl, pyrimidinyl, pyrazinyl, 1,2,3-triazinyl, 1,2,4-triazinyl, 1,3,5-triazinyl, thiadiazinyl, azepinyl, azecinyl, and the like.

Heteroaryl is also intended to include bicyclic heterocyclic aromatic rings containing one or more heteroatoms selected from oxygen, nitrogen, and sulfur (O, N, and S). Representative examples are indolyl, isoindolyl, benzofuranyl, benzothiophenyl, indazolyl, benzopyranyl, benzimidazolyl, benzothiazolyl, benzisothiazolyl, benzoxazolyl, benzisoxazolyl, benzoxazinyl, benzotriazolyl, naphthyridinyl, phthalazinyl, pteridinyl, purinyl, quinazolinyl, cinnolinyl, quinolinyl, isoquinolinyl, quinoxalinyl, oxazolopyridinyl, isooxazolopyridinyl, pyrrolopyridinyl, furopyridinyl, thienopyridinyl, imidazopyridinyl, imidazopyrimidinyl, pyrazolopyridinyl, pyrazolopyrimidinyl, pyrazolotriazinyl, thiazolopyridinyl, thiazolopyrimidinyl, imdazothiazolyl, triazolopyridinyl, triazolopyrimidinyl, and the like.

Heteroaryl is also intended to include polycyclic heterocyclic aromatic rings containing one or more heteroatoms selected from oxygen, nitrogen, and sulfur (O, N, and S). Representative examples are carbazolyl, phenoxazinyl, phenazinyl, acridinyl, phenothiazinyl, carbolinyl, phenanthrolinyl, and the like.

The heteroaryl ring structure may be substituted by one or more substituents. The substituents can themselves be optionally substituted. The heteroaryl ring may be bonded via a carbon atom or heteroatom.

The term “5 or 6 membered heteroaryl” is to be construed accordingly.

Examples of 5 or 6 membered heteroaryl include, but are not limited to, furan, indolyl, pyridinyl, pyrimidinyl, pyridinonyl, pyridazinyl, triazolyl, (e.g., 1,2,4-triazolyl), pyrazolyl, thiazolyl, oxazolyl, isooxazolyl, pyrrolyl, oxadiazolyl, (e.g., 1,2,3-oxadiazolyl, 1,2,4-oxadiazolyl, 1,2,5-oxadiazolyl, 1,3,4-oxadiazolyl), imidazolyl, thiophenyl, thiadiazolyl (e.g., 1,2,3-thiadiazolyl, 1,2,4-thiadiazolyl, 1,2,5-thiadiazolyl, 1,3,4-thiadiazolyl), pyrazinyl, isooxazolopyridinyl, dihydropyridooxazinyl and tetrazolyl. The term “5 or 6 membered heteroaryl ring having 1-2 heteroatoms independently selected from N, O, and S” is to be construed accordingly.

As used herein “modulator”, means, for example, a compound of the disclosure, that modulates, decreases, or reduces the levels of a specific protein (e.g., complement factor B). The amount of a specific protein (e.g., complement factor B) modulated can be measured by comparing the amount of the specific protein (e.g., complement factor B) remaining after treatment with a compound of the disclosure as compared to the initial amount or level of the specific protein (e.g., complement factor B) present as measured prior to treatment with a compound of the disclosure.

As used herein “inhibitor”, means, for example, a compound of the disclosure, that inhibits or reduces the activity of at least one component of the complement pathway, e.g., the compound inhibits binding of one component to another component of the pathway. For example, the complement pathway is the alternative complement pathway. For example, the component of the complement pathway is complement factor B.

As used herein, the term “inhibit”, “inhibition” or “inhibiting” refers to the reduction or suppression of a given condition, symptom, or disorder, or disease, or a significant decrease in the baseline activity of a biological activity or process.

The term “effective amount” of the compounds described herein, refers to that amount of a therapeutic compound necessary or sufficient to perform its intended function within a mammal. An effective amount of the therapeutic compound can vary according to factors such as the amount of the causative agent already present in the mammal, the age, sex, and weight of the mammal, and the ability of the therapeutic compounds of the present disclosure to treat the conditions wherein complement factor B plays a role.

Thus, the term “a therapeutically effective amount” of a compound of the disclosure refers to an amount of the compound of the disclosure that will elicit the biological or medical response of a subject, for example, reduction or inhibition of an enzyme or a protein activity, or ameliorate symptoms, alleviate conditions, slow or delay disease progression, or prevent a disease, etc. In one embodiment, the term “a therapeutically effective amount” refers to the amount of the compound of the disclosure that, when administered to a subject, is effective to (1) at least partially alleviate, prevent and/or ameliorate a condition, or a disorder or a disease (i) mediated by complement factor B, or (ii) associated with complement factor B activity, or (iii) characterized by activity (normal or abnormal) of complement factor B: (2) reduce or inhibit the activity of complement factor B; or (3) reduce or inhibit the expression of complement factor B. In another embodiment, the term “a therapeutically effective amount” refers to the amount of the compound of the disclosure that, when administered to a cell, or a tissue, or a non-cellular biological material, or a medium, is effective to at least partially reducing or inhibiting the activity of complement factor B; or at least partially reducing or inhibiting the expression of complement factor B.

As used herein, the term “treat”, “treating” or “treatment” in connection to a disease or disorder refers in some embodiments, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those, which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder or a symptom thereof.

As used herein, the term “subject” or “patient” refers to human and non-human mammals, including but, not limited to, primates, rabbits, pigs, horses, dogs, cats, sheep, and cows. In particular embodiments, a subject or patient is a human. In some embodiments, the term “patient” or “subject” refers to a human being who is diseased with the condition (i.e., disease or disorder) described herein and who would benefit from the treatment. As used herein, a subject is “in need of” a treatment if such subject (patient) would benefit biologically, medically or in quality of life from such treatment.

In particular embodiments, the subject is an adult human at least about 18 years of age. In particular embodiments, the subject is an adult human from about 18 to about 75 years of age. In some embodiments, the subject is a human child up to about 18 years of age.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein, the term “about” refers to a range of values +/−10% of a specified value.

As used herein, the term “a,” “an,” “the” and similar terms used in the context of the disclosure (especially in the context of the claims) are to be construed to cover both the singular and plural unless otherwise indicated herein or clearly contradicted by the context.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

Various enumerated embodiments of the disclosure are described herein. It will be recognized that features specified in each embodiment may be combined with other specified features to provide further embodiments of the disclosure.

### ENUMERATED EMBODIMENTS

Embodiment 1. A compound of formula (I) or a pharmaceutically acceptable salt thereof,

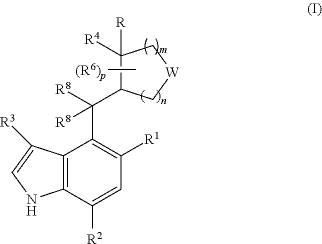

wherein: m is 0, 1, 2, or 3; n is 0, 1, 2, or 3, with the proviso that both m and n are not 0;
R is hydrogen, halogen, C₁-C₄alkyl, haloC₁-C₄alkyl, or hydroxyC₁-C₄alkyl;
R¹ is hydrogen, halogen, hydroxy, C₁-C₆alkyl, C₂-C₆alkenyl, C₃-C₆cycloalkyl, C₁-C₆alkoxy, haloC₁-C₆alkyl, haloC₃-C₆cycloalkyl, haloC₁-C₆alkoxy, hydroxyC₁-C₆alkyl, hydroxyC₃-C₆cycloalkyl aminoC₁-C₆alkyl, C₁-C₆alkoxyC₁-C₆alkyl, C₁-C₆alkoxyC₁-C₆alkoxy, C₃-C₆cycloalkylC₁-C₆alkoxy, haloC₁-C₆alkoxy, —S—C₁-C₆alkyl, —S(O)C₁-C₆alkyl, —S(O)₂C₁-C₆alkyl, CH₂NHC(O)C₁-C₄alkyl or —OCH₂C(O)R^{1a};
   R^{1a} is hydroxy, C₁-C₆alkoxy, amino or mono- and di-C₁-C₆alkylamino;
R² is C₁-C₃alkyl, C₃cycloalkyl, C₁-C₃alkoxy, hydroxyC₁-C₆alkyl or halogen, wherein the C₁-C₃alkyl, C₃cycloalkyl, or C₁-C₆alkoxy are unsubstituted or substituted with 1, 2, or 3 halogen substituents;
p is 0, 1, 2, or 3;
R³ is hydrogen, halogen, or cyano;
R⁴ is phenyl, naphthyl or heteroaryl, where the heteroaryl is a five to 10 member heteroaryl having 1, 2 or 3 ring heteroatoms independently selected from N, O, or S, and where the phenyl or heteroaryl is unsubstituted or substituted with 1 R⁵ and substituted with 0, 1, or 2 R^{5′}; wherein
R⁵ is selected from —CO₂R^{5b}, —CON(R^{5c})₂, C₁-C₆alkyl, C₁-C₆alkoxyC₁-C₆alkyl, —CH₂CO₂R^{5b}, —CH₂CON(R^{5c})₂, —C(O)NHSO₂C₁-C₄alkyl, —SO₂NHC(O)C₁-C₄alkyl, —SO₂N(H)ₘ(C₁-C₄alkyl)₂₋ₘ, —SO₂C₁-C₄alkyl, cyano, hydroxy, halogen, 5- to 6-membered heteroaryl having 1-4 heteroatoms independently selected from N, O, and S, and 4- to 6-membered heterocyclyl having 1-2 heteroatoms independently selected from N, O, and S(O)_{q}, wherein the 5- to 6-membered heteroaryl and 4- to 6-membered heterocyclyl are unsubstituted or substituted with 1, 2, or 3 R^{5a};
each R^{5a} is independently selected from fluoro, hydroxyl and C₁-C₆alkyl that is unsubstituted or substituted with —COOH or 1, 2, or 3 fluoro;
   wherein when R⁵ is a 4- to 6-membered heterocyclyl, two R^{5a} are not fluoro and hydroxyl substituted on the same position;
each R^{5b} is independently selected from hydrogen or C₁-C₆alkyl;
each R^{5c} is independently selected from hydrogen, C₁-C₄alkyl unsubstituted or substituted with halogen, hydroxy or C₁-C₄alkyl;
each R⁵ is independently selected from the group consisting of haloC₁-C₄alkyl, C₁-C₄alkoxy, haloC₁-C₄alkoxy, halogen, cyanomethyl, hydroxyC₁-C₆alkyl, and C₃-C₅cycloalkyl;
W is O, C(R⁶)₂, or NR⁷;
R⁶ is independently selected at each occurrence from the group consisting of hydrogen, halogen, hydroxy, amino, mono- and di-C₁-C₄alkylamino, C₁-C₆alkyl, haloC₁-C₆alkyl, hydroxyC₁-C₆alkyl, cyanoC₁-C₆alkyl, C₁-C₆alkoxy, or haloC₁-C₆alkoxy; or two R⁶ in combination with the carbon atom to which they are attached form a spirocyclic carbocycle having 3 to 6 ring atoms, wherein the spirocyclic carbocycle is unsubstituted or substituted with 1 or 2 substituents selected from halogen, C₁-C₆alkyl, or C₁-C₆alkoxy; or two R⁶ in combination with the carbon atom to which they are attached form a spirocyclic heterocycle having 1 or 2 ring heteroatoms independently selected from N, O, or S(O)_{q}, wherein the spirocyclic heterocycle is unsubstituted or substituted with 1 or 2 halogen substituents, C₁-C₆alkyl, haloC₁-C₆alkyl, C₃-C₆cycloalkyl, haloC₃-C₆cycloalkyl;
q is 0, 1, or 2;
R⁷ is hydrogen, C₁-C₆alkyl, haloC₁-C₆alkyl, hydroxyC₂-C₆alkyl, C₃-C₆cycloalkyl, C₃-C₆cycloalkylC₁-C₃alkyl, haloC₃-C₆cycloalkyl, C₁-C₆alkoxyC₁-C₆alkyl, haloC₁-C₆alkoxyC₁-C₆alkyl, aryl, 4- to 6-membered heterocyclyl having 1-2 heteroatoms independently selected from N, O, and S(O)_{q}, or 5- to 6-membered heteroaryl having 1-4 heteroatoms independently selected from N, O, and S, wherein C₁-C₆alkyl is unsubstituted or substituted with 4- to 6-membered heterocyclyl having 1-2 heteroatoms independently selected from N, O, and S(O)_{q}, wherein the 4- to 6-membered heterocyclyl is unsubstituted or substituted with 1, 2, or 3 halogen, further wherein the aryl or heteroaryl is unsubstituted or substituted with halo, C₁-C₆alkyl, haloC₁-C₆alkyl, C₃-C₅cycloalkyl, haloC₃-C₅cycloalkyl; 10 each R⁸ is independently hydrogen, halogen, C₁-C₄alkyl, haloC₁-C₄alkyl, or two R⁸ together with the carbon atom to which they are attached form a C₃-C₅cycloalkyl or haloC₃-C₅cycloalkyl.
Embodiment 2. The compound of formula (I) according to Embodiment 1, or a pharmaceutically acceptable salt thereof, of of formula (I-A)

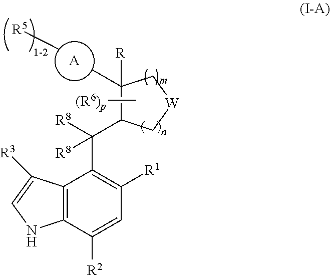

wherein A is selected from phenyl, furanyl, thiophenyl, pyrrolyl, oxazolyl, isoxazolyl, thiazolyl, pyrazolyl, imidazolyl, pyridyl, triazolyl, tetrazolyl, oxadiazolyl, isoxadiazolyl, pyrimidinyl, pyrazinyl, and pyridazinyl.
Embodiment 3. The compound I-A according to Embodiment 1, or a pharmaceutically acceptable salt thereof, of formula (I-B)

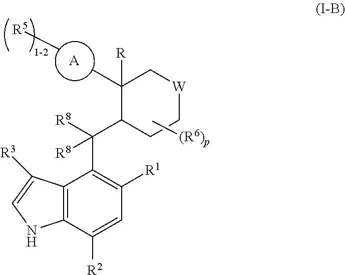

wherein A is selected from phenyl, furanyl, thiophenyl, pyrrolyl, oxazolyl, isoxazolyl, thiazolyl, pyrazolyl, imidazolyl, pyridyl, triazolyl, tetrazolyl, oxadiazolyl, isoxadiazolyl, pyrimidinyl, pyrazinyl, and pyridazinyl.
Embodiment 4. The compound according to Embodiments 2 or 3, or a pharmaceutically acceptable salt thereof, wherein wherein A is selected from:

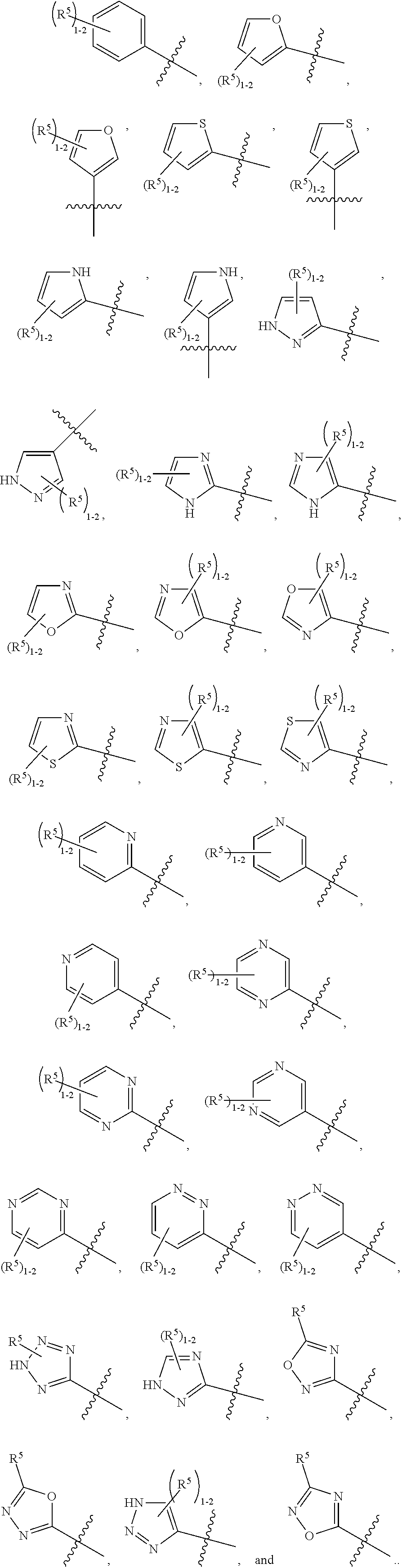

Embodiment 5. The compound of according to any one of the preceding Embodiments, or a pharmaceutically acceptable salt thereof wherein W is C(R⁶)₂, or NR⁷.
Embodiment 6. The compound of according to any one of the preceding Embodiments, or a pharmaceutically acceptable salt thereof wherein W is C(R⁶)₂.
Embodiment 7. The compound of according to any one of the preceding Embodiments, or a pharmaceutically acceptable salt thereof wherein W is NR⁷.
Embodiment 8. The compound according to any one of the preceding Embodiments, or a pharmaceutically acceptable salt thereof, wherein R⁷ is C₁-C₆alkyl, haloC₁-C₆alkyl, C₃-C₆cycloalkyl, or haloC₃-C₆cycloalkyl.
Embodiment 9. The compound of any one of the preceding Embodiments, or a pharmaceutically acceptable salt thereof, wherein A is a phenyl ring.
Embodiment 10. The compound according Embodiment 9, or a pharmaceutically acceptable salt thereof, wherein R⁵ is substituted on the para position of the phenyl ring.
Embodiment 11. The compound according to Embodiment or 10, or a pharmaceutically acceptable salt thereof, of formula (I-C):

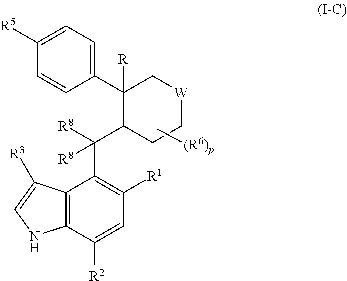

Embodiment 12. The compound according to any one of the preceding embodiments, or a pharmaceutically acceptable salt thereof, of formula (I-D):

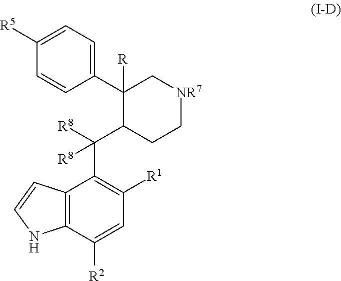

Embodiment 13. The compound according to any one of the preceding Embodiments, or a pharmaceutically acceptable salt thereof, wherein R¹ is selected from hydrogen, halogen, C₁-C₆alkyl, C₃-C₆cycloalkyl, and C₁-C₆alkoxy.
Embodiment 14. The compound according to any one of the preceding Embodiments, or a pharmaceutically acceptable salt thereof, wherein R² is selected from halogen, methyl, ethyl, and cyclopropyl.
Embodiment 15. The compound according to any one of the preceding Embodiments, or a pharmaceutically acceptable salt thereof, wherein R² is selected from methyl, ethyl, and cyclopropyl.
Embodiment 16. The compound according to any one of the preceding Embodiments, or a pharmaceutically acceptable salt thereof, wherein wherein R² is methyl.
Embodiment 17. The compound according to any one of the preceding Embodiments, or a pharmaceutically acceptable salt thereof, wherein R is hydrogen, halogen, or C₁-C₄alkyl.
Embodiment 18. The compound according to any one of the preceding Embodiments, or a pharmaceutically acceptable salt thereof, wherein R³ is hydrogen or chloro.
Embodiment 19. The compound according to any one of the preceding Embodiments, or a pharmaceutically acceptable salt thereof, wherein R³ is hydrogen.
Embodiment 20. The compound according to any one of the preceding Embodiments, or a pharmaceutically acceptable salt thereof, wherein R⁵ is independently selected from hydrogen, —CO₂R^{5b}, —CON(R^{5c})₂, C₁-C₆alkyl, —CH₂CO₂R^{5b}, —CH₂CON(R^{5c})₂, —C(O)NHSO₂C₁-C₄alkyl, —SO₂NHC(O)C₁-C₄alkyl, —SO₂N(H)ₘ(C₁-C₄alkyl)₂₋ₘ, —SO₂C₁-C₄alkyl, cyano, halogen, hydroxyC₁-C₆alkyl, C₃-C₅cycloalkyl, 5- to 6-membered heteroaryl having 1-4 heteroatoms independently selected from N, O, and S, and 4- to 6-membered heterocyclyl having 1-2 heteroatoms independently selected from N, O, and S, wherein the 5- to 6-membered heteroaryl and 4- to 6-membered heterocyclyl are unsubstituted or substituted with 1 or 2 R^{5a}.
Embodiment 21. The compound according to any one of the preceding Embodiments, or a pharmaceutically acceptable salt thereof, wherein R⁵ is selected from —CO₂H, hydroxyC₁-C₆alkyl, 5- to 6-membered heteroaryl having 1-4 heteroatoms independently selected from N, O, and S, and 4- to 6-membered heterocyclyl having 1 O heteroatom, wherein the 4- to 6-membered heterocyclyl is substituted with 0-1 R^{5a}.
Embodiment 22. The compound according to any one of the preceding Embodiments, or a pharmaceutically acceptable salt thereof, wherein R⁵ is selected from CO₂H, hydroxyC₁-C₆alkyl, 5-membered heteroaryl having 2 N heteroatoms, and 4- to 6-membered heterocyclyl having 1 O heteroatom, wherein 4- to 6-membered heterocyclyl is unsubstituted or substituted with 1 R^{5a}, wherein R^{5a} is hydroxyl.
Embodiment 23. The compound according to Embodiment 18, or a pharmaceutically acceptable salt thereof, wherein R⁵ is-CO₂H.
Embodiment 24. The compound according to any one of the preceding Embodiments, or a pharmaceutically acceptable salt thereof, wherein R⁵ is tetrazolyl.
Embodiment 25. The compound according to any one of the preceding Embodiments, or a pharmaceutically acceptable salt thereof, wherein R⁴ is substituted with 1 or 2 R^{5′} independently selected from the group consisting of haloC₁-C₄alkyl and halogen.
Embodiment 26. The compound according to any one of the preceding Embodiments, or a pharmaceutically acceptable salt thereof, wherein R⁶ is independently selected at each occurrence from the group consisting of hydrogen, halogen, haloC₁-C₆alkoxy, C₁-C₆alkoxy, or two R⁶ in combination with the carbon atom to which they are attached form a spirocyclic carbocycle having 3 to 6 ring atoms, wherein the spirocyclic carbocycle is unsubstituted or substituted with 1 or 2 substituents selected from halogen or C₁-C₆alkoxy.
Embodiment 27. The compound according to any one of the preceding Embodiments, with the proviso that both m and n are not 3.
Embodiment 28. The compound of formula (I) according to Embodiment 1, or a pharmaceutically acceptable salt thereof, selected from:
4-((3R,4R)-4-((5,7-dimethyl-1H-indol-4-yl)methyl)-1-(2,2,2-trifluoroethyl)piperidin-3-yl)benzoic acid;
4-(4-((5,7-dimethyl-1H-indol-4-yl)methyl)-1-(2,2,2-trifluoroethyl)piperidin-3-yl)benzoic acid;
4-((3R,4R)-4-((5-methoxy-7-methyl-1H-indol-4-yl)methyl)-1-(2,2,2-trifluoroethyl)piperidin-3-yl)benzoic acid;
4-(4-((5-methoxy-7-methyl-1H-indol-4-yl)methyl)-1-(2,2,2-trifluoroethyl)piperidin-3-yl)benzoic acid;
4-((3R,4R)-4-((5,7-dimethyl-1H-indol-4-yl)methyl)-1-methylpiperidin-3-yl)benzoic acid;
4-(4-((5,7-dimethyl-1H-indol-4-yl)methyl)-1-methylpiperidin-3-yl)benzoic acid;
4-((3R,4R)-4-((5-methoxy-7-methyl-1H-indol-4-yl)methyl)-1-methylpiperidin-3-yl)benzoic acid;
4-(4-((5-methoxy-7-methyl-1H-indol-4-yl)methyl)-1-methylpiperidin-3-yl)benzoic acid;
4-((3R,4R)-1-(2,2-difluoroethyl)-4-((5,7-dimethyl-1H-indol-4-yl)methyl)piperidin-3-yl)benzoic acid;
4-(1-(2,2-difluoroethyl)-4-((5,7-dimethyl-1H-indol-4-yl)methyl)piperidin-3-yl)benzoic acid;
4-((3R,4R)-4-((5-cyclopropyl-7-methyl-1H-indol-4-yl)methyl)-1-(2,2,2-trifluoroethyl)piperidin-3-yl)benzoic acid;
4-(4-((5-cyclopropyl-7-methyl-1H-indol-4-yl)methyl)-1-(2,2,2-trifluoroethyl)piperidin-3-yl)benzoic acid;
4-((3R,4R)-4-((5,7-dimethyl-1H-indol-4-yl)methyl)-1-(2-fluoroethyl)piperidin-3-yl)benzoic acid;
4-(4-((5,7-dimethyl-1H-indol-4-yl)methyl)-1-(2-fluoroethyl)piperidin-3-yl)benzoic acid;
4-((3R,4R)-4-((5-cyclopropyl-7-methyl-1H-indol-4-yl)methyl)-1-methylpiperidin-3-yl)benzoic acid;
4-(4-((5-cyclopropyl-7-methyl-1H-indol-4-yl)methyl)-1-methylpiperidin-3-yl)benzoic acid;
4-((3R,4R)-4-((5,7-dimethyl-1H-indol-4-yl)methyl)-1-isobutylpiperidin-3-yl)benzoic acid;
4-(4-((5,7-dimethyl-1H-indol-4-yl)methyl)-1-isobutylpiperidin-3-yl)benzoic acid;
4-((3R,4R)-1-(cyclopropylmethyl)-4-((5,7-dimethyl-1H-indol-4-yl)methyl)piperidin-3-yl)benzoic acid;
4-(1-(cyclopropylmethyl)-4-((5,7-dimethyl-1H-indol-4-yl)methyl)piperidin-3-yl)benzoic acid;
4-((3R,4R)-4-((5,7-dimethyl-1H-indol-4-yl)methyl)-1-(2-methoxyethyl)piperidin-3-yl)benzoic acid;
4-(4-((5,7-dimethyl-1H-indol-4-yl)methyl)-1-(2-methoxyethyl)piperidin-3-yl)benzoic acid;
4-(((3R,4R)-3-(4-(1H-tetrazol-5-yl)phenyl)-1-(2,2,2-trifluoroethyl)piperidin-4-yl)methyl)-5,7-dimethyl-1H-indole;
4-((3-(4-(1H-tetrazol-5-yl)phenyl)-1-(2,2,2-trifluoroethyl)piperidin-4-yl)methyl)-5,7-dimethyl-1H-indole;
4-((1R,2S)-2-((5,7-dimethyl-1H-indol-4-yl)methyl)-5,5-difluorocyclohexyl)benzoic acid;
4-(2-((5,7-dimethyl-1H-indol-4-yl)methyl)-5,5-difluorocyclohexyl)benzoic acid;
4-((6R,7S)-7-((5,7-dimethyl-1H-indol-4-yl)methyl)spiro[3.5]nonan-6-yl)benzoic acid;
4-(7-((5,7-dimethyl-1H-indol-4-yl)methyl)spiro[3.5]nonan-6-yl)benzoic acid;
4-((6R,7S)-7-((5,7-dimethyl-1H-indol-4-yl)methyl)-2,2-difluorospiro[3.5]nonan-6-yl)benzoic acid;
4-(7-((5,7-dimethyl-1H-indol-4-yl)methyl)-2,2-difluorospiro[3.5]nonan-6-yl)benzoic acid;
4-((1R,2S,5S)-2-((5,7-dimethyl-1H-indol-4-yl)methyl)-5-ethoxycyclohexyl)benzoic acid;
4-(2-((5,7-dimethyl-1H-indol-4-yl)methyl)-5-ethoxycyclohexyl)benzoic acid;
4-((1R,2S,5S)-5-(2,2-difluoroethoxy)-2-((5,7-dimethyl-1H-indol-4-yl)methyl)cyclohexyl)benzoic acid;
4-(5-(2,2-difluoroethoxy)-2-((5,7-dimethyl-1H-indol-4-yl)methyl)cyclohexyl)benzoic acid;
4-((1R,2S,5S)-5-(cyclopropylmethoxy)-2-((5,7-dimethyl-1H-indol-4-yl)methyl)cyclohexyl)benzoic acid;
4-(5-(cyclopropylmethoxy)-2-((5,7-dimethyl-1H-indol-4-yl)methyl)cyclohexyl)benzoic acid;
4-((3R,4S)-4-((5,7-dimethyl-1H-indol-4-yl)methyl)-1-(2,2,2-trifluoroethyl)pyrrolidin-3-yl)benzoic acid;
4-(4-((5,7-dimethyl-1H-indol-4-yl)methyl)-1-(2,2,2-trifluoroethyl)pyrrolidin-3-yl)benzoic acid;
4-((3R,4S)-4-((5,7-dimethyl-1H-indol-4-yl)methyl)-1-(2,2,2-trifluoroethyl)azepan-3-yl)benzoic acid;
4-(4-((5,7-dimethyl-1H-indol-4-yl)methyl)-1-(2,2,2-trifluoroethyl)azepan-3-yl)benzoic acid;
2-(4-((3R,4R)-4-((5,7-dimethyl-1H-indol-4-yl)methyl)-1-(2,2,2-trifluoroethyl)piperidin-3-yl)-1H-pyrazol-1-yl) acetic acid;
2-(4-(4-((5,7-dimethyl-1H-indol-4-yl)methyl)-1-(2,2,2-trifluoroethyl)piperidin-3-yl)-1H-pyrazol-1-yl) acetic acid;
4-(((3R,4R)-3-(1-(2-methoxyethyl)-1H-pyrazol-4-yl)-1-(2,2,2-trifluoroethyl)piperidin-4-yl)methyl)-5,7-dimethyl-1H-indole;
4-((3-(1-(2-methoxyethyl)-1H-pyrazol-4-yl)-1-(2,2,2-trifluoroethyl)piperidin-4-yl)methyl)-5,7-dimethyl-1H-indole;
4-((3R,4R)-4-((5,7-dimethyl-1H-indol-4-yl)methyl)piperidin-3-yl)benzoic acid;
4-(4-((5,7-dimethyl-1H-indol-4-yl)methyl)piperidin-3-yl)benzoic acid;
4-((3R,4R)-4-((5,7-dimethyl-1H-indol-4-yl)methyl)-1-ethylpiperidin-3-yl)benzoic acid;
4-(-((5,7-dimethyl-1H-indol-4-yl)methyl)-1-ethylpiperidin-3-yl)benzoic acid;
4-((3R,4R)-4-((5,7-dimethyl-1H-indol-4-yl)methyl)-1-isopropylpiperidin-3-yl)benzoic acid;
4-(4-((5,7-dimethyl-1H-indol-4-yl)methyl)-1-isopropylpiperidin-3-yl)benzoic acid;
4-((3R,4R)-4-((5,7-dimethyl-1H-indol-4-yl)methyl)-1-(2-hydroxy-2-methylpropyl)piperidin-3-yl)benzoic acid;
4-(4-((5,7-dimethyl-1H-indol-4-yl)methyl)-1-(2-hydroxy-2-methylpropyl)piperidin-3-yl)benzoic acid;
4-((3R,4R)-4-((5,7-dimethyl-1H-indol-4-yl)methyl)-1-((3-fluorooxetan-3-yl)methyl)piperidin-3-yl)benzoic acid;
4-(4-((5,7-dimethyl-1H-indol-4-yl)methyl)-1-((3-fluorooxetan-3-yl)methyl)piperidin-3-yl)benzoic acid;
4-((3R,4R)-1-(3,3-difluorocyclobutyl)-4-((5,7-dimethyl-1H-indol-4-yl)methyl)piperidin-3-yl)benzoic acid;
4-(1-(3,3-difluorocyclobutyl)-4-((5,7-dimethyl-1H-indol-4-yl)methyl)piperidin-3-yl)benzoic acid;
4-((3R,4R)-4-((5,7-dimethyl-1H-indol-4-yl)methyl)-1-(2-(trifluoromethoxy)ethyl)piperidin-3-yl)benzoic acid;
4-(4-((5,7-dimethyl-1H-indol-4-yl)methyl)-1-(2-(trifluoromethoxy)ethyl)piperidin-3-yl)benzoic acid;
4-((3R,4R)-4-((5,7-dimethyl-1H-indol-4-yl)methyl)-1-propylpiperidin-3-yl)benzoic acid;
4-(4-((5,7-dimethyl-1H-indol-4-yl)methyl)-1-propylpiperidin-3-yl)benzoic acid;
4-((3R,4R)-4-((5,7-dimethyl-1H-indol-4-yl)methyl)-1-(oxetan-3-yl)piperidin-3-yl)benzoic acid;
4-(4-((5,7-dimethyl-1H-indol-4-yl)methyl)-1-(oxetan-3-yl)piperidin-3-yl)benzoic acid;
4-((3R,4R)-4-((5,7-dimethyl-1H-indol-4-yl)methyl)-1-((1r,3R)-3-fluorocyclobutyl)piperidin-3-yl)benzoic acid;
4-(4-((5,7-dimethyl-1H-indol-4-yl)methyl)-1-(3-fluorocyclobutyl)piperidin-3-yl)benzoic acid;
4-((3R,4R)-1-(3,3-difluoropropyl)-4-((5,7-dimethyl-1H-indol-4-yl)methyl)piperidin-3-yl)benzoic acid;
4-(1-(3,3-difluoropropyl)-4-((5,7-dimethyl-1H-indol-4-yl)methyl)piperidin-3-yl)benzoic acid;
4-((3R,4S)-4-((5,7-dimethyl-1H-indol-4-yl)methyl)-1-methylpyrrolidin-3-yl)benzoic acid;
4-(4-((5,7-dimethyl-1H-indol-4-yl)methyl)-1-methylpyrrolidin-3-yl)benzoic acid;
4-((3R,4R)-4-((5,7-dimethyl-1H-indol-4-yl)methyl)-1-methylpiperidin-3-yl)benzonitrile;
4-(4-((5,7-dimethyl-1H-indol-4-yl)methyl)-1-methylpiperidin-3-yl)benzonitrile;
4-((3R,4S)-4-((5,7-dimethyl-1H-indol-4-yl)methyl)-1-methylazepan-3-yl)benzoic acid;
4-(4-((5,7-dimethyl-1H-indol-4-yl)methyl)-1-methylazepan-3-yl)benzoic acid;
4-(4-((5,7-dimethyl-1H-indol-4-yl)methyl)-1-(2,2,2-trifluoroethyl)azepan-3-yl)benzoic acid;
4-((3R,4S)-4-((5,7-dimethyl-1H-indol-4-yl)methyl)-1-ethylazepan-3-yl)benzoic acid;
4-(4-((5,7-dimethyl-1H-indol-4-yl)methyl)-1-ethylazepan-3-yl)benzoic acid;
4-((4R,5R)-5-((5,7-dimethyl-1H-indol-4-yl)methyl)-1-methylazepan-4-yl)benzoic acid;
4-(5-((5,7-dimethyl-1H-indol-4-yl)methyl)-1-methylazepan-4-yl)benzoic acid;
4-((3R,4R)-4-((5-fluoro-7-methyl-1H-indol-4-yl)methyl)-1-methylpiperidin-3-yl)benzoic acid;
4-(4-((5-fluoro-7-methyl-1H-indol-4-yl)methyl)-1-methylpiperidin-3-yl)benzoic acid;
4-((4R,5R)-1-(3,3-difluoropropyl)-5-((5,7-dimethyl-1H-indol-4-yl)methyl)azepan-4-yl)benzoic acid;
4-(1-(3,3-difluoropropyl)-5-((5,7-dimethyl-1H-indol-4-yl)methyl)azepan-4-yl)benzoic acid;
4-((3R,4R)-1-methyl-4-((7-methyl-1H-indol-4-yl)methyl)piperidin-3-yl)benzoic acid;
4-(1-methyl-4-((7-methyl-1H-indol-4-yl)methyl)piperidin-3-yl)benzoic acid;
4-((3R,4R)-4-((5-chloro-7-methyl-1H-indol-4-yl)methyl)-1-methylpiperidin-3-yl)benzoic acid;
4-(4-((5-chloro-7-methyl-1H-indol-4-yl)methyl)-1-methylpiperidin-3-yl)benzoic acid;
4-((3R,4R)-4-((5,7-dichloro-1H-indol-4-yl)methyl)-1-methylpiperidin-3-yl)benzoic acid;
4-(4-((5,7-dichloro-1H-indol-4-yl)methyl)-1-methylpiperidin-3-yl)benzoic acid;
4-((3R,4R)-4-((7-chloro-5-methyl-1H-indol-4-yl)methyl)-1-methylpiperidin-3-yl)benzoic acid;
4-(4-((7-chloro-5-methyl-1H-indol-4-yl)methyl)-1-methylpiperidin-3-yl)benzoic acid;
4-((3R,4R)-4-((3-chloro-5,7-dimethyl-1H-indol-4-yl)methyl)-1-methylpiperidin-3-yl)benzoic acid;
4-(4-((3-chloro-5,7-dimethyl-1H-indol-4-yl)methyl)-1-methylpiperidin-3-yl)benzoic acid;
4-((3R,4R)-1-(3,3-difluoropropyl)-4-((5-methoxy-7-methyl-1H-indol-4-yl)methyl)piperidin-3-yl)benzoic acid;
4-(1-(3,3-difluoropropyl)-4-((5-methoxy-7-methyl-1H-indol-4-yl)methyl)piperidin-3-yl)benzoic acid;
4-(((3R,4R)-3-(4-(1H-tetrazol-5-yl)phenyl)-1-methylpiperidin-4-yl)methyl)-5,7-dimethyl-1H-indole;
4-((3-(4-(1H-tetrazol-5-yl)phenyl)-1-methylpiperidin-4-yl)methyl)-5,7-dimethyl-1H-indole;
4-((3R,4R)-4-((5,7-dimethyl-1H-indol-4-yl)methyl)-1-(2,2,2-trifluoroethyl)piperidin-3-yl)benzonitrile;
4-(4-((5,7-dimethyl-1H-indol-4-yl)methyl)-1-(2,2,2-trifluoroethyl)piperidin-3-yl)benzonitrile;
4-(((3R,4R)-3-(4-(1H-pyrazol-1-yl)phenyl)-1-methylpiperidin-4-yl)methyl)-5,7-dimethyl-1H-indole;
4-((3-(4-(1H-pyrazol-1-yl)phenyl)-1-methylpiperidin-4-yl)methyl)-5,7-dimethyl-1H-indole;
4-(((3R,4R)-3-(4-(1H-pyrazol-1-yl)phenyl)-1-(2,2,2-trifluoroethyl)piperidin-4-yl)methyl)-5,7-dimethyl-1H-indole;
4-((3-(4-(1H-pyrazol-1-yl)phenyl)-1-(2,2,2-trifluoroethyl)piperidin-4-yl)methyl)-5,7-dimethyl-1H-indole;
4-(((3R,4R)-3-(4-(1H-pyrazol-1-yl)phenyl)-1-(3,3-difluoropropyl)piperidin-4-yl)methyl)-5,7-dimethyl-1H-indole;
4-((3-(4-(1H-pyrazol-1-yl)phenyl)-1-(3,3-difluoropropyl)piperidin-4-yl)methyl)-5,7-dimethyl-1H-indole;
4-(((3R,4R)-3-(4-(1H-pyrazol-1-yl)phenyl)-1-((3-fluorooxetan-3-yl)methyl)piperidin-4-yl)methyl)-5,7-dimethyl-1H-indole;
4-((3-(4-(1H-pyrazol-1-yl)phenyl)-1-((3-fluorooxetan-3-yl)methyl)piperidin-4-yl)methyl)-5,7-dimethyl-1H-indole;
4-(((3R,4R)-3-(4-(1H-pyrazol-1-yl)phenyl)-1-(oxetan-3-yl)piperidin-4-yl)methyl)-5,7-dimethyl-1H-indole;
4-((3-(4-(1H-pyrazol-1-yl)phenyl)-1-(oxetan-3-yl)piperidin-4-yl)methyl)-5,7-dimethyl-1H-indole;
4-(((3R,4R)-3-(4-(1H-pyrazol-1-yl)phenyl)-1-(1,3-difluoropropan-2-yl)piperidin-4-yl)methyl)-5,7-dimethyl-1H-indole;
4-((3-(4-(1H-pyrazol-1-yl)phenyl)-1-(1,3-difluoropropan-2-yl)piperidin-4-yl)methyl)-5,7-dimethyl-1H-indole;
5,7-dimethyl-4-(((3R,4R)-1-methyl-3-(1-methyl-1H-pyrazol-4-yl)piperidin-4-yl)methyl)-1H-indole;
5,7-dimethyl-4-((1-methyl-3-(1-methyl-1H-pyrazol-4-yl)piperidin-4-yl)methyl)-1H-indole;
5,7-dimethyl-4-(((3R,4R)-3-(1-methyl-1H-pyrazol-4-yl)-1-(2,2,2-trifluoroethyl)piperidin-4-yl)methyl)-1H-indole;
5,7-dimethyl-4-((3-(1-methyl-1H-pyrazol-4-yl)-1-(2,2,2-trifluoroethyl)piperidin-4-yl)methyl)-1H-indole;
4-(((3R,4R)-1-(2-fluoroethyl)-3-(1-methyl-1H-pyrazol-4-yl)piperidin-4-yl)methyl)-5,7-dimethyl-1H-indole;
4-((1-(2-fluoroethyl)-3-(1-methyl-1H-pyrazol-4-yl)piperidin-4-yl)methyl)-5,7-dimethyl-1H-indole;
4-(((3R,4R)-1-(2,2-difluoroethyl)-3-(1-methyl-1H-pyrazol-4-yl)piperidin-4-yl)methyl)-5,7-dimethyl-1H-indole;
4-((1-(2,2-difluoroethyl)-3-(1-methyl-1H-pyrazol-4-yl)piperidin-4-yl)methyl)-5,7-dimethyl-1H-indole;
4-(((3R,4R)-1-(3,3-difluoropropyl)-3-(1-methyl-1H-pyrazol-4-yl)piperidin-4-yl)methyl)-5,7-dimethyl-1H-indole;
4-((1-(3,3-difluoropropyl)-3-(1-methyl-1H-pyrazol-4-yl)piperidin-4-yl)methyl)-5,7-dimethyl-1H-indole;
4-(((3R,4R)-1-(1,3-difluoropropan-2-yl)-3-(1-methyl-1H-pyrazol-4-yl)piperidin-4-yl)methyl)-5,7-dimethyl-1H-indole;
4-((1-(1,3-difluoropropan-2-yl)-3-(1-methyl-1H-pyrazol-4-yl)piperidin-4-yl)methyl)-5,7-dimethyl-1H-indole;
4-((3R,4S)-4-((5,7-dimethyl-1H-indol-4-yl)methyl)-1-methylpyrrolidin-3-yl)benzamide;
4-(4-((5,7-dimethyl-1H-indol-4-yl)methyl)-1-methylpyrrolidin-3-yl)benzamide;
4-((3R,4S)-4-((5-cyclopropyl-7-methyl-1H-indol-4-yl)methyl)-1-methylpyrrolidin-3-yl)benzoic acid;
4-(4-((5-cyclopropyl-7-methyl-1H-indol-4-yl)methyl)-1-methylpyrrolidin-3-yl)benzoic acid;
4-(((3S,4R)-4-(4-(1H-tetrazol-5-yl)phenyl)-1-methylpyrrolidin-3-yl)methyl)-5,7-dimethyl-1H-indole;
4-((4-(4-(1H-tetrazol-5-yl)phenyl)-1-methylpyrrolidin-3-yl)methyl)-5,7-dimethyl-1H-indole;
4-(((3S,4R)-4-(4-(1H-tetrazol-5-yl)phenyl)-1-ethylpyrrolidin-3-yl)methyl)-5-cyclopropyl-7-methyl-1H-indole; and
4-((4-(4-(1H-tetrazol-5-yl)phenyl)-1-ethylpyrrolidin-3-yl)methyl)-5-cyclopropyl-7-methyl-1H-indole.
   Embodiment 29. The compound of formula (I) according to Embodiment 1, or a pharmaceutically acceptable salt thereof, selected from:

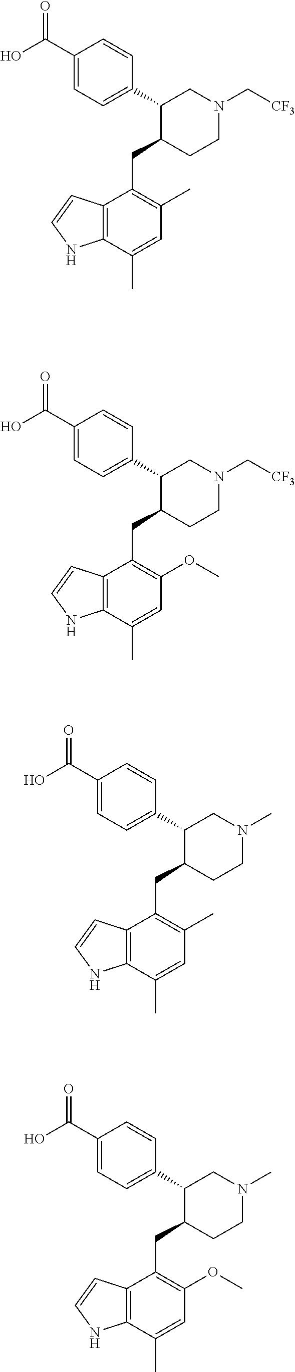 [SMILES: Cc1cc(C)c2[nH]ccc2c1C[C@@H]1CCN(CC(F)(F)F)C[C@H]1c1ccc(C(=O)O)cc1 ; COc1cc(C)c2[nH]ccc2c1C[C@@H]1CCN(CC(F)(F)F)C[C@H]1c1ccc(C(=O)O)cc1 ; Cc1cc(C)c2[nH]ccc2c1C[C@@H]1CCN(C)C[C@H]1c1ccc(C(=O)O)cc1 ; COc1cc(C)c2[nH]ccc2c1C[C@@H]1CCN(C)C[C@H]1c1ccc(C(=O)O)cc1]

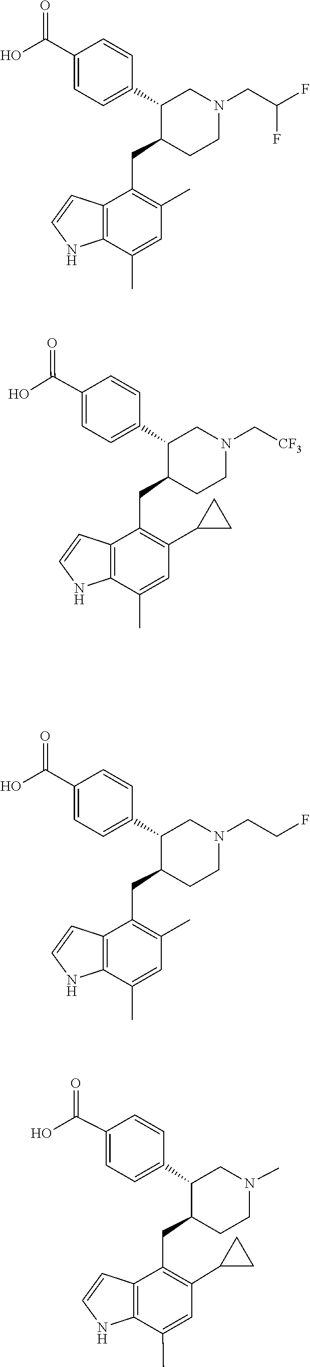 [SMILES: Cc1cc(C)c2[nH]ccc2c1C[C@@H]1CCN(CC(F)F)C[C@H]1c1ccc(C(=O)O)cc1 ; Cc1cc(C2CC2)c(C[C@@H]2CCN(CC(F)(F)F)C[C@H]2c2ccc(C(=O)O)cc2)c2cc[nH]c12 ; Cc1cc(C)c2[nH]ccc2c1C[C@@H]1CCN(CCF)C[C@H]1c1ccc(C(=O)O)cc1 ; Cc1cc(C2CC2)c(C[C@@H]2CCN(C)C[C@H]2c2ccc(C(=O)O)cc2)c2cc[nH]c12]

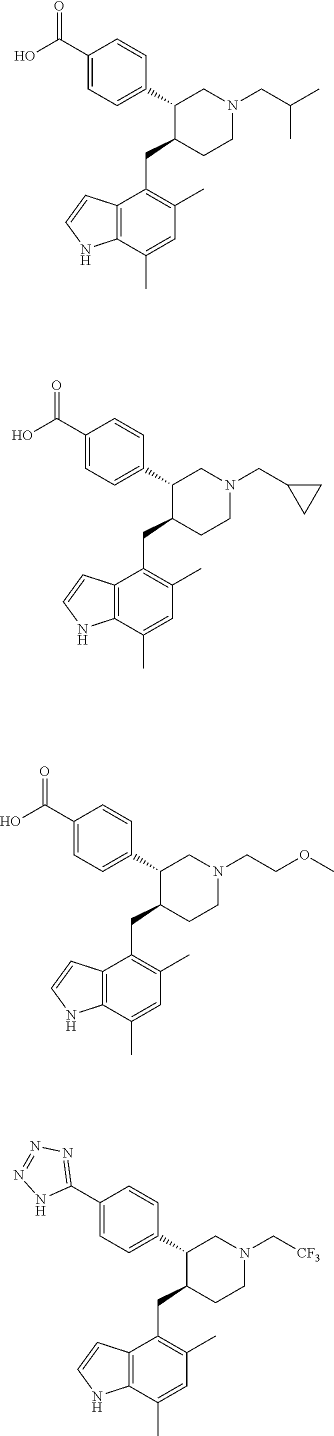 [SMILES: Cc1cc(C)c2[nH]ccc2c1C[C@@H]1CCN(CC(C)C)C[C@H]1c1ccc(C(=O)O)cc1 ; Cc1cc(C)c2[nH]ccc2c1C[C@@H]1CCN(CC2CC2)C[C@H]1c1ccc(C(=O)O)cc1 ; COCCN1CC[C@@H](Cc2c(C)cc(C)c3[nH]ccc23)[C@H](c2ccc(C(=O)O)cc2)C1 ; Cc1cc(C)c2[nH]ccc2c1C[C@@H]1CCN(CC(F)(F)F)C[C@H]1c1ccc(-c2nnn[nH]2)cc1]

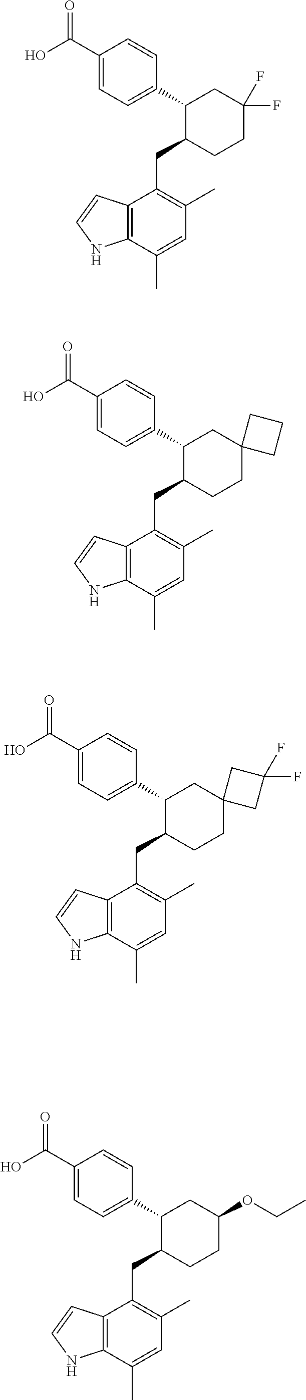 [SMILES: Cc1cc(C)c2[nH]ccc2c1C[C@@H]1CCC(F)(F)C[C@H]1c1ccc(C(=O)O)cc1 ; Cc1cc(C)c2[nH]ccc2c1C[C@@H]1CCC2(CCC2)C[C@H]1c1ccc(C(=O)O)cc1 ; Cc1cc(C)c2[nH]ccc2c1C[C@@H]1CCC2(C[C@H]1c1ccc(C(=O)O)cc1)CC(F)(F)C2 ; CCO[C@H]1CC[C@@H](Cc2c(C)cc(C)c3[nH]ccc23)[C@H](c2ccc(C(=O)O)cc2)C1]

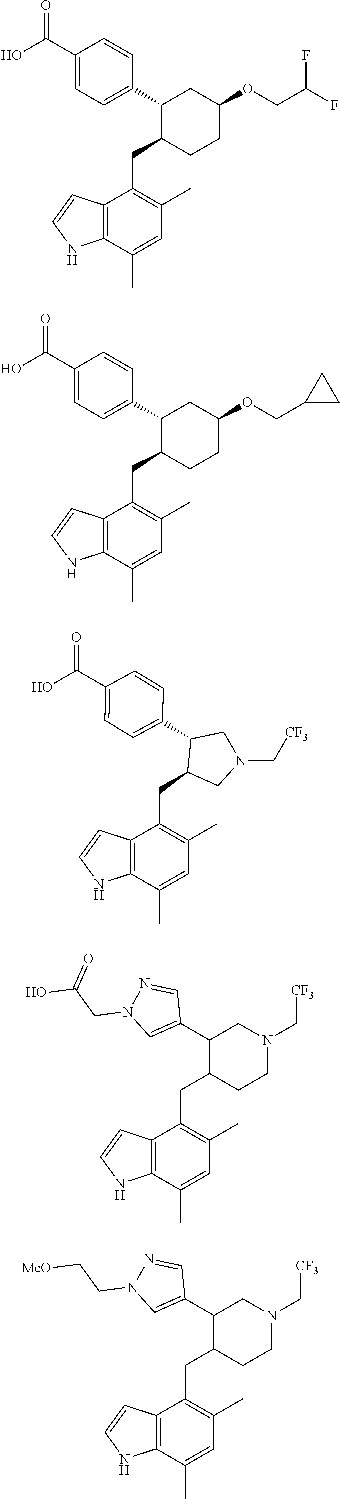

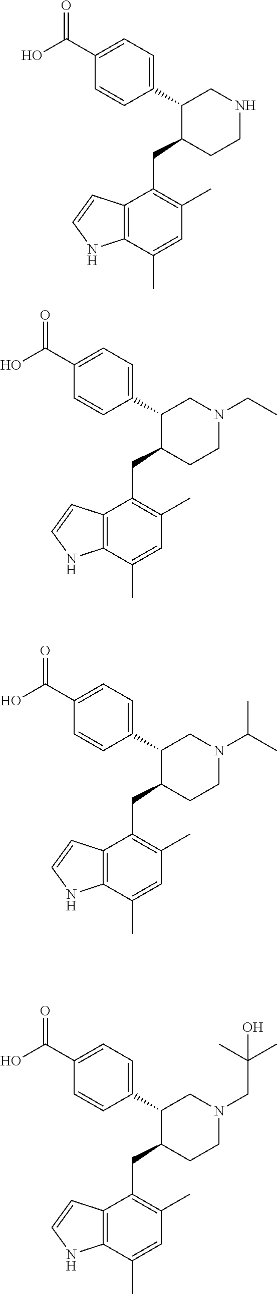 [SMILES: Cc1cc(C)c2[nH]ccc2c1C[C@@H]1CCNC[C@H]1c1ccc(C(=O)O)cc1 ; CCN1CC[C@@H](Cc2c(C)cc(C)c3[nH]ccc23)[C@H](c2ccc(C(=O)O)cc2)C1 ; Cc1cc(C)c2[nH]ccc2c1C[C@@H]1CCN(C(C)C)C[C@H]1c1ccc(C(=O)O)cc1 ; Cc1cc(C)c2[nH]ccc2c1C[C@@H]1CCN(CC(C)(C)O)C[C@H]1c1ccc(C(=O)O)cc1]

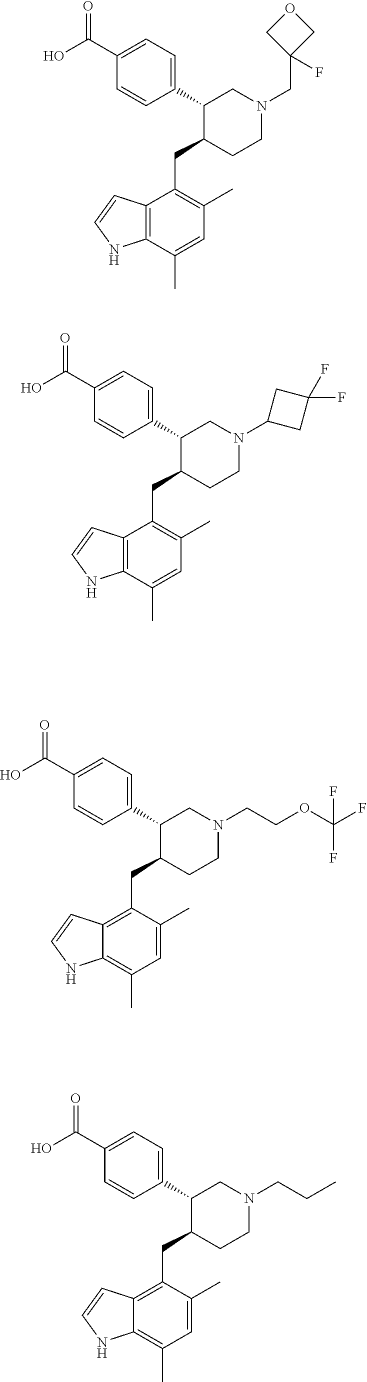 [SMILES: Cc1cc(C)c2[nH]ccc2c1C[C@@H]1CCN(CC2(F)COC2)C[C@H]1c1ccc(C(=O)O)cc1 ; Cc1cc(C)c2[nH]ccc2c1C[C@@H]1CCN(C2CC(F)(F)C2)C[C@H]1c1ccc(C(=O)O)cc1 ; Cc1cc(C)c2[nH]ccc2c1C[C@@H]1CCN(CCOC(F)(F)F)C[C@H]1c1ccc(C(=O)O)cc1 ; CCCN1CC[C@@H](Cc2c(C)cc(C)c3[nH]ccc23)[C@H](c2ccc(C(=O)O)cc2)C1]

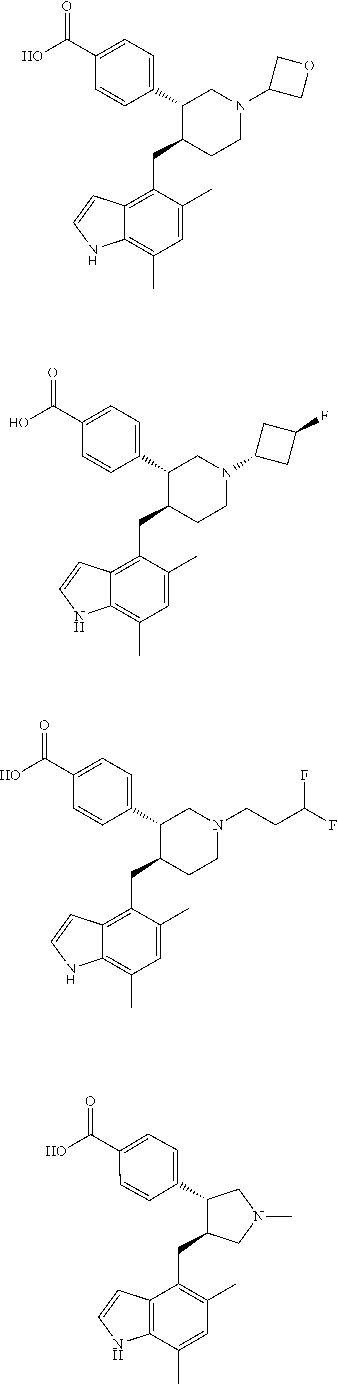 [SMILES: Cc1cc(C)c2[nH]ccc2c1C[C@@H]1CCN(C2COC2)C[C@H]1c1ccc(C(=O)O)cc1 ; Cc1cc(C)c2[nH]ccc2c1C[C@@H]1CCN([C@H]2C[C@H](F)C2)C[C@H]1c1ccc(C(=O)O)cc1 ; Cc1cc(C)c2[nH]ccc2c1C[C@@H]1CCN(CCC(F)F)C[C@H]1c1ccc(C(=O)O)cc1 ; Cc1cc(C)c2[nH]ccc2c1C[C@@H]1CN(C)C[C@H]1c1ccc(C(=O)O)cc1]

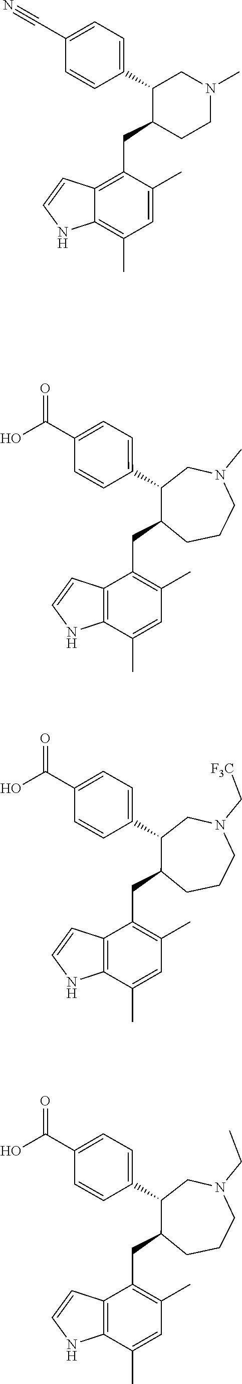 [SMILES: Cc1cc(C)c2[nH]ccc2c1C[C@@H]1CCN(C)C[C@H]1c1ccc(C#N)cc1 ; Cc1cc(C)c2[nH]ccc2c1C[C@@H]1CCCN(C)C[C@H]1c1ccc(C(=O)O)cc1 ; Cc1cc(C)c2[nH]ccc2c1C[C@@H]1CCCN(CC(F)(F)F)C[C@H]1c1ccc(C(=O)O)cc1 ; CCN1CCC[C@@H](Cc2c(C)cc(C)c3[nH]ccc23)[C@H](c2ccc(C(=O)O)cc2)C1]

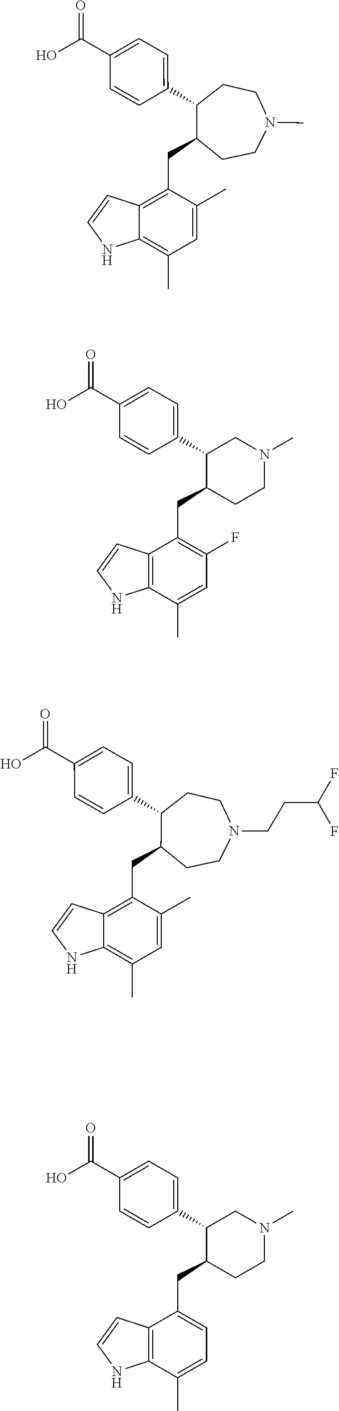 [SMILES: Cc1cc(C)c2[nH]ccc2c1C[C@@H]1CCN(C)CC[C@H]1c1ccc(C(=O)O)cc1 ; Cc1cc(F)c(C[C@@H]2CCN(C)C[C@H]2c2ccc(C(=O)O)cc2)c2cc[nH]c12 ; Cc1cc(C)c2[nH]ccc2c1C[C@@H]1CCN(CCC(F)F)CC[C@H]1c1ccc(C(=O)O)cc1 ; Cc1ccc(C[C@@H]2CCN(C)C[C@H]2c2ccc(C(=O)O)cc2)c2cc[nH]c12]

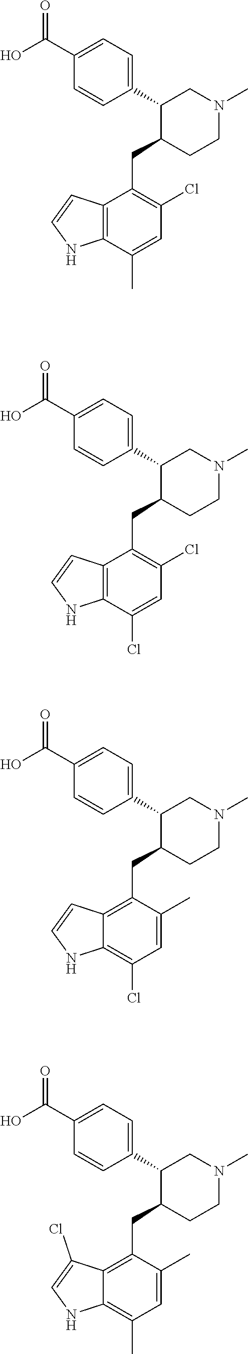 [SMILES: Cc1cc(Cl)c(C[C@@H]2CCN(C)C[C@H]2c2ccc(C(=O)O)cc2)c2cc[nH]c12 ; CN1CC[C@@H](Cc2c(Cl)cc(Cl)c3[nH]ccc23)[C@H](c2ccc(C(=O)O)cc2)C1 ; Cc1cc(Cl)c2[nH]ccc2c1C[C@@H]1CCN(C)C[C@H]1c1ccc(C(=O)O)cc1 ; Cc1cc(C)c2[nH]cc(Cl)c2c1C[C@@H]1CCN(C)C[C@H]1c1ccc(C(=O)O)cc1]

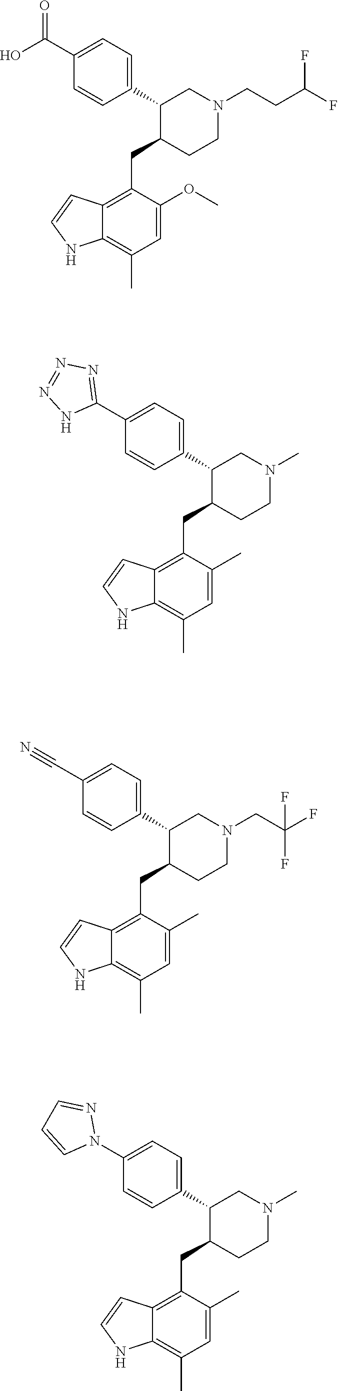 [SMILES: COc1cc(C)c2[nH]ccc2c1C[C@@H]1CCN(CCC(F)F)C[C@H]1c1ccc(C(=O)O)cc1 ; Cc1cc(C)c2[nH]ccc2c1C[C@@H]1CCN(C)C[C@H]1c1ccc(-c2nnn[nH]2)cc1 ; Cc1cc(C)c2[nH]ccc2c1C[C@@H]1CCN(CC(F)(F)F)C[C@H]1c1ccc(C#N)cc1 ; Cc1cc(C)c2[nH]ccc2c1C[C@@H]1CCN(C)C[C@H]1c1ccc(-n2cccn2)cc1]

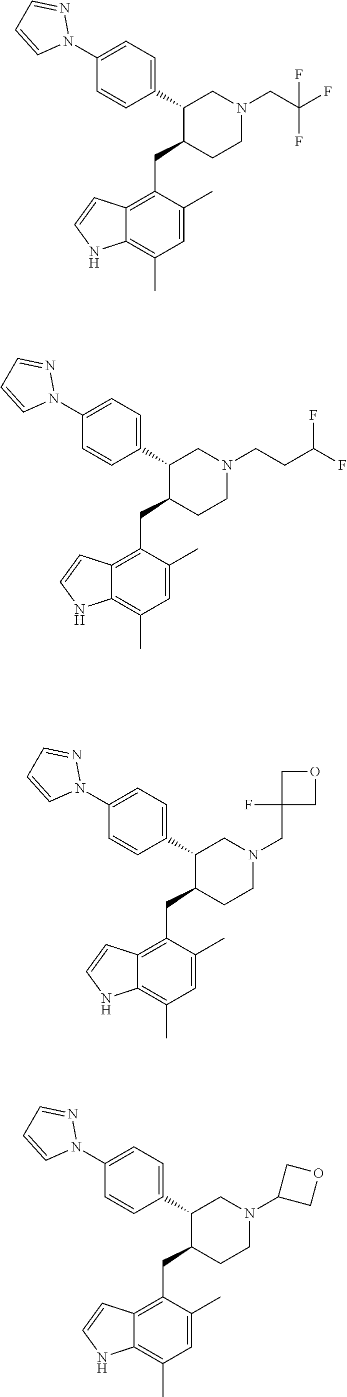 [SMILES: Cc1cc(C)c2[nH]ccc2c1C[C@@H]1CCN(CC(F)(F)F)C[C@H]1c1ccc(-n2cccn2)cc1 ; Cc1cc(C)c2[nH]ccc2c1C[C@@H]1CCN(CCC(F)F)C[C@H]1c1ccc(-n2cccn2)cc1 ; Cc1cc(C)c2[nH]ccc2c1C[C@@H]1CCN(CC2(F)COC2)C[C@H]1c1ccc(-n2cccn2)cc1 ; Cc1cc(C)c2[nH]ccc2c1C[C@@H]1CCN(C2COC2)C[C@H]1c1ccc(-n2cccn2)cc1]

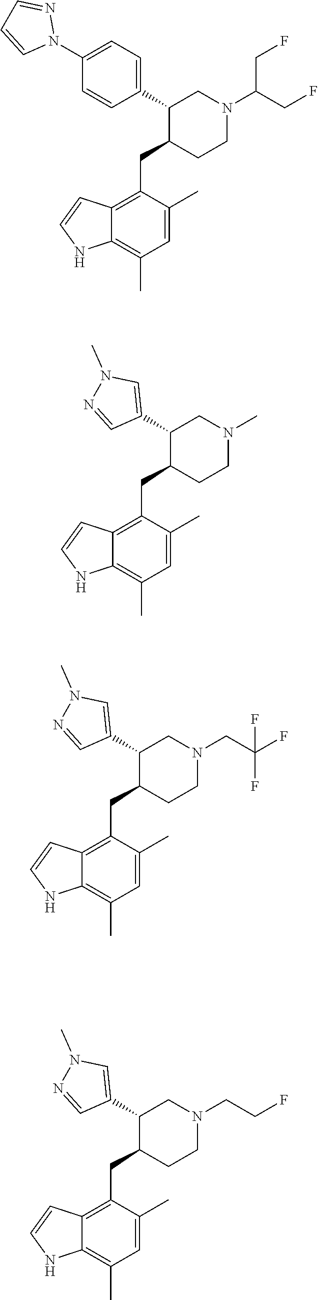 [SMILES: Cc1cc(C)c2[nH]ccc2c1C[C@@H]1CCN(C(CF)CF)C[C@H]1c1ccc(-n2cccn2)cc1 ; Cc1cc(C)c2[nH]ccc2c1C[C@@H]1CCN(C)C[C@H]1c1cnn(C)c1 ; Cc1cc(C)c2[nH]ccc2c1C[C@@H]1CCN(CC(F)(F)F)C[C@H]1c1cnn(C)c1 ; Cc1cc(C)c2[nH]ccc2c1C[C@@H]1CCN(CCF)C[C@H]1c1cnn(C)c1]

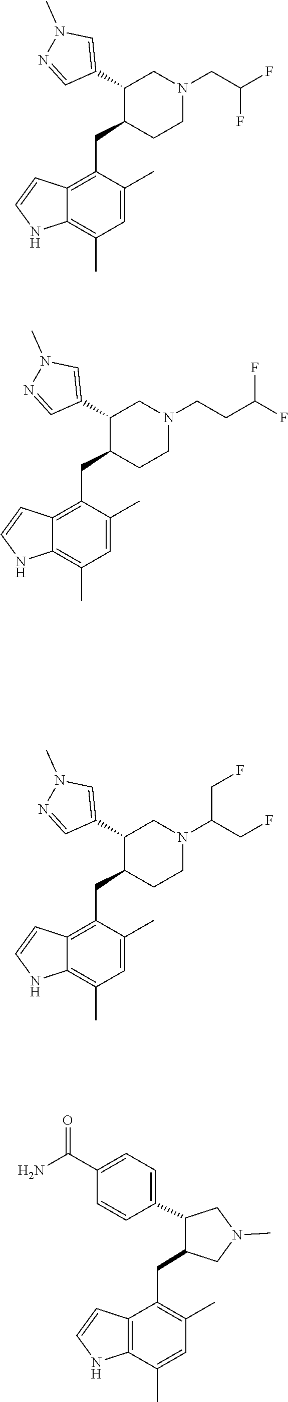

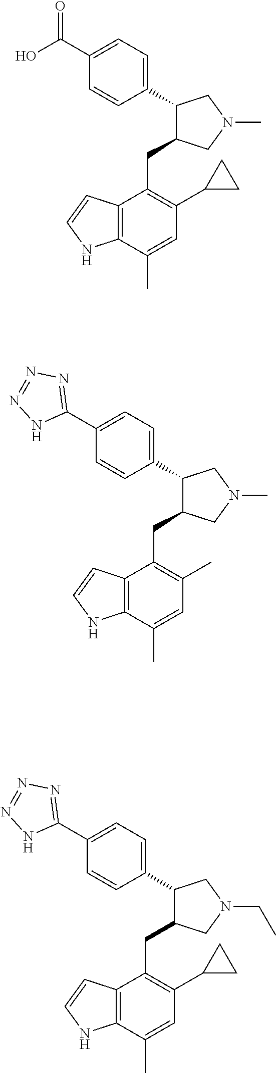

Embodiment 30. A pharmaceutical composition comprising a therapeutically effective amount of a compound according to any one of the preceding Embodiments, or a pharmaceutically acceptable salt thereof, and a pharmaceutically acceptable carrier or excipient.
Embodiment 31. A method of treating or preventing a disease or disorder in a subject in need thereof, the method comprising administering to the subject a therapeutically effective amount of a compound according to any one of Embodiments 1 to 29, or a pharmaceutically acceptable salt thereof.
Embodiment 32. A method of modulating the complement alternative pathway activity in a subject, the method comprising administering to the subject a therapeutically effective amount of a compound according to any one of Embodiments 1 to 29, or a pharmaceutically acceptable salt thereof.
Embodiment 33. A method of treating a disease or disorder mediated by complement activation, in particular mediated by activation of the complement alternative pathway, in a subject in need thereof, the method comprising administering to the subject a therapeutically effective amount of a compound according to any one of Embodiments 1 to 29, or a pharmaceutically acceptable salt thereof.
Embodiment 34. A method of treating a disease or disorder that is affected by the modulation of complement alternative pathway comprising administering to the subject a therapeutically effective amount of a compound according to any one of Embodiments 1 to 29, or a pharmaceutically acceptable salt thereof.
Embodiment 35. A method of treating a disease or disorder associated with dysregulation of the complement alternative pathway comprising administering to the subject a therapeutically effective amount of a compound according to any one of Embodiments 1 to 29, or a pharmaceutically acceptable salt thereof.
Embodiment 36. A method of inhibiting the expression or activity of complement factor B, the method comprising administering to the subject a compound according to any one of Embodiments 1 to 29, or a pharmaceutically acceptable salt thereof.
Embodiment 37. The method according to any one of Embodiments 31 and 33 to 35, wherein the disease or disorder is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammation, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), immune complex membranoproliferative glomerulonephritis (IC-MPGN), IgA nephropathy, membranous nephropathy including idiopathic membranous nephropathy, diabetic nephropathy, atypical hemolytic uremic syndrome (aHUS), Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, neuromyelitis optica (NMO), IgA vasculitis (formerly known as Henoch-Schonlein purpura or HSP), CHAPLE syndrome, hematopoietic stem cell transplantation-associated thrombotic microangiopathy (HSCT-TMA), adult respiratory distress syndrome (ARDS,), myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury mesenteric artery reperfusion after aortic reconstruction, infectious disease or sepsis; COVID-19, immune complex disorders and autoimmune diseases, rheumatoid arthritis, osteoarthritis, Spondyloarthropathies, psoriatic arthritis, systemic lupus erythematosus (SLE), lupus nephritis, SLE nephritis, proliferative nephritis, myasthenia gravis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, antineutrophil cytoplasmic antibody (ANCA)-associated vasculitides (AAV), Buerger's vasculitis, cryoglobulinemia, Kawasaki disease, Takayasu arteritis, cryoglobulinemia, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenia purpura (TTP), and abdominal aortic aneurisms, and Grave's disease.
Embodiment 38. A method of treating age-related macular degeneration comprising administering to a subject in need thereof an effective amount of a composition comprising a compound according to any one of Embodiments 1 to 29, or a pharmaceutically acceptable salt thereof.
Embodiment 39. A compound according to any one of Embodiments 1 to 29, or a pharmaceutically acceptable salt thereof, for use as a medicament.
Embodiment 40. A compound according to any one of Embodiments 1 to 29, or a pharmaceutically acceptable salt thereof, for use in inhibiting the expression or activity of complement factor B, in a subject in need thereof.
Embodiment 41. A compound according to any one of Embodiments 1 to 29, or a pharmaceutically acceptable salt thereof, for use in treating a disease or disorder associated with dysregulation of the complement alternative pathway.
Embodiment 42. The compound for use according to Embodiment 41, or a pharmaceutically acceptable salt thereof, wherein the disease or disorder is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammation, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), immune complex membranoproliferative glomerulonephritis (IC-MPGN), IgA nephropathy, membranous nephropathy including idiopathic membranous nephropathy, diabetic nephropathy, atypical hemolytic uremic syndrome (aHUS), Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, neuromyelitis optica (NMO), IgA vasculitis (formerly known as Henoch-Schonlein purpura or HSP), CHAPLE syndrome, hematopoietic stem cell transplantation-associated thrombotic microangiopathy (HSCT-TMA), adult respiratory distress syndrome (ARDS,), myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury mesenteric artery reperfusion after aortic reconstruction, infectious disease or sepsis; COVID-19, immune complex disorders and autoimmune diseases, rheumatoid arthritis, osteoarthritis, Spondyloarthropathies, psoriatic arthritis, systemic lupus erythematosus (SLE), lupus nephritis, SLE nephritis, proliferative nephritis, myasthenia gravis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, antineutrophil cytoplasmic antibody (ANCA)-associated vasculitides (AAV), Buerger's vasculitis, cryoglobulinemia, Kawasaki disease, Takayasu arteritis, cryoglobulinemia, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenia purpura (TTP), and abdominal aortic aneurisms, and Grave's disease.
Embodiment 43. Use of a compound according to any one of Embodiments 1 to 29, or a pharmaceutically acceptable salt thereof, in the manufacture of a medicament for the treatment of a disease or disorder mediated by complement activation or activation of the complement alternative pathway.
Embodiment 44. Use of a compound according to any one of Embodiments 1 to 29, or a pharmaceutically acceptable salt thereof, in the manufacture of a medicament for the treatment of a disease or disorder selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammation, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), immune complex membranoproliferative glomerulonephritis (IC-MPGN), IgA nephropathy, membranous nephropathy including idiopathic membranous nephropathy, diabetic nephropathy, atypical hemolytic uremic syndrome (aHUS), Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, neuromyelitis optica (NMO), IgA vasculitis (formerly known as Henoch-Schonlein purpura or HSP), CHAPLE syndrome, hematopoietic stem cell transplantation-associated thrombotic microangiopathy (HSCT-TMA), adult respiratory distress syndrome (ARDS,), myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury mesenteric artery reperfusion after aortic reconstruction, infectious disease or sepsis; COVID-19, immune complex disorders and autoimmune diseases, rheumatoid arthritis, osteoarthritis, Spondyloarthropathies, psoriatic arthritis, systemic lupus erythematosus (SLE), lupus nephritis, SLE nephritis, proliferative nephritis, myasthenia gravis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, antineutrophil cytoplasmic antibody (ANCA)-associated vasculitides (AAV), Buerger's vasculitis, cryoglobulinemia, Kawasaki disease, Takayasu arteritis, cryoglobulinemia, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenia purpura (TTP), and abdominal aortic aneurisms, and Grave's disease.
Embodiment 45. Use of a compound according to any one of Embodiments 1 to 29, or a pharmaceutically acceptable salt thereof, for the treatment of a disease or disorder mediated by complement activation or activation of the complement alternative pathway.
Embodiment 46. Use of a compound according to any one of Embodiments 1 to 29, or a pharmaceutically acceptable salt thereof, for the treatment of a disease or disorder that is affected by the modulation of complement alternative pathway.
Embodiment 47. Use of a compound according to any one of Embodiments 1 to 29, or a pharmaceutically acceptable salt thereof, for the treatment of a disease or disorder selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammation, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), immune complex membranoproliferative glomerulonephritis (IC-MPGN), IgA nephropathy, membranous nephropathy including idiopathic membranous nephropathy, diabetic nephropathy, atypical hemolytic uremic syndrome (aHUS), Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, neuromyelitis optica (NMO), IgA vasculitis (formerly known as Henoch-Schonlein purpura or HSP), CHAPLE syndrome, hematopoietic stem cell transplantation-associated thrombotic microangiopathy (HSCT-TMA), adult respiratory distress syndrome (ARDS,), myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury mesenteric artery reperfusion after aortic reconstruction, infectious disease or sepsis; COVID-19, immune complex disorders and autoimmune diseases, rheumatoid arthritis, osteoarthritis, Spondyloarthropathies, psoriatic arthritis, systemic lupus erythematosus (SLE), lupus nephritis, SLE nephritis, proliferative nephritis, myasthenia gravis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, antineutrophil cytoplasmic antibody (ANCA)-associated vasculitides (AAV), Buerger's vasculitis, cryoglobulinemia, Kawasaki disease, Takayasu arteritis, cryoglobulinemia, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenia purpura (TTP), and abdominal aortic aneurisms, and Grave's disease.
Embodiment 48. Use of a compound according to any one of Embodiments 1 to 29, or a pharmaceutically acceptable salt thereof, for the treatment of age-related macular degeneration.
Embodiment 49. A pharmaceutical combination comprising a compound according to any one of Embodiments 1 to 29, or a pharmaceutically acceptable salt thereof, and one or more additional therapeutic agent(s).

Depending on the choice of the starting materials and procedures, the compounds can be present in the form of one of the possible isomers or as mixtures thereof, for example as pure optical isomers, or as isomer mixtures, such as racemates and diastereomeric mixtures, depending on the number of asymmetric centres. The disclosure is meant to include all such possible isomers, including racemic mixtures, enantiomerically enriched mixtures, diastereomeric mixtures and optically pure forms. Optically active (R)- and (S)-isomers may be prepared using chiral synthons or chiral reagents, or resolved using conventional techniques. If the compound contains a disubstituted or trisubstituted cycloalkyl, the cycloalkyl substituent(s) may have a cis- or trans-configuration. The disclosure includes cis and trans configurations of substituted cycloalkyl groups, e.g., cyclobutyl group, as well as mixtures thereof. All tautomeric forms are also intended to be included. In particular, where a heteroaryl ring containing N as a ring atom is 2-pyridone, for example, tautomers where the carbonyl is depicted as a hydroxy (e.g., 2-hydroxypyridine) are included.

Separation of cis and trans isomers can be achieved according to methods known to a person of skill in the art, such as chromatographic methods, with tools such as HPLC (High Performance Liquid Chromatography), Thin Layer Chromatography, SFC (Supercritical Fluid Chromatography), GC (Gas Chromatography), or recrystallization techniques.

### Pharmaceutically Acceptable Salts

As used herein, the terms “salt” or “salts” refers to an acid addition or base addition salt of a compound of the disclosure. “Salts” include in particular “pharmaceutically acceptable salts”. The term “pharmaceutically acceptable salts” refers to salts that retain the biological effectiveness and properties of the compounds of this disclosure and, which typically are not biologically or otherwise undesirable. The compounds of the disclosure may be capable of forming acid and/or base salts by virtue of the presence of amino and/or carboxyl groups or groups similar thereto.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, formic acid, trifluoroacetic acid, and the like. In an embodiment, the compounds of Formula (I) are in HCl or formic acid salt form.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the disclosure provides compounds in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

In another aspect, the disclosure provides compounds in sodium, potassium, ammonium, calcium, magnesium, iron, silver, zinc, copper, isopropylamine, benzathine, cholinate, diethanolamine, diethylamine, lysine, meglumine, piperazine or tromethamine salt form.

### Isotopically Labelled Compounds

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, sulfur, fluorine, chlorine and iodine, such as ²H, ³H, ¹¹C, ¹³C, ¹⁴C, ¹⁸O, ¹⁵N, ¹⁸F, ¹⁷O, ¹⁸O, ³⁵S, ³⁶Cl, ¹²³I, ¹²⁴I, ¹²⁵I respectively. The disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F compound may be particularly desirable for PET or SPECT studies. Isotopically-labeled compounds of formula (I), or sub-formulae thereof, can generally be prepared by conventional techniques known to those skilled in the art or by processes analogous to those described in the accompanying Examples and General Schemes using an appropriate isotopically-labeled reagent in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I), or any of the sub-formulae thereof. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the disclosure include those wherein the solvent of crystallization may be isotopically substituted, e.g., D₂O, d₆-acetone, d₆-DMSO.

Compounds of the disclosure, i.e., compounds of formulae (I), (I-A), (I-B), that contain groups capable of acting as donors and/or acceptors for hydrogen bonds may be capable of forming co-crystals with suitable co-crystal formers. These co-crystals may be prepared from compounds of formula (I), or sub-formulae thereof, by known co-crystal forming procedures. Such procedures include grinding, heating, co-subliming, co-melting, or contacting in solution compounds of (I), (I-A), (I-B), with the co-crystal former under crystallization conditions and isolating co-crystals thereby formed. Suitable co-crystal formers include those described in WO 2004/078163.

All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed.

Any asymmetric center (e.g., carbon or the like) of the compound(s) of the disclosure can be present in racemic or enantiomerically enriched, for example the (R)-, (S)- or (R,S)-configuration. In certain embodiments, for example, as a mixture of enantiomers, each asymmetric center is present in at least 10% enantiomeric excess, at least 20% enantiomeric excess, at least 30% enantiomeric excess, at least 40% enantiomeric excess, at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess. In certain embodiments, for example, in enantiomerically enriched form, each asymmetric center is present in at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess. Thus, compounds of the disclosure can be present in a racemic mixture or in enantiomerically enriched form or in an enantiopure form or as a mixture of diastereoisomers.

In an embodiment, there is provided a compound of formula (I), or a pharmaceutically acceptable salt thereof, present in at least 90% enantiomeric excess, at least 95% enantiomeric excess, or at least 99% enantiomeric excess.

In an embodiment, there is provided a compound of formula (I), or a pharmaceutically acceptable salt thereof, present in at least 90% diastereomeric excess, at least 95% diastereomeric excess, or at least 99% diastereomeric excess.

In one embodiment, the compound of formula (I) is a compound of formula (I-A) or a pharmaceutically acceptable salt thereof:

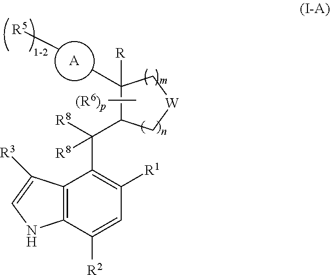

wherein A is selected from phenyl, furanyl, thiophenyl, pyrrolyl, oxazolyl, isoxazolyl, thiazolyl, pyrazolyl, imidazolyl, pyridyl, triazolyl, tetrazolyl, oxadiazolyl, isoxadiazolyl, pyrimidinyl, pyrazinyl, and pyridazinyl, and wherein R, R¹, R², R³, R⁵, R⁶, R⁸, W, m, n, and p are defined according to Embodiment 1. In particular, R, R¹, R², R³, R⁵, R⁶, R⁸, W, m, n, and p may be defined according to any of Embodiments 1 to 26.

In another embodiment, the compound of formula (I) is a compound of formula (I-B) or a pharmaceutically acceptable salt thereof:

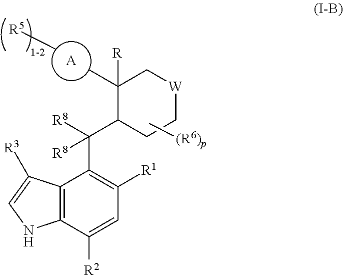

wherein A is selected from phenyl, furanyl, thiophenyl, pyrrolyl, oxazolyl, isoxazolyl, thiazolyl, pyrazolyl, imiadazolyl, pyridyl, triazolyl, tetrazolyl, oxadiazolyl, isoxadiazolyl, pyrimidinyl, pyrazinyl, and pyridazinyl wherein R, R¹, R², R³, R⁵, R⁶, R⁸, W, m, n, and p are defined according to Embodiment 1. In particular, R, R¹, R², R³, R⁵, R⁶, R⁸, W, m, n, and p may be defined according to any of Embodiments 1 to 26.

In another embodiment, the compound of formula (I) is a compound of formula (I-C) or a pharmaceutically acceptable salt thereof:

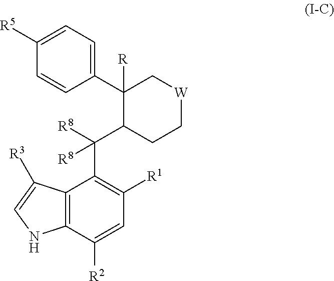

wherein R, R¹, R², R³, R⁵, R⁶, R⁸, W, and p are defined according to Embodiment 1. In particular, R, R¹, R², R⁵, R⁶, R⁸, W, and p may be defined according to any of Embodiments 1 to 26.

In another embodiment, the compound of formula (I) is a compound of formula (I-D) or a pharmaceutically acceptable salt thereof:

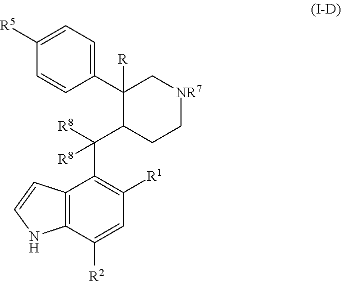

wherein R, R¹, R², R⁵, R⁷, and R⁸ are defined according to Embodiment 1. In particular, R, R¹, R², R⁵, R⁷, and R⁸ may be defined according to any of Embodiments 1 to 26.

In the formulae of the present application the term “ [symbol] ” on a C-sp³ indicates the absolute stereochemistry, either (R) or (S). In the formulae of the present application the term “ [symbol] ” on a C-sp³ indicates the absolute stereochemistry, either (R) or (S). In the formulae of the present application the term “ [symbol] ” on a C-sp³ represents a covalent bond wherein the stereochemistry of the bond is not defined. This means that the term “ [symbol] ” on a C-sp³ comprises an (S) configuration or an (R) configuration of the respective chiral centre. Furthermore, mixtures may also be present. Therefore, mixtures of stereoisomers, e.g., mixtures of enantiomers, such as racemates, and/or mixtures of diastereoisomers are encompassed by the present disclosure.

For the avoidance of doubt, where compound structures are drawn with undefined stereochemistry with respect to any R group, as represented by a bond ( [symbol] ), this means the asymmetric center has either a (R)- or (S)-configuration, or exists as a mixture thereof and stated as such.

For the avoidance of doubt, in any of formulae of the present application when the R⁵ group is shown with attachment to ring A, this means that the R⁵ group(s) can be bonded via a carbon atom or heteroatom, e.g., nitrogen.

For the avoidance of doubt, the compounds structures shown herein, when appropriate, may exist in their zwitterionic form.

Accordingly, as used herein a compound of the disclosure can be in the form of one of the possible stereoisomers, rotamers, atropisomers, tautomers or mixtures thereof, for example, as substantially pure geometric (cis or trans) stereoisomers, diastereomers, optical isomers, racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the disclosure or of intermediates can be resolved into the optical isomers (enantiomers) by known methods, e.g., by separation of the diastereomeric salts thereof, obtained with an optically active acid or base, and liberating the optically active acidic or basic compound. In particular, a basic moiety may thus be employed to resolve the compounds of the disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the disclosure or racemic intermediates can also be resolved by chiral chromatography, e.g., high pressure liquid chromatography (HPLC) using a chiral adsorbent.

Furthermore, the compounds of the disclosure, including their salts, can also be obtained in the form of their hydrates, or include other solvents used for their crystallization. The compounds of the disclosure may inherently or by design form solvates with pharmaceutically acceptable solvents (including water); therefore, it is intended that the disclosure embrace both solvated and unsolvated forms. The term “solvate” refers to a molecular complex of a compound of the disclosure (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. The presence of solvates can be identified by a person of skill in the art with tools such as NMR.

The compounds of the disclosure, including salts, hydrates and solvates thereof, may inherently or by design form polymorphs.

### Methods of Making

The compounds of the disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art.

Generally, the compounds of formula (I) can be prepared according to the Schemes provided infra.

Compounds provided herein can be prepared according to the following Examples. In the following Schemes, R, R¹, R², R³, R⁵, R⁶, R⁸, W, m, n, and p are defined according to enumerated Embodiment 1. In an embodiment, R, R¹, R², R³, R⁵, R⁶, R⁸, W, m, n, and p are defined according to any one of enumerated Embodiments 1 to 23. PG refers to protecting groups. Suitable protecting groups are known to one skilled in the art and the same or different protecting groups may be used in any Scheme. Additional definitions are provided as applicable in the General Schemes below.

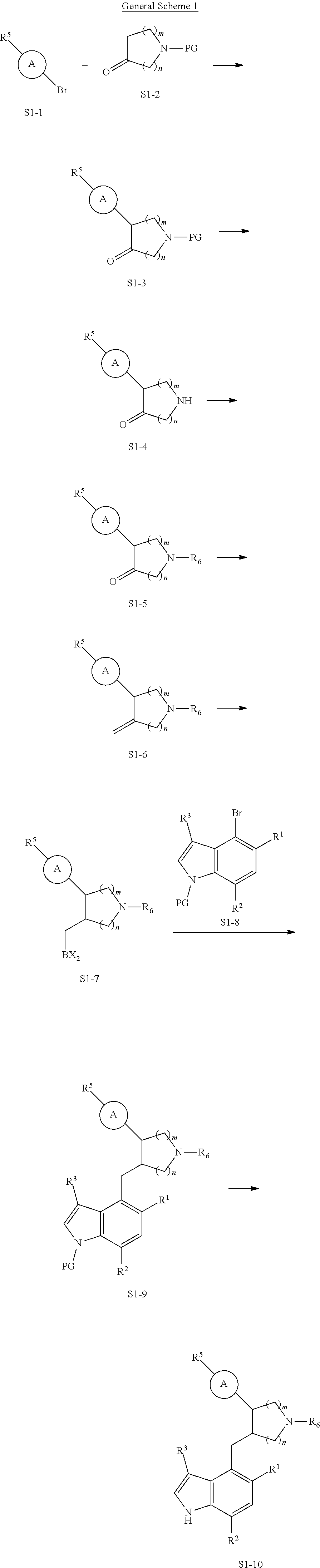

Examples S1-10 can be prepared as outlined in General Scheme 1. Arylation of ketone S1-2 with aryl bromide S1-1 can be performed utilizing Buchwald coupling conditions. Resulting intermediate S1-3 can be deprotected to give intermediate S1-4 which in turn can be reacted with a suitable electrophile, e. g. an alkyl halogenide or triflate, in the presence of base to give S1-5. Conversion of S1-5 into the corresponding methylene derivative S1-6 can be achieved via Wittig olefination or using diiodomethane, titanium tetrachloride and zinc. Hydroboration of resulting S1-6 is performed using 9-BBN to give boronate S1-7 which is coupled to an N-protected 4-Br indole derivative S1-8 (prepared by reacting the corresponding unprotected indole with a suitable electrophile in the presence of DMAP) utilizing a Suzuki coupling to give intermediate S1-9 which is in turn deprotected to give the final Examples S1-10.

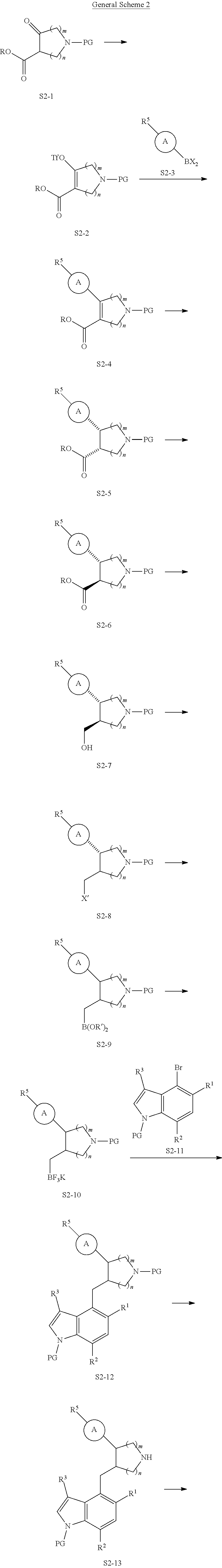

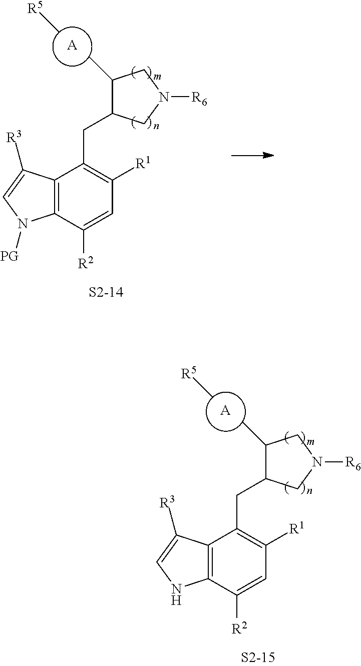

Examples S2-15 can be prepared as outlined in General Scheme 2.

Beta-ketoester S2-1 can be transformed into vinyl triflate S2-2 with trifluoromethanesulfonic anhydride and base. Suzuki coupling with boronic acid or ester S2-3 furnishes S2-4. Hydrogenation of the alkene provides S2-5, and base-mediated epimerization furnishes S2-6 with trans configuration between the vicinal stereocenters. Ester reduction with a reducing agent such as lithium borohydride provides the primary alcohol S2-7, where reduction is chemoselective for the alkyl ester when R⁵ is also an ester. The alcohol can be transformed into primary halide S2-8, for example X′═Br, using an electrophilic reagent such as carbon tetrabromide. Palladium-catalyzed borylation furnishes boronic ester S2-9, which is then converted to S2-10 using potassium bifluoride. BF₃K-salt S2-10 is coupled to an N-protected 4-Br indole derivative S2-11 (prepared by reacting the corresponding unprotected indole with a suitable electrophile in the presence of DMAP) utilizing a Suzuki coupling to give intermediate S2-12. Intermediate S2-12 can be selectively deprotected to give intermediate S2-13 which in turn can be reacted with a suitable electrophile, e. g. an alkyl halogenide or triflate, in the presence of base or a suitable aldehyde or ketone, in the presence of a reducing agent, e.g. sodium triacetoxyboronhydride or sodium cyanoborohydride, to give S2-14. Deprotection gives the final Examples S2-15.

In a further aspect, the disclosure provides a process for the preparation of a compound of formula (I), in free form or in pharmaceutically acceptable salt form, comprising the step as described above.

### Pharmaceutical Compositions

In another aspect, the disclosure provides a pharmaceutical composition comprising one or more compounds of described herein or a pharmaceutically acceptable salt thereof, and one or more pharmaceutically acceptable carriers.

In a further embodiment, the composition comprises at least one or two pharmaceutically acceptable carriers, such as those described herein. For purposes of the disclosure, unless designated otherwise, solvates and hydrates are generally considered compositions.

The compounds of Formula (I), and subformulae thereof, described herein may be administered alone or as an active ingredient of a pharmaceutical composition. Accordingly, provided herein are pharmaceutical compositions comprising a compounds of Formula (I), or subformulae thereof, or a pharmaceutically acceptable salt thereof, and one or more pharmaceutically acceptable carriers.

Methods of preparing various pharmaceutical compositions are known to those of skill in the art and may be described in, for example, in the Handbook of Pharmaceutical Excipients, American Pharmaceutical Association (current edition); Pharmaceutical Dosage Forms Tablets (Lieberman, Lachman and Schwartz, editors) current edition, published by Marcel Dekker, Inc., as well as Remington's Pharmaceutical Sciences (Arthur Osol, editor), 1553-1593 (current edition).

The pharmaceutical composition can be formulated for particular routes of administration such as oral administration, topical administration, parenteral administration, and rectal administration, etc. In addition, the pharmaceutical compositions of the disclosure can be made up in a solid form (including without limitation capsules, tablets, pills, granules, powders or suppositories), or in a liquid form (including without limitation solutions, gels, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone;
d) disintegrants, e.g., starches, agar, alginic acid or its sodium salt, or effervescent mixtures; and
e) absorbents, colorants, flavors and sweeteners.

In an embodiment, the pharmaceutical compositions are capsules comprising the active ingredient only.

Tablets may be either film coated or enteric coated according to methods known in the art.

The mode of administration and pharmaceutical composition are closely related to the therapeutic amounts of the compounds or compositions which are desirable and efficacious for the given treatment application. Pharmaceutical compositions provided herein can be formulated for ophthalmic, ocular, topical, and transdermal administration. In particular embodiments, the pharmaceutical compositions provided herein are suitable for ocular administration. To prepare pharmaceutical compositions, the active ingredient may be mixed with one or more pharmaceutically acceptable carrier(s) according to conventional pharmaceutical compounding techniques. The carrier(s) may take a wide variety of forms depending on the form of preparation desired for administration.

Suitable compositions for oral administration include an effective amount of a compound of the disclosure in the form of tablets, lozenges, aqueous or oily suspensions, dispersible powders or granules, emulsion, hard or soft capsules, or syrups or elixirs, solutions or solid dispersion. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients, which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the disclosure with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

In certain embodiments, the pharmaceutical compositions provided herein are formulated as solutions, suspensions, gels, creams, ointments, liposomes, ocular inserts or other pharmaceutical compositions suitable, in particular embodiments, for topical administration to the ocular surface, the cornea, the eyelid, margins of the eye, eyelashes and/or eye lid margin in order to deliver the composition to the eye. In some embodiments, liquid (aqueous or non-aqeuous) solutions may be used. In certain embodiments the pharmaceutical compositions are formulated as eye drops for topical administration to the ocular surface, the cornea, the eyelid, eye lid margins, eyelashes and/or margins of the eye in order to deliver the composition to the eye. Application of the pharmaceutical composition may be performed with an applicator, such as the subject's finger, a Week-Cel®, Q-tip®, or other device capable of delivering a formulation to the eyelid, eyelashes and/or eyelid margin in order to deliver the formulation to the eye. The pharmaceutical compositions provided herein may be viscous or semi-viscous; liquid, solid, or semi-solid; aqueous or non-aqueous, depending on the site of application, dose, solubility of drug, and a variety of other factors that are considered by those of skill in the art.

Any of a variety of carriers may be used in a pharmaceutical composition provided herein. In one embodiment, the pharmaceutically acceptable carrier is a non-aqueous carrier (e.g., oil, or oil mixture) having a viscosity in a range from about 50 cps to about 1000 cps, about 50 cps to about 500 cps, about 50 cps to about 200 cps, or about 60 cps to about 120 cps. In certain embodiments, the non-aqueous carrier comprises an oil, e.g., vegetable oils, silicone oils, mineral oil or any combination thereof. In some embodiments, the carrier may be liquid paraffin, white petrolatum, purified lanolin, gelation hydrocarbon, polyethylene glycol, hydrophilic ointment base, white ointment base, absorptive ointment base, Macrogol ointment base, simple ointment base, and the like. In certain embodiments, the pharmaceutical composition may include a monomeric polyol such as, glycerol, propylene glycol, and ethylene glycol, polymeric polyols such as polyethylene glycol, cellulose esters such hydroxypropylmethyl cellulose, carboxy methylcellulose sodium and hydroxy propylcellulose; dextrans such as dextran 70; water soluble proteins such as gelatin, polymers such as polyvinyl alcohol, polyvinylpyrrolidone, and povidone; carbomers, such as carbomer 934P. carbomer 941, carbomer 940 and carbomer 974P; and gums such as HP-guar.

Additional excipients may optionally be included in the pharmaceutical compositions provided herein. Examples of additional excipients include, for example, tonicity enhancers, preservatives, solubilizers, non-toxic excipients, demulcents, sequestering agents, pH adjusting agents, co-solvents, viscosity building agents, and combinations thereof.

The pharmaceutical composition of the disclosure may be in the form of an aqueous suspension or an aqueous solution. In one embodiment, the aqueous pharmaceutical composition of the disclosure is in the form of an aqueous suspension.

Aqueous pharmaceutical compositions according to the disclosure can be prepared using standard procedures that are familiar to the person skilled in the art, e.g., by admixture of the various components, suitably at ambient temperature and atmospheric pressure. In one embodiment, the aqueous pharmaceutical compositions of the disclosure are suitable for ocular administration.

In a further embodiment, the pharmaceutical composition of the disclosure is in the form of eye ointment, eye gel, eye cream, or eye drops.

In a further embodiment, the pharmaceutical composition of the disclosure is administered to the subject topically in the eyes.

The compounds of formula (I), in free form or in pharmaceutically acceptable salt form, exhibit valuable pharmacological properties, e.g., complement Factor B modulating properties e.g., as indicated in the in vitro tests as provided in the examples, and are therefore indicated for therapy or for use as research chemicals, e.g., as tool compounds.

Additional properties of the disclosed compounds include having good potency in the biological assays described herein, favorable safety profile, and possess favorable pharmacokinetic properties

### Diseases and Disorders and Methods of Use

In a further aspect, the disclosure provides a compound of formula (I), or a pharmaceutically acceptable salt thereof, for use in therapy.

In a further aspect, the disclosure provides a compound of formula (I), or a pharmaceutically acceptable salt thereof, for use in the treatment of a disease or disorder for which complement factor B is indicated. In one embodiment, the disease or disorder is affected by the inhibition of complement factor B activity.

Compounds of formula (I) and their pharmaceutically acceptable salts have complement factor B modulating and/or inhibitory activity and are believed to be of potential use for the treatment or prophylaxis of certain diseases or disorders, such as age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammation, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), immune complex membranoproliferative glomerulonephritis (IC-MPGN), IgA nephropathy, membranous nephropathy including idiopathic membranous nephropathy, diabetic nephropathy, atypical hemolytic uremic syndrome (aHUS), Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, neuromyelitis optica (NMO), IgA vasculitis (formerly known as Henoch-Schonlein purpura or HSP), CHAPLE syndrome, hematopoietic stem cell transplantation-associated thrombotic microangiopathy (HSCT-TMA), adult respiratory distress syndrome (ARDS,), myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury mesenteric artery reperfusion after aortic reconstruction, infectious disease or sepsis; COVID-19, immune complex disorders and autoimmune diseases, rheumatoid arthritis, osteoarthritis, Spondyloarthropathies, psoriatic arthritis, systemic lupus erythematosus (SLE), lupus nephritis, SLE nephritis, proliferative nephritis, myasthenia gravis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, antineutrophil cytoplasmic antibody (ANCA)-associated vasculitides (AAV), Buerger's vasculitis, cryoglobulinemia, Kawasaki disease, Takayasu arteritis, cryoglobulinemia, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenia purpura (TTP), and abdominal aortic aneurisms, and Grave's disease.

Having regard to their activity as complement factor B modulator or inhibitors, compounds of formula (I) and sub-formulae thereof, in free or pharmaceutically acceptable salt form, are useful in the treatment of conditions which may be treated by inhibition of complement factor B activity. In one aspect, the disclosure provides a method of treating or preventing a disease or disorder in a subject in need thereof, the method comprising administering to the subject a therapeutically effective amount of a compound of formula (I) or sub-formula thereof, or a pharmaceutically acceptable salt thereof.

In another aspect, the disclosure provides a method of modulating the complement alternative pathway activity in a subject, the method comprising administering to the subject a therapeutically effective amount of a compound of formula (I) or sub-formula thereof, or a pharmaceutically acceptable salt thereof.

In another aspect, the disclosure provides a method of treating a disease or disorder mediated by complement activation, in particular mediated by activation of the complement alternative pathway, in a subject in need thereof, the method comprising administering to the subject a therapeutically effective amount of a compound of formula (I) or sub-formula thereof, or a pharmaceutically acceptable salt thereof.

In another aspect, the disclosure provides a method of treating a disease or disorder that is affected by the modulation of complement alternative pathway comprising administering to the subject a therapeutically effective amount of a compound of formula (I) or sub-formula thereof, or a pharmaceutically acceptable salt thereof.

In another aspect, the disclosure provides a method of treating a disease or disorder associated with dysregulation of the complement alternative pathway comprising administering to the subject a therapeutically effective amount of a compound of formula (I) or sub-formula thereof, or a pharmaceutically acceptable salt thereof.

In another aspect, the disclosure provides a method of inhibiting the expression or activity of complement factor B, the method comprising administering to the subject a compound of formula (I) or sub-formula thereof, or a pharmaceutically acceptable salt thereof.

In another aspect, the disclosure provides a method of treating disease or disorder selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammation, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), immune complex membranoproliferative glomerulonephritis (IC-MPGN), IgA nephropathy, membranous nephropathy including idiopathic membranous nephropathy, diabetic nephropathy, atypical hemolytic uremic syndrome (aHUS), Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, neuromyelitis optica (NMO), IgA vasculitis (formerly known as Henoch-Schonlein purpura or HSP), CHAPLE syndrome, hematopoietic stem cell transplantation-associated thrombotic microangiopathy (HSCT-TMA), adult respiratory distress syndrome (ARDS,), myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury mesenteric artery reperfusion after aortic reconstruction, infectious disease or sepsis; COVID-19, immune complex disorders and autoimmune diseases, rheumatoid arthritis, osteoarthritis, Spondyloarthropathies, psoriatic arthritis, systemic lupus erythematosus (SLE), lupus nephritis, SLE nephritis, proliferative nephritis, myasthenia gravis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, antineutrophil cytoplasmic antibody (ANCA)-associated vasculitides (AAV), Buerger's vasculitis, cryoglobulinemia, Kawasaki disease, Takayasu arteritis, cryoglobulinemia, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenia purpura (TTP), and abdominal aortic aneurisms, and Grave's disease.

In certain aspects, methods are provided for the treatment of diseases associated with increased activity of the C3 amplification loop of the complement pathway. In certain embodiments, methods of treating or preventing compelment mediated diseases are provided in which the complement activation is induced by antibody-antigen interactions, by a component of an autoimmune disease, or by ischemic damage.

In a specific embodiment, the present disclosure provides a method of treating or preventing age-related macular degeneration (AMD) by administering to a subject in need thereof an effective amount of the compound of Formula (I) of the disclosure. In certain embodiments, patients who are currently asymptomatic but are at risk of developing a symptomatic macular degeneration related disorder are suitable for administration with a compound of the disclosure. The methods of treating or preventing AMD include, but are not limited to, methods of treating or preventing one or more symptoms or aspects of AMD selected from formation of ocular drusen, inflammation of the eye or eye tissue, loss of photoreceptor cells, loss of vision (including loss of visual acuity or visual field), neovascularization (including CNV), retinal detachment, photoreceptor degeneration, RPE degeneration, retinal degeneration, chorioretinal degeneration, cone degeneration, retinal dysfunction, retinal damage in response to light exposure, damage of the Bruch's membrane, and/or loss of RPE function.

The compound of Formula (I) of the disclosure can be used, inter alia, to prevent the onset of AMD, to prevent the progression of early AMD to advanced forms of AMD including neovascular AMD or geographic atrophy, to slow and/or prevent progression of geographic atrophy, to treat or prevent macular edema from AMD or other conditions (such as diabetic retinopathy, uveitis, or post surgical or non-surgical trauma), to prevent or reduce the loss of vision from AMD, and to improve vision lost due to pre-existing early or advanced AMD. It can also be used in combination with anti-VEGF therapies for the treatment of neovascular AMD patients or for the prevention of neovascular AMD.

All the aforementioned embodiments relating to the methods of treatment of the aforementioned diseases are equally applicable to:
a compound of formula (I) or sub-formula thereof, or a pharmaceutically acceptable salt thereof, for use in the treatment of the aforementioned diseases according to the present disclosure;
use of a compound of formula (I) or sub-formula thereof, or a pharmaceutically acceptable salt thereof, in the manufacture of a medicament for the treatment of the aforementioned diseases according to the present disclosure;
use of a compound of formula (I) or sub-formula thereof, or a pharmaceutically acceptable salt thereof, for the treatment of the aforementioned diseases according to the present disclosure; and
a pharmaceutical composition comprising a compound of formula (I) or sub-formula thereof, or a pharmaceutically acceptable salt thereof, and one or more pharmaceutically acceptable carriers, for use in the treatment of the aforementioned diseases according to the present disclosure.

### Dosage

The pharmaceutical composition or combination of the disclosure can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated.

The above-cited dosage properties are demonstrable in vitro and in vivo tests using advantageously mammals, e.g., mice, rats, dogs, monkeys or isolated organs, tissues and preparations thereof. The compounds of the disclosure can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, e.g., between about 0.1-500 mg/kg.

The therapeutically effective dosage of a compound, the pharmaceutical composition, or the combinations thereof, is dependent on the species of the subject, the body weight, age and individual condition, the disorder or disease or the severity thereof being treated.

The activity of a compound according to the disclosure can be assessed by the in vitro methods described in the Examples.

### Combination Therapy

In another aspect, the disclosure provides a pharmaceutical combination comprising a compound of formula (I), or subformulae thereof, or a pharmaceutically acceptable salt thereof, and one or more additional therapeutic agent(s) for simultaneous, separate or sequential use in therapy.

The compound of the disclosure may be administered either simultaneously with, or before or after, one or more other therapeutic agent. The compound of the disclosure may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the disclosure. Thus, in one embodiment, the disclosure provides a combination comprising a therapeutically effective amount of a compound of formula or subformulae thereof, or a pharmaceutically acceptable salt thereof, and one or more therapeutically active agents.

In certain embodiments, a compound of Formula (I), or subformulae thereof, or a pharmaceutically acceptable salt thereof, may be administered with an additional therapeutic agent.

The pharmaceutical compositions of the disclosure, e.g., a pharmaceutical composition comprising a compound of formula (I), can be administered alone or in combination with other molecules known to have a beneficial effect on retinal attachment or damaged retinal tissue, including molecules capable of tissue repair and regeneration and/or inhibiting inflammation. Examples of useful, cofactors include complement inhibitors (such as inhibitors of Factor D, C5a receptor and antibody or Fabs against C5, C3, properidin, factor H, and the like), anti-VEGF agents (such as an antibody or FAB against VEGF, e.g., Lucentis or Avastin), basic fibroblast growth factor (bFGF), ciliary neurotrophic factor (CNTF), axokine (a mutein of CNTF), leukemia inhibitory factor (LIF), neutrotrophin 3 (NT-3), neurotrophin-4 (NT-4), nerve growth factor (NGF), insulin-like growth factor II, prostaglandin E2, 30 kD survival factor, taurine, and vitamin A. Other useful cofactors include symptom-alleviating cofactors, including antiseptics, antibiotics, antiviral and antifungal agents and analgesics and anesthetics. Suitable agents for combination treatment with the compounds of the disclosure include agents known in the art that are able to modulate the activities of complement components.

In some embodiments, the present disclosure provide a combination therapy for preventing and/or treating AMD or another complement related ocular disease as described above with a compound of the disclosure and an anti-angiogenic, such as anti-VEGF agent (including Lucentis Avastin and VEGF-R2 inhibitors including pazopanib, sutent, inifanib, and VEGF-R2 inhibitors disclosed in WO2010/066684) or photodynamic therapy (such as as verteporfin).

In some embodiments, the present disclosure provide a combination therapy for preventing and/or treating autoimmune disease as described above with a compound of the disclosure and a B-Cell or T-Cell modulating agent (for example cyclosporine or analogs thereof, rapamycin, RAD001 or analogs thereof, and the like). In particular, for multiple sclerosis therapy may include the combination of a compound of the disclosure and a second MS agent selected from fingolimod, cladribine, tysarbi, laquinimod, rebif, avonex and the like. A non-limiting list of such agents incudes pharmaceutical agents effective in the treatment of diseases and conditions in which vanilloid receptor activation plays a role or is implicated, including cyclooxygenase-2 (COX-2) inhibitors, such as specific COX-2 inhibitors, e.g., celecoxib and rofecoxib; and non-steroidal anti-inflammatory drugs (NSAIDs), e.g., acetylsalicylic acid and propionic acid derivatives; tricyclic anti-depressants, e.g., Anafranil®, Asendin®, Aventyl®, Elavil®, Endep®, Norfranil®, Norpramin®, Pamelor®, Sinequan®, Surmontil®, Tipramine®, Tofranil®, Vivactil®, Tofranil-PM®; anti-convulsants, e.g., carbamazepine, oxcarbazepine and gabapentin; bradykinin B1 or B2 antagonists; and GABAB agonists, e.g., L-baclofen.

In certain embodiments, further therapeutic agents may include, for instance, other compounds and antibodies useful for treating ocular disorders. A non-limiting list of such agents incudes retinoid X receptor agonists, such as vitamin A, retinoic acid, phytanic acid, lithocholic acid, bexarotene, docosahexaenoic acid, or flurobexarotene. Other additional therapeutic agents include ophthalmic steroids such as, dexamethasone, fluocinolone, loteprednol, difluprednate, fluorometholone, prednisolone, prednisone, medrysone, triamcinolone, betamethasone, rimexolone, or pharmaceutically acceptable salts thereof. In addition, other additional therapeutic agents include those used to target ocular surface disease disorders, such as dry eye disease. Non-limiting example of such additional therapeutic agents include Xiidra® (lifitegrast), Restasis® (cyclosporine), minocycline, doxycycline, or other tetracycline antibiotics. Other examples include keratolytic agents such as selenium disulfide, salicylic acid, glycolic acid etc., or pharmaceutically acceptable salts thereof.

In certain embodiments, further therapeutic agents may include, for instance, other compounds useful in the treatment of pain. In an embodiment, a compound of Formula (I), or subformula thereof, or a pharmaceutically acceptable salt thereof, may be administered with an additional analgesic agent. Such anagelsic agent may be an NSAID (e.g., acetylsalicylic acid and propionic acid derivatives, e.g., Aleve®), opioid or steroid.

### Preparation of Compounds

It is understood that in the following description, combinations of substituents and/or variables of the depicted formulae are permissible only if such combinations result in stable compounds.

It will also be appreciated by those skilled in the art that in the processes described below, the functional groups of intermediate compounds may need to be protected by suitable protecting groups. Such functional groups include hydroxy, phenol, amino and carboxylic acid. Suitable protecting groups for hydroxy or phenol include trialkylsilyl or diarylalkylsilyl (e.g., tert-butyldimethylsilyl, tert-butyldiphenylsilyl or trimethylsilyl), tetrahydropyranyl, benzyl, substituted benzyl, methyl, and the like. Suitable protecting groups for amino, amidino and guanidino include t-butoxycarbonyl, benzyloxycarbonyl, and the like. Suitable protecting groups for carboxylic acid include alkyl, aryl or arylalkyl esters.

Protecting groups may be added or removed in accordance with standard techniques, which are well-known to those skilled in the art and as described herein. The use of protecting groups is described in detail in J. F. W. McOmie, “Protective Groups in Organic Chemistry”, Plenum Press, London and New York 1973; T. W. Greene and P. G. M. Wuts, “Greene's Protective Groups in Organic Synthesis”, Fourth Edition, Wiley, New York 2007; P. J. Kocienski, “Protecting Groups”, Third Edition, Georg Thieme Verlag, Stuttgart and New York 2005; and in “Methoden der organischen Chemie” (Methods of Organic Chemistry), Houben Weyl, 4th edition, Volume 15/l, Georg Thieme Verlag, Stuttgart 1974.

The protecting group may also be a polymer resin, such as a Wang resin or a 2-chlorotrityl-chloride resin.

The following reaction Examples illustrate methods to make compounds of this disclosure. It is understood that one skilled in the art would be able to make these compounds by similar methods or by methods known to one skilled in the art. In general, starting components and reagents may be obtained from sources such as Sigma Aldrich, Lancaster Synthesis, Inc., Maybridge, Matrix Scientific, TCI, and Fluorochem USA, Strem, other commercial vendors, or synthesized according to sources known to those skilled in the art, or prepared as described in this disclosure.

### Analytical Methods, Materials, and Instrumentation

Unless otherwise noted, reagents and solvents were used as received from commercial suppliers. Proton nuclear magnetic resonance (NMR) spectra were obtained on either Bruker Avance, Avance III or Avance Neo spectrometer 400 MHZ, Varian Oxford 400 MHZ, or Varian Mercury 300 MHz spectrometer unless otherwise noted. Spectra are given in ppm (δ) and coupling constants, J, are reported in Hertz. Tetramethylsilane (TMS) was used as an internal standard. Chemical shifts are reported in ppm relative to dimethyl sulfoxide (δ 2.50), methanol (δ 3.31), chloroform (δ 7.26) or other solvent as indicated in NMR spectral data. A small amount of the dry sample (2-5 mg) is dissolved in an appropriate deuterated solvent (1 mL). The chemical names were generated using ChemBioDraw Ultra v19 from CambridgeSoft.

Mass spectra (ESI-MS) were collected using a Waters System (Acquity UPLC and a Micromass ZQ mass spectrometer) or Agilent-1260 Infinity (6120 Quadrupole); all masses reported are the m/z of the protonated parent ions unless recorded otherwise. The sample was dissolved in a suitable solvent such as MeCN, DMSO, or MeOH and was injected directly into the column using an automated sample handler.

### Abbreviations

9-BBN 9-Borabicyclo[3.3.1]nonane
ACN acetonitrile
AcOH acetic acid
aq aqueous
Boc tert-butyl carboxy
br broad
c concentration
CBr₄ tetrabromomethane
CO₂ carbon dioxide
d doublet
DAD diode-array detection
DBU 1,8-diazabicyclo[5.4.0]undec-7-ene
DCE 1,2-dichloroethane
DCM dichloromethane
dd doublet of doublets
DIPEA diisopropylethylamine
DMAP N,N-dimethylaminopyridine
DMF N,N-dimethylformamide
DMSO dimethylsulfoxide
ee enantiomeric excess
eq equivalent
EtOAc ethyl acetate
EtOH ethanol
h hour(s)
H₂ hydrogen
HCl hydrochloric acid
HPLC high performance liquid chromatography
IPA iso-propanol
KHF₂ potassium hydrogen fluoride
K₂CO₃ potassium carbonate K₃PO₄ potassium phosphate, tribasic
LC-MS liquid chromatography and mass spectrometry
m multiplet
m/z mass to charge ratio
MeOH methanol
Me-THF 2-methyltetrahydrofuran
MgSO₄ magnesium sulfate
min minutes
MS mass spectrometry
N₂ nitrogen
NaCNBH₃ sodium cyanoborohydride
Na₂CO₃ sodium carbonate
NaHCO₃ sodium bicarbonate
Na₂SO₄ sodium sulfate
NaOH sodium hydroxide
NH₄OH ammonium hydroxide
NMR nuclear magnetic resonance
NP normal phase
org organic
Pd₂(dba)₃ tris(dibenzylideneacetone)dipalladium(0)
PdCl₂(dppf) 1,1′-bis(diphenylphosphino)ferrocene-palladium(II) dichloride
PdCl₂(dtbpf) [1,1′-bis(di-tert-butylphosphino)ferrocene]palladium(II) dichloride
PE petroleum ether
PPh₃ triphenylphosphine
ppm parts per million
rac racemic
RM reaction mixture
RP reversed phase
Rt retention time
RT room temperature
s singlet
sat. saturated
scCO₂ super critical carbon dioxide
SFC Supercritical Fluid Chromatography
SiO₂ silica gel
soln. solution
t triplet
TBAF tetra-n-butylammonium fluoride
TBME methyl tert-butyl ether
tBu tertiary butyl
tert-Bu tertiary butyl
tert-BuOH tert-butanol
TFA trifluoroacetic acid
TFAA tifluoroacetic acid anhydride
Tf trifluoromethanesulfonate
THF tetrahydrofuran
TLC thin layer chromatography
UPLC ultra performance liquid chromatography
Xantphos 4,5-bis(diphenylphosphino)-9,9-dimethylxanthene
Xphos Pd G2 Chloro(2-dicyclohexylphosphino-2′,4′,6′-triisopropyl-1,1′-biphenyl)[2-(2′-amino-1,1′-biphnyl)]palladium (II)
wt % weight percent

### INTERMEDIATES

### Intermediate I-1

### tert-butyl 4-bromo-5,7-dimethyl-1H-indole-1-carboxylate

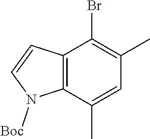 [SMILES: Cc1cc(C)c2c(ccn2C(=O)OC(C)(C)C)c1Br]

DMAP (242 mg, 1.98 mmol) was added to a suspension of 4-bromo-5,7-dimethyl-1H-indole (CAS #1167056-53-2, 8.87 g, 39.6 mmol) and di-tert-butyl dicarbonate (10.4 g, 47.5 mmol) in DCM (122 mL). The RM was stirred at RT for 40 min, then evaporated to dryness to give a brown residue that was purified over a silica gel (330 g) using an automated purification system (NP; Teledyne ISCO®; collected at 256/294 nm; flow 200 mL/min; eluent: heptane+0 to 4.5% EtOAc in 23 min) to give after concentration under reduced pressure at 50° C. the title compound as off-white crystals (9.05 g). LC-MS Method B-2: Rt=8.55 min; MS m/z [M-Boc+H]⁺=223.9/225.9.

### Intermediate I-2

### tert-butyl 4-bromo-5-methoxy-7-methyl-1H-indole-1-carboxylate

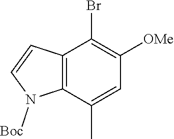 [SMILES: COc1cc(C)c2c(ccn2C(=O)OC(C)(C)C)c1Br]

DMAP (25.95 mg, 212.4 μmol) was added to a soln. of 4-bromo-5-methoxy-7-methyl-1H-indole (CAS #1352393-65-7, 1.020 g, 4.248 mmol) and di-tert-butyl dicarbonate (1.113 g, 1.17 mL, 5.098 mmol) in DCM (14 mL). The RM was stirred at RT for 2 h, then concentrated under reduced pressure to dryness. The residue was purified over silica gel (40 g) using an automated purification system (NP; Teledyne ISCO®; collected at 256/294 nm; flow 40 mL/min; eluent: heptane+0.0 to 8.7% EtOAc in 15.2 min) to give after concentration under reduced pressure at 50° C. the title compound as a colorless oil (1.42 g). LC-MS Method B-1: Rt=1.54 min; MS m/z [M+H]⁺=340.0/342.0.

### Intermediate I-3

### tert-butyl 4-bromo-5-cyclopropyl-7-methyl-1H-indole-1-carboxylate

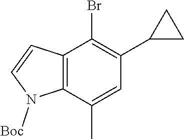 [SMILES: Cc1cc(C2CC2)c(Br)c2ccn(C(=O)OC(C)(C)C)c12]

### Step 1: 2-cyclopropyl-4-methyl-5-nitroaniline (Intermediate I-3-1)

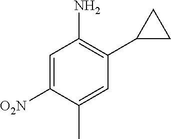

To a solution of 2-bromo-4-methyl-5-nitroaniline (CAS #102169-99-3, 50 g, 268.74 mmol) in dioxane (200 mL) and H₂O (66 mL) were added cyclopropylboronic acid (CAS #411235-57-9 11.15 g, 129.84 mmol), K₃PO₄ (55.12 g, 260 mmol), and Pd(dppf)Cl₂-DCM (3.53 g, 4.33 mmol). The resulting mixture was stirred at 90° C. for 16 h. The RM was filtered, and the filtrate was extracted with EtOAc (3×200 mL), dried over Na₂SO₄, filtered, and concentrated under reduced pressure. The residue was purified by column chromatography over silica gel (EtOAc/PE=0/100 to 30/70) to give 2-cyclopropyl-4-methyl-5-nitroaniline (12.0 g) as a black solid. ¹H NMR (400 MHZ, DMSO-d₆) δ [ppm] 7.27 (s, 1H), 6.81 (s, 1H), 5.48-5.39 (m, 2H), 2.33 (s, 3H), 1.75 (tt, J=5.4, 8.3 Hz, 1H), 0.97-0.88 (m, 2H), 0.65-0.56 (m, 2H).

### Step 2: 1-bromo-2-cyclopropyl-4-methyl-5-nitrobenzene (Intermediate I-3-2)

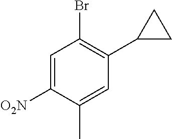

A solution of 2-cyclopropyl-4-methyl-5-nitroaniline (Intermediate I-3-1, 120 g, 624.3 mmol) and tert-butyl nitrite (83.66 g, 378.58 mmol) in MeCN (1.2 L) was stirred at 0° C. for 0.5 h. To the mixture was added CuBr₂ (83.66 g, 378.58 mmol) at 0° C. and stirring was continued at ˜25° C. for 2 h. The RM was diluted with H₂O (1 L) and then extracted with EtOAc (3×500 mL). The combined org layers were washed with brine (1 L), dried by Na₂SO₄, filtered, and concentrated under reduced pressure to give 1-bromo-2-cyclopropyl-4-methyl-5-nitrobenzene (120 g) as a yellow solid which was used in the next step without further purification. ¹H NMR (400 MHZ, chloroform-d) δ [ppm] 8.23 (s, 1H), 6.80 (s, 1H), 2.54 (s, 3H), 2.22 (tt, J=5.3, 8.4 Hz, 1H), 1.18-1.11 (m, 2H), 0.81-0.74 (m, 2H).

### Step 3: 4-bromo-5-cyclopropyl-7-methyl-1H-indole (Intermediate I-3-3)

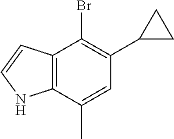

To a solution of 1-bromo-2-cyclopropyl-4-methyl-5-nitrobenzene (Intermediate I-3-2, 117 g, 144.5 mmol) in THF (1.2 L) was added vinylmagnesium bromide (3.2 L, 3.20 mol) at −78° C. After the addition was complete, the RM was stirred at −40° C. for 2 h. The RM was quenched with H₂O (3 L) and then extracted with EtOAc (3×3 L). The combined org layers were washed with brine (5 L), dried over Na₂SO₄, filtered, and concentrated under reduced pressure. The residue was purified by column chromatography over silica gel (EtOAc/PE=0/100 to 10/90) to give 4-bromo-5-cyclopropyl-7-methyl-1H-indole (35 g) as a yellow solid. ¹H NMR (400 MHZ, chloroform-d) δ [ppm] 8.12 (br s, 1H), 7.25-2.22 (m, 1H), 6.64-6.58 (m, 2H), 2.43 (d, J=0.8 Hz, 3H), 2.26 (tt, J=5.3, 8.5 Hz, 1H), 1.05-0.98 (m, 2H), 0.74-0.66 (m, 2H).

### Step 4: tert-butyl 4-bromo-5-cyclopropyl-7-methyl-1H-indole-1-carboxylate (Intermediate I-3)

To a mixture of 4-bromo-5-cyclopropyl-7-methyl-1H-indole (35 g, 126 mmol) and di-tert-butyl dicarbonate (148.24 g, 700 mmol) was added DMAP (1.68 g, 14 mmol). The RM was stirred at 40° C. for 2 h and then cooled to 25° C. N¹,N¹-Dimethylethane-1,2-diamine (105 mL) was added and the mixture was stirred at 25° C. for 10 min. The mixture was diluted with H₂O (100 mL) and then extracted with EtOAc (3×50 mL). The combined org layers were washed with brine (100 mL), dried over Na₂SO₄, filtered, and concentrated under reduced pressure to give tert-butyl 4-bromo-5-cyclopropyl-7-methyl-1H-indole-1-carboxylate (38 g) as a yellow solid which was directly used without further purification. ¹H NMR (400 MHZ, chloroform-d) δ [ppm] 7.54 (d, J=3.8 Hz, 1H), 6.67 (s, 1H), 6.65 (d, J=3.8 Hz, 1H), 2.56 (s, 3H), 2.22 (tt, J=5.3, 8.5 Hz, 1H), 1.64 (s, 9H), 1.06-0.97 (m, 2H), 0.76-0.66 (m, 2H).

The following Intermediates were synthesized from the appropriate starting material by applying similar methods described above:

### Intermediate I-4

### tert-butyl 4-bromo-7-methyl-1H-indole-1-carboxylate

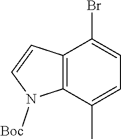 [SMILES: Cc1ccc(Br)c2ccn(C(=O)OC(C)(C)C)c12]

Intermediate I-4 was prepared from 4-bromo-7-methyl-1H-indole (CAS #936092-87-4). ¹H NMR (400 MHZ, chloroform-d) δ [ppm] 7.62 (d, J=3.8 Hz, 1H), 7.29 (s, 1H), 6.93 (d, J=9.9 Hz, 1H), 6.65 (d, J=3.8 Hz, 1H), 2.61 (s, 3H), 1.67 (s, 9H).

### Intermediate I-5

### tert-butyl 4-bromo-5-fluoro-7-methyl-1H-indole-1-carboxylate

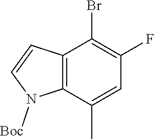 [SMILES: Cc1cc(F)c(Br)c2ccn(C(=O)OC(C)(C)C)c12]

Intermediate I-5 was prepared from 1-bromo-2-fluoro-4-methyl-5-nitrobenzene (CAS #224185-19-7). ¹H NMR (400 MHZ, chloroform-d) δ [ppm] 7.62 (d, J=3.8 Hz, 1H), 6.93 (d, J=9.9 Hz, 1H), 6.65 (d, J=3.8 Hz, 1H), 2.61 (s, 3H), 1.67 (s, 9H).

### Intermediate I-6

### tert-butyl 4-bromo-5-chloro-7-methyl-1H-indole-1-carboxylate

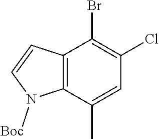 [SMILES: Cc1cc(Cl)c(Br)c2ccn(C(=O)OC(C)(C)C)c12]

Intermediate I-6 was prepared from 1-bromo-2-chloro-4-methyl-5-nitrobenzene (CAS #1126367-34-7). ¹H NMR (400 MHZ, chloroform-d) δ [ppm] 7.49 (d, J=3.6 Hz, 1H), 7.14-7.05 (m, 1H), 6.55 (d, J=3.8 Hz, 1H), 2.50 (d, J=0.8 Hz, 3H), 1.56 (s, 9H).

### Intermediate I-7

### tert-butyl 4-bromo-5,7-dichloro-1H-indole-1-carboxylate

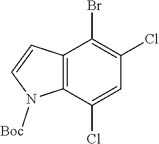 [SMILES: CC(C)(C)OC(=O)n1ccc2c(Br)c(Cl)cc(Cl)c21]

Intermediate I-7 was prepared from 1-bromo-2,4-dichloro-5-nitrobenzene (CAS #65001-80-1). ¹H NMR (400 MHZ, chloroform-d) δ [ppm] 7.54 (d, J=3.7 Hz, 1H), 7.35 (s, 1H), 6.59 (d, J=3.7 Hz, 1H), 1.58 (s, 9H).

### Intermediate I-8

### tert-butyl 4-bromo-3-chloro-5,7-dimethyl-1H-indole-1-carboxylate

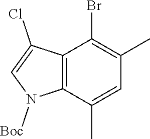 [SMILES: Cc1cc(C)c2c(c(Cl)cn2C(=O)OC(C)(C)C)c1Br]

### Step 1: 4-bromo-3-chloro-5,7-dimethyl-1H-indole (Intermediate I-8-1)

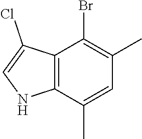

To a solution of 4-bromo-5,7-dimethyl-1H-indole (CAS #1167056-53-2, 345 mg, 1.54 mmol) in DMF (4.0 mL) at 0° C. was added a solution of N-chlorosuccinimide (246.7 mg, 1.85 mg) in DMF (1 mL) in a dropwise manner. The RM was stirred for additional 10 min at 0° C. and then diluted with H₂O. The formed precipitate was filtered off and dried under reduced pressure. Purification by column chromatography over silica gel (heptane/EtOAc=80/20) provided 4-bromo-3-chloro-5,7-dimethyl-1H-indole (185 mg) as dark greenish solid. ¹H NMR (400 MHZ, chloroform-d) δ [ppm] 7.97 (s, 1H), 7.18 (d, J=2.7 Hz, 1H), 6.92 (s, 1H), 2.48 (s, 3H), 2.39 (s, 3H).

### Step 2: tert-butyl 4-bromo-3-chloro-5,7-dimethyl-1H-indole-1-carboxylate (Intermediate I-8)

Intermediate I-8 was prepared from Intermediate I-8-1 following a similar method as described above for Intermediate I-1. ¹H NMR (400 MHZ, chloroform-d) δ [ppm] 7.54 (s, 1H), 7.05 (s, 1H), 2.51 (s, 3H), 2.47 (s, 3H), 1.65 (s, 9H).

### Intermediate A-1: methyl 4-(4-methylene-1-(2,2,2-trifluoroethyl)piperidin-3-yl)benzoate

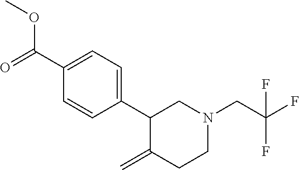 [SMILES: C=C1CCN(CC(F)(F)F)CC1c1ccc(C(=O)OC)cc1]

### Step 1: tert-butyl 3-(4-(methoxycarbonyl)phenyl)-4-oxopiperidine-1-carboxylate (Intermediate A-1-1)

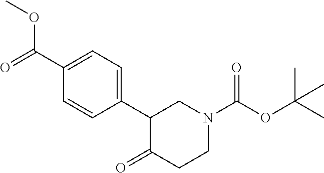 [SMILES: COC(=O)c1ccc(C2CN(C(=O)OC(C)(C)C)CCC2=O)cc1]

K₃PO₄ (40.86 g, 15.94 mL, 192.5 mmol) was added to a soln. of tert-butyl 4-oxopiperidine-1-carboxylate (30.02 g, 150.7 mmol), methyl 4-bromobenzoate (18.0 g, 83.7 mmol) and Xantphos (2.131 g, 3.68 mmol) in toluene (160 mL). The mixture was evacuated/back-filled with N₂ twice. Pd₂(dba)₃ (1.533 g, 1.674 mmol) was added, the mixture was again evacuated/back-filled with N₂ twice and then heated to 80° C. under a N₂ atmosphere overnight. The RM was cooled to RT and partitioned between toluene and water. The slurry was filtered over celite and the filtrate was washed with sat. aq NaHCO₃ soln., brine, and toluene. The combined org layers were dried over MgSO₄, filtered and the volatiles were removed under reduced pressure at 50° C. The resulting residue was purified over silica gel (330 g) using an automated purification system (NP; Teledyne ISCO®; monitored with 250 nm; flow 200 mL/min; eluent: heptanes+5 to 40% EtOAc in 31.0 min) and then re-purified over a silica gel (330 g) using an automated purification system (normal phase; Teledyne ISCO®; monitored with 250 nm; flow 200 mL/min; eluent: dichloromethane+0.0 to 4.7% EtOAc in 26.2 min, then re-purified in two portions on a RediSep C18 column (360 g each) using an automated purification system (Teledyne ISCO®; collected at 240 nm; flow 150 mL/min; eluent: water+25 to 60% ACN. Pure fractions were combined and the ACN was removed under reduced pressure at 50° C. to give a slurry of water. The mixture was extracted with DCM (3×) and the combined org layers were dried over MgSO₄, filtered and the volatiles were removed under reduced pressure at 50° C. to give the title compound as a white solid (9.20 g). LC-MS Method B-1: Rt=1.02 min; MS m/z [M−H]⁻=332.0.

### Step 2: methyl 4-(4-oxopiperidin-3-yl)benzoate (Intermediate A-1-2)

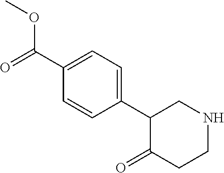 [SMILES: COC(=O)c1ccc(C2CNCCC2=O)cc1]

tert-Butyl 3-(4-(methoxycarbonyl)phenyl)-4-oxopiperidine-1-carboxylate (Intermediate A-1-1, 9.20 g, 27.6 mmol) was treated with HCl (4M in dioxane, 140.0 mL). The initial soln. turned into a thick, white suspension that was stirred at RT for 60 min. About ¾ of the volatiles were removed under reduced pressure at 50° C. to give a slurry in dioxane. The mixture was diluted with TBME and filtered. The solid was washed with TBME (3×) and dried under reduced pressure at 50° C. to give the title compound as an off-white solid (HCl salt, 7.60 g, ˜85% purity). ¹H NMR (400 MHZ, DMSO-d₆) δ [ppm] 9.94-9.54 (s, 2H), 7.97-7.92 (m, 2H), 7.41-7.35 (m, 2H), 4.33 (dd, J=11.1, 7.9 Hz, 1H), 3.86 (s, 3H), 3.69-3.59 (m, 3H), 3.57-3.47 (m, 1H), 3.06-2.93 (m 1H), 2.63-2.49 (m, 1H, partially overlapping with DMSO peak).

### Step 3: methyl 4-(4-oxo-1-(2,2,2-trifluoroethyl)piperidin-3-yl)benzoate (Intermediate A-1-3)

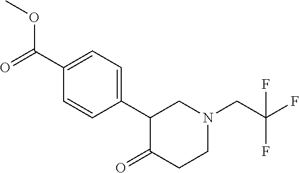 [SMILES: COC(=O)c1ccc(C2CN(CC(F)(F)F)CCC2=O)cc1]

DIPEA (10.83 g, 14.6 mL, 83.8 mmol) was added to suspension of methyl 4-(4-oxopiperidin-3-yl)benzoate HCl salt (Intermediate A-1-2, 7.60 g, ˜85% purity) in ACN (93 mL). 2,2,2-Trifluoroethyl trifluoromethanesulfonate (6.90 mL, 47.9 mmol) was added and the RM was stirred at 50° C. overnight, then cooled to RT. The volatiles were removed under reduced pressure at 50° C. and the residue was partitioned between TBME and water. The layers were separated and washed with brine and TBME. The combined organic layers were dried over MgSO₄, filtered and the volatiles were removed under reduced pressure at 50° C. to give an orange oil that was purified over silica gel (220 g) using an automated purification system (NP; Teledyne ISCO®; collected at 245/260 nm; flow 150 mL/min; eluent: heptane+5.0 to 30% EtOAc in 24 min) to give after concentration under reduced pressure the title compound as a yellow oil (7.29 g). LC-MS Method B-1: Rt=0.95 min; MS m/z [M+H]⁺=316.3.

### Step 4: methyl 4-(4-methylene-1-(2,2,2-trifluoroethyl)piperidin-3-yl)benzoate (Intermediate A-1)

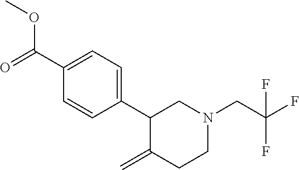 [SMILES: C=C1CCN(CC(F)(F)F)CC1c1ccc(C(=O)OC)cc1]

A suspension of zinc dust (9.63 g, 147.3 mmol) in THF (160 mL) was carefully evacuated/back-filled with N₂ twice. Diiodomethane (21.92 g, 6.60 mL, 81.83 mmol) was added in a drop-wise manner. The RM was stirred at RT for 30 min. Then the RM was cooled to 0 to 5° C. and titanium tetrachloride (1M in DCM, 16.37 mL, 16.37 mmol) was added in a drop-wise manner while keeping the temperature below 18° C. After the exothermic reaction had ceased, the suspension was stirred without cooling bath for additional 30 min at ambient temperature. A soln. of methyl 4-(4-oxo-1-(2,2,2-trifluoroethyl)piperidin-3-yl)benzoate (Intermediate A-1-3, 5.160 g, 16.37 mmol) in THF (10.3 mL) was added in a drop-wise manner. The RM was stirred at RT for 1.5 h, then poured into a well stirred mixture of sat. aq NaHCO₃ soln. and TBME. The resulting slurry was filtered over celite and the solids were washed with TBME (3×). The layers of the filtrate were separated and washed with brine and TBME. The combined org layers were dried over MgSO₄, filtered and the volatiles were removed under reduced pressure at 50° C. The resulting residue was purified over silica gel (220 g) using an automated purification system (NP; Teledyne ISCO®; collected at 238/254 nm; flow 150 mL/min; eluent: heptanes+30 to 70% DCM in 29.0 min). Pure fractions were combined and the volatiles were removed under reduced pressure at 50° C. to give the title compound as a colorless oil which solidified upon standing (3.27 g). LC-MS Method B-1: Rt=1.42 min; MS m/z [M+H]⁺=314.1.

### Intermediate A-2

### tert-butyl (3R,4R)-3-(4-(tert-butoxycarbonyl)phenyl)-4-(hydroxymethyl)piperidine-1-carboxylate

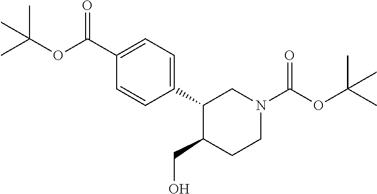 [SMILES: CC(C)(C)OC(=O)c1ccc([C@@H]2CN(C(=O)OC(C)(C)C)CC[C@H]2CO)cc1]

### Step 1: 1-(tert-butyl) 4-ethyl 5-(((trifluoromethyl)sulfonyl)oxy)-3,6-dihydropyridine-1,4(2H)-dicarboxylate (Intermediate A-2-1)

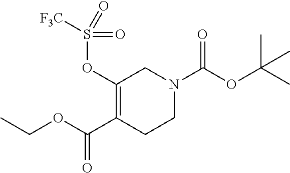 [SMILES: CCOC(=O)C1=C(OS(=O)(=O)C(F)(F)F)CN(C(=O)OC(C)(C)C)CC1]

To a stirred solution of 1-(tert-butyl) 4-ethyl 3-oxopiperidine-1,4-dicarboxylate (CAS #71233-25-5, 30.23 g, 111.4 mmol) in DCM (893 mL) at −78° C. under N₂ atmosphere was added slowly DIPEA (18.72 g, 144.8 mmol) followed by Tf₂O (37.72 g, 22.59 mL, 133.7 mmol) in a dropwise manner. The resulting mixture was stirred at RT for at ˜1 h. The RM was diluted with DCM (150 mL) and washed with sat. aq NaHCO₃ solution (300 mL). The separated aq layer was extracted with DCM (300 mL) and the combined org phases were dried over Na₂SO₄. The mixture was passed through a plug of Florisil® and concentrated under reduced pressure to afford the crude 1-(tert-butyl) 4-ethyl 5-(((trifluoromethyl)sulfonyl)oxy)-3,6-dihydropyridine-1,4(2H)-dicarboxylate which was directly used without further purification. LC-MS Method C-1: Rt=1.29 min; MS m/z [M-Boc+H]⁺=304.1.

### Step 2: 1-(tert-butyl) 4-ethyl 5-(4-(tert-butoxycarbonyl)phenyl)-3,6-dihydropyridine-1,4(2H)-dicarboxylate (Intermediate A-2-2)

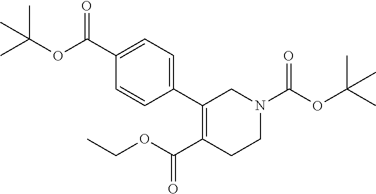 [SMILES: CCOC(=O)C1=C(c2ccc(C(=O)OC(C)(C)C)cc2)CN(C(=O)OC(C)(C)C)CC1]

A solution of K₃PO₄ (70.94 g, 334.2 mmol) in H₂O (248.4 mL) was added to a solution of crude 1-(tert-butyl) 4-ethyl 5-(((trifluoromethyl)sulfonyl)oxy)-3,6-dihydropyridine-1,4(2H)-dicarboxylate (Intermediate A-2-1, 44.94 g) and (4-(tert-butoxycarbonyl)phenyl) boronic acid (32.16 g, 144.8 mmol) in dioxane (922 mL). The mixture was evacuated/backfilled with N₂ twice. PdCl₂ (dtbpf) (3.63 g, 5.57 mmol) was added and the mixture was again evacuated with N₂. The RM was stirred at 40° C. for ˜16 h. The RM was filtered through Florisil® and the filtrate was partitioned between EtOAc and sat. aq NaHCO₃ solution (700 mL each). The separated org layer was washed with sat. aq NaHCO₃ solution and brine. The separated aq layer was extracted with EtOAc (700 mL). The combined org layers were dried over Na₂SO₄, filtered, and the filtrate was reduced to approx. ⅓ of its volume under reduced pressure. The mixture was stirred over Silicycle SiliaMet®-S DMT (20 g) for ˜1 h, filtered, and the filtrate was concentrated under reduced pressure. The crude material was purified by column chromatography over silica gel [750 g, EtOAc/heptane=0/100 to 70/30] to provide 1-(tert-butyl) 4-ethyl 5-(4-(tert-butoxycarbonyl)phenyl)-3,6-dihydropyridine-1,4(2H)-dicarboxylate (37.28 g) as a light yellow oil that solidified upon standing. ¹H NMR (400 MHZ, chloroform-d) δ [ppm] 7.96 (d, J=7.9 Hz, 2H), 7.23 (d, J=8.4 Hz, 2H), 4.18-4.07 (m, 2H), 3.92 (q, J=7.1 Hz, 2H), 3.66-3.55 (m, 2H), 2.60-2.51 (m, 2H), 1.59 (s, 9H), 1.48 (s, 9H), 0.91 (t, J=7.1 Hz, 3H).

### Step 3: racemic 1-(tert-butyl) 4-ethyl (3R*,4S*)-3-(4-(tert-butoxycarbonyl)phenyl)piperidine-1,4-dicarboxylate (Intermediate A-2-3)

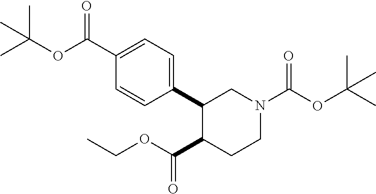

A mixture of (tert-butyl) 4-ethyl 5-(4-(tert-butoxycarbonyl)phenyl)-3,6-dihydropyridine-1,4(2H)-dicarboxylate (Intermediate A-2-2, 3.50 g, 8.11 mmol) and Pd/C (10 wt %, Degussa Type, 50% wet, 863.1 mg) in EtOH (150 mL) was degassed and then shaken under hydrogen atmosphere (45 psi; 500 mL Parr bottle) at ambient temperature for ˜14 h. The mixture was filtered through a Celite® pad (2×), and the pad was rinsed/washed with EtOH. The filtrate was concentrated under reduced pressure, and the resulting residue was purified by column chromatography over silica gel [80 g, EtOAc/heptane=0/100 to 60/40] to provide racemic 1-(tert-butyl) 4-ethyl (3R*,4S*)-3-(4-(tert-butoxycarbonyl)phenyl)piperidine-1,4-dicarboxylate (2.94 g) as a white solid.

¹H NMR (400 MHZ, chloroform-d) δ [ppm] 7.92-7.84 (m, 2H), 7.28-7.24 (m, 2H), 4.02 (dd, J=13.3, 8.1 Hz, 1H), 3.93 (dddd, J=18.0, 10.9, 7.1, 3.7 Hz, 2H), 3.84-3.66 (m, 2H), 3.59-3.48 (m, 1H), 3.21-3.13 (m, 1H), 3.02-2.93 (m, 1H), 2.04-1.93 (m, 1H), 1.90-1.82 (m, 1H), 1.58 (s, 9H), 1.44 (s, 9H), 1.05 (t, J=7.2 Hz, 3H).

### Step 4: racemic 1-(tert-butyl) 4-ethyl (3R*,4R*)-3-(4-(tert-butoxycarbonyl)phenyl)piperidine-1,4-dicarboxylate (Intermediate A-2-4)

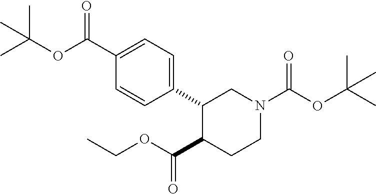

A solution of racemic 1-(tert-butyl) 4-ethyl (3R*,4S*)-3-(4-(tert-butoxycarbonyl)phenyl)piperidine-1,4-dicarboxylate (Intermediate A-2-3, 2.53 g, 5.84 mmol) in degassed EtOH (60 mL) was purged with N₂. A sodium ethoxide solution in EtOH (436 μL, 21 wt %, 1.167 mmol) was added and the resulting mixture was stirred at 50° C. for ˜8 h. The RM was allowed to cool to RT, stirred over night, and then concentrated under reduced pressure to ˜½ of its volume. The resulting mixture was diluted with sat. aq KH₂PO₄ solution (60 mL) and brine (20 mL) and then extracted with Me-THF (2×60 mL). The combined org layers were washed with brine (20 mL), dried over Na₂SO₄, filtered, and concentrated under reduced pressure resulting in two phases. The residue was diluted with Me-THF and the separated org phase was dried again over Na₂SO₄, filtered, and concentrated under reduced pressure. The residue was purified by column chromatography over silica gel [40 g, EtOAc/heptane=0/100 to 80/20] to provide racemic 1-(tert-butyl) 4-ethyl (3R*,4R*)-3-(4-(tert-butoxycarbonyl)phenyl)piperidine-1,4-dicarboxylate (1.93 g; containing ˜3% of bis-ethylester) as a colorless oil that solidified upon standing. ¹H NMR (400 MHZ, chloroform-d) δ [ppm] 7.93-7.91 (m, 2H), 7.28-7.25 (m, 2H), 4.39-4.04 (m, 2H), 3.98-3.85 (m, 2H), 3.03-2.97 (m, 1H), 2.84-2.65 (m, 3H), 2.01-1.96 (m, 1H), 1.81-1.70 (m, 1H), 1.57 (s, 9H), 1.45 (s, 9H), 0.99 (t, J=7.1 Hz, 3H).

### Step 5: racemic tert-butyl (3R*,4R*)-3-(4-(tert-butoxycarbonyl)phenyl)-4-(hydroxymethyl)piperidine-1-carboxylate (Intermediate A-2-5)

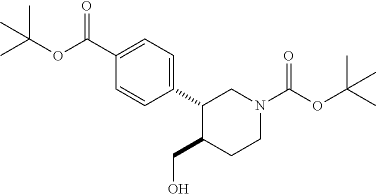

To a solution of racemic 1-(tert-butyl) 4-ethyl (3R*,4R*)-3-(4-(tert-butoxycarbonyl)phenyl)piperidine-1,4-dicarboxylate (Intermediate A-2-4, 1.92 g, 4.43 mmol) in THF (23 mL) and MeOH (269 μL, 6.643 mmol) was added a freshly prepared LiBH₄ solution in THF (2M, 5.54 mL, 11.07 mmol) at 0° C. The mixture was stirred at ambient temperature and warmed up to 35° C. for ˜14 h. MeOH (50 μL) and LiBH₄ solution in THF (2M, 1 mL, 2.0 mmol) were added, and stirring was continued for 2 h. The RM was cooled to 0° C. and was carefully diluted with sat. aq KH₂PO₄ solution (80 mL) and brine (30 mL). The separated aq layer was extracted with EtOAc (2×60 mL, 30 mL). The combined org layers were dried over Na₂SO₄, filtered, and concentrated under reduced pressure. The residue was purified by column chromatography over silica gel [40 g, EtOAc/heptane=0/100 to 60/40] to provide racemic tert-butyl (3R*,4R*)-3-(4-(tert-butoxycarbonyl)phenyl)-4-(hydroxymethyl)piperidine-1-carboxylate (1.22 g) as a white solid and recovered Intermediate A-2-4 (335 mg). ¹H NMR (400 MHZ, chloroform-d) δ [ppm] 7.95-7.93 (m, 2H), 7.26 (d, J=8.4 Hz, 2H), 4.37-4.04 (m, 2H), 3.44-3.40 (m, 1H), 3.27-3.23 (m, 1H), 2.86-2.73 (m, 2H), 2.62-2.55 (m, 1H), 1.97-1.87 (m, 2H), 1.59 (s, 9H), 1.48-1.07 (m, 10H). Reported 32 of 33 protons.

### Step 6: tert-butyl (3R,4R-3-(4-(tert-butoxycarbonyl)phenyl)-4-(hydroxymethyl)piperidine-1-carboxylate (Intermediate A-2) and tert-butyl (3S,4S-3-(4-(tert-butoxycarbonyl)phenyl)-4-(hydroxymethyl)piperidine-1-carboxylate (Intermediate A-2-6)

Enantiomer separation and analytics according to Method SFC-3. racemic tert-butyl (3R*,4R*)-3-(4-(tert-butoxycarbonyl)phenyl)-4-(hydroxymethyl)piperidine-1-carboxylate (Intermediate A-2-5, 3.70 g) provided Intermediate A-2 (Peak 1): 1.70 g, white solid. LC-MS Method B-1: Rt=1.31 min; MS m/z [M+Na]⁺=414.3; analytical chiral HPLC: Rt=2.35 min; 99.5% ee. Intermediate A-2-6 (Peak 2): 1.73 g, white solid. LC-MS Method B-1: Rt=1.30 min; MS m/z [M+Na]⁺=414.2; analytical chiral HPLC: Rt=1.42 min; 99.5% ee.

### Intermediate rac-A-3

### racemic tert-butyl (3R*,4S*)-3-(4-cyanophenyl)-4-(hydroxymethyl)pyrrolidine-1-carboxylate

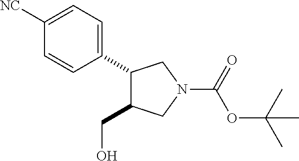

Racemic (3S*,4R*)-1-(tert-butoxycarbonyl)-4-(4-cyanophenyl)pyrrolidine-3-carboxylic acid (CAS #1217702-50-5, 1.03 g, 3.25 mmol) was dissolved in THF (10 mL) and cooled to 0° C. Borane-methyl sulfide complex (0.61 mL, 6.5 mmol) was added in a dropwise manner and the RM was stirred at 0° C. for 30 min, then stirring was continued at RT for 3 h. The mixture was cooled to 0° C., quenched with MeOH (10 mL), and the resulting mixture was concentrated under reduced pressure. The residue was purified by chromatography over silica gel (24 g, EtOAc/heptane=0/100 to 80/20) to afford racemic tert-butyl (3R*,4S*)-3-(4-cyanophenyl)-4-(hydroxymethyl)pyrrolidine-1-carboxylate (1.01 g) as a white solid. LCMS Method C-2: RT=0.93 min; MS m/z [M-tBu+H]⁺=246.9. ¹H NMR (400 MHZ, chloroform-d) δ [ppm] 7.68-7.60 (m, 2H), 7.43-7.34 (m, 2H), 3.87 (dd, J=11.0, 7.9 Hz, 1H), 3.77 (dd, J=11.0, 7.9 Hz, 1H), 3.68 (dd, J=10.8, 4.7 Hz, 1H), 3.57 (dd, J=10.8, 6.3 Hz, 1H), 3.46-3.19 (m, 3H), 2.62-2.41 (m, 1H), 1.49 (s, 9H).

### Intermediate A-4

### tert-butyl (3R,4R)-3-(4-(tert-butoxycarbonyl)phenyl)-4-(hydroxymethyl)azepane-1-carboxylate

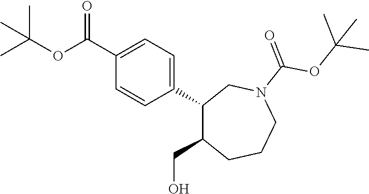 [SMILES: CC(C)(C)OC(=O)c1ccc([C@@H]2CN(C(=O)OC(C)(C)C)CCC[C@H]2CO)cc1]

### Step 1: 1-(tert-butyl) 4-ethyl (3R,4R)-3-(4-(tert-butoxycarbonyl)phenyl)azepane-1,4-dicarboxylate (Intermediate A-4-1)

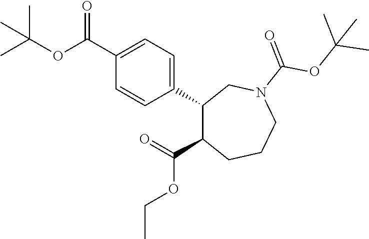 [SMILES: CCOC(=O)[C@@H]1CCCN(C(=O)OC(C)(C)C)C[C@H]1c1ccc(C(=O)OC(C)(C)C)cc1]

Step 1-a: To a solution of 1-(tert-butyl) 4-ethyl 2,5,6,7-tetrahydro-1H-azepine-1,4-dicarboxylate (CAS #2254213-29-9; prepared from 1-tert-butyl 4-ethyl 3-oxoazepane-1,4-dicarboxylate, CAS #98977-38-9, similar to as described in Tetrahedron Lett. 2018, 59, 4611; 2.370 g, 8.799 mmol) in degassed MeOH/water (4/1, 50 mL) were added (4-(tert-butoxycarbonyl)phenyl) boronic acid (3.517 g, 15.84 mmol), (1S,4S)-2,5-diphenylbicyclo[2.2.2]octa-2,5-diene (CAS #850409-83-5, 68.20 mg, 0.264 mmol), 1,1,3,3-tetramethylguanidine (608.1 and mg, 5.280 mmol) acetylacetonatobis(ethylene)rhodium(I) (CAS #12082-47-2, 68.14 mg, 0.264 mmol). The yellow solution was heated to 65° C. inner temperature under N₂ atmosphere and stirred for 45 min. The mixture was allowed to cool to RT and then diluted with TBME (100 mL) and brine (100 mL). The separated org layer was washed with brine (100 mL), and the separated aq layer was washed with TBME (100 mL). The combined org layers were dried over Na₂SO₄, filtered, and concentrated under reduced pressure at 45° C. The residue was purified by column chromatography over silica gel (220 g, EtOAc/heptane=0/100 to 50/50) to provide 1-(tert-butyl) 4-ethyl (3R)-3-(4-(tert-butoxycarbonyl)phenyl)azepane-1,4-dicarboxylate (4.10 g) as a yellow oil and a trans/cis mixture in a ratio of 65/35. LC-MS Method B-1: Rt=1.49/1.54 min; MS m/z [M-Boc+H]⁺=348.4/348.3.

Step 1-b: To a solution of the trans/cis mixture 1-(tert-butyl) 4-ethyl (3R)-3-(4-(tert-butoxycarbonyl)phenyl)azepane-1,4-dicarboxylate from Step 1-a (4.10 g, 9.16 mmol) in THF (80 mL) at −60° C. inner temperature was added a solution of potassium tert-butoxide in THF (1M, 4.58 mL, 4.58 mmol) dropwise over 1 min. The RM was stirred at −60° C. for 30 min, then poured into a stirred mixture of sat. aq NaHCO₃ solution (100 mL) and TBME (100 mL). After stirring for 2 min. the separated org layer was washed with brine (100 mL), and the the separated aq layer was extracted with TBME (100 mL). The combined org layers were dried over Na₂SO₄, filtered, and concentrated under reduced pressure at 45° C. to provide enriched 1-(tert-butyl) 4-ethyl (3R,4R)-3-(4-(tert-butoxycarbonyl)phenyl)azepane-1,4-dicarboxylate (4.11 g) as a pale yellow oil and as a trans/cis mixture in a ratio of 94/6 which was directly used without further purification. LC-MS Method B-1: Rt=1.51/1.46 min; MS m/z [M-Boc+H]⁺=348.3/348.3.

### Step 2 tert-butyl (3R,4R)-3-(4-(tert-butoxycarbonyl)phenyl)-4-(hydroxymethyl)azepane-1-carboxylate (Intermediate A-4)

Step 2-a: To a solution of enriched 1-(tert-butyl) 4-ethyl (3R,4R)-3-(4-(tert-butoxycarbonyl)phenyl)azepane-1,4-dicarboxylate (Intermediate A-4-1, 4.10 g, 9.17 mmol) in THF (50 mL) was added MeOH (926 μL, 22.90 mmol). The mixture was cooled to 0° C. and then a solution of lithium borohydride in THF (2M, 11.45 mL, 22.9 mmol) was added dropwise over 2 min. The RM was heated to 35-38° C. (internal temperature) and stirred overnight. To the RM was added more MeOH (926 μL), and stirring was continued for 1 h at 38° C. Additional MeOH (926 μL) was added. and stirring was continued for an additional 1 h at 38° C. The RM was cooled to ˜10° C., poured into a stirred mixture of TBME (100 mL) and sat. aq NaHCO₃ solution (100 mL), and stirred for 15 min at ambient temperature. The separated org phase was washed with sat. aq NaHCO₃ solution (100 mL) and with brine (100 mL), and the separated aq layers was washed with TBME (100 mL). The combined org layers were dried over Na₂SO₄, filtered, and concentrated under reduced pressure. The residue was purified by column chromatography over silica gel (220 g, EtOAc/heptane=15/85 to 100/0) to provide enriched tert-butyl (3R,4R)-3-(4-(tert-butoxycarbonyl)phenyl)-4-(hydroxymethyl)azepane-1-carboxylate (2.81 g) as a white foam. LC-MS Method B-1: Rt=1.31 min; MS m/z [M-Boc+H]⁺=306.4.

Step 2-b: Chiral Purification and analytics according to Method SFC-2: Enriched tert-butyl (3R,4R)-3-(4-(tert-butoxycarbonyl)phenyl)-4-(hydroxymethyl)azepane-1-carboxylate from Step 2-a (2.75 g) provided Intermediate A4 (2.29 g) as white crystalline material. LC-MS Method B-2: Rt=6.33 min; MS m/z [M-Boc+H-tBu+H]⁺=250.1. Chiral HPLC Method SFC-2: Rt=2.11 min; 100% ee. ¹H NMR (600 MHZ, DMSO-d₆) δ [ppm] 7.86-7.81 (m, 2H), 7.33 (d, J=8.03 Hz, 2H), 4.37-4.30 (m, 1H), 3.73-3.56 (m, 1H), 3.46-3.39 (m, 1H), 3.18-3.00 (m, 3H), 2.91-2.85 (m, 1H), 2.67-2.59 (m, 1H), 2.08-2.00 (m, 1H), 2.00-1.95 (m, 1H), 1.76-1.71 (m, 2H), 1.54 (s, 9H), 1.42-1.37 (m, 9H), 1.30-1.15 (m, 1H).

### Intermediate A-5

### tert-butyl (4R,5R)-4-(4-(tert-butoxycarbonyl)phenyl)-5-(hydroxymethyl)azepane-1-carboxylate

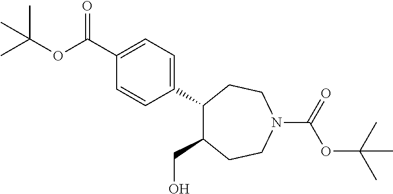 [SMILES: CC(C)(C)OC(=O)c1ccc([C@@H]2CCN(C(=O)OC(C)(C)C)CC[C@H]2CO)cc1]

Intermediate A-5 was prepared according to the method described for Intermediate A-4 herein above, using 1-(tert-butyl) 4-ethyl 2,3,6,7-tetrahydro-1H-azepine-1,4-dicarboxylate (CAS #912444-89-4).

Chiral Purification and analytics according to Method SFC-5:

LC-MS Method B-2: Rt=6.41 min; MS m/z [M-Boc+H]⁺=306.2.

Chiral HPLC Method: Rt=2.79 min; 100% ee.

¹H NMR (600 MHZ, DMSO-d₆) δ [ppm] 7.81 (dd, J=8.3, 2.7 Hz, 2H), 7.35-7.18 (m, 2H), 4.39-4.28 (m, 1H), 3.71-3.49 (m, 2H), 3.41-3.30 (m, 1H, partially overlapping with water peak), 3.26-3.12 (m, 1H), 3.00-2.91 (m, 1H), 2.84-2.77 (m, 1H), 2.55-2.47 (m, 1H, partially overlapping with solvent peak), 2.08 (d, J=4.3 Hz, 1H), 1.87-1.77 (m, 1H), 1.77-1.72 (m, 1H), 1.72-1.67 (m, 1H), 1.64-1.55 (m, 1H), 1.53 (s, 9H), 1.41 (d, J=12.1 Hz, 9H).

### Intermediate A-6

### benzyl (3R,4R)-3-(4-cyanophenyl)-4-(hydroxymethyl)piperidine-1-carboxylate

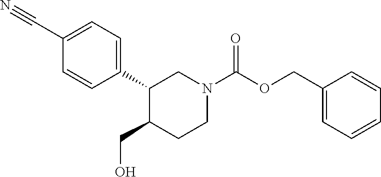 [SMILES: N#Cc1ccc([C@@H]2CN(C(=O)OCc3ccccc3)CC[C@H]2CO)cc1]

### Step 1: 1-benzyl 4-methyl 3-(4-carbamoylphenyl)piperidine-1,4-dicarboxylate (Intermediate A-6-1)

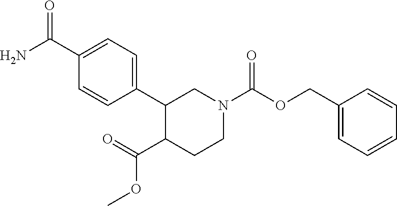 [SMILES: COC(=O)C1CCN(C(=O)OCc2ccccc2)CC1c1ccc(C(N)=O)cc1]

Intermediate A-6-1 was prepared according to the method described for Intermediate A-4 (Step 1-a) herein above, using 1-benzyl 4-methyl 3,6-dihydropyridine-1,4(2H)-dicarboxylate (CAS #1784422-91-8) and (4-carbamoylphenyl) boronic acid (CAS #123088-59-5). LC-MS Method B-2: Rt=3.95/4.09 min (diastereomeric mixture); MS m/z [M+H]⁺=397.1.

### Step 2: 1-benzyl 4-methyl 3-(4-cyanophenyl)piperidine-1,4-dicarboxylate (Intermediate A-6-2)

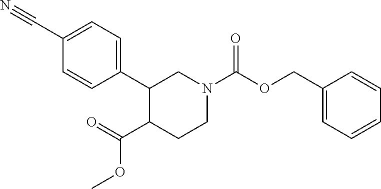 [SMILES: COC(=O)C1CCN(C(=O)OCc2ccccc2)CC1c1ccc(C#N)cc1]

To a solution of the cis/trans mixture of 1-benzyl 4-methyl 3-(4-carbamoylphenyl)piperidine-1,4-dicarboxylate (Intermediate A-6-1, 4.250 g, 10.72 mmol), and TEA (4.339 g, 42.88 mmol) in DCM (150 mL) at 0 to 5° C. was added TFAA (4.50 g, 21.44 mmol) in a dropwise manner over ˜5 min. The RM was stirred additional 5 min at 0 to 5° C., the ice bath was removed, and stirring was continued for ˜1 h at ambient temperature. The mixture was poured into sat. aq NaHCO₃ solution (200 mL) and vigorously stirred for 5 min. The separated org phase was washed with sat. aq NaHCO₃ solution (100 mL), and the aq layer was washed DCM (100 mL). The combined organic layers were dried over Na₂SO₄, filtered, and concentrated under reduced pressure at 45° C. The residue was purified by column chromatography over silica gel (220 g, EtOAc/heptane=15/85 to 100/00) to provide the cis/trans mixture of 1-benzyl 4-methyl 3-(4-cyanophenyl)piperidine-1,4-dicarboxylate (4.28 g) as a yellow oil. LC-MS Method B-2: Rt=4.93/5.04 min; MS m/z [M+H]⁺=379.3.

### Step 3: benzyl (3R,4R)-3-(4-cyanophenyl)-4-(hydroxymethyl)piperidine-1-carboxylate (Intermediate A-6)

Intermediate A-6 was prepared according to the method described for Intermediate A-4 herein above, Step 1-a and Step 2.

Chiral Purification and analytics according to Method SFC-6:

LC-MS Method B-2: Rt=4.50 min; MS m/z [M+H]⁺=351.2.

Chiral HPLC Method: Rt=1.88 min; 100% ee.

¹H NMR (500 MHZ, DMSO-d₆) δ [ppm] ¹H NMR (500 MHZ, DMSO-d₆) δ [ppm] 7.79 (d, J=7.7 Hz, 2H), 7.48 (d, J=7.8 Hz, 2H), 7.44-7.25 (m, 5H), 5.09 (s, 2H), 4.43-4.34 (m, 1H), 4.13 (d, J=13.1 Hz, 1H), 4.00-3.83 (m, 1H), 3.14-3.07 (m, 1H), 3.06-2.83 (m, 3H), 2.64-2.56 (m, 1H), 1.99-1.80 (m, 2H), 1.44-1.22 (m, 1H).

### Intermediate A-7

### benzyl (3R,4R)-3-(4-bromophenyl)-4-(hydroxymethyl)piperidine-1-carboxylate

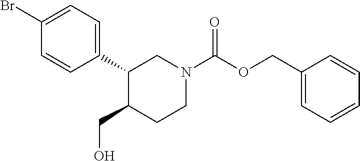 [SMILES: O=C(OCc1ccccc1)N1CC[C@@H](CO)[C@H](c2ccc(Br)cc2)C1]

Intermediate A-7 was prepared according to the method described for Intermediate A-4 herein above, using 1-benzyl 4-methyl 3,6-dihydropyridine-1,4(2H)-dicarboxylate (CAS #1784422-91-8) and (4-bromophenyl) boronic acid (CAS #5467-74-3).

Chiral Purification and analytics according to Method SFC-7:

LC-MS Method B-2: Rt=6.11 min; MS m/z [M+H]⁺=404.0.

Chiral HPLC Method: Rt=1.54 min; 100% ee.

¹H NMR (500 MHZ, DMSO-d₆) δ [ppm] 7.50 (d, J=8.0 Hz, 2H), 7.43-7.27 (m, 5H), 7.22 (d, J=7.9 Hz, 2H), 5.08 (s, 2H), 4.38-4.29 (m, 1H), 4.23-4.05 (m, 1H), 3.90 (d, J=12.8 Hz, 1H), 3.12 (ddd, J=10.6, 4.8, 2.8 Hz, 1H), 3.04-2.79 (m, 3H), 2.46 (dd, J=11.3, 4.2 Hz, 1H), 1.94-1.75 (m, 2H), 1.42-1.20 (m, 1H).

### Intermediate A-8

### benzyl (3R,4R)-3-(4-(1H-pyrazol-1-yl)phenyl)-4-(hydroxymethyl)piperidine-1-carboxylate

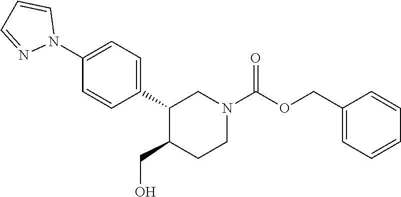 [SMILES: O=C(OCc1ccccc1)N1CC[C@@H](CO)[C@H](c2ccc(-n3cccn3)cc2)C1]

A mixture of benzyl (3R,4R)-3-(4-bromophenyl)-4-(hydroxymethyl)piperidine-1-carboxylate (Intermediate A-7, 1.810 g, 4.477 mmol), 1H-pyrazole (914.4 mg, 13.43 mmol), rac-trans-N,N′dimethylcyclohexane-1,2-diamine (254.7 mg, 0.895 mmol), cesium carbonate (4.376, 13.43 mmol), and DMF (18.0 mL) in a sealed vial was evacuated/back-filled with N₂ twice. Copper iodide (170.5 mg, 0.895 mmol) was added, and the mixture was evacuated/back-filled with N₂ once more. The RM was stirred at 120° C. over 3 days. The mixture was cooled to RT, diluted with TBME and water, and filtered over Celite®. The layers of the filtrate were separated, and the org phase was washed with 1M aq NaOH solution, water, brine, and the aq phase was extracted with TBME. The combined organic layers were dried over MgSO₄, filtered, and the volatiles were removed under reduced pressure at 50° C. The residue was purified by column chromatography over silica gel (80 g, heptane/EtOAc=90/10 t0 30/70) to provide benzyl (3R,4R)-3-(4-(1H-pyrazol-1-yl)phenyl)-4-(hydroxymethyl)piperidine-1-carboxylate (1.06 g) as a yellow oil. LC-MS Method B-1: Rt=1.01 min; MS m/z [M+H]⁺=392.2.

### Intermediate A-9

### tert-butyl (3R,4R)-4-(hydroxymethyl)-3-(1-methyl-1H-pyrazol-4-yl)piperidine-1-carboxylate

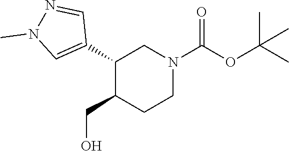 [SMILES: Cn1cc([C@@H]2CN(C(=O)OC(C)(C)C)CC[C@H]2CO)cn1]

### Step 1: 1-(tert-butyl) 4-ethyl 5-(1-methyl-1H-pyrazol-4-yl)-3,6-dihydropyridine-1,4(2H)-dicarboxylate (Intermediate A-9-1)

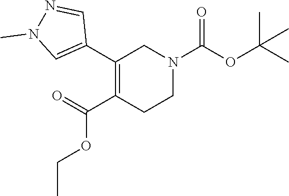 [SMILES: CCOC(=O)C1=C(c2cnn(C)c2)CN(C(=O)OC(C)(C)C)CC1]

A solution 1-(tert-butyl) 4-ethyl 5-(((trifluoromethyl)sulfonyl)oxy)-3,6-dihydropyridine-1,4(2H)-dicarboxylate (Intermediate A-2-1, 5.9 g, 14.64 mmol), (1-methyl-1H-pyrazol-4-yl) boronic acid (CAS #847818-55-7, 4.55 g, 21.9 mmol), and cesium carbonate (9.5 g, 29.2 mmol) in dioxane (50 mL) and H₂O (6 mL) was degassed with N₂ for 30 min. XPhos Pd G2 (CAS #1310584-14-5, 0.57 g, 0.73 mmol) was added, and the RM was stirred for 12 h at 80° C. The mixture was filtered through Celite®, washing with water. The organic phase was dried over Na₂SO₄, concentrated, and purified via chromatography over silica gel (40 g, MeOH/DCM 0-10%) to provide 1-(tert-butyl) 4-ethyl 5-(1-methyl-1H-pyrazol-4-yl)-3,6-dihydropyridine-1,4(2H)-dicarboxylate (4.8 g) as a yellow oil. LC-MS Method A-1: Rt=1.69 min; MS m/z [M+H]⁺=336.9.

### Step 2: racemic 1-(tert-butyl) 4-ethyl (3R*,4S*)-3-(1-methyl-1H-pyrazol-4-yl)piperidine-1,4-dicarboxylate (Intermediate A-9-2)

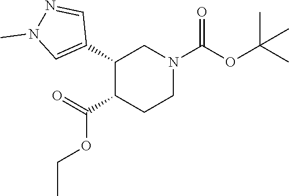 [SMILES: CCOC(=O)[C@H]1CCN(C(=O)OC(C)(C)C)C[C@H]1c1cnn(C)c1]

A mixture of 1-(tert-butyl) 4-ethyl 5-(1-methyl-1H-pyrazol-4-yl)-3,6-dihydropyridine-1,4(2H)-dicarboxylate (Intermediate A-9-1) and platinum oxide (10 wt % on carbon, 5 g) in EtOH (48 mL) was stirred under 1 atm hydrogen pressure for 16 hr at RT. The RM was filtered through a pad of Celite®, concentrated, and purified via chromatography over silica gel (40 g, EtOAc/heptane 0-20%) to yield racemic 1-(tert-butyl) 4-ethyl (3R*,4S*)-3-(1-methyl-1H-pyrazol-4-yl)piperidine-1,4-dicarboxylate (4.1 g) as a yellow oil. LC-MS Method A-1: Rt=1.78 min; MS m/z [M+H]⁺=338.5.

### Step 3: racemic 1-(tert-butyl) 4-ethyl (3R*,4R*)-3-(1-methyl-1H-pyrazol-4-yl)piperidine-1,4-dicarboxylate (Intermediate A-9-3)

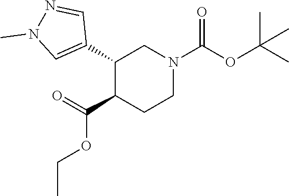 [SMILES: CCOC(=O)[C@@H]1CCN(C(=O)OC(C)(C)C)C[C@H]1c1cnn(C)c1]

To a solution of racemic 1-(tert-butyl) 4-ethyl (3R*,4R*)-3-(1-methyl-1H-pyrazol-4-yl)piperidine-1,4-dicarboxylate (Intermediate A-9-2, 3.2 g, 9.78 mmol) in toluene (30 mL) was added DBU (4.1 mL, 29.34 mmol). The RM was stirred at 110° C. for 16 h and then quenched with water. The mixture was extracted with EtOAc. The organic phase was dried over Na₂SO₄, concentrated under reduced pressure, and purified by chromatography over silica gel (40 g, EtOAc/heptane 0-20%) to yield racemic 1-(tert-butyl) 4-ethyl (3R*,4R*)-3-(1-methyl-1H-pyrazol-4-yl)piperidine-1,4-dicarboxylate as a colorless oil and as a diastereomeric mixture with recovered Intermediate A-9-2 which was directly used without further purification. LC-MS Method A-2: Rt=2.28 min; MS m/z [M+H]⁺=338.2.

### Step 4

### tert-butyl (3R,4R)-4-(hydroxymethyl)-3-(1-methyl-1H-pyrazol-4-yl)piperidine-1-carboxylate (Intermediate A-9)

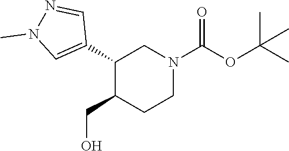 [SMILES: Cn1cc([C@@H]2CN(C(=O)OC(C)(C)C)CC[C@H]2CO)cn1]

A solution of racemic 1-(tert-butyl) 4-ethyl (3R*,4R*)-3-(1-methyl-1H-pyrazol-4-yl)piperidine-1,4-dicarboxylate (Intermediate A-9-3, as a mixture with Intermediate A-9-2, 3 g, 8.89 mmol) in THF (30 mL) was cooled to 0° C. under a N₂ atmosphere. Lithium aluminium hydride solution (1M in THF, 10 mL, 10 mmol) was added, and the RM was stirred at 0° C. for 2 h. The mixture was quenched with EtOAc at 0° C., and then H₂O was added. The organic phase was collected, and the aq phase was extracted with EtOAc. The combined organic phases were dried over Na₂SO₄ and concentrated to yield racemic tert-butyl (3R*,4R*)-4-(hydroxymethyl)-3-(1-methyl-1H-pyrazol-4-yl)piperidine-1-carboxylate as a mixture with racemic tert-butyl (3R*,4S*)-4-(hydroxymethyl)-3-(1-methyl-1H-pyrazol-4-yl)piperidine-1-carboxylate.

Chiral purification and analytics according to Method C-10:

LC-MS Method A-1: Rt=1.70 min; MS m/z [M+H]⁺=296.5.

Chiral HPLC Method: Rt=3.61 min; 97.9% ee.

¹H NMR (400 MHZ, DMSO-d₆) δ [ppm] 7.51 (s, 1H), 7.27 (s, 1H), 4.32-4.28 (m, 1H) 4.06-3.95 (m, 1H), 3.93-3.82 (m, 1H), 3.77 (s, 3H) 3.30-3.24 (m, 1H), 3.12-3.02 (m, 1H) 2.86-2.62 (m, 2H), 2.39-2.28 (m, 1H), 1.73-1.83 (m, 1H), 1.59-1.45 (m, 1H), 1.38 (s, 9H), 1.29-1.22 (m, 1H)

### Intermediate F-1

### potassium (((3R,4R)-1-(tert-butoxycarbonyl)-3-(4-(tert-butoxycarbonyl)phenyl)piperidin-4-yl)methyl)trifluoroborate

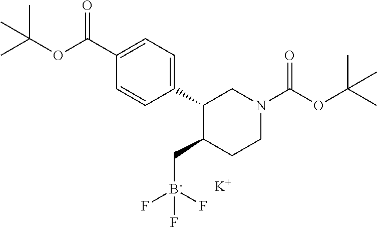 [SMILES: CC(C)(C)OC(=O)c1ccc([C@@H]2CN(C(=O)OC(C)(C)C)CC[C@H]2C[B-](F)(F)F)cc1 ; [K+]]

### Step 1: tert-butyl (3R,4R)-4-(bromomethyl)-3-(4-(tert-butoxycarbonyl)phenyl)piperidine-1-carboxylate (Intermediate F-1-1)

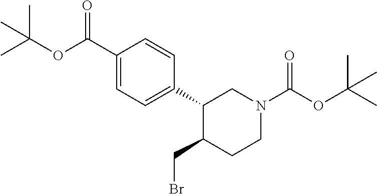 [SMILES: CC(C)(C)OC(=O)c1ccc([C@@H]2CN(C(=O)OC(C)(C)C)CC[C@H]2CBr)cc1]

A suspension of tert-butyl (3R,4R)-3-(4-(tert-butoxycarbonyl)phenyl)-4-(hydroxymethyl)-piperidine-1-carboxylate (Intermediate A-2, 1.630 g, 4.163 mmol) in ACN (64.5 mL) and DCM (5.0 mL) was dissolved by heating the mixture to about 45° C. The mixture was allowed to cool to RT and PPh₃ (1.420 g, 5.412 mmol) was added. The mixture was cooled to 3° C. and CBr₄ (1.795 g, 5.412 mmol) was added in one portion. The RM was stirred in the ice bath for 30 min, then for 2 h 45 min at RT. Additional PPh₃ (328 mg) and CBr₄ (414 mg) were added and stirring was continued for 90 min at RT. The volatiles were removed under reduced pressure at 35° C. The residue was purified over silica gel (40 g) using an automated purification system (NP; Teledyne ISCO®; collected at 245/260 nm; flow 40 mL/min; eluent: heptane+5.0 to 25% EtOAc in 20 min) to provide tert-butyl (3R,4R)-4-(bromomethyl)-3-(4-(tert-butoxycarbonyl)phenyl)piperidine-1-carboxylate (1.38 g) as a colorless oil. LC-MS Method B-2: Rt=7.95 min; MS m/z [M-tBu+H-tBu+H]⁺=342.0/344.0.

### Step 2: tert-butyl (3R,4R)-3-(4-(tert-butoxycarbonyl)phenyl)-4-((4,4,5,5-tetramethyl-1,3,2-dioxaborolan-2-yl)methyl)piperidine-1-carboxylate (Intermediate F-1-2)

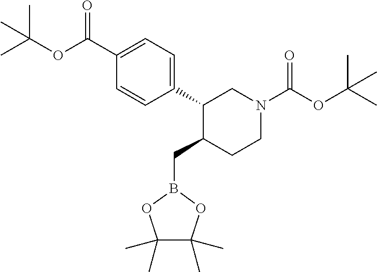 [SMILES: CC(C)(C)OC(=O)c1ccc([C@@H]2CN(C(=O)OC(C)(C)C)CC[C@H]2CB2OC(C)(C)C(C)(C)O2)cc1]

Water (875.6 mg, 875.6 μL, 48.59 mmol) was added to a mixture of tert-butyl (3R,4R)-4-(bromomethyl)-3-(4-(tert-butoxycarbonyl)phenyl)piperidine-1-carboxylate (Intermediate F-1-1, 1.380 g, 3.037 mmol) and bis(pinacolato)diborane (2.121 g, 8.352 mmol) in tert-BuOH (10.50 mL). The mixture was evacuated/back-filled with N₂. Bis(1,1-dimethylethyl)(methyl)phosphine tetrafluoroborate (CAS #479094-62-7, 135.6 mg, 546.7 μmol), Pd₂(dba)₃ (83.43 mg, 91.11 μmol) and K₃PO₄ (1.289 g, 6.074 mmol) were added subsequently. The RM was evacuated/back-filled with N₂ twice and heated under N₂ atmosphere at 65° C. overnight. The mixture was allowed to cool to RT, diluted with TBME, and poured into a well stirred mixture of aq NH₄Cl soln. (10 wt %, 10 mL) and TBME (10 mL). The resulting black slurry was filtered over Celite® and the solid was rinsed with TBME (2×). The layers of the filtrate were separated and washed with brine and TBME. The combined org layers were dried over MgSO₄, filtered, and concentrated under reduced pressure at 50° C. The residue was purified over silica gel (40 g) using an automated purification system (NP; Teledyne ISCO®; collected at 250/260 nm; flow 40 mL/min; eluent: heptane+3.0 to 19.5% EtOAc in 15 min) to provide tert-butyl (3R,4R)-3-(4-(tert-butoxycarbonyl)phenyl)-4-((4,4,5,5-tetramethyl-1,3,2-dioxaborolan-2-yl)methyl)piperidine-1-carboxylate as a yellow foam (1.35 g). LC-MS Method B-2: Rt=8.14 min; MS m/z [M-Boc+H-tBu+H]⁺=346.4.

### Step 3: potassium (((3R,4R)-1-(tert-butoxycarbonyl)-3-(4-(tert-butoxycarbonyl)phenyl)piperidin-4-yl)methyl)trifluoroborate (Intermediate F-1)

A soln. of tert-butyl (3R,4R)-3-(4-(tert-butoxycarbonyl)phenyl)-4-((4,4,5,5-tetramethyl-1,3,2-dioxaborolan-2-yl)methyl)piperidine-1-carboxylate (Intermediate F-1-2, 1.220 g, 2.433 mmol) in MeOH (20.0 mL) was put into a water bath at RT. A soln. of KHF₂ (950.0 mg, 439.0 μL, 12.16 mmol) in H₂O (10.0 mL) was added via dropping-funnel and additional MeOH (5 mL) was added to ensure stirring. After stirring for 90 min at RT, the RM was diluted with MeOH, evaporated to dryness, the residue was taken up in MeOH, and the volatiles were removed under reduced pressure at 50° C. The crude yellow solid was suspended in acetone, heated to 50° C., treated with ultrasound, filtered, and the volatiles of the filtrate were removed under reduced pressure at 50° C. to give potassium (((3R,4R)-1-(tert-butoxycarbonyl)-3-(4-(tert-butoxycarbonyl)phenyl)piperidin-4-yl)methyl)trifluoroborate (1.13 g) as an off-white solid. LC-MS Method B-2: Rt=6.40 min; MS m/z [M-K+Na]⁻=464.3.

### Intermediate rac-F-2

### Racemic potassium (((3S*,4R*)-1-(tert-butoxycarbonyl)-4-4-cyanophenyl)pyrrolidin-3-yl)methyl)trifluoroborate

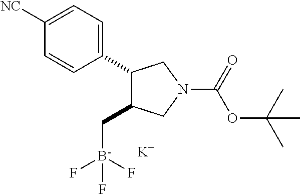

### Step 1: racemic tert-butyl (3S*,4R*)-3-(bromomethyl)-4-(4-cyanophenyl)pyrrolidine-1-carboxylate (Intermediate rac-F-2-1)

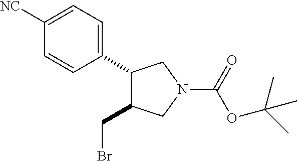

Racemic tert-butyl (3R*,4S*)-3-(4-cyanophenyl)-4-(hydroxymethyl)pyrrolidine-1-carboxylate (Intermediate rac-A-3, 984 mg, 3.25 mmol) was dissolved in DCM (10 mL) and triphenylphosphine (1.11 g, 4.23 mmol) was added. The RM was cooled to 0° C., CBr₄ (1.40 g, 4.23 mmol) was added, and the RM was stirred at 0° C. for 10 min, then at RT for 2 h. The RM was concentrated under reduced pressure and the residue was purified by chromatography over silica gel (40 g, EtOAc/heptane=0/100 to 60/40) to afford racemic tert-butyl (3S*,4R*)-3-(bromomethyl)-4-(4-cyanophenyl)pyrrolidine-1-carboxylate (977 mg) as a colorless oil. LCMS Method C-1: RT=1.17 min; MS m/z [M-tBu+H]⁺=311.2. ¹H NMR (400 MHZ, chloroform-d) δ [ppm] 7.71-7.63 (m, 2H), 7.44-7.35 (m, 2H), 3.97-3.86 (m, 1H), 3.87-3.78 (m, 1H), 3.51-3.40 (m, 2H), 3.40-3.22 (m, 3H), 2.70-2.56 (m, 1H), 1.45 (s, 9H).

### Step 2: racemic tert-butyl (3R*,4S*)-3-(4-cyanophenyl)-4-((4,4,5,5-tetramethyl-1,3,2-dioxaborolan-2-yl)methyl)pyrrolidine-1-carboxylate (Intermediate rac-F-2-2)

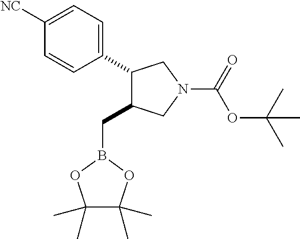

To racemic tert-butyl (3S*,4R*)-3-(bromomethyl)-4-(4-cyanophenyl)pyrrolidine-1-carboxylate (Intermediate rac-F-2-1, 777 mg, 2.13 mmol), bis(pinacolato)diboron (902 mg, 3.55 mmol), K₃PO₄ (993 mg, 4.68 mmol), Pdz (dba)₃ (58 mg, 64 μmol) and bis(1,1-dimethylethyl)(methyl)phosphine tetrafluoroborate (95 mg, 38 μmol) in a vial and under N₂ atmosphere were added tert-BuOH (7 mL) and water (0.6 mL). The RM was purged with N₂, then stirred at 65° C. overnight. The RM was filtered through a syringe filter (WHATMAN® 0.45 mm PTFE w/GMF 25 mm diameter), then the filtrate was diluted with DCM and sat. aq NaHCO₃ soln. The separated aq layer was extracted with DCM (3×). The combined org layers were dried though an Isolute® phase separator and concentrated under reduced pressure. The residue was purified by chromatography over silica gel (24 g, EtOAc/heptane=0/100 to 50/50) to afford enriched racemic tert-butyl (3R*,4S*)-3-(4-cyanophenyl)-4-((4,4,5,5-tetramethyl-1,3,2-dioxaborolan-2-yl)methyl)pyrrolidine-1-carboxylate (937.0 mg) which was directly used in the next step without further purification. LCMS Method C-1: RT=1.17 min; MS m/z [M-Boc+H]⁺=313.2.

### Step 3: Racemic potassium (((3S*,4R*)-1-(tert-butoxycarbonyl)-4-(4-cyanophenyl)pyrrolidin-3-yl)methyl)trifluoroborate (Intermediate rac-F-2)

Enriched racemic tert-butyl (3R*,4S*)-3-(4-cyanophenyl)-4-((4,4,5,5-tetramethyl-1,3,2-dioxaborolan-2-yl)methyl)pyrrolidine-1-carboxylate (Intermediate rac-F-2-2, 937 mg, 2.27 mmol) was dissolved in MeOH (8.0 mL) and a solution of KHF₂ (710 mg, 328 μL, 9.09 mmol) in H₂O (4.0 mL) was added. The RM was stirred at RT for 2 h and then concentrated under reduced pressure. The residue was suspended in a solution of MeOH/acetone (20/80), filtered, and the solids rinsed twice with MeOH/acetone (20/80). The filtrate was concentrated under reduced pressure, and the resulting residue was triturated with Et₂O at ambient temperature. The precipitate was filtered off, washed with Et₂O, and dried under reduced pressure at 40° C. to afford the title compound (634 mg) as a pale yellow solid. ¹H NMR (400 MHZ, DMSO-d₆) δ [ppm] 7.83-7.62 (m, 2H), 7.56-7.31 (m, 2H), 3.67-3.53 (m, 2H), 3.21-3.09 (m, 1H), 2.87-2.69 (m, 2H), 2.13-1.97 (m, 1H), 1.45-1.35 (m, 9H), 0.10-−0.08 (m, 1H), −0.13-−0.36 (m, 1H).

### Intermediate F-3

### potassium (((3R,4R)-1-(tert-butoxycarbonyl)-3-(4-(tert-butoxycarbonyl)phenyl)azepan-4-yl)methyl)trifluoroborate

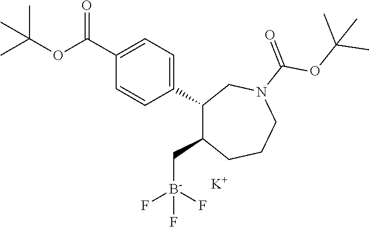 [SMILES: CC(C)(C)OC(=O)c1ccc([C@@H]2CN(C(=O)OC(C)(C)C)CCC[C@H]2C[B-](F)(F)F)cc1 ; [K+]]

Intermediate F-3 was prepared according to the method described for Intermediate F-1 herein above, using Intermediate A-4. LC-MS Method B-2: Rt=6.42 min; MS m/z [M-K+Na]⁻=478.1.

### Intermediate F-4

### potassium (((4R,5R)-1-(tert-butoxycarbonyl)-5-(4-(tert-butoxycarbonyl)phenyl)azepan-4-yl)methyl)trifluoroborate

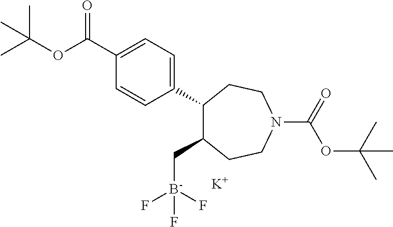 [SMILES: CC(C)(C)OC(=O)c1ccc([C@@H]2CCN(C(=O)OC(C)(C)C)CC[C@H]2C[B-](F)(F)F)cc1 ; [K+]]

Intermediate F-4 was prepared according to the method described for Intermediate F-1 herein above, using Intermediate A-5. LC-MS Method B-2: Rt=6.59 min; MS m/z [M-K+Na]⁻=478.4.

### Intermediate F-5

### potassium (((3R,4R)-1-((benzyloxy)carbonyl)-3-(4-cyanophenyl)piperidin-4-yl)methyl)trifluoroborate

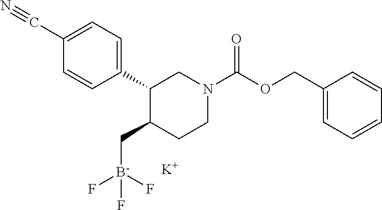 [SMILES: N#Cc1ccc([C@@H]2CN(C(=O)OCc3ccccc3)CC[C@H]2C[B-](F)(F)F)cc1 ; [K+]]

Intermediate F-5 was prepared according to the method described for Intermediate F-1 herein above, using Intermediate A-6. LC-MS Method B-2: Rt=4.73 min; MS m/z [M-K+Na]⁻=423.2.

### Intermediate F-6

### potassium (((3R,4R)-3-(4-(1H-pyrazol-1-yl)phenyl)-1-((benzyloxy)carbonyl)piperidin-4-yl)methyl)trifluoroborate

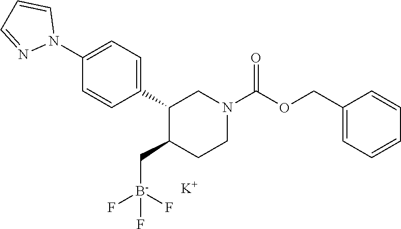 [SMILES: O=C(OCc1ccccc1)N1CC[C@@H](C[B-](F)(F)F)[C@H](c2ccc(-n3cccn3)cc2)C1 ; [K+]]

Intermediate F-6 was prepared according to the method described for Intermediate F-1 herein above, using Intermediate A-8. LC-MS Method B-2: Rt=4.98 min; MS m/z [M-K+Na]⁻=464.3.

### Intermediate F-7

### potassium (((3R,4R)-1-(tert-butoxycarbonyl)-3-(1-methyl-1H-pyrazol-4-yl)piperidin-4-yl)methyl)trifluoroborate

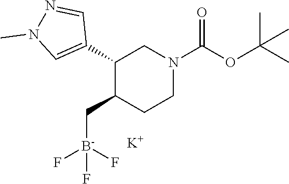 [SMILES: Cn1cc([C@@H]2CN(C(=O)OC(C)(C)C)CC[C@H]2C[B-](F)(F)F)cn1 ; [K+]]

Intermediate F-7 was prepared according to the method described for Intermediate F-1 herein above, using Intermediate A-9. LC-MS Method B-2: Rt=3.59 min; MS m/z [M-K+Na]⁻=368.2.

### Intermediate rac-B-1: tert-butyl 4-(((3RS,4RS)-3-(4-(methoxycarbonyl)phenyl)-1-(2,2,2-trifluoroethyl)piperidin-4-yl)methyl)-5,7-dimethyl-1H-indole-1-carboxylate

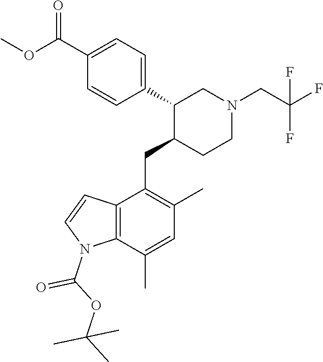 [SMILES: COC(=O)c1ccc([C@@H]2CN(CC(F)(F)F)CC[C@H]2Cc2c(C)cc(C)c3c2ccn3C(=O)OC(C)(C)C)cc1]

A soln. of methyl 4-(4-methylene-1-(2,2,2-trifluoroethyl)piperidin-3-yl)benzoate (Intermediate A-1, 1.382 g, 4.41 mmol) in THF (16.0 mL) was evacuated/backfilled with N₂ twice. 9-BBN (0.5M in THF, 8.82 mL, 4.41 mmol) was added slowly and the RM was stirred in a capped vial at 50° C. for 4 h. The RM was then cooled to RT. A degassed soln. of K₃PO₄ (2.553 g, 12.03 mmol) in water (8.00 mL) was added slowly to the mixture. tert-Butyl 4-bromo-5,7-dimethyl-1H-indole-1-carboxylate (Intermediate I-1, 1.30 g, 4.01 mmol) and PdCl₂ (dtbpf) (130.7 mg, 200.5 μmol) were then added as solids. The mixture was evacuated/backfilled with N₂ and the capped vial was stirred at 50° C. for 2 h. The RM was allowed to cool to RT and then partitioned between water and TBME. The layers were separated and washed with brine and TBME. The combined org layers were dried over MgSO₄, filtered and the volatiles were removed under reduced pressure at 50° C. to give a brown oil that was purified over silica gel (220 g) using an automated purification system (NP; Teledyne ISCO®; collected at 245/263 nm; flow 150 mL/min; eluent: heptane+1 to 9.7% EtOAc in 25.9 min). Pure fractions were combined and the volatiles were removed under reduced pressure at 50° C. to give a) recovered starting material (Intermediate A-1, 308 mg) and b) cis-isomer as a white foam (1.34 g); LC-MS Method B-2: Rt=9.19 min; MS m/z [M+H]⁺=559.2; ¹H NMR (400 MHZ, DMSO-d₆) δ [ppm] 7.95 (d, J=8.1 Hz, 2H), 7.74 (d, J=8.1 Hz, 2H), 7.50 (d, J=3.8 Hz, 1H), 6.82 (s, 1H), 6.28 (d, J=3.8 Hz, 1H), 3.86 (s, 3H), 3.30-3.09 (m, 4H), 2.98-2.90 (m, 1H), 2.84 (dd, J=11.4, 3.5 Hz, 1H), 2.56-2.49 (m, 1H, partially overlapping with DMSO peak), 2.48-2.30 (m, 5H), 2.04 (s, 4H), 1.56 (s, 9H), 1.43-1.30 (m, 2H); c) the title compound (trans-isomer) as an amber oil (211 mg); LC-MS Method B-2: Rt=8.92 min; MS m/z [M+H]⁺=559.4; ¹H NMR (400 MHZ, DMSO-d₆) δ [ppm] 7.97 (d, J=8.2 Hz, 2H), 7.58-7.51 (m, 3H), 6.81 (s, 1H), 6.47 (d, J=3.8 Hz, 1H), 3.86 (s, 3H), 3.24-3.09 (m, 2H), 2.92-2.78 (m, 3H), 2.59-2.45 (m, 2H, partially overlapping with DMSO peak), 2.43-2.37 (m, 4H), 2.30-2.21 (m, 1H), 2.09 (s, 3H), 1.89-1.77 (m, 1H), 1.57 (s, 9H), 1.51-1.37 (m, 1H), 1.29-1.20 (m, 1H).

### Intermediate B-2: tert-butyl 4-(((3S,4S)-3-(4-(methoxycarbonyl)phenyl)-1-(2,2,2-trifluoroethyl)piperidin-4-yl)methyl)-5,7-dimethyl-1H-indole-1-carboxylate and Intermediate B-3: tert-butyl 4-(((3R,4R)-3-(4-(methoxycarbonyl)phenyl)-1-(2,2,2-trifluoroethyl)piperidin-4-yl)methyl)-5,7-dimethyl-1H-indole-1-carboxylate

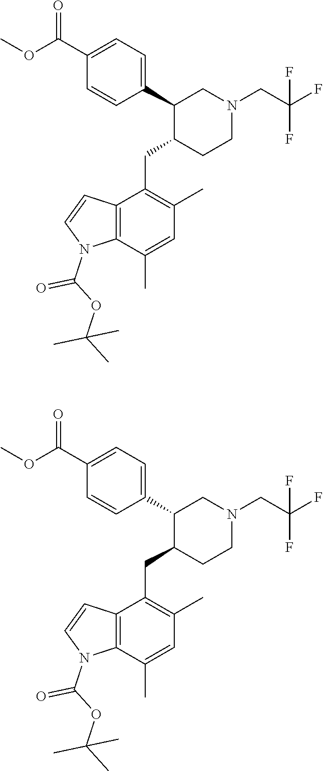 [SMILES: COC(=O)c1ccc([C@H]2CN(CC(F)(F)F)CC[C@@H]2Cc2c(C)cc(C)c3c2ccn3C(=O)OC(C)(C)C)cc1 ; COC(=O)c1ccc([C@@H]2CN(CC(F)(F)F)CC[C@H]2Cc2c(C)cc(C)c3c2ccn3C(=O)OC(C)(C)C)cc1]

Enantiomer separation and analytics according to Method SFC-1: tert-Butyl 4-(((3RS,4RS)-3-(4-(methoxycarbonyl)phenyl)-1-(2,2,2-trifluoroethyl)piperidin-4-yl)methyl)-5,7-dimethyl-1H-indole-1-carboxylate (Intermediate rac-B-1, 210 mg, 375.9 μmol). The volatiles were removed under reduced pressure at 50° C., the residue was triturated multiple times with hexane and DCM and then was dried under reduced pressure at 50° C. providing: Intermediate B-2 (Peak 1): 96.3 mg, white foam. LC-MS Method B-2: Rt=9.01 min; MS m/z [M+H]⁺=559.3; analytical chiral HPLC: Rt=3.19 min; 99.5% ee. Intermediate B-3 (Peak 2), 107.7 mg, amber oil. LC-MS Method B-2: Rt=9.04 min; MS m/z [M+H]⁺=559.2; analytical chiral HPLC: Rt=3.62 min; 96.6% ee.

### Intermediate rac-B-4: tert-butyl 5-cyclopropyl-4-(((3RS,4RS)-3-(4-(methoxycarbonyl)phenyl)-1-(2,2,2-trifluoroethyl)piperidin-4-yl)methyl)-7-methyl-1H-indole-1-carboxylate

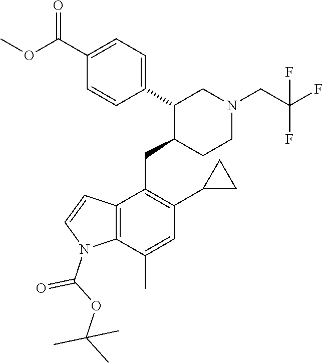 [SMILES: COC(=O)c1ccc([C@@H]2CN(CC(F)(F)F)CC[C@H]2Cc2c(C3CC3)cc(C)c3c2ccn3C(=O)OC(C)(C)C)cc1]

Intermediate rac-B-4 was prepared according to the method described for Intermediate rac-B-4 herein above, using methyl 4-(4-methylene-1-(2,2,2-trifluoroethyl)piperidin-3-yl)benzoate (Intermediate A-1) and tert-butyl 4-bromo-5-cyclopropyl-7-methyl-1H-indole-1-carboxylate (Intermediate I-3) LC-MS Method B-2: Rt=9.17 min; MS m/z [M+H]⁺=585.4.

### Intermediate B-5: tert-butyl 5-cyclopropyl-4-(((3S,4S)-3-(4-(methoxycarbonyl)phenyl)-1-(2,2,2-trifluoroethyl)piperidin-4-yl)methyl)-7-methyl-1H-indole-1-carboxylate and

### Intermediate B-6: tert-butyl 5-cyclopropyl-4-(((3R,4R)-3-4-(methoxycarbonyl)phenyl)-1-(2,2,2-trifluoroethyl)piperidin-4-yl)methyl)-7-methyl-1H-indole-1-carboxylate

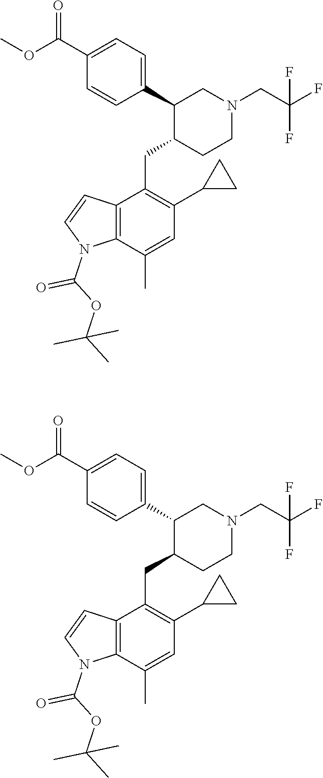 [SMILES: COC(=O)c1ccc([C@H]2CN(CC(F)(F)F)CC[C@@H]2Cc2c(C3CC3)cc(C)c3c2ccn3C(=O)OC(C)(C)C)cc1 ; COC(=O)c1ccc([C@@H]2CN(CC(F)(F)F)CC[C@H]2Cc2c(C3CC3)cc(C)c3c2ccn3C(=O)OC(C)(C)C)cc1]

Enantiomer separation of Intermediate rac-B-4 and analytics according to Method SFC-8:

Intermediate B-5 (Peak 1): off-white foam. LC-MS Method B-2: Rt=9.11 min; MS m/z [M+H]⁺=585.4; analytical chiral HPLC: Rt=1.12 min; 99.5% ee.

Intermediate B-6 (Peak 2): off-white. LC-MS Method B-2: Rt=9.17 min; MS m/z [M+H]⁺=585.3 analytical chiral HPLC: Rt=1.52 min; 99.5% ee.

### Intermediate rac-B-7: tert-butyl 5-methoxy-4-((3-(4-(methoxycarbonyl)phenyl)-1-(2,2,2-trifluoroethyl)piperidin-4-yl)methyl)-7-methyl-1H-indole-1-carboxylate

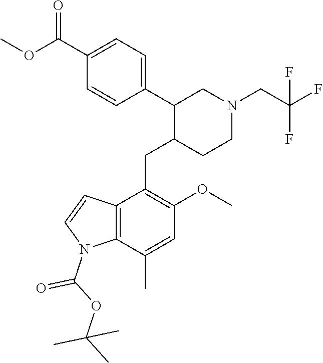 [SMILES: COC(=O)c1ccc(C2CN(CC(F)(F)F)CCC2Cc2c(OC)cc(C)c3c2ccn3C(=O)OC(C)(C)C)cc1]

A diatereomeric mixture of Intermediate rac-B-7 was prepared according to the method described for Intermediate rac-B-4 herein above, using methyl 4-(4-methylene-1-(2,2,2-trifluoroethyl)piperidin-3-yl)benzoate (Intermediate A-1) and tert-butyl 4-bromo-5-methoxy-7-methyl-1H-indole-1-carboxylate

(Intermediate I-2) LC-MS Method B-2: Rt=8.44 min; MS m/z [M+H]⁺=575.3.

### Intermediate B-8: tert-butyl 5-methoxy-4-(((3S,4S)-3-(4-(methoxycarbonyl)phenyl)-1-(2,2,2-trifluoroethyl)piperidin-4-yl)methyl)-7-methyl-1H-indole-1-carboxylate and

### Intermediate B-9: tert-butyl 5-methoxy-4-(((3R,4R)-3-(4-(methoxycarbonyl)phenyl)-1-(2,2,2-trifluoroethyl)piperidin-4-yl)methyl)-7-methyl-1H-indole-1-carboxylate

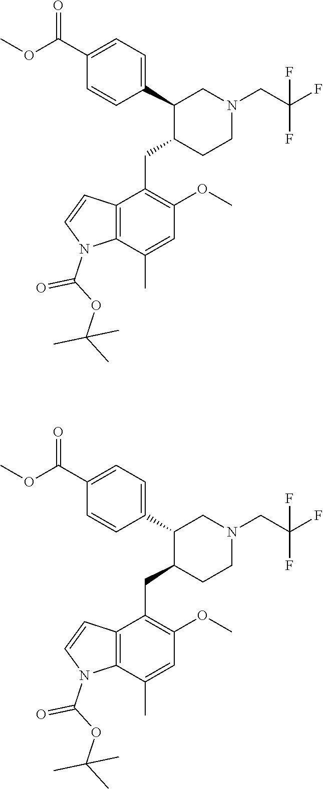 [SMILES: COC(=O)c1ccc([C@H]2CN(CC(F)(F)F)CC[C@@H]2Cc2c(OC)cc(C)c3c2ccn3C(=O)OC(C)(C)C)cc1 ; COC(=O)c1ccc([C@@H]2CN(CC(F)(F)F)CC[C@H]2Cc2c(OC)cc(C)c3c2ccn3C(=O)OC(C)(C)C)cc1]

Diastereomer and enantiomer separation of the diatereomeric mixture Intermediate rac-B-7 and analytics according to Method SFC-9:

Intermediate B-8 (Peak 1): white foam. LC-MS Method B-2: Rt=8.51 min; MS m/z [M+H]⁺=575.3; analytical chiral HPLC: Rt=1.67 min; 99.5% ee.

Intermediate B-9 (Peak 4): white foam. LC-MS Method B-2: Rt=8.51 min; MS m/z [M+H]⁺=575.3 analytical chiral HPLC: Rt=2.72 min; 99.5% ee. The isolated Peak 4 was re-purified by column chromatography over silica gel (12 g, heptane/EtOAc=100/0 to 88/12) before use in the next reaction.

### Intermediate B-10

### tert-butyl 4-(((3R,4R)-1-(tert-butoxycarbonyl)-3-(4-(tert-butoxycarbonyl)phenyl)piperidin-4-yl)methyl)-5,7-dimethyl-1H-indole-1-carboxylate

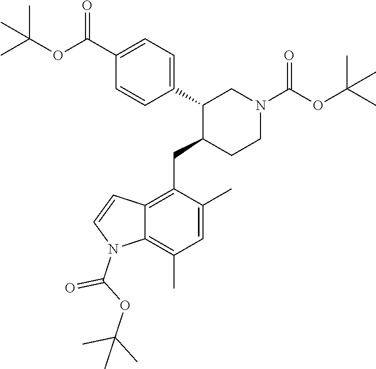 [SMILES: Cc1cc(C)c2c(ccn2C(=O)OC(C)(C)C)c1C[C@@H]1CCN(C(=O)OC(C)(C)C)C[C@H]1c1ccc(C(=O)OC(C)(C)C)cc1]

A mixture of potassium (((3R,4R)-1-(tert-butoxycarbonyl)-3-(4-(tert-butoxycarbonyl)phenyl)piperidin-4-yl)methyl)trifluoroborate (Intermediate F-1, 1.010 g, 2.098 mmol) and tert-butyl 4-bromo-5,7-dimethyl-1H-indole-1-carboxylate (Intermediate I-1, 884.3 mg, 2.727 mmol) in toluene (16.60 mL) was placed in a vial. A soln. of K₃PO₄ (1.470 g, 6.924 mmol) in water (4.15 mL) was added. The mixture was evacuated/back-filled with N₂. PdCl₂ (dppf) (153.5 mg, 209.8 μmol) was added and the mixture was evacuated/back-filled with N₂ again. The RM was stirred in capped vial at 95° C. overnight and then allowed to cool to RT, diluted with TBME/water, and filtered over Celite®. The layers of the filtrate were separated and washed with brine and TBME. The combined org layers were dried over MgSO₄, filtered, and concentrated under reduced pressure at 50° C. The residue was purified over silica gel (40 g) using an automated purification system (NP; Teledyne ISCO®; collected at 245/260 nm; flow 40 mL/min; eluent: heptane+0 to 9.6% EtOAc in 20 min) to provide tert-butyl 4-(((3R,4R)-1-(tert-butoxycarbonyl)-3-(4-(tert-butoxycarbonyl)phenyl)piperidin-4-yl)methyl)-5,7-dimethyl-1H-indole-1-carboxylate (1.03 g) as an off-white foam. LC-MS Method B-2: Rt=9.48 min; MS m/z [M+H]⁺619.3.

### Intermediate B-11

### tert-butyl 4-(((3R,4R)-3-(4-(tert-butoxycarbonyl)phenyl)piperidin-4-yl)methyl)-5,7-dimethyl-1H-indole-1-carboxylate

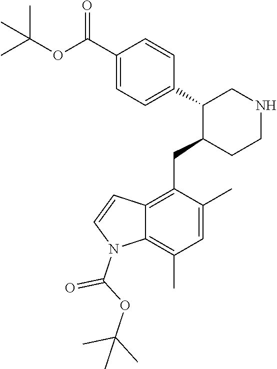 [SMILES: Cc1cc(C)c2c(ccn2C(=O)OC(C)(C)C)c1C[C@@H]1CCNC[C@H]1c1ccc(C(=O)OC(C)(C)C)cc1]

HCl in 1,4-dioxane (4M, 3.085 mL, 12.34 mmol) was added to a soln. of tert-butyl 4-(((3R,4R)-1-(tert-butoxycarbonyl)-3-(4-(tert-butoxycarbonyl)phenyl)piperidin-4-yl)methyl)-5,7-dimethyl-1H-indole-1-carboxylate (Intermediate B-10, 509.0 mg, 822.5 μmol) in dioxane (15.0 mL). The RM was stirred at RT for 70 min, additional HCl in dioxane (4M, 3.03 mL, 12.12 mmol) was added, and stirring was continued for additional 140 min. The RM was poured into a well stirred mixture of aq Na₂CO₃ soln. (10 wt %) and EtOAc. The layers were separated and washed with EtOAc and brine. The combined org layers were dried over MgSO₄, filtered, and concentrated under reduced pressure at 50° C. The residue was purified over silica gel (24 g) using an automated purification system (NP; Teledyne ISCO®; collected at 233/263 nm; flow 35 mL/min; eluent: DCM+0 to 10% {EtOH/aq NH₄OH 25%=9/1} in 25 min) to provide tert-butyl 4-(((3R,4R)-3-(4-(tert-butoxycarbonyl)phenyl)piperidin-4-yl)methyl)-5,7-dimethyl-1H-indole-1-carboxylate (263 mg) as a slightly amber foam. LC-MS Method B-1: Rt=1.44 min; MS m/z [M+H]⁺=519.3.

### Intermediate B-12

### tert-butyl 4-(((3R,4R)-3-(4-(tert-butoxycarbonyl)phenyl)-1-methylpiperidin-4-yl)methyl)-5,7-dimethyl-1H-indole-1-carboxylate

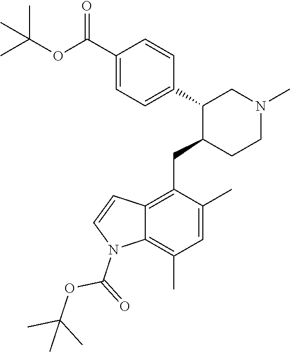 [SMILES: Cc1cc(C)c2c(ccn2C(=O)OC(C)(C)C)c1C[C@@H]1CCN(C)C[C@H]1c1ccc(C(=O)OC(C)(C)C)cc1]

An aq formaldehyde soln. (37% wt, 110.5 μL, 1.484 mmol) was added to a soln. of tert-butyl 4-(((3R,4R)-3-(4-(tert-butoxycarbonyl)phenyl)piperidin-4-yl)methyl)-5,7-dimethyl-1H-indole-1-carboxylate (Intermediate B-11, 77.0 mg, 148.4 μmol) in MeOH (1.70 mL). AcOH (13.37 mg, 12.75 μL, 222.7 μmol) was added. The RM was placed in a water bath (RT), sodium cyanoborohydride (18.66 mg, 296.9 μmol) was added, and the RM was stirred for 1 h. The mixture was partitioned between TBME and sat. aq NaHCO₃ soln. and water; the layers were separated and washed with brine and TBME. The combined org layers were dried over MgSO₄, filtered, and the volatiles were removed under reduced pressure at 50° C. The residue was purified by preparative TLC (MERCK 1.05744.0001, PLC silica gel 60 F254, 0.5 mm; UV detection; eluent: DCM/MeOH=92/8; elution of the band with DCM/MeOH=92/8 and 80/20) to give tert-butyl 4-(((3R,4R)-3-(4-(tert-butoxycarbonyl)phenyl)-1-methylpiperidin-4-yl)methyl)-5,7-dimethyl-1H-indole-1-carboxylate as a white foam (68.6 mg). LC-MS Method B-2: Rt=6.41 min; MS m/z [M+H]⁺=533.3.

### Intermediate B-13

### tert-butyl 4-(((3R,4R)-3-(4-(tert-butoxycarbonyl)phenyl)-1-(2-fluoroethyl)piperidin-4-yl)methyl)-5,7-dimethyl-1H-indole-1-carboxylate

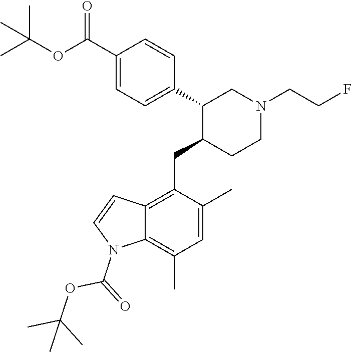 [SMILES: Cc1cc(C)c2c(ccn2C(=O)OC(C)(C)C)c1C[C@@H]1CCN(CCF)C[C@H]1c1ccc(C(=O)OC(C)(C)C)cc1]

A soln. of tert-butyl 4-(((3R,4R)-3-(4-(tert-butoxycarbonyl)phenyl)piperidin-4-yl)methyl)-5,7-dimethyl-1H-indole-1-carboxylate (Intermediate B-11, 50.0 mg, 96.4 μmol) in ACN (0.5 mL) was placed in a microwave vial. DIPEA (31.15 mg, 42.0 μL, 241.0 μmol) and 1-fluoro-2-iodoethane (41.92 mg, 19.63 μL, 241.0 μmol) were added. The capped vial was stirred at 45° C. for 24 h. The RM was cooled to RT, partitioned between TBME and water. The layers were separated and washed with brine and TBME. The combined org layers were dried over MgSO₄, filtered, and the volatiles were removed under reduced pressure at 50° C. to give a yellow oil that was purified by prep. TLC (MERCK 1.05744.0001, PLC silica gel 60 F254, 0.5 mm; eluent: DCM/MeOH=94/6; elution of the band with DCM/MeOH=90/10). The volatiles of the filtrate were removed under reduced pressure at 50° C. to give tert-butyl 4-(((3R,4R)-3-(4-(tert-butoxycarbonyl)phenyl)-1-(2-fluoroethyl)piperidin-4-yl)methyl)-5,7-dimethyl-1H-indole-1-carboxylate (49.5 mg) as a white foam. LC-MS Method B-1: Rt=1.34 min; MS m/z [M+H]⁺=565.4.

### Intermediate B-14

### tert-butyl 4-(((3R,4R)-3-(4-(tert-butoxycarbonyl)phenyl)-1-ethylpiperidin-4-yl)methyl)-5,7-dimethyl-1H-indole-1-carboxylate

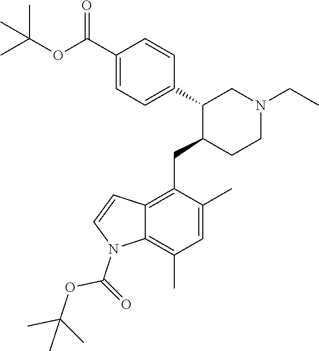 [SMILES: CCN1CC[C@@H](Cc2c(C)cc(C)c3c2ccn3C(=O)OC(C)(C)C)[C@H](c2ccc(C(=O)OC(C)(C)C)cc2)C1]

To a solution of tert-butyl 4-(((3R,4R)-3-(4-(tert-butoxycarbonyl)phenyl)piperidin-4-yl)methyl)-5,7-dimethyl-1H-indole-1-carboxylate (Intermediate B-11) (50.0 mg, 96.4 μmol) in ACN (0.50 mL) were added DIPEA (31.15 mg, 42 μL, 241 μmol) and iodoethane (37.59 mg, 19.42 μL, 241.0 μmol). The RM was stirred at 45° C. for 6 h in a capped vial. The mixture was allowed to cool to RT, then partitioned between TBME and water. The layers were separated and washed with brine and TBME. The combined org layers were dried over MgSO₄, filtered, and the volatiles were removed under reduced pressure at 50° C. The residue was purified by preparative TLC (MERCK 1.05744.0001, PLC silica gel 60 F254, 0.5 mm; UV detection; eluent=DCM/MeOH 95/5; elution of the band with DCM/MeOH=85/15) to give tert-butyl 4-(((3R,4R)-3-(4-(tert-butoxycarbonyl)phenyl)-1-ethylpiperidin-4-yl)methyl)-5,7-dimethyl-1H-indole-1-carboxylate, (30.2) mg as a white foam. LC-MS Method B-2: Rt=6.28 min; MS m/z [M+H]⁺=547.5.

### Intermediate B-15

### tert-butyl 4-(((3R,4R)-3-(4-cyanophenyl)piperidin-4-yl)methyl)-5,7-dimethyl-1H-indole-1-carboxylate

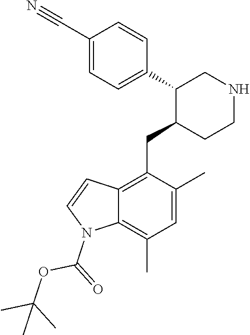 [SMILES: Cc1cc(C)c2c(ccn2C(=O)OC(C)(C)C)c1C[C@@H]1CCNC[C@H]1c1ccc(C#N)cc1]

A mixture of tert-butyl 4-(((3R,4R)-1-((benzyloxy)carbonyl)-3-(4-cyanophenyl)piperidin-4-yl)methyl)-5,7-dimethyl-1H-indole-1-carboxylate (Intermediate B-66, 565 mg, 978 μmol), Pd/C (72.85 mg, 10% wt, 68.4 μmol), and EtOH (20 mL) was stirred under H₂ atmosphere (balloon) for ˜2.5 h. The mixture was filtered over celite and the solids were rinsed with DCM (3×). The volatiles of the filtrate were removed under reduced pressure at 50° C. The residue was dissolved in TBME and the resulting solution was treated with activated charcoal and then filtered over Celite®, and the solids rinsed with TBME (2×). The filtrate was diluted with n-hexane and concentrated under reduced pressure at 50° C. to give crude tert-butyl 4-(((3R,4R)-1-((benzyloxy)carbonyl)-3-(4-cyanophenyl)piperidin-4-yl)methyl)-5,7-dimethyl-1H-indole-1-carboxylate (421 mg) as an off-white foam, which was directly used in the next step without further purification. LC-MS Method B-2: Rt=5.00 min; [M+H]⁺=444.3. ¹H NMR (400 MHZ, DMSO-d₆) δ [ppm] 7.80 (d, J=7.9 Hz, 2H), 7.57 (d, J=8.0 Hz, 2H), 7.52 (d, J=3.7 Hz, 1H), 6.81 (s, 1H), 6.41 (d, J=3.8 Hz, 1H), 2.91-2.79 (m, 2H), 2.65-2.55 (m, 2H), 2.47-2.42 (m, 1H), 2.42-2.30 (m, 5H), 2.10 (s, 3H), 1.99-1.87 (m, 1H), 1.57 (s, 9H), 1.26-1.20 (m, 2H). 32 of 33H reported.

### Intermediate rac-B-16

### racemic tert-butyl 4-(((3S*,4R*)-1-(tert-butoxycarbonyl)-4-(4-cyanophenyl)pyrrolidin-3-yl)methyl)-5,7-dimethyl-1H-indole-1-carboxylate

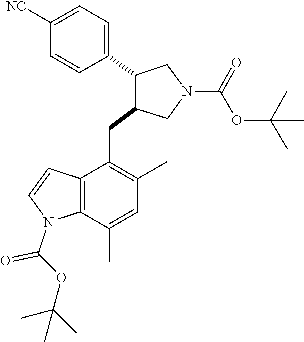 [SMILES: Cc1cc(C)c2c(ccn2C(=O)OC(C)(C)C)c1C[C@@H]1CN(C(=O)OC(C)(C)C)C[C@H]1c1ccc(C#N)cc1]

To tert-butyl 4-bromo-5,7-dimethyl-1H-indole-1-carboxylate (Intermediate I-1, 27 mg, 83 μmol), racemic potassium (((3S*,4R*)-1-(tert-butoxycarbonyl)-4-(4-cyanophenyl)pyrrolidin-3-yl)methyl)trifluoroborate (Intermediate rac-F-2, 21 mg, 53 μmol), K₃PO₄ (34 mg, 161 μmol), and PdCl₂ (dppf)-CH₂Cl₂ adduct (6.1 mg, 7.5 μmol) in a vial under N₂ atmosphere were added toluene (0.5 mL) and water (125 μL), and the mixture was heated at 95° C. overnight. The RM was allowed to cool to RT and then diluted with water. The mixture was extracted with DCM (3×) and the combined organic layers were dried with an Isolute® phase separator and concentrated under reduced pressure. The residue was purified by chromatography over silica gel (40 g, EtOAc/heptane=0/100 to 50/50) to afford racemic tert-butyl 4-(((3S*,4R*)-1-(tert-butoxycarbonyl)-4-(4-cyanophenyl)pyrrolidin-3-yl)methyl)-5,7-dimethyl-1H-indole-1-carboxylate (19.8 mg). LC-MS Method C-1: RT=1.48 min; MS m/z [M-tBu+H]⁺=474.3.

### Intermediate rac-B-17

### racemic tert-butyl 4-(((3S*,4R*)-4-(4-cyanophenyl)pyrrolidin-3-yl)methyl)-5,7-dimethyl-1H-indole-1-carboxylate

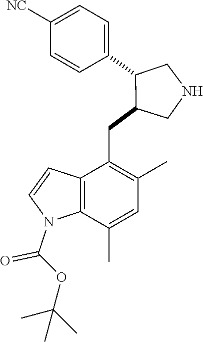 [SMILES: Cc1cc(C)c2c(ccn2C(=O)OC(C)(C)C)c1C[C@@H]1CNC[C@H]1c1ccc(C#N)cc1]

Racemic tert-butyl 4-(((3S*,4R*)-1-(tert-butoxycarbonyl)-4-(4-cyanophenyl)pyrrolidin-3-yl)methyl)-5,7-dimethyl-1H-indole-1-carboxylate (Intermediate rac-B-16, 91 mg) was suspended in MeOH (2.0 mL) and HCl in dioxane (4M, 0.5 mL, 2 mmol) was added. The RM was stirred at RT for 4 h, then concentrated under reduced pressure. The residue was dissolved in DCM, washed with sat. aq NaHCO₃ solution, dried with an Isolute® phase separator and concentrated to afford crude racemic tert-butyl 4-(((3S*,4R*)-4-(4-cyanophenyl)pyrrolidin-3-yl)methyl)-5,7-dimethyl-1H-indole-1-carboxylate (73 mg; partially contaminated with its indole N—H form) as a pale yellow oil. LC-MS Method C-1: RT=1.00 min; MS m/z [M+H]⁺=430.4.

### Intermediate rac-B-18

### racemic tert-butyl 4-(((3S*,4R*)-4-(4-cyanophenyl)-1-methylpyrrolidin-3-yl)methyl)-5,7-dimethyl-1H-indole-1-carboxylate

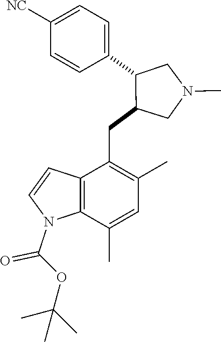 [SMILES: Cc1cc(C)c2c(ccn2C(=O)OC(C)(C)C)c1C[C@@H]1CN(C)C[C@H]1c1ccc(C#N)cc1]

To crude and enriched racemic tert-butyl 4-(((3S*,4R*)-4-(4-cyanophenyl)pyrrolidin-3-yl)methyl)-5,7-dimethyl-1H-indole-1-carboxylate (Intermediate rac-B-17, 73 mg) in MeOH (2.0 mL) and acetic acid (30 μL, 524 μmol) was added a solution of formaldehyde in water (37 wt %, 2.0 mL). The mixture was stirred at RT for 15 min, then sodium triacetoxyborohydride (144 mg, 680 μmol) was added. The RM was stirred at RT overnight. Additional solution of formaldehyde in water (37 wt %, 1.0 mL) and sodium triacetoxyborohydride (100 mg, 472 μmol) were added, and the RM was stirred at RT for 4 h. The mixture was concentrated under reduced pressure and the residue was diluted with DCM and a sat. aq NaHCO₃ solution. The separated aq layer was extracted with DCM (3×) and the combined organic layers were dried with an Isolute® phase separator and concentrated under reduced pressure. The residue was purified by chromatography over silica gel (12 g, {EtOAc/EtOH 3/1}/heptane=0/100 to 100/0) to afford racemic tert-butyl 4-(((3S*,4R*)-4-(4-cyanophenyl)-1-methylpyrrolidin-3-yl)methyl)-5,7-dimethyl-1H-indole-1-carboxylate (48 mg) as a pale yellow oil. LC-MS Method C-1: RT=0.90 min; MS m/z [M+H]⁺=444.5.

The following examples were synthesized from the appropriate starting material by applying similar methods described in the examples above.

| | | | LC-MS |
|---|---|---|---|
| | Chemical Structure | Name | MS m/z |
| Intermediate B-19 | 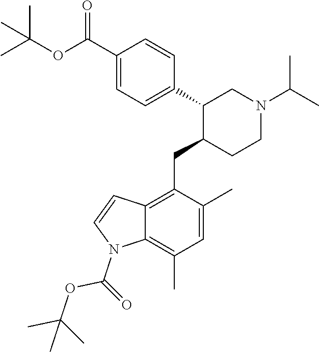 [SMILES: Cc1cc(C)c2c(ccn2C(=O)OC(C)(C)C)c1C[C@@H]1CCN(C(C)C)C[C@H]1c1ccc(C(=O)OC(C)(C)C)cc1] | tert-butyl 4- (((3R,4R)-3-(4-(tert- butoxycarbonyl) phenyl)-1- isopropylpiperidin-4- yl)methyl)-5,7- dimethyl-1H-indole- 1-carboxylate | Method B-2: Rt = 6.15 min; [M + H]⁺ = 561.6 |
| Intermediate B-20 | 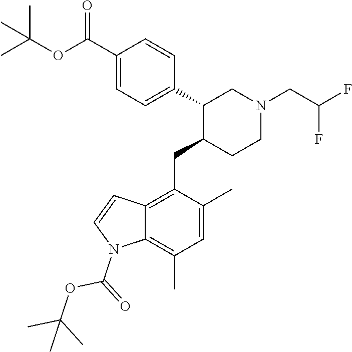 [SMILES: Cc1cc(C)c2c(ccn2C(=O)OC(C)(C)C)c1C[C@@H]1CCN(CC(F)F)C[C@H]1c1ccc(C(=O)OC(C)(C)C)cc1] | tert-butyl 4- (((3R,4R)-3-(4-(tert- butoxycarbonyl) phenyl)-1-(2,2- difluoroethyl) piperidin-4-yl) methyl)-5,7- dimethyl-1H-indole- 1-carboxylate | Method B-2: Rt = 9.05 min; [M + H]⁺ = 583.4 |
| Intermediate B-21 | 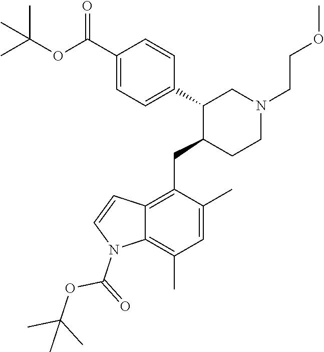 [SMILES: COCCN1CC[C@@H](Cc2c(C)cc(C)c3c2ccn3C(=O)OC(C)(C)C)[C@H](c2ccc(C(=O)OC(C)(C)C)cc2)C1] | tert-butyl 4- (((3R,4R)-3-(4-(tert- butoxycarbonyl) phenyl)-1-(2- methoxyethyl) piperidin-4-yl) methyl)-5,7- dimethyl-1H-indole- 1-carboxylate | Method B-1: Rt = 1.34 min; [M + H]⁺ = 577.4 |
| Intermediate B-22 | 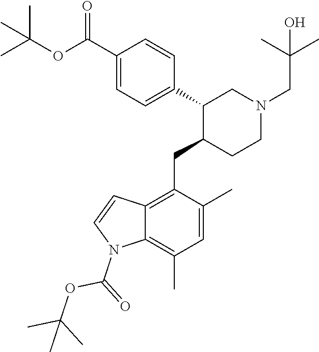 [SMILES: Cc1cc(C)c2c(ccn2C(=O)OC(C)(C)C)c1C[C@@H]1CCN(CC(C)(C)O)C[C@H]1c1ccc(C(=O)OC(C)(C)C)cc1] | tert-butyl 4- (((3R,4R)-3-(4-(tert- butoxycarbonyl) phenyl)-1-(2- hydroxy-2- methylpropyl) piperidin-4-yl) methyl)-5,7- dimethyl-1H-indole- 1-carboxylate | Method B-2: Rt = 6.32 min; [M + H]⁺ = 591.5 |
| Intermediate B-23 | 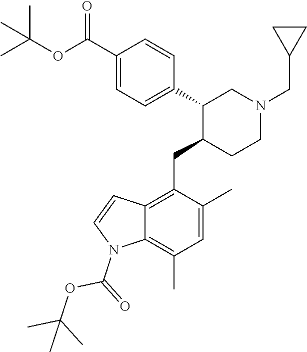 [SMILES: Cc1cc(C)c2c(ccn2C(=O)OC(C)(C)C)c1C[C@@H]1CCN(CC2CC2)C[C@H]1c1ccc(C(=O)OC(C)(C)C)cc1] | tert-butyl 4- (((3R,4R)-3-(4-(tert- butoxycarbonyl) phenyl)-1- (cyclopropylmethyl) piperidin-4-yl) methyl)- 5,7-dimethyl-1H- indole-1-carboxylate | Method B-2: Rt = 6.35 min; [M + H]⁺ = 573.5 |
| Intermediate B-24 | 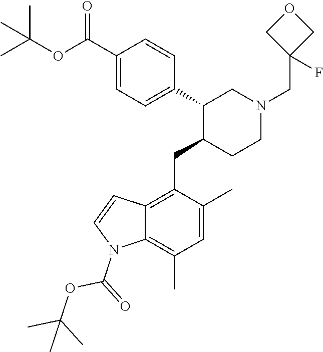 [SMILES: Cc1cc(C)c2c(ccn2C(=O)OC(C)(C)C)c1C[C@@H]1CCN(CC2(F)COC2)C[C@H]1c1ccc(C(=O)OC(C)(C)C)cc1] | tert-butyl 4- (((3R,4R)-3-(4-(tert- butoxycarbonyl) phenyl)-1-((3- fluorooxetan-3- yl)methyl)piperidin- 4-yl)methyl)-5,7- dimethyl-1H-indole- 1-carboxylate | Method B-2: Rt = 7.60 min; [M + H]⁺ = 607.4 |
| Intermediate B-25 | 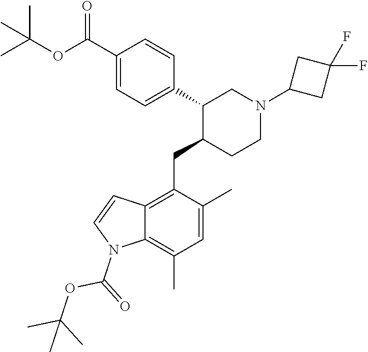 [SMILES: Cc1cc(C)c2c(ccn2C(=O)OC(C)(C)C)c1C[C@@H]1CCN(C2CC(F)(F)C2)C[C@H]1c1ccc(C(=O)OC(C)(C)C)cc1] | tert-butyl 4- (((3R,4R)-3-(4-(tert- butoxycarbonyl) phenyl)-1-(3,3- difluorocyclobutyl) piperidin-4-yl) methyl)- 5,7-dimethyl-1H- indole-1-carboxylate | Method B-2: Rt = 8.42 min; [M + H]⁺ = 609.5 |
| Intermediate B-26 | 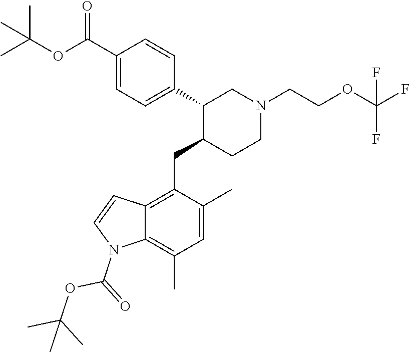 [SMILES: Cc1cc(C)c2c(ccn2C(=O)OC(C)(C)C)c1C[C@@H]1CCN(CCOC(F)(F)F)C[C@H]1c1ccc(C(=O)OC(C)(C)C)cc1] | tert-butyl 4- (((3R,4R)-3-(4-(tert- butoxycarbonyl) phenyl)-1-(2- (trifluoromethoxy) ethyl)piperidin-4- yl)methyl)-5,7- dimethyl-1H-indole- 1-carboxylate | Method B-1: Rt = 1.68 min; [M + H]⁺ = 631.5 |
| Intermediate B-27 | 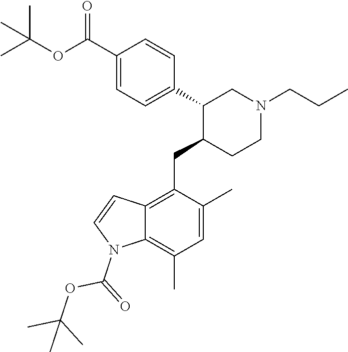 [SMILES: CCCN1CC[C@@H](Cc2c(C)cc(C)c3c2ccn3C(=O)OC(C)(C)C)[C@H](c2ccc(C(=O)OC(C)(C)C)cc2)C1] | tert-butyl 4- (((3R,4R)-3-(4-(tert- butoxycarbonyl) phenyl)-1- propylpiperidin-4- yl)methyl)-5,7- dimethyl-1 H-indole- 1-carboxylate | Method B-2: Rt = 6.34 min; [M + H]⁺ = 561.4 |
| Intermediate B-28 | 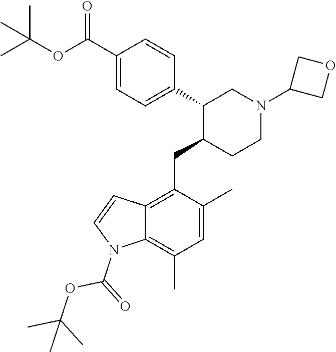 [SMILES: Cc1cc(C)c2c(ccn2C(=O)OC(C)(C)C)c1C[C@@H]1CCN(C2COC2)C[C@H]1c1ccc(C(=O)OC(C)(C)C)cc1] | tert-butyl 4- (((3R,4R)-3-(4-(tert- butoxycarbonyl) phenyl)-1-(oxetan-3- yl)piperidin-4- yl)methyl)-5,7- dimethyl-1 H-indole- 1-carboxylate | Method B-2: Rt = 7.12 min; [M + H]⁺ = 575.4 |
| Intermediate B-29 | 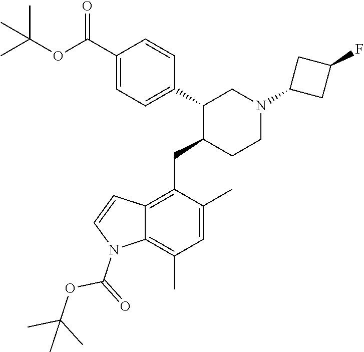 [SMILES: Cc1cc(C)c2c(ccn2C(=O)OC(C)(C)C)c1C[C@@H]1CCN([C@H]2C[C@H](F)C2)C[C@H]1c1ccc(C(=O)OC(C)(C)C)cc1] | tert-butyl 4- (((3R,4R)-3-(4-(tert- butoxycarbonyl) phenyl)-1-((1r,3R)- 3-fluorocyclobutyl) piperidin-4-yl) methyl)- 5,7-dimethyl-1H- indole-1-carboxylate | Method B-2: Rt = 6.59 min; [M + H]⁺ = 591.3 |
| Intermediate B-30 | 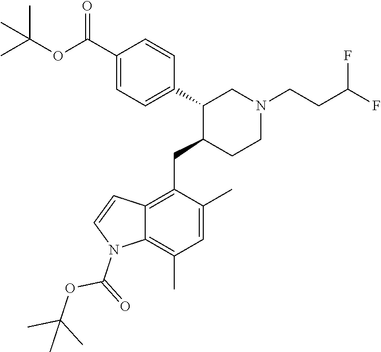 [SMILES: Cc1cc(C)c2c(ccn2C(=O)OC(C)(C)C)c1C[C@@H]1CCN(CCC(F)F)C[C@H]1c1ccc(C(=O)OC(C)(C)C)cc1] | tert-butyl 4- (((3R,4R)-3-(4-(tert- butoxycarbonyl) phenyl)-1-(3,3- difluoropropyl) piperidin-4-yl) methyl)-5,7- dimethyl-1H-indole- 1-carboxylate | Method B-2: Rt = 6.62 min; [M + H]⁺ = 597.4 |
| Intermediate B-31 | 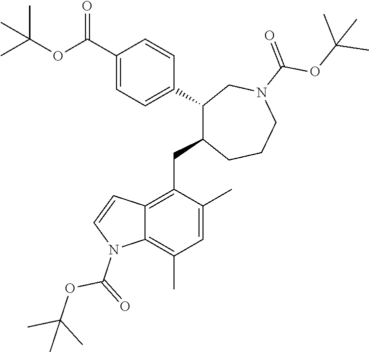 [SMILES: Cc1cc(C)c2c(ccn2C(=O)OC(C)(C)C)c1C[C@@H]1CCCN(C(=O)OC(C)(C)C)C[C@H]1c1ccc(C(=O)OC(C)(C)C)cc1] | tert-butyl 4- (((3R,4S)-1-(tert- butoxycarbonyl)-3- (4-(tert- butoxycarbonyl) phenyl)azepan-4- yl)methyl)-5,7- dimethyl-1H-indole- 1-carboxylate | Method B-2: Rt = 9.39 min; [M − Boc + H − tBu + H − tBu + H]⁺ = 421.3 |
| Intermediate B-32 | 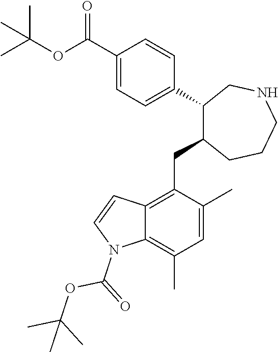 [SMILES: Cc1cc(C)c2c(ccn2C(=O)OC(C)(C)C)c1C[C@@H]1CCCNC[C@H]1c1ccc(C(=O)OC(C)(C)C)cc1] | tert-butyl 4- (((3R,4S)-3-(4-(tert- butoxycarbonyl) phenyl)azepan-4- yl)methyl)-5,7- dimethyl-1H-indole- 1-carboxylate | Method B-2: Rt = 6.27 min; [M + H]⁺ = 533.4 |
| Intermediate B-33 | 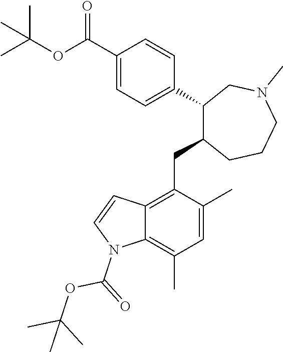 [SMILES: Cc1cc(C)c2c(ccn2C(=O)OC(C)(C)C)c1C[C@@H]1CCCN(C)C[C@H]1c1ccc(C(=O)OC(C)(C)C)cc1] | tert-butyl 4- (((3R,4S)-3-(4-(tert- butoxycarbonyl) phenyl)-1- methylazepan-4- yl)methyl)-5,7- dimethyl-1H-indole- 1-carboxylate | Method B-2: Rt = 6.31 min; [M + H]⁺ = 547.3 |
| Intermediate B-34 | 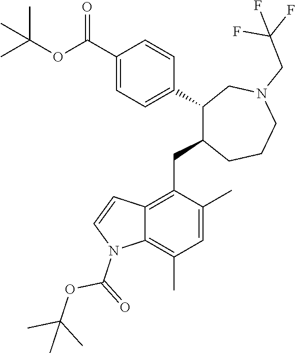 [SMILES: Cc1cc(C)c2c(ccn2C(=O)OC(C)(C)C)c1C[C@@H]1CCCN(CC(F)(F)F)C[C@H]1c1ccc(C(=O)OC(C)(C)C)cc1] | tert-butyl 4- (((3R,4S)-3-(4-(tert- butoxycarbonyl) phenyl)-1-(2,2,2- trifluoroethyl) azepan-4-yl) methyl)-5,7- dimethyl-1H-indole- 1-carboxylate | Method B-2: Rt = 9.65 min; [M + H]⁺ = 615.3 |
| Intermediate B-35 | 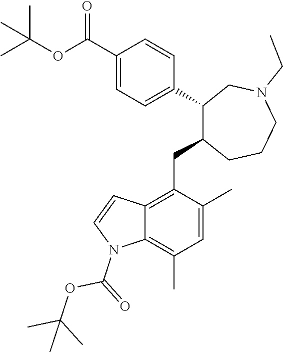 [SMILES: CCN1CCC[C@@H](Cc2c(C)cc(C)c3c2ccn3C(=O)OC(C)(C)C)[C@H](c2ccc(C(=O)OC(C)(C)C)cc2)C1] | tert-butyl 4- (((3R,4S)-3-(4-(tert- butoxycarbonyl) phenyl)-1- ethylazepan- 4-yl)methyl)-5,7- dimethyl-1H-indole- 1-carboxylate | Method B-2: Rt = 6.39 min; [M + H]⁺ = 561.4 |
| Intermediate B-36 | 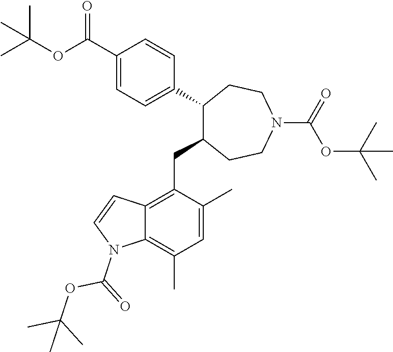 [SMILES: Cc1cc(C)c2c(ccn2C(=O)OC(C)(C)C)c1C[C@@H]1CCN(C(=O)OC(C)(C)C)CC[C@H]1c1ccc(C(=O)OC(C)(C)C)cc1] | tert-butyl 4- (((4R,5R)-1-(tert- butoxycarbonyl)-5- (4-(tert- butoxycarbonyl) phenyl)azepan-4- yl)methyl)-5,7- dimethyl-1H-indole- 1-carboxylate | Method B-2: Rt = 9.42 min; [M − Boc + H − tBu + H − tBu + H]⁺ = 421.3 |
| Intermediate B-37 | 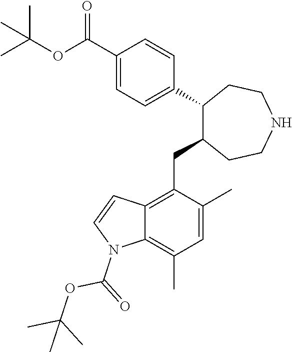 [SMILES: Cc1cc(C)c2c(ccn2C(=O)OC(C)(C)C)c1C[C@@H]1CCNCC[C@H]1c1ccc(C(=O)OC(C)(C)C)cc1] | tert-butyl 4- (((4R,5R)-5-(4-(tert- butoxycarbonyl) phenyl)azepan-4- yl)methyl)-5,7- dimethyl-1H-indole- 1-carboxylate | Method B-2: Rt = 6.39 min; [M + H]⁺ = 533.4 |
| Intermediate B-38 | 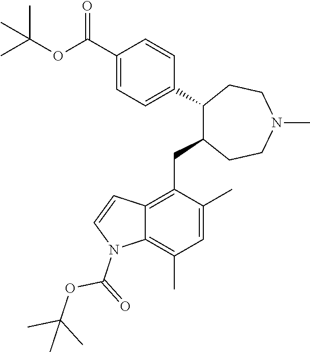 [SMILES: Cc1cc(C)c2c(ccn2C(=O)OC(C)(C)C)c1C[C@@H]1CCN(C)CC[C@H]1c1ccc(C(=O)OC(C)(C)C)cc1] | tert-butyl 4- (((4R,5R)-5-(4-(tert- butoxycarbonyl) phenyl)-1- methylazepan-4- yl)methyl)-5,7- dimethyl-1H-indole- 1-carboxylate | Method B-2: Rt = 6.24 min; [M + H]⁺ = 547.5 |
| Intermediate B-39 | 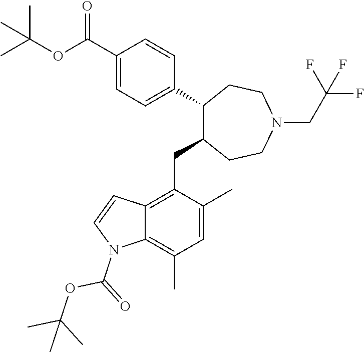 [SMILES: Cc1cc(C)c2c(ccn2C(=O)OC(C)(C)C)c1C[C@@H]1CCN(CC(F)(F)F)CC[C@H]1c1ccc(C(=O)OC(C)(C)C)cc1] | tert-butyl 4- (((4R,5R)-5-(4-(tert- butoxycarbonyl) phenyl)-1-(2,2,2- trifluoroethyl) azepan-4-yl) methyl)-5,7- dimethyl-1H-indole- 1-carboxylate | Method B-2: Rt = 9.62 min; [M + H]⁺ = 615.3 |
| Intermediate B-40 | 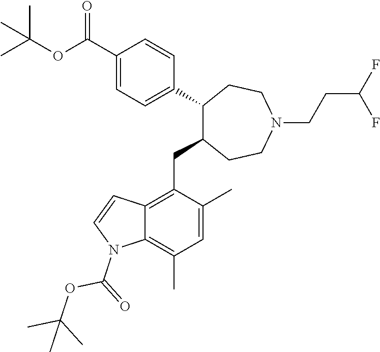 [SMILES: Cc1cc(C)c2c(ccn2C(=O)OC(C)(C)C)c1C[C@@H]1CCN(CCC(F)F)CC[C@H]1c1ccc(C(=O)OC(C)(C)C)cc1] | tert-butyl 4- (((4R,5R)-5-(4-(tert- butoxycarbonyl) phenyl)-1-(3,3- difluoropropyl) azepan- 4-yl)methyl)-5,7- dimethyl-1H-indole- 1-carboxylate | Method B-2: Rt = 6.32 min; [M + H]⁺ = 611.5 |
| Intermediate B-41 | 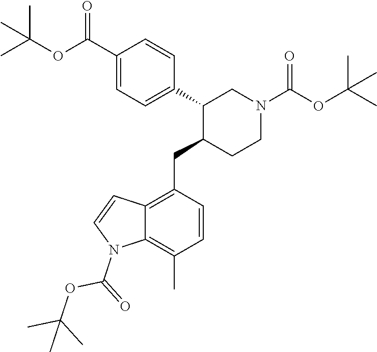 [SMILES: Cc1ccc(C[C@@H]2CCN(C(=O)OC(C)(C)C)C[C@H]2c2ccc(C(=O)OC(C)(C)C)cc2)c2ccn(C(=O)OC(C)(C)C)c12] | tert-butyl 4- (((3R,4R)-1-(tert- butoxycarbonyl)-3- (4-(tert- butoxycarbonyl) phenyl)piperidin-4- yl)methyl)-7-methyl- 1H-indole-1- carboxylate | Method B-2: Rt = 9.42 min; [M − Boc + H − tBu + H − tBu + H]⁺ = 393.2 |
| Intermediate B-42 | 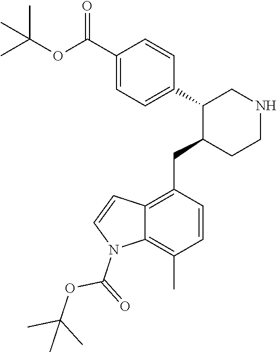 [SMILES: Cc1ccc(C[C@@H]2CCNC[C@H]2c2ccc(C(=O)OC(C)(C)C)cc2)c2ccn(C(=O)OC(C)(C)C)c12] | tert-butyl 4- (((3R,4R)-3-(4-(tert- butoxycarbonyl) phenyl)piperidin-4- yl)methyl)-7-methyl- 1H-indole-1- carboxylate | Method B-2: Rt = 6.08 min; [M + H]⁺ = 505.3 |
| Intermediate B-43 | 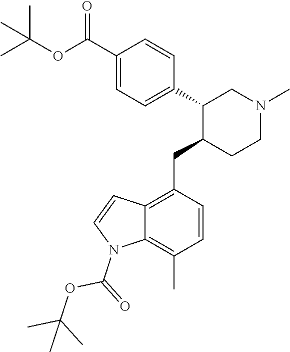 [SMILES: Cc1ccc(C[C@@H]2CCN(C)C[C@H]2c2ccc(C(=O)OC(C)(C)C)cc2)c2ccn(C(=O)OC(C)(C)C)c12] | tert-butyl 4- (((3R,4R)-3-(4-(tert- butoxycarbonyl) phenyl)-1- methylpiperidin-4- yl)methyl)-7-methyl- 1H-indole-1- carboxylate | Method B-2: Rt = 6.07 min; [M + H]⁺ = 519.3 |
| Intermediate B-44 | 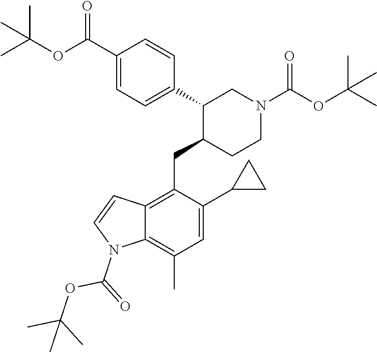 [SMILES: Cc1cc(C2CC2)c(C[C@@H]2CCN(C(=O)OC(C)(C)C)C[C@H]2c2ccc(C(=O)OC(C)(C)C)cc2)c2ccn(C(=O)OC(C)(C)C)c12] | tert-butyl 4- (((3R,4R)-1-(tert- butoxycarbonyl)-3- (4-(tert- butoxycarbonyl) phenyl)piperidin-4- yl)methyl)-5- cyclopropyl-7- methyl-1H-indole-1- carboxylate | Method B-2: Rt = 9.54 min; [M − Boc + H − tBu + H − tBu + H]⁺ = 433.2 |
| Intermediate B-45 | 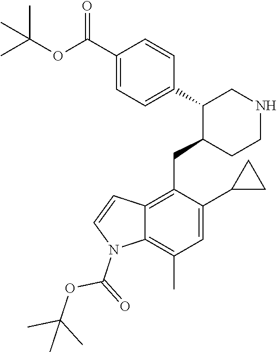 [SMILES: Cc1cc(C2CC2)c(C[C@@H]2CCNC[C@H]2c2ccc(C(=O)OC(C)(C)C)cc2)c2ccn(C(=O)OC(C)(C)C)c12] | tert-butyl 4- (((3R,4R)-3-(4-(tert- butoxycarbonyl) phenyl)piperidin-4- yl)methyl)-5- cyclopropyl-7- methyl-1H-indole-1- carboxylate | Method B-2: Rt = 6.44 min; [M + H]⁺ = 545.4 |
| Intermediate B-46 | 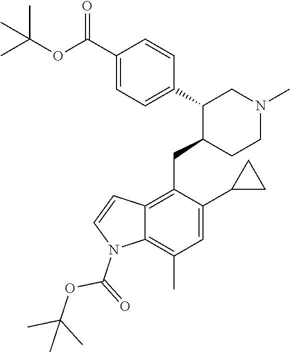 [SMILES: Cc1cc(C2CC2)c(C[C@@H]2CCN(C)C[C@H]2c2ccc(C(=O)OC(C)(C)C)cc2)c2ccn(C(=O)OC(C)(C)C)c12] | tert-butyl 4- (((3R,4R)-3-(4-(tert- butoxycarbonyl) phenyl)-1- methylpiperidin-4- yl)methyl)-5- cyclopropyl-7- methyl-1H-indole-1- carboxylate | Method B-2: Rt = 6.61 min; [M + H]⁺ = 559.3 |
| Intermediate B-47 | 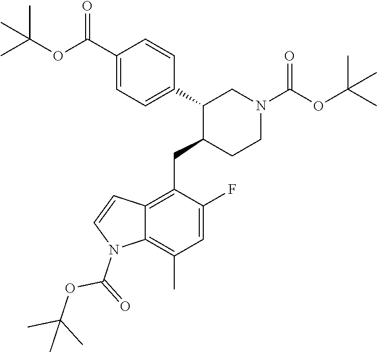 [SMILES: Cc1cc(F)c(C[C@@H]2CCN(C(=O)OC(C)(C)C)C[C@H]2c2ccc(C(=O)OC(C)(C)C)cc2)c2ccn(C(=O)OC(C)(C)C)c12] | tert-butyl 4- (((3R,4R)-1-(tert- butoxycarbonyl)-3- (4-(tert- butoxycarbonyl) phenyl)piperidin-4- yl)methyl)-5-fluoro- 7-methyl-1H-indole- 1-carboxylate | Method B-2: Rt = 9.38 min; [M − Boc + H − tBu + H − tBu + H]⁺ = 411.3 |
| Intermediate B-48 | 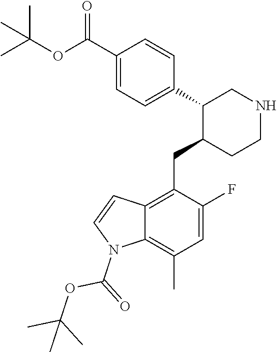 [SMILES: Cc1cc(F)c(C[C@@H]2CCNC[C@H]2c2ccc(C(=O)OC(C)(C)C)cc2)c2ccn(C(=O)OC(C)(C)C)c12] | tert-butyl 4- (((3R,4R)-3-(4-(tert- butoxycarbonyl) phenyl)piperidin-4- yl)methyl)-5-fluoro- 7-methyl-1H-indole- 1-carboxylate | Method B-2: Rt = 6.19 min; [M + H]⁺ = 523.5 |
| Intermediate B-49 | 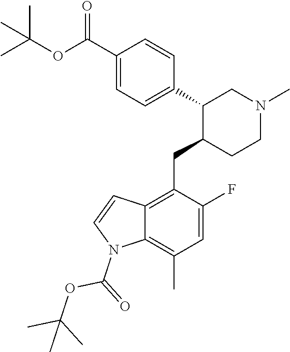 [SMILES: Cc1cc(F)c(C[C@@H]2CCN(C)C[C@H]2c2ccc(C(=O)OC(C)(C)C)cc2)c2ccn(C(=O)OC(C)(C)C)c12] | tert-butyl 4- (((3R,4R)-3-(4-(tert- butoxycarbonyl) phenyl)-1- methylpiperidin-4- yl)methyl)-5-fluoro- 7-methyl-1H-indole- 1-carboxylate | Method B-2: Rt = 6.14 min; [M + H]⁺ = 537.4 |
| Intermediate B-50 | 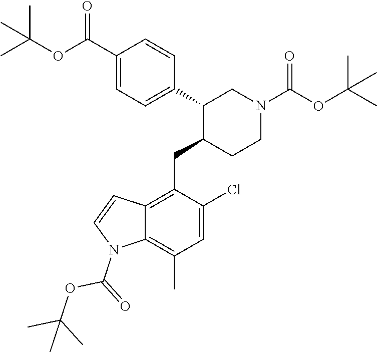 [SMILES: Cc1cc(Cl)c(C[C@@H]2CCN(C(=O)OC(C)(C)C)C[C@H]2c2ccc(C(=O)OC(C)(C)C)cc2)c2ccn(C(=O)OC(C)(C)C)c12] | tert-butyl 4- (((3R,4R)-1-(tert- butoxycarbonyl)-3- (4-(tert- butoxycarbonyl) phenyl)piperidin-4- yl)methyl)-5-chloro- 7-methyl-1H-indole- 1-carboxylate | Method B-2: Rt = 9.42 min; [M − Boc + H]⁺ = 539.3/541.3 |
| Intermediate B-51 | 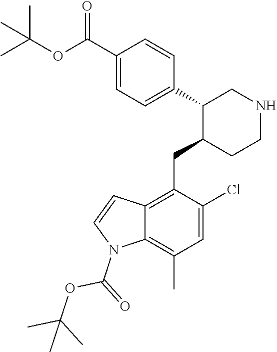 [SMILES: Cc1cc(Cl)c(C[C@@H]2CCNC[C@H]2c2ccc(C(=O)OC(C)(C)C)cc2)c2ccn(C(=O)OC(C)(C)C)c12] | tert-butyl 4- (((3R,4R)-3-(4-(tert- butoxycarbonyl) phenyl)piperidin-4- yl)methyl)-5-chloro- 7-methyl-1H-indole- 1-carboxylate | Method B-2: Rt = 6.54 min; [M + H]⁺ = 539.3/541.2 |
| Intermediate B-52 | 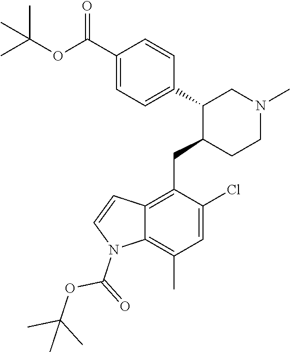 [SMILES: Cc1cc(Cl)c(C[C@@H]2CCN(C)C[C@H]2c2ccc(C(=O)OC(C)(C)C)cc2)c2ccn(C(=O)OC(C)(C)C)c12] | tert-butyl 4- (((3R,4R)-3-(4-(tert- butoxycarbonyl) phenyl)-1- methylpiperidin-4- yl)methyl)-5-chloro- 7-methyl-1H-indole- 1-carboxylate | Method B-2: Rt = 6.17 min; [M + H]⁺ = 553.3/555.3 |
| Intermediate B-53 | 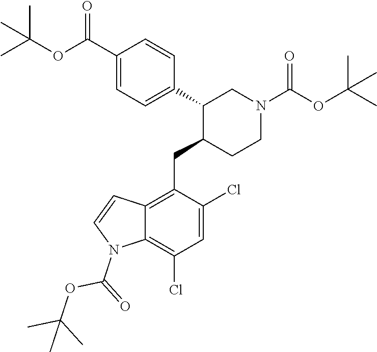 [SMILES: CC(C)(C)OC(=O)c1ccc([C@@H]2CN(C(=O)OC(C)(C)C)CC[C@H]2Cc2c(Cl)cc(Cl)c3c2ccn3C(=O)OC(C)(C)C)cc1] | tert-butyl 4- (((3R,4R)-1-(tert- butoxycarbonyl)-3- (4-(tert- butoxycarbonyl) phenyl)piperidin-4- yl)methyl)-5,7- dichloro-1H-indole- 1-carboxylate | Method B-2: Rt = 9.37 min; [M − H]⁻ = 657.3/659.2/ 661.4 |
| Intermediate B-54 | 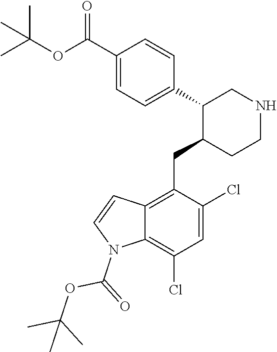 [SMILES: CC(C)(C)OC(=O)c1ccc([C@@H]2CNCC[C@H]2Cc2c(Cl)cc(Cl)c3c2ccn3C(=O)OC(C)(C)C)cc1] | tert-butyl 4- (((3R,4R)-3-(4-(tert- butoxycarbonyl) phenyl)piperidin-4- yl)methyl)-5,7- dichloro-1H-indole- 1-carboxylate | Method B-2: Rt = 6.36 min; [M + H]⁺ = 559.2/561.2/ 563.2 |
| Intermediate B-55 | 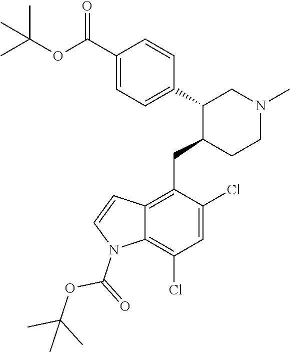 [SMILES: CN1CC[C@@H](Cc2c(Cl)cc(Cl)c3c2ccn3C(=O)OC(C)(C)C)[C@H](c2ccc(C(=O)OC(C)(C)C)cc2)C1] | tert-butyl 4- (((3R,4R)-3-(4-(tert- butoxycarbonyl) phenyl)-1- methylpiperidin-4- yl)methyl)-5,7- dichloro-1H-indole- 1-carboxylate | Method B-2: Rt = 6.19 min; [M + H]⁺ = 573.4/575.3/ 577.4 |
| Intermediate B-56 | 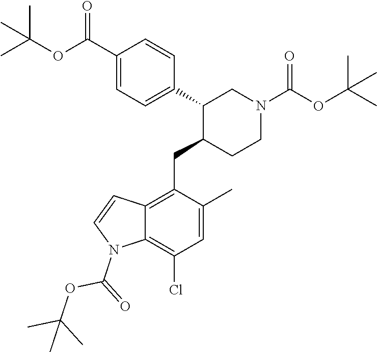 [SMILES: Cc1cc(Cl)c2c(ccn2C(=O)OC(C)(C)C)c1C[C@@H]1CCN(C(=O)OC(C)(C)C)C[C@H]1c1ccc(C(=O)OC(C)(C)C)cc1] | tert-butyl 4- (((3R,4R)-1-(tert- butoxycarbonyl)-3- (4-(tert- butoxycarbonyl) phenyl)piperidin-4- yl)methyl)-7-chloro- 5-methyl-1H-indole- 1-carboxylate | Method B-2: Rt = 9.32 min; [M − H]⁻ = 637.4/639.5 |
| Intermediate B-57 | 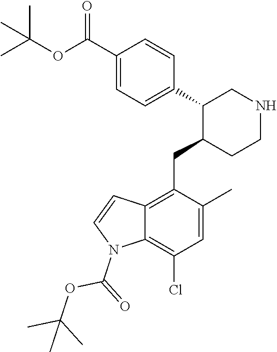 [SMILES: Cc1cc(Cl)c2c(ccn2C(=O)OC(C)(C)C)c1C[C@@H]1CCNC[C@H]1c1ccc(C(=O)OC(C)(C)C)cc1] | tert-butyl 4- (((3R,4R)-3-(4-(tert- butoxycarbonyl) phenyl)piperidin-4- yl)methyl)-7-chloro- 5-methyl-1H-indole- 1-carboxylate | Method B-2: Rt = 6.15 min; [M + H]⁺ = 539.4/541.4 |
| Intermediate B-58 | 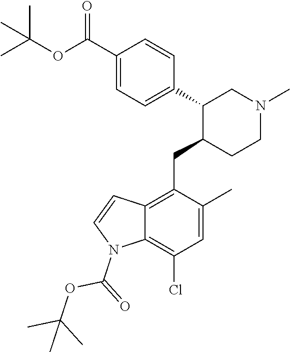 [SMILES: Cc1cc(Cl)c2c(ccn2C(=O)OC(C)(C)C)c1C[C@@H]1CCN(C)C[C@H]1c1ccc(C(=O)OC(C)(C)C)cc1] | tert-butyl 4- (((3R,4R)-3-(4-(tert- butoxycarbonyl) phenyl)-1- methylpiperidin-4- yl)methyl)-7-chloro- 5-methyl-1H-indole- 1-carboxylate | Method B-2: Rt = 6.00 min; [M + H]⁺ = 553.3/555.4 |
| Intermediate B-59 | 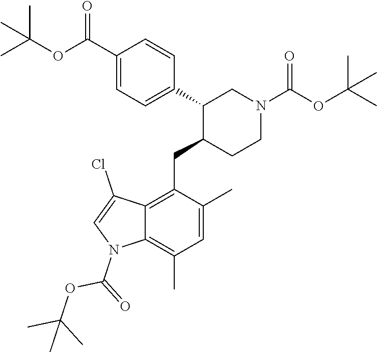 [SMILES: Cc1cc(C)c2c(c(Cl)cn2C(=O)OC(C)(C)C)c1C[C@@H]1CCN(C(=O)OC(C)(C)C)C[C@H]1c1ccc(C(=O)OC(C)(C)C)cc1] | tert-butyl 4- (((3R,4R)-1-(tert- butoxycarbonyl)-3- (4-(tert- butoxycarbonyl) phenyl)piperidin-4- yl)methyl)-3-chloro- 5,7-dimethyl-1H- indole-1-carboxylate | Method B-2: Rt = 9.50 min; [M − Boc + H − tBu + H − tBu + H]⁺ = 441.3/443.3 |
| Intermediate B-60 | 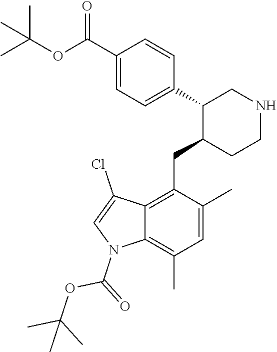 [SMILES: Cc1cc(C)c2c(c(Cl)cn2C(=O)OC(C)(C)C)c1C[C@@H]1CCNC[C@H]1c1ccc(C(=O)OC(C)(C)C)cc1] | tert-butyl 4- (((3R,4R)-3-(4-(tert- butoxycarbonyl) phenyl)piperidin-4- yl)methyl)-3-chloro- 5,7-dimethyl-1H- indole-1-carboxylate | Method B-2: Rt = 6.44 min; [M + H]⁺ = 553.3/555.3 |
| Intermediate B-61 | 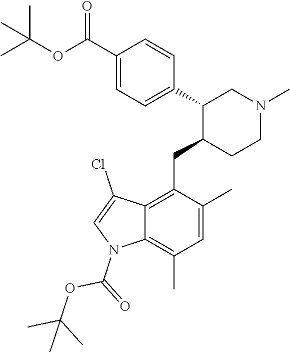 [SMILES: Cc1cc(C)c2c(c(Cl)cn2C(=O)OC(C)(C)C)c1C[C@@H]1CCN(C)C[C@H]1c1ccc(C(=O)OC(C)(C)C)cc1] | tert-butyl 4- (((3R,4R)-3-(4-(tert- butoxycarbonyl) phenyl)-1- methylpiperidin-4- yl)methyl)-3-chloro- 5,7-dimethyl-1H- indole-1-carboxylate | Method B-2: Rt = 6.29 min; [M + H]⁺ = 567.3/569.3 |
| Intermediate B-62 | 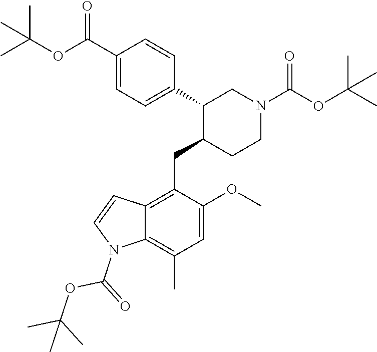 [SMILES: COc1cc(C)c2c(ccn2C(=O)OC(C)(C)C)c1C[C@@H]1CCN(C(=O)OC(C)(C)C)C[C@H]1c1ccc(C(=O)OC(C)(C)C)cc1] | tert-butyl 4- (((3R,4R)-1-(tert- butoxycarbonyl)-3- (4-(tert- butoxycarbonyl) phenyl)piperidin-4- yl)methyl)-5- methoxy-7-methyl- 1H-indole-1- carboxylate | Method B-2: Rt = 9.05 min; [M − Boc + H − Bu + H − tBu + H]⁺ = 423.2 |
| Intermediate B-63 | 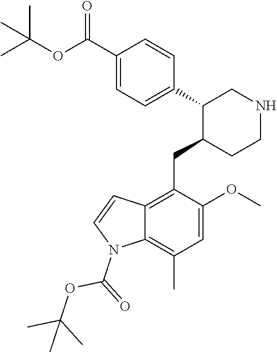 [SMILES: COc1cc(C)c2c(ccn2C(=O)OC(C)(C)C)c1C[C@@H]1CCNC[C@H]1c1ccc(C(=O)OC(C)(C)C)cc1] | tert-butyl 4- (((3R,4R)-3-(4-(tert- butoxycarbonyl) phenyl)piperidin-4- yl)methyl)-5- methoxy-7-methyl- 1H-indole-1- carboxylate | Method B-2: Rt = 5.93 min; [M + H]⁺ = 535.4 |
| Intermediate B-64 | 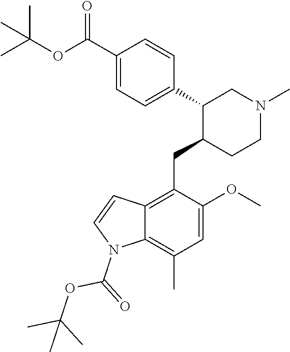 [SMILES: COc1cc(C)c2c(ccn2C(=O)OC(C)(C)C)c1C[C@@H]1CCN(C)C[C@H]1c1ccc(C(=O)OC(C)(C)C)cc1] | tert-butyl 4- (((3R,4R)-3-(4-(tert- butoxycarbonyl) phenyl)-1- methylpiperidin-4- yl)methyl)-5- methoxy-7-methyl- 1H-indole-1- carboxylate | Method B-2: Rt = 6.03 min; [M + H]⁺ = 549.5 |
| Intermediate B-65 | 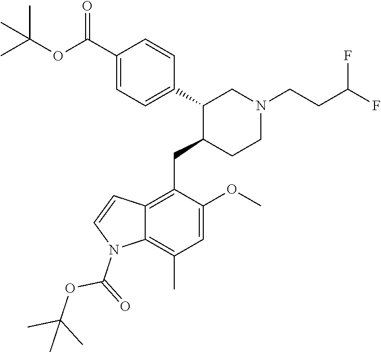 [SMILES: COc1cc(C)c2c(ccn2C(=O)OC(C)(C)C)c1C[C@@H]1CCN(CCC(F)F)C[C@H]1c1ccc(C(=O)OC(C)(C)C)cc1] | tert-butyl 4- (((3R,4R)-3-(4-(tert- butoxycarbonyl) phenyl)-1-(3,3- difluoropropyl) piperidin-4-yl) methyl)-5- methoxy-7-methyl- 1H-indole-1- carboxylate | Method B-2: Rt = 6.21 min; [M + H]⁺ = 613.3 |
| Intermediate B-66 | 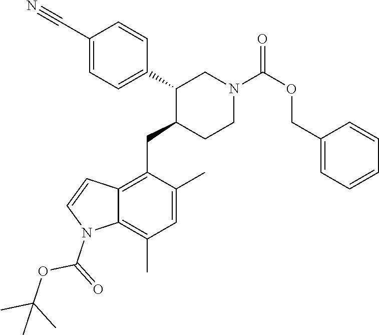 [SMILES: Cc1cc(C)c2c(ccn2C(=O)OC(C)(C)C)c1C[C@@H]1CCN(C(=O)OCc2ccccc2)C[C@H]1c1ccc(C#N)cc1] | tert-butyl 4- (((3R,4R)-1- ((benzyloxy) carbonyl)-3-(4- cyanophenyl) piperidin-4-yl) methyl)-5,7- dimethyl-1H-indole- 1-carboxylate | Method B-2: Rt = 8.31 min; [M − H]⁻ = 576.3 |
| Intermediate B-67 | 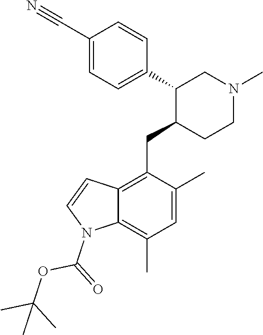 [SMILES: Cc1cc(C)c2c(ccn2C(=O)OC(C)(C)C)c1C[C@@H]1CCN(C)C[C@H]1c1ccc(C#N)cc1] | tert-butyl 4- (((3R,4R)-3-(4- cyanophenyl)-1- methylpiperidin-4- yl)methyl)-5,7- dimethyl-1H-indole- 1-carboxylate | Method B-1: Rt = 1.03 min; [M + H]⁺ = 458.3 |
| Intermediate B-68 | 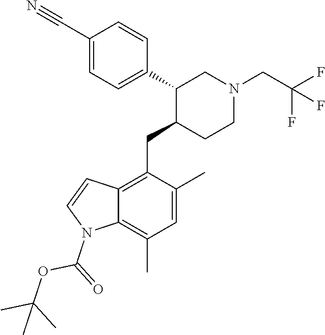 [SMILES: Cc1cc(C)c2c(ccn2C(=O)OC(C)(C)C)c1C[C@@H]1CCN(CC(F)(F)F)C[C@H]1c1ccc(C#N)cc1] | tert-butyl 4- (((3R,4R)-3-(4- cyanophenyl)-1- (2,2,2- trifluoroethyl) piperidin-4-yl) methyl)-5,7- dimethyl-1H-indole- 1-carboxylate | Method B-2: Rt = 8.63 min; [M + H]⁺ = 526.3 |
| Intermediate B-69 | 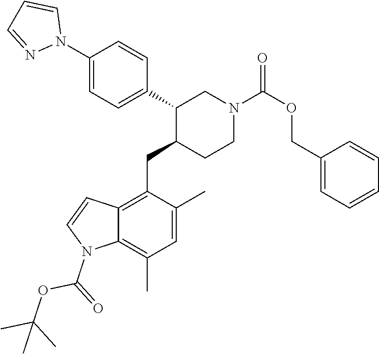 [SMILES: Cc1cc(C)c2c(ccn2C(=O)OC(C)(C)C)c1C[C@@H]1CCN(C(=O)OCc2ccccc2)C[C@H]1c1ccc(-n2cccn2)cc1] | tert-butyl 4- (((3R,4R)-3-(4-(1H- pyrazol-1- yl)phenyl)-1- ((benzyloxy) carbonyl) piperidin-4- yl)methyl)-5,7- dimethyl-1H-indole- 1-carboxylate | Method B-2: Rt = 8.58 min; [M + H]⁺ = 619.5 |
| Intermediate B-70 | 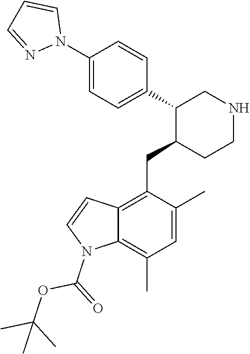 [SMILES: Cc1cc(C)c2c(ccn2C(=O)OC(C)(C)C)c1C[C@@H]1CCNC[C@H]1c1ccc(-n2cccn2)cc1] | tert-butyl 4- (((3R,4R)-3-(4-(1H- pyrazol-1- yl)phenyl)piperidin- 4-yl)methyl)-5,7- dimethyl-1H-indole- 1-carboxylate | Method B-2: Rt = 5.46 min; [M + H]⁺ = 485.6 |
| Intermediate B-71 | 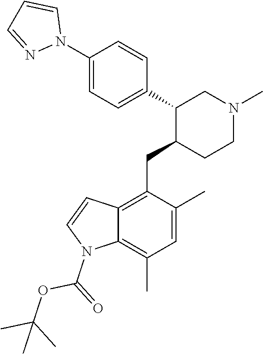 [SMILES: Cc1cc(C)c2c(ccn2C(=O)OC(C)(C)C)c1C[C@@H]1CCN(C)C[C@H]1c1ccc(-n2cccn2)cc1] | tert-butyl 4- (((3R,4R)-3-(4-(1H- pyrazol-1-yl)phenyl)- 1-methylpiperidin-4- yl)methyl)-5,7- dimethyl-1H-indole- 1-carboxylate | Method B-1: Rt = 1.10 min; [M + H]⁺ = 499.4 |
| Intermediate B-72 | 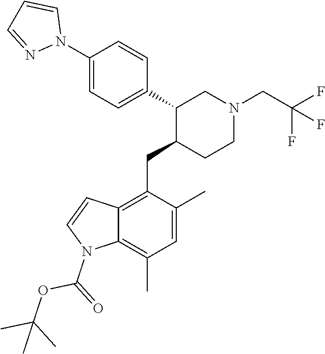 [SMILES: Cc1cc(C)c2c(ccn2C(=O)OC(C)(C)C)c1C[C@@H]1CCN(CC(F)(F)F)C[C@H]1c1ccc(-n2cccn2)cc1] | tert-butyl 4- (((3R,4R)-3-(4-(1H- pyrazol-1-yl)phenyl)- 1-(2,2,2- trifluoroethyl) piperidin-4-yl) methyl)-5,7- dimethyl-1H-indole- 1-carboxylate | Method B-2: Rt = 8.77 min; [M + H]⁺ = 567.3 |
| Intermediate B-73 | 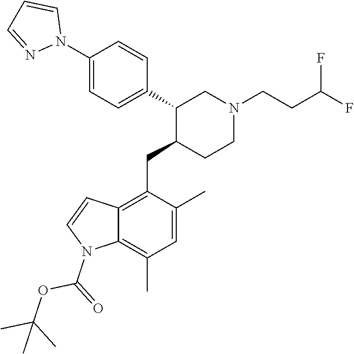 [SMILES: Cc1cc(C)c2c(ccn2C(=O)OC(C)(C)C)c1C[C@@H]1CCN(CCC(F)F)C[C@H]1c1ccc(-n2cccn2)cc1] | tert-butyl 4- (((3R,4R)-3-(4-(1H- pyrazol-1-yl)phenyl)- 1-(3,3- difluoropropyl) piperidin-4-yl) methyl)-5,7- dimethyl-1 H-indole- 1-carboxylate | Method B-2: Rt = 5.45 min; [M + H]⁺ = 563.5 |
| Intermediate B-74 | 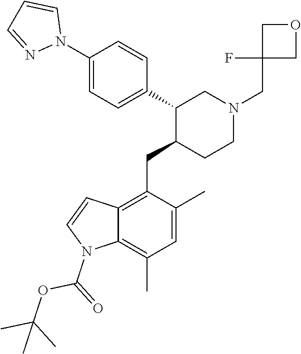 [SMILES: Cc1cc(C)c2c(ccn2C(=O)OC(C)(C)C)c1C[C@@H]1CCN(CC2(F)COC2)C[C@H]1c1ccc(-n2cccn2)cc1] | tert-butyl 4- (((3R,4R)-3-(4-(1H- pyrazol-1-yl)phenyl)- 1-((3-fluorooxetan-3- yl)methyl)piperidin- 4-yl)methyl)-5,7- dimethyl-1H-indole- 1-carboxylate | Method B-1: Rt = 1.32 min; [M + H]⁺ = 573.5 |
| Intermediate B-75 | 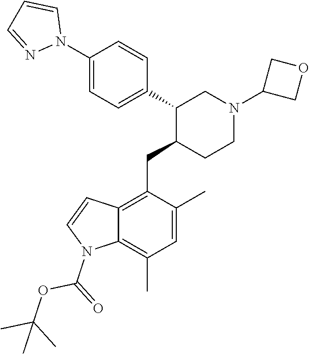 [SMILES: Cc1cc(C)c2c(ccn2C(=O)OC(C)(C)C)c1C[C@@H]1CCN(C2COC2)C[C@H]1c1ccc(-n2cccn2)cc1] | tert-butyl 4- (((3R,4R)-3-(4-(1H- pyrazol-1-yl)phenyl)- 1-(oxetan-3- yl)piperidin-4- yl)methyl)-5,7- dimethyl-1H-indole- 1-carboxylate | Method B-1: Rt = 1.27 min; [M + H]⁺ = 541.2 |
| Intermediate B-76 | 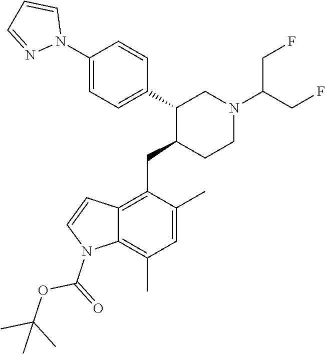 [SMILES: Cc1cc(C)c2c(ccn2C(=O)OC(C)(C)C)c1C[C@@H]1CCN(C(CF)CF)C[C@H]1c1ccc(-n2cccn2)cc1] | tert-butyl 4- (((3R,4R)-3-(4-(1H- pyrazol-1-yl)phenyl)- 1-(1,3- difluoropropan-2- yl)piperidin-4- yl)methyl)-5,7- dimethyl-1H-indole- 1-carboxylate | Method B-1: Rt = 1.52 min; [M + H]⁺ = 563.4 |
| Intermediate B-77 | 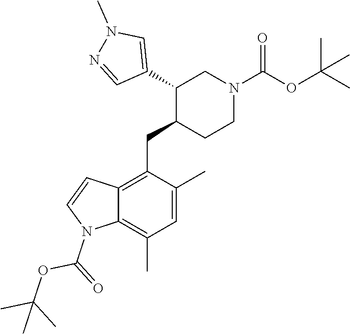 [SMILES: Cc1cc(C)c2c(ccn2C(=O)OC(C)(C)C)c1C[C@@H]1CCN(C(=O)OC(C)(C)C)C[C@H]1c1cnn(C)c1] | tert-butyl 4- (((3R,4R)-1-(tert- butoxycarbonyl)-3- (1-methyl-1H- pyrazol-4- yl)piperidin-4- yl)methyl)-5,7- dimethyl-1 H-indole- 1-carboxylate | Method B-2: Rt = 7.73 min; [M + H]⁺ = 523.4 |
| Intermediate B-78 | 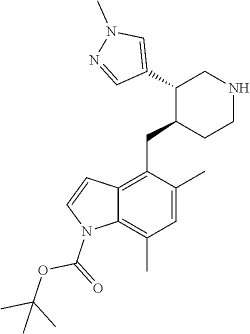 [SMILES: Cc1cc(C)c2c(ccn2C(=O)OC(C)(C)C)c1C[C@@H]1CCNC[C@H]1c1cnn(C)c1] | tert-butyl 5,7- dimethyl-4- (((3R,4R)-3-(1- methyl-1H-pyrazol- 4-yl)piperidin-4- yl)methyl)-1H- indole-1-carboxylate | Method B-2: Rt = 4.44 min; [M + H]⁺ = 423.1 |
| Intermediate B-79 | 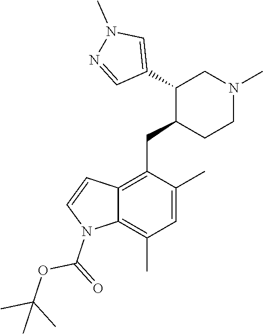 [SMILES: Cc1cc(C)c2c(ccn2C(=O)OC(C)(C)C)c1C[C@@H]1CCN(C)C[C@H]1c1cnn(C)c1] | tert-butyl 5,7- dimethyl-4- (((3R,4R)-1-methyl- 3-(1-methyl-1H- pyrazol-4- yl)piperidin-4- yl)methyl)-1H- indole-1-carboxylate | Method B-2: Rt = 4.32 min; [M + H]⁺ = 437.4 |
| Intermediate B-80 | 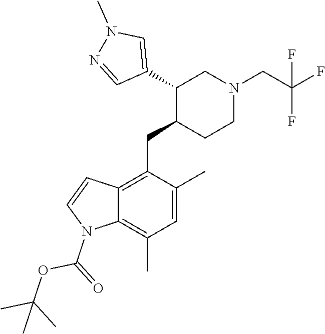 [SMILES: Cc1cc(C)c2c(ccn2C(=O)OC(C)(C)C)c1C[C@@H]1CCN(CC(F)(F)F)C[C@H]1c1cnn(C)c1] | tert-butyl 5,7- dimethyl-4- (((3R,4R)-3-(1- methyl-1H-pyrazol- 4-yl)-1-(2,2,2- trifluoroethyl) piperidin-4-yl) methyl)-1H- indole-1-carboxylate | Method B-2: Rt = 7.81 min; [M + H]⁺ = 505.4 |
| Intermediate B-81 | 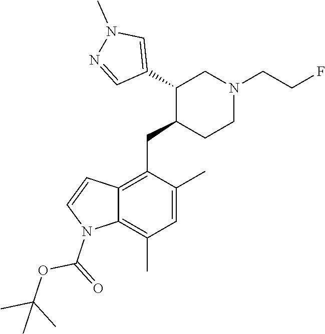 [SMILES: Cc1cc(C)c2c(ccn2C(=O)OC(C)(C)C)c1C[C@@H]1CCN(CCF)C[C@H]1c1cnn(C)c1] | tert-butyl 4- (((3R,4R)-1-(2- fluoroethyl)-3-(1- methyl-1H-pyrazol- 4-yl)piperidin-4- yl)methyl)-5,7- dimethyl-1H-indole- 1-carboxylate | Method B-1: Rt = 0.97 min; [M + H]⁺ = 469.3 |
| Intermediate B-82 | 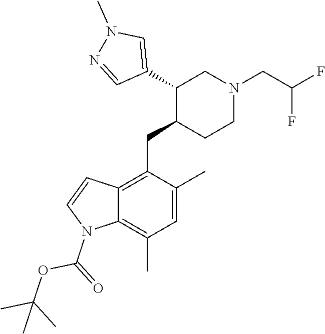 [SMILES: Cc1cc(C)c2c(ccn2C(=O)OC(C)(C)C)c1C[C@@H]1CCN(CC(F)F)C[C@H]1c1cnn(C)c1] | tert-butyl 4- (((3R,4R)-1-(2,2- difluoroethyl)-3-(1- methyl-1H-pyrazol- 4-yl)piperidin-4- yl)methyl)-5,7- dimethyl-1H-indole- 1-carboxylate | Method B-1: Rt = 1.33 min; [M + H]⁺ = 487.1 |
| Intermediate B-83 | 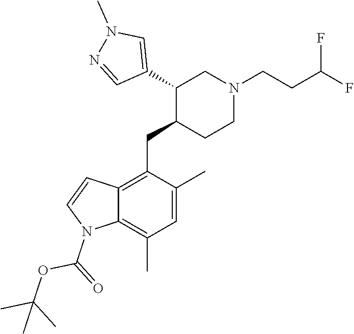 [SMILES: Cc1cc(C)c2c(ccn2C(=O)OC(C)(C)C)c1C[C@@H]1CCN(CCC(F)F)C[C@H]1c1cnn(C)c1] | tert-butyl 4- (((3R,4R)-1-(3,3- difluoropropyl)-3-(1- methyl-1H-pyrazol- 4-yl)piperidin-4- yl)methyl)-5,7- dimethyl-1H-indole- 1-carboxylate | Method B-1: Rt = 0.98 min; [M + H]⁺ = 501.3 |
| Intermediate B-84 | 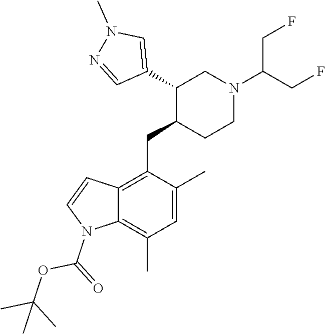 [SMILES: Cc1cc(C)c2c(ccn2C(=O)OC(C)(C)C)c1C[C@@H]1CCN(C(CF)CF)C[C@H]1c1cnn(C)c1] | tert-butyl 4- (((3R,4R)-1-(1,3- difluoropropan-2-yl)- 3-(1-methyl-1H- pyrazol-4- yl)piperidin-4- yl)methyl)-5,7- dimethyl-1H-indole- 1-carboxylate | Method B-1: Rt = 1.19 min; [M + H]⁺ = 501.5 |
| Intermediate B-85 | 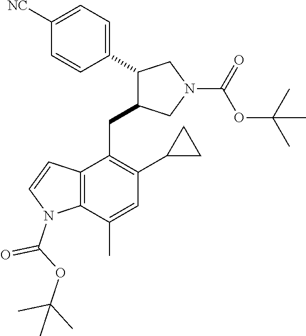 [SMILES: Cc1cc(C2CC2)c(C[C@@H]2CN(C(=O)OC(C)(C)C)C[C@H]2c2ccc(C#N)cc2)c2ccn(C(=O)OC(C)(C)C)c12] | Racemic tert-butyl 4- (((3S,4R)-1-(tert- butoxycarbonyl)-4- (4-cyanophenyl) pyrrolidin-3-yl) methyl)-5- cyclopropyl-7- methyl-1H-indole-1- carboxylate | Method C-1: Rt = 1.51 min; [M − tBu + H]⁺ = 500.4 |
| Intermediate B-86 | 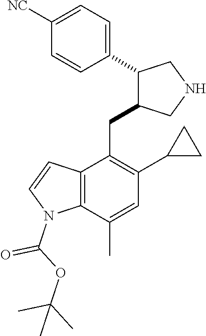 [SMILES: Cc1cc(C2CC2)c(C[C@@H]2CNC[C@H]2c2ccc(C#N)cc2)c2ccn(C(=O)OC(C)(C)C)c12] | Racemic tert-butyl 4- (((3S,4R)-4-(4- cyanophenyl) pyrrolidin-3-yl) methyl)-5- cyclopropyl-7- methyl-1H-indole-1- carboxylate | Method C-1: Rt = 0.99 min; [M + H]⁺ = 456.2 |
| Intermediate B-87 | 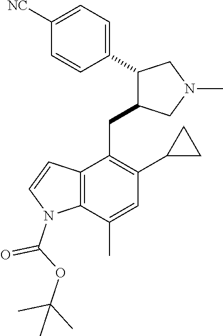 [SMILES: Cc1cc(C2CC2)c(C[C@@H]2CN(C)C[C@H]2c2ccc(C#N)cc2)c2ccn(C(=O)OC(C)(C)C)c12] | Racemic tert-butyl 4- (((3S,4R)-4-(4- cyanophenyl)-1- methylpyrrolidin-3- yl)methyl)-5- cyclopropyl-7- methyl-1H-indole-1- carboxylate | Method C-1: Rt = 0.99 min; [M + H]⁺ = 470.4 |
| Intermediate B-88 | 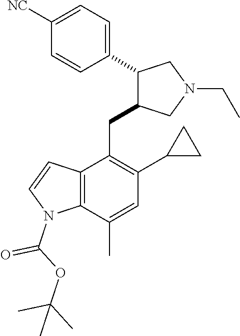 [SMILES: CCN1C[C@@H](Cc2c(C3CC3)cc(C)c3c2ccn3C(=O)OC(C)(C)C)[C@H](c2ccc(C#N)cc2)C1] | Racemic tert-butyl 4- (((3S,4R)-4-(4- cyanophenyl)-1- ethylpyrrolidin-3- yl)methyl)-5- cyclopropyl-7- methyl-1H-indole-1- carboxylate | Method C-1: Rt = 1.12 min; [M + H]⁺ = 484.3 |

### EXAMPLES

### Example Ex-1: 4-((3R,4R)-4-((5,7-dimethyl-1H-indol-4-yl)methyl)-1-(2,2,2-trifluoroethyl)piperidin-3-yl)benzoic acid

An aq NaOH soln. (4M, 482 μL, 1.928 mmol) was added to a soln. of tert-butyl 4-(((3R,4R)-3-(4-(methoxycarbonyl)phenyl)-1-(2,2,2-trifluoroethyl)piperidin-4-yl)methyl)-5,7-dimethyl-1H-indole-1-carboxylate (Intermediate B-3, 107.7 mg, 192.8 μmol) in MeOH (1.00 mL) and THF (0.70 mL). The RM was heated to 50° C. for 4.5 h, then cooled to RT and diluted with an aq HCl soln. (4M, 482 μL, 1.928 mmol). EtOAc, brine and a small amount of water were added. The layers were separated and washed with brine and EtOAc. The combined org layers were dried over MgSO₄, filtered and the volatiles were removed under reduced pressure at 50° C. to give a slightly yellow oil that was purified over silica gel (12 g) using an automated purification system (NP; Teledyne ISCO®; collected at 240/269 nm; flow 30 mL/min; eluent: DCM+0 to 4.7% MeOH in 9.4 min). Pure fractions were combined and the volatiles were removed under reduced pressure at 50° C. to give the title compound (72.9 mg) as an off-white foam.

### Example 2: 4-((3S,4S)-4-((5,7-dimethyl-1H-indol-4-yl)methyl)-1-(2,2,2-trifluoro-ethyl)piperidin-3-yl)benzoic acid

Example 2 (72.8 mg, off-white foam) was prepared similar to Example 1, as described above, using tert-butyl 4-(((3S,4S)-3-(4-(methoxycarbonyl)phenyl)-1-(2,2,2-trifluoroethyl)piperidin-4-yl)methyl)-5,7-dimethyl-1H-indole-1-carboxylate (Intermediate B-2, 96.3 mg).

### Example Ex-8

### 4-((3R,4R)-4-((5,7-dimethyl-1H-indol-4-yl)methyl)-1-methylpiperidin-3-yl)benzoic acid

Aq NaOH soln. (4M, 511.5 μL, 2.046 mmol) was added to a soln. of tert-butyl 4-(((3R,4R)-3-(4-(tert-butoxycarbonyl)phenyl)-1-methylpiperidin-4-yl)methyl)-5,7-dimethyl-1H-indole-1-carboxylate (Intermediate B-11, 109.0 mg, 204.6 μmol) in MeOH (1.8 mL) and THF (0.5 mL). The resulting milky RM was heated to 55° C. which resulted in a clear soln. and stirring was continued at 55° C. for 23 h. The RM was allowed to cool to RT and quenched with aq HCl soln. (4M, 511.5 μL, 2.046 mmol). EtOAc, brine, and a small amount of water were added. The layers were separated, Me-THF was added, the layers were separated, and the aq layer was washed with Me-THF (3×). The combined org layers were dried over MgSO₄, filtered, and the volatiles were removed under reduced pressure at 50° C. The residue was triturated with ACN/water (9/1), filtered, and the solid was dried under reduced pressure at 50° C. to give the title compound (57.5 mg) as a white solid.

### Example Ex-10

### 4-((3R,4R)-4-((5,7-dimethyl-1H-indol-4-yl)methyl)-1-ethylpiperidin-3-yl)benzoic acid

An aq NaOH soln. (4M, 138.1 μL, 552.4 μmol) was added to a soln. of tert-butyl 4-(((3R,4R)-3-(4-(tert-butoxycarbonyl)phenyl)-1-ethylpiperidin-4-yl)methyl)-5,7-dimethyl-1H-indole-1-carboxylate (Intermediate B-14, 30.2 mg, 55.2 μmol) in MeOH (0.60 mL) and THF (0.20 mL). The RM was stirred at RT for 48 h, then for additional 24 h at 50° C. The mixture was allowed to cool to RT and quenched with aq HCl soln. (4M, 138 μL, 552.4 μmol) and then diluted with MeOH. Most of the volatiles were removed under reduced pressure at 50° C. The resulting slurry was brought into solution with MeOH and directly purified by preparative HPLC on a XBridge C18 column (30×100 mm, 5 μm) eluenting with 5 to 60% ACN in aq NH₄OH (0.1%) to provide 4-((3R,4R)-4-((5,7-dimethyl-1H-indol-4-yl)methyl)-1-ethylpiperidin-3-yl)benzoic acid (17.8 mg) as a white solid after concentration under reduced pressure.

### Example Ex-12

### 4-((3R,4R)-4-((5,7-dimethyl-1H-indol-4-yl)methyl)-1-(2-fluoroethyl)piperidin-3-yl)benzoic acid

TFA (0.3 mL) was added to a soln. of tert-butyl 4-(((3R,4R)-3-(4-(tert-butoxycarbonyl)phenyl)-1-(2-fluoroethyl)piperidin-4-yl)methyl)-5,7-dimethyl-1H-indole-1-carboxylate (Intermediate B-13, 48.0 mg, 84.99 μmol) in DCM (0.9 mL). The RM was stirred at RT for 1 h, diluted with DCE and the volatiles were removed under reduced pressure at 40° C. The crude residue was purified over C₁₈ (26 g) using an automated purification system (RP, Teledyne ISCO®; collected at 220/270 nm; flow 35 mL/min; eluent: water+10 to 60% ACN in 10 min). Pure fractions were combined, and ACN was removed under reduced pressure at 50° C. The resulting aq slurry was lyophilized overnight to give the title compound as a white solid (16.8 mg).

### Example Ex-15

### 4-((3R,4R)-4-((5,7-dimethyl-1H-indol-4-yl)methyl)-1-(2-hydroxy-2-methylpropyl)piperidin-3-yl)benzoic acid

To a solution of tert-butyl 4-(((3R,4R)-3-(4-(tert-butoxycarbonyl)phenyl)-1-(2-hydroxy-2-methylpropyl)piperidin-4-yl)methyl)-5,7-dimethyl-1H-indole-1-carboxylate (Intermediate B-22, 69.0 mg, 116.8 mmol) in MeOH (0.80 mL) and THF (0.30 mL) was added aq NaOH solution (4M, 292 μL, 1.168 mmol) and the resulting RM was stirred at 50° C. overnight. To the mixture was added aq HCl solution (4M, 297 μL, 1.168 mmol) and MeOH, and the resulting mixture was concentrated to a slurry under reduced pressure. Additional MeOH and two drops of water were added and the mixture was purified by preparative HPLC on a XBridge C18 column (30×100 mm, 5 μm) eluting with 5% to 28% ACN in aq NH₄OH (0.1%) to provide 4-((3R,4R)-4-((5,7-dimethyl-1H-indol-4-yl)methyl)-1-(2-hydroxy-2-methylpropyl)piperidin-3-yl)benzoic acid (43.8 mg) as a white solid after lyophilization.

### Example Ex-24 and Example Ex-25

### 4-((3R,4S)-4-((5,7-dimethyl-1H-indol-4-yl)methyl)-1-methylpyrrolidin-3-yl)benzoic acid and

### 4-((3S,4R)-4-((5,7-dimethyl-1H-indol-4-yl)methyl)-1-methylpyrrolidin-3-yl)benzoic acid

Step 1-a: To a solution of racemic tert-butyl 4-(((3S*,4R*)-4-(4-cyanophenyl)-1-methylpyrrolidin-3-yl)methyl)-5,7-dimethyl-1H-indole-1-carboxylate (Intermediate rac-B-18, 48 mg, 19 μmol) in IPA (2.5 mL) were added an aq KOH solution (5M, 250 μL, 1.25 mmol) and water (1.0 mL). The RM was heated at 150° C. for 4.5 h using microwave irradiation (Biotage® Initiator⁺). Additional aq KOH solution (5M, 75 μL) was added and heating was continued at 150° C. for 3 h, then at 165° C. for 1 h using microwave irradiation (Biotage® Initiator⁺). The mixture was concentrated under reduced pressure, and the crude residue was purified by preparative HPLC on a XBridge C18 OBD column (30×50 mm, 5 μm) eluting with 5% to 20% ACN in aq NH₄OH (5 mM) to provide racemic 4-((3R*,4S*)-4-((5,7-dimethyl-1H-indol-4-yl)methyl)-1-methylpyrrolidin-3-yl)benzoic acid (23.4 mg) as a white solid. LC-MS Method C-1: RT=0.70 min; MS m/z [M+H]⁺=363.2.

Step 1-b: Enantiomer separation and analytics according to Method SFC-4: racemic 4-((3R*,4S*)-4-((5,7-dimethyl-1H-indol-4-yl)methyl)-1-methylpyrrolidin-3-yl)benzoic acid (from Step 1-a, above, 23 mg) providing Example Ex-25 (Peak 1): 6.2 mg, white solid. LC-MS Method C-3: Rt=1.22 min; MS m/z [M+H]⁺=363.2; analytical chiral HPLC: Rt=2.35 min; 100% ee. Example Ex-24 (Peak 2): 6.3 mg, white solid. LC-MS Method C-3: Rt=1.24 min; MS m/z [M+H]⁺=363.2; analytical chiral HPLC: Rt=3.02 min; 98.6% ee.

### Example Ex-42

### 4-(((3R,4R)-3-(4-(1H-tetrazol-5-yl)phenyl)-1-methylpiperidin-4-yl)methyl)-5,7-dimethyl-1H-indole

Sodium azide (16.13 mg, 248.1 μmol) and ammonium chloride (12.39 mg, 231.6 μmol) were added to a solution of tert-butyl 4-(((3R,4R)-3-(4-cyanophenyl)-1-methylpiperidin-4-yl)methyl)-5,7-dimethyl-1H-indole-1-carboxylate (Intermediate B-67, 75.70 mg, 165.4 μmol) in DMF (0.750 mL) and the resulting mixture was stirred in a capped vial at 100° C. for ˜1.5 days. The RM was allowed to cool to RT and MeOH (2.0 mL) and K₂CO₃ (325 mesh 114.3 mg, 827.1 μmol) were added. Heating was continued at 55° C. for 18 h and stirring was continued at ambient temperature for ˜2 days. The mixture was diluted with MeOH, concentrated HCl (12M, 137.9 μL, 1.654 mmol) was added, and the resulting mixture was concentrated to a slurry under reduced pressure 50° C. Water was added and the mixture was purified by preparative HPLC on a XBridge C18 column (30×100 mm, 5 μm) eluting with 5% to 36.2% ACN in aq NH₄OH (0.1%) to provide 4-(((3R,4R)-3-(4-(1H-tetrazol-5-yl)phenyl)-1-methylpiperidin-4-yl)methyl)-5,7-dimethyl-1H-indole (26.1 mg) as a light yellowish, fluffy solid after concentration under reduced pressure, followed by lyophilization.

### Example Ex-44

### 4-((3R,4R)-4-((5,7-dimethyl-1H-indol-4-yl)methyl)-1-methylpiperidin-3-yl)benzonitrile

tert-Butyl 4-(((3R,4R)-3-(4-cyanophenyl)-1-methylpiperidin-4-yl)methyl)-5,7-dimethyl-1H-indole-1-carboxylate (Intermediate B-67, 22.0 mg, 48.07 μmol) was treated with DCM/TFA (3/1, 0.60 mL) and the resulting RM was stirred at RT for 30 min. The mixture was diluted with DCM and poured into aq Na₂CO₃ solution (10 wt %). The layers were then separated and the aq layer was extracted with DCM (2×). The combined org layers were dried over MgSO₄, filtered, and concentrated under reduced pressure at 50° C. The residue was purified by preparative TLC plate (MERCK 1.05744.0001, PLC silica gel 60 F254, 0.5 mm; DCM/{MeOH/NH₄OH 25%, 9/1}=92/8) provide to 4-((3R,4R)-4-((5,7-dimethyl-1H-indol-4-yl)methyl)-1-methylpiperidin-3-yl)benzonitrile (12.8 mg) as a white foam.

### Example Ex-52

### 5,7-dimethyl-4-(((3R,4R)-1-methyl-3-(1-methyl-1H-pyrazol-4-yl)piperidin-4-yl)methyl)-1H-indole

To a solution of tert-butyl 5,7-dimethyl-4-(((3R,4R)-1-methyl-3-(1-methyl-1H-pyrazol-4-yl)piperidin-4-yl)methyl)-1H-indole-1-carboxylate (Intermediate B-77, 31.7 mg, 72.6 μmol) in MeOH (1.20 mL) was added K₂CO₃ (325 mesh, 50.2 mg, 363 μmol) and the resulting mixture was stirred at 50° C. overnight. The volatiles were removed under reduced pressure at 50° C., and the residue was partitioned between water and DCM. The separated aq layer was extracted with DCM (2×). The combined org layers were dried over MgSO₄, filtered, and concentrated under reduced pressure at 50° C. The residue was purified by preparative TLC plate (MERCK 1.05744.0001, PLC silica gel 60 F254, 0.5 mm; DCM/(MeOH:NH₄OH 25% 9:1)=88/12) to provide 5,7-dimethyl-4-(((3R,4R)-1-methyl-3-(1-methyl-1H-pyrazol-4-yl)piperidin-4-yl)methyl)-1H-indole (21.0 mg) as an off-white solid.

### Example Ex-58

### Racemic 4-((3R*,4S*)-4-((5,7-dimethyl-1H-indol-4-yl)methyl)-1-methylpyrrolidin-3-yl)benzamide

To a solution of racemic tert-butyl 4-(((3S*,4R*)-4-(4-cyanophenyl)-1-methylpyrrolidin-3-yl)methyl)-5,7-dimethyl-1H-indole-1-carboxylate (Intermediate rac-B-18, 68 mg, 15 μmol) in tert-amyl alcohol (2.5 mL) was added an aq NaOH solution (1M, 1.2 mL, 1.20 mmol). The RM was heated at 110° C. for 84 h and then at 160° C. for 2 h using microwave irradiation (Biotage® Initiator⁺). Solid lithium hydroxide (50 mg, 2.09 mmol) was added, and the RM was heated at 165° C. for 16 h using microwave irradiation (Biotage® Initiator*). The mixture was concentrated under reduced pressure and the crude residue was purified by preparative HPLC on a XBridge C18 OBD column (30×50 mm, 5 μm) eluting with 10% to 0% ACN in aq NH₄OH (5 mM) to provide racemic 4-((3R*,4S*)-4-((5,7-dimethyl-1H-indol-4-yl)methyl)-1-methylpyrrolidin-3-yl)benzamide (3.0 mg) as an off-white solid. LC-MS Method C-3: Rt=0.92 min; [M+H]⁺=362.4

### Example Ex-59

### 4-((3R,4S)-4-((5-cyclopropyl-7-methyl-1H-indol-4-yl)methyl)-1-methylpyrrolidin-3-yl)benzoic acid

Step 1-a: Racemic 4-((3R*,4S*)-4-((5-cyclopropyl-7-methyl-1H-indol-4-yl)methyl)-1-methylpyrrolidin-3-yl)benzoic acid was prepared according to Step 1-a of the method described for Example Ex-24 herein above, using racemic tert-butyl 4-(((3S*,4R*)-4-(4-cyanophenyl)-1-methylpyrrolidin-3-yl)methyl)-5-cyclopropyl-7-methyl-1H-indole-1-carboxylate (Intermediate B-87).

Step 1-b: Enantiomer separation and analytics according to Method SFC-12: racemic 4-((3R*,4S*)-4-((5-cyclopropyl-7-methyl-1H-indol-4-yl)methyl)-1-methylpyrrolidin-3-yl)benzoic acid

(from Step 1-a, above, 40 mg) providing Example Ex-59 (Peak 2): 16.3 mg, white solid. LC-MS Method C-3: Rt=1.44 min; [M+H]⁺=389.3; analytical chiral HPLC: Rt=3.10 min; 95.9% ee.

### Example Ex-60

### 4-(((3S,4R)-4-(4-(2H-tetrazol-5-yl)phenyl)-1-methylpyrrolidin-3-yl)methyl)-5,7-dimethyl-1H-indole

Step 1-a: Racemic 4-(((3S*,4R*)-4-(4-(2H-tetrazol-5-yl)phenyl)-1-methylpyrrolidin-3-yl)methyl)-5,7-dimethyl-1H-indole was prepared according to the method described for Example Ex-42 herein above, using racemic tert-butyl 4-(((3S*,4R*)-4-(4-cyanophenyl)-1-methylpyrrolidin-3-yl)methyl)-5-cyclopropyl-7-methyl-1H-indole-1-carboxylate (Intermediate rac-B-18).

Step 1-b: Enantiomer separation and analytics according to Method SFC-13: racemic 4-(((3S*,4R*)-4-(4-(2H-tetrazol-5-yl)phenyl)-1-methylpyrrolidin-3-yl)methyl)-5,7-dimethyl-1H-indole

(25 mg) providing Example Ex-60 (Peak 2): 3.5 mg, white solid. LC-MS Method C-3: Rt=1.15 min; [M+H]⁺=387.5; analytical chiral HPLC: Rt=3.48 min; 98.9% ee.

The following table includes examples described above and those synthesized using the appropriate starting material and applying similar methods described in the examples above.

| | | | LC-MS |
|---|---|---|---|
| Example | Chemical Structure | ¹H NMR | MS m/z |
| Ex-1 | 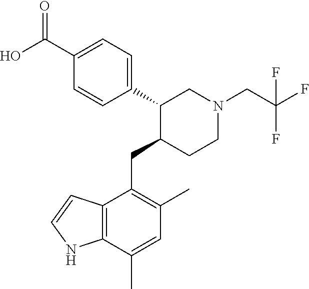 [SMILES: Cc1cc(C)c2[nH]ccc2c1C[C@@H]1CCN(CC(F)(F)F)C[C@H]1c1ccc(C(=O)O)cc1] | 400 MHZ, DMSO-d₆ δ [ppm] 12.90 (br s, 1H), 10.79 (s, 1H), 7.96 (d, J = 8.1 Hz, 2H), 7.54 (d, J = 8.1 Hz, 2H), 7.19-7.16 (m, 1H), 6.61 (s, 1H), 6.17 (dd, J = 3.1, 1.9 Hz, 1H), 3.24-3.09 (m, 2H, partially overlapping with water peak), 2.92-2.75 (m, 3H), 2.58-2.51 (m, 1H, partially overlapping with DMSO peak), 2.49-2.41 (m, 2H, partially overlapping with DMSO peak), 2.34 (s, 3H), 2.27-2.17 (m, 1H), 2.10 (s, 3H), 1.93-1.82 (m, 1H), 1.50-1.37 (m, 1H), 1.30 (m, 1H). 27 of 27H reported | Method B-2: Rt = 6.46 min; [M + H]⁺ = 445.4 |
| Ex-2 | 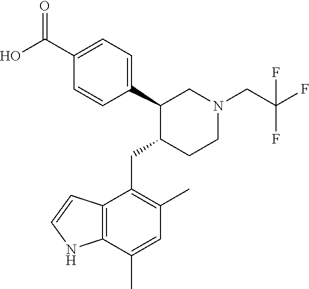 [SMILES: Cc1cc(C)c2[nH]ccc2c1C[C@H]1CCN(CC(F)(F)F)C[C@@H]1c1ccc(C(=O)O)cc1] | 400 MHZ, DMSO-d₆ δ [ppm] 12.88 (br s, 1H), 10.79 (s, 1H), 7.96 (d, J = 8.1 Hz, 2H), 7.54 (d, J = 8.1 Hz, 2H), 7.20-7.16 (m, 1H), 6.61 (s, 1H), 6.17 (dd, J = 3.1, 1.9 Hz, 1H), 3.23-3.07 (m, 2H, partially overlapping with water peak), 2.93-2.75 (m, 3H), 2.59-2.51 (m, 1H, partially overlapping with DMSO peak), 2.48-2.40 (m, 2H, partially overlapping with DMSO peak), 2.34 (s, 3H), 2.27-2.21 (m, 1H), 2.10 (s, 3H), 1.95-1.81 (m, 1H), 1.49-1.36 (m, 1H), 1.35-1.26 (m, 1H). 27 of 27H reported | Method B-2: Rt = 6.50 min; [M + H]⁺ = 445.1 |
| Ex-3 | 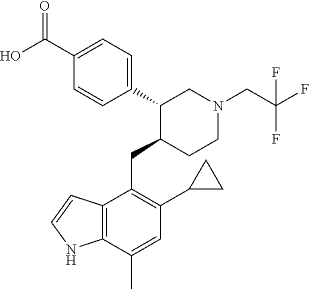 [SMILES: Cc1cc(C2CC2)c(C[C@@H]2CCN(CC(F)(F)F)C[C@H]2c2ccc(C(=O)O)cc2)c2cc[nH]c12] | 400 MHZ, DMSO-d₆ δ [ppm] 12.88 (br s, 1H), 10.79 (s, 1H), 7.95 (d, J = 8.3 Hz, 2H), 7.53 (d, J = 8.3 Hz, 2H), 7.21-7.18 (m, 1H), 6.41 (s, 1H), 6.22 (dd, J = 3.1, 1.9 Hz, 1H), 3.24-3.07 (m, 2H), 2.94-2.76 (m, 3H), 2.70-2.51 (m, 3H), 2.33 (s, 3H), 2.28-2.19 (m, 1H), 2.01-1.89 (m, 1H), 1.79-1.69 (m, 1H), 1.54-1.40 (m, 1H), 1.35 (dd, J = 13.5, 3.5 Hz, 1H), 0.88-0.78 (m, 1H), 0.55- 0.43 (m, 2H), 0.35-0.24 (m, 1H). 29 of 29H reported | Method B-2: Rt = 6.66 min; [M + H]⁺ = 471.2 |
| Ex-4 | 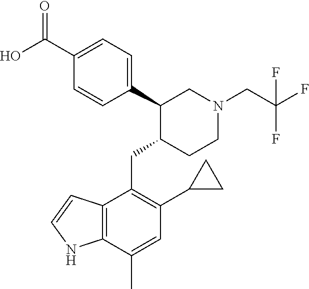 [SMILES: Cc1cc(C2CC2)c(C[C@H]2CCN(CC(F)(F)F)C[C@@H]2c2ccc(C(=O)O)cc2)c2cc[nH]c12] | 400 MHZ, DMSO-d₆ δ [ppm] 12.88 (br s, 1H), 10.79 (s, 1H), 7.95 (d, J = 8.2 Hz, 2H), 7.53 (d, J = 8.2 Hz, 2H), 7.21-7.18 (m, 1H), 6.41 (s, 1H), 6.22 (dd, J = 3.1, 1.9 Hz, 1H), 3.26-3.06 (m, 2H), 2.96-2.75 (m, 3H), 2.71-2.52 (m, 3H), 2.33 (s, 3H), 2.27-2.19 (m, 1H), 2.00-1.87 (m, 1H), 1.80-1.70 (m, 1H), 1.53-1.41 (m, 1H), 1.35 (dd, J = 13.5, 3.5 Hz, 1H), 0.87-0.78 (m, 1H), 0.57- 0.44 (m, 2H), 0.35-0.26 (m, 1H). 29 of 29H reported | Method B-2: Rt = 6.68 min; [M + H]⁺ = 471.1 |
| Ex-5 | 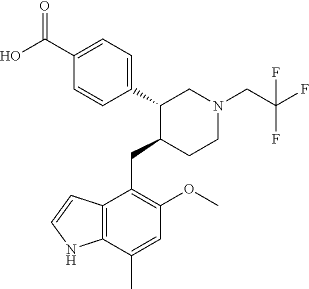 [SMILES: COc1cc(C)c2[nH]ccc2c1C[C@@H]1CCN(CC(F)(F)F)C[C@H]1c1ccc(C(=O)O)cc1] | 400 MHZ, DMSO-d₆ δ [ppm] 12.84 (br s, 1H), 10.68 (s, 1H), 7.95 (d, J = 8.1 Hz, 2H), 7.51 (d, J = 8.1 Hz, 2H), 7.19-7.14 (m, 1H), 6.82 (s, 1H), 6.26 (dd, J = 3.0, 1.8 Hz, 1H), 3.69 (s, 3H), 3.24-3.07 (m, 2H), 2.92-2.84 (m, 2H), 2.77-2.69 (m, 1H), 2.48-2.41 (m, 1H), 2.36 (d, J = 7.1 Hz, 2H), 2.29-2.20 (m, 4H), 1.92-1.81 (m, 1H), 1.48-1.30 (m, 2H). 27 of 27H reported | Method B-2: Rt = 6.10 min; [M + H]⁺ = 461.1 |
| Ex-6 | 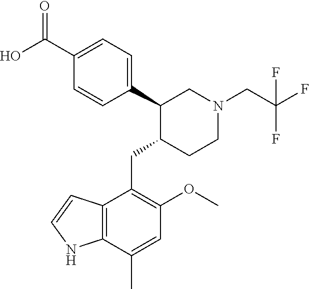 [SMILES: COc1cc(C)c2[nH]ccc2c1C[C@H]1CCN(CC(F)(F)F)C[C@@H]1c1ccc(C(=O)O)cc1] | 400 MHZ, DMSO-d₆ δ [ppm] 12.84 (br s, 1H), 10.68 (s, 1H), 7.95 (d, J = 8.1 Hz, 2H), 7.51 (d, J = 8.1 Hz, 2H), 7.18-7.14 (m, 1H), 6.82 (s, 1H), 6.26 (dd, J = 3.0, 1.8 Hz, 1H), 3.69 (s, 3H), 3.24-3.07 (m, 2H), 2.92-2.84 (m, 2H), 2.77-2.69 (m, 1H), 2.48-2.41 (m, 1H), 2.36 (d, J = 7.1 Hz, 2H), 2.29-2.20 (m, 4H), 1.92-1.81 (m, 1H), 1.48-1.30 (m, 2H). 27 of 27H reported | Method B-2: Rt = 6.10 min; [M + H]⁺ = 461.4 |
| Ex-7 | 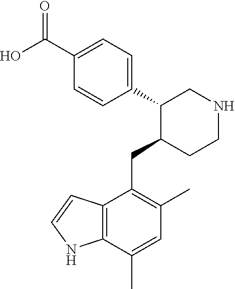 [SMILES: Cc1cc(C)c2[nH]ccc2c1C[C@@H]1CCNC[C@H]1c1ccc(C(=O)O)cc1] | 600 MHz, DMSO-d₆ δ [ppm] 10.83 (s, 1H), 7.94 (d, J = 7.7 Hz, 2H), 7.44 (d, J = 7.8 Hz, 2H), 7.20-7.10 (m, 1H), 6.61 (s, 1H), 6.13-6.10 (m, 1H), 3.09 (d, J = 12.0 Hz, 1H), 3.03 (d, J = 12.3 Hz, 1H), 2.89 (t, J = 12.2 Hz, 1H), 2.78-2.70 (m, 1H), 2.56 (d, J = 11.9 Hz, 1H), 2.50-2.45 (m, 1H, partially overlapping with DMSO-peak), 2.39 (t, J = 12.5 Hz, 1H), 2.33 (s, 3H), 2.09 (d, J = 16.5 Hz, 4H), 1.46-1.31 (m, 2H). 24 of 26H reported (—COOH, piperidine-NH not observed) | Method B-2: Rt = 2.55 min; [M + H]⁺ = 363.3 |
| Ex-8 | 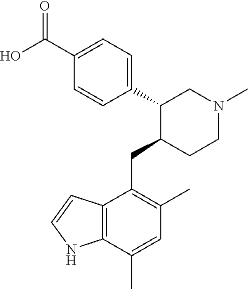 [SMILES: Cc1cc(C)c2[nH]ccc2c1C[C@@H]1CCN(C)C[C@H]1c1ccc(C(=O)O)cc1] | 600 MHZ, DMSO-d₆ δ [ppm] 10.81 (s, 1H), 7.96 (d, J = 7.8 Hz, 2H), 7.51 (d, J = 7.8 Hz, 2H), 7.20-7.15 (m, 1H), 6.61 (s, 1H), 6.17-6.11 (m, 1H), 2.84-2.70 (m, 3H), 2.49-2.38 (m, 2H), 2.34 (s, 3H), 2.20-2.07 (m, 7H), 1.89- 1.74 (m, 2H), 1.47-1.38 (m, 1H), 1.32 (d, J = 13.0 Hz, 1H). 27 of 28H reported (—COOH not observed) | Method B-2: Rt = 2.41 min; [M + H]⁺ = 377.2 Analytical Method SFC- 11: Rt = 3.73 min |
| Ex-9 | 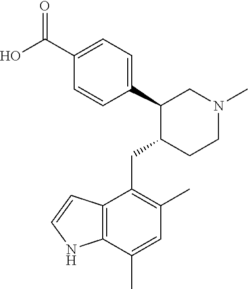 [SMILES: Cc1cc(C)c2[nH]ccc2c1C[C@H]1CCN(C)C[C@@H]1c1ccc(C(=O)O)cc1] | 400 MHZ, DMSO-d₆ δ [ppm] 10.79 (s, 1H), 7.96 (d, J = 7.9 Hz, 2H), 7.52 (d, J = 7.9 Hz, 2H), 7.22-7.14 (m, 1H), 6.61 (s, 1H), 6.16-6.12 (m, 1H), 2.83-2.68 (m, 3H), 2.50-2.37 (m, partially overlapping with DMSO-peak, 2H), 2.34 (s, 3H), 2.16 (s, 3H), 2.14- 2.04 (m, 4H), 1.89-1.72 (m, 2H), 1.48- 1.29 (m, 2H). 27 of 28H reported (—COOH not observed) | LC-MS Method B-2: Rt = 2.39 min; MS m/z [M + H]⁺ = 377.4 Analytical Method SFC- 11: Rt = 0.87 min |
| Ex-10 | 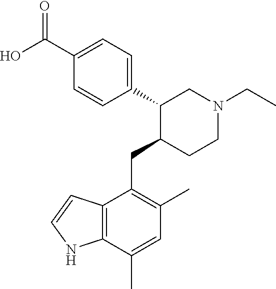 [SMILES: CCN1CC[C@@H](Cc2c(C)cc(C)c3[nH]ccc23)[C@H](c2ccc(C(=O)O)cc2)C1] | 600 MHZ, DMSO-d₆ δ [ppm] 10.81 (s, 1H), 7.96 (d, J = 7.8 Hz, 2H), 7.52 (d, J = 7.9 Hz, 2H), 7.19-7.17 (m, 1H), 6.61 (s, 1H), 6.15 (dd, J = 3.1, 1.9 Hz, 1H), 2.87 (dd, J = 20.9, 11.3 Hz, 2H), 2.80-2.73 (m, 1H), 2.49-2.38 (m, 2H), 2.37-2.31 (m, 5H), 2.16-2.07 (m, 4H), 1.91-1.83 (m, 1H), 1.82-1.73 (m, 1H), 1.45-1.37 (m, 1H), 1.37-1.31 (m, 1H), 0.97 (t, J = 7.1 Hz, 3H). 29 of 30H reported (COOH not observed) | Method B-2: Rt = 2.49 min; [M + H]⁺ = 391.2 |
| Ex-11 | 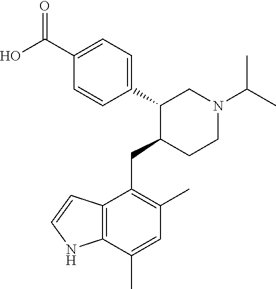 [SMILES: Cc1cc(C)c2[nH]ccc2c1C[C@@H]1CCN(C(C)C)C[C@H]1c1ccc(C(=O)O)cc1] | 600 MHZ, DMSO-d₆ δ [ppm] 10.80 (s, 1H), 7.96 (d, J = 7.9 Hz, 2H), 7.52 (d, J = 7.8 Hz, 2H), 7.19-7.17 (m, 1H), 6.60 (s, 1H), 6.15 (dd, J = 3.1, 1.9 Hz, 1H), 2.81-2.69 (m, 4H), 2.48-2.38 (m, 2H), 2.37-2.31 (m, 4H), 2.10 (s, 3H), 2.05-1.98 (m, 1H), 1.90-1.82 (m, 1H), 1.41-1.31 (m, 2H), 0.93 (d, J = 6.5 Hz, 6H). 31 of 32H reported (—COOH not observed) | Method B-2: Rt = 2.56 min; [M + H]⁺ = 405.3 |
| Ex-12 | 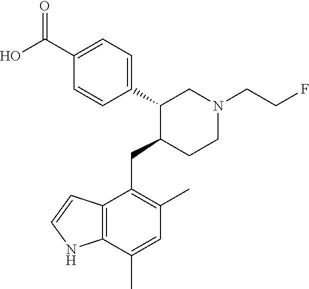 [SMILES: Cc1cc(C)c2[nH]ccc2c1C[C@@H]1CCN(CCF)C[C@H]1c1ccc(C(=O)O)cc1] | 600 MHZ, DMSO-d₆ δ [ppm] 10.81 (s, 1H), 7.96 (d, J = 7.9 Hz, 2H), 7.54 (d, J = 7.9 Hz, 2H), 7.19-7.17 (m, 1H), 6.61 (s, 1H), 6.16 (dd, J = 3.1, 1.9 Hz, 1H), 4.49 (dt, J = 47.7, 4.9 Hz, 2H), 4.45 (t, J = 4.9 Hz, 1H), 2.90-2.86 (m, 1H), 2.83 (d, J = 11.0 Hz, 1H), 2.80- 2.74 (m, 1H), 2.66-2.52 (m, 2H), 2.48- 2.39 (m, 2H), 2.34 (s, 3H), 2.24-2.18 (m, 1H), 2.10 (s, 3H), 1.92-1.81 (m, 2H), 1.45-1.36 (m, 1H), 1.36-1.29 (m, 1H). TFA salt; 28 of 30H reported (—COOH, N + H not observed) | Method B-2: Rt = 2.58 min; [M + H]⁺ = 409.2 |
| Ex-13 | 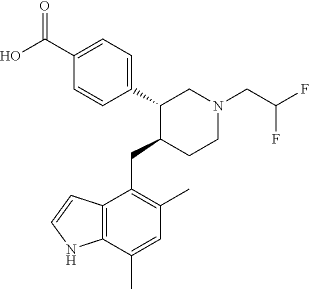 [SMILES: Cc1cc(C)c2[nH]ccc2c1C[C@@H]1CCN(CC(F)F)C[C@H]1c1ccc(C(=O)O)cc1] | 600 MHZ, DMSO-d₆ δ [ppm] 10.79 (s, 1H), 7.96 (d, J = 7.8 Hz, 2H), 7.53 (d, J = 7.9 Hz, 2H), 7.19-7.17 (m, 1H), 6.61 (s, 1H), 6.24-5.97 (m, 2H), 2.93-2.87 (m, 1H), 2.84 (d, J = 11.5 Hz, 1H), 2.81-2.64 (m, 3H), 2.49-2.39 (m, 2H), 2.39-2.30 (m, 4H), 2.10 (s, 3H), 2.08-2.00 (m, 1H), 1.91-1.81 (m, 1H), 1.46-1.36 (m, 1H), 1.34-1.27 (m, 1H). 27 of 28H reported (COOH not observed) | Method B-2: Rt = 4.27 min; [M + H]⁺ = 427.3 |
| Ex-14 | 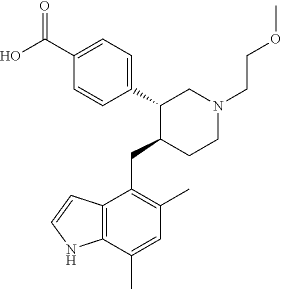 [SMILES: COCCN1CC[C@@H](Cc2c(C)cc(C)c3[nH]ccc23)[C@H](c2ccc(C(=O)O)cc2)C1] | 600 MHZ, DMSO-d₆ δ [ppm] 10.80 (s, 1H), 7.95 (d, J = 7.9 Hz, 2H), 7.52 (d, J = 7.9 Hz, 2H), 7.19-7.16 (m, 1H), 6.60 (s, 1H), 6.15 (dd, J = 3.1, 1.9 Hz, 1H), 3.39 (t, J = 5.8 Hz, 2H, partially overlapping with water peak), 3.18 (s, 3H, partially overlapping with water peak), 2.89-2.84 (m, 1H), 2.81 (d, J = 11.4 Hz, 1H), 2.77 ~ 2.72 (m, 1H), 2.47- 2.39 (m, 4H), 2.34 (s, 3H), 2.18-7.13 (m, 1H), 2.10 (s, 3H), 1.87-1.79 (m, 2H), 1.43-1.35 (m, 1H), 1.33-1.28 (m, 1H). 31 of 32H reported (—COOH not observed) | Method B-2: Rt = 2.57 min; [M + H]⁺ = 421.4 |
| Ex-15 | 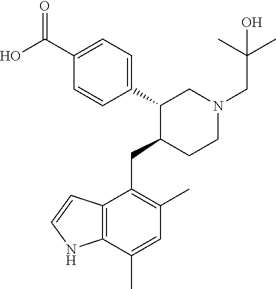 [SMILES: Cc1cc(C)c2[nH]ccc2c1C[C@@H]1CCN(CC(C)(C)O)C[C@H]1c1ccc(C(=O)O)cc1] | 600 MHZ, DMSO-d₆ δ [ppm] 10.80 (s, 1H), 7.95 (d, J = 7.9 Hz, 2H), 7.51 (d, J = 7.8 Hz, 2H), 7.17-7.18 (m, 1H), 6.60 (s, 1H), 6.14 (dd, J = 3.1, 1.9 Hz, 1H), 2.99-2.95 (m, 1H), 2.85 (d, J = 11.5 Hz, 1H), 2.80-2.74 (m, 1H), 2.47- 2.39 (m, 2H), 2.34 (s, 3H), 2.31-2.26 (m, 1H), 2.17 (s, 2H), 2.10 (s, 3H), 2.02- 1.95 (m, 1H), 1.85-1.76 (m, 1H), 1.47- 1.38 (m, 1H), 1.30-1.23 (m, 1H), 1.06 (d, J = 6.0 Hz, 6H). 32 of 34H reported (—COOH, —OH not observed) | Method B-2: Rt = 2.51 min; [M + H]⁺ = 435.4 |
| Ex-16 | 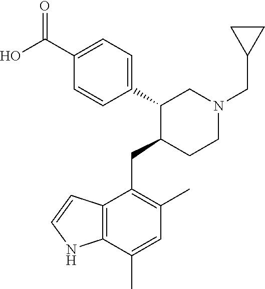 [SMILES: Cc1cc(C)c2[nH]ccc2c1C[C@@H]1CCN(CC2CC2)C[C@H]1c1ccc(C(=O)O)cc1] | 600 MHZ, DMSO-d₆ δ [ppm] 10.80 (s, 1H), 7.96 (d, J = 7.9 Hz, 2H), 7.52 (d, J = 7.9 Hz, 2H), 7.19-7.17 (m, 1H), 6.61 (s, 1H), 6.15 (dd, J = 3.1, 1.9 Hz, 1H), 2.99 (dd, J = 11.9, 3.9 Hz, 1H), 2.92 (d, J = 11.3 Hz, 1H), 2.82-2.74 (m, 1H), 2.48-2.40 (m, 2H), 2.34 (s, 3H), 2.24-2.12 (m, 3H), 2.10 (s, 3H), 1.90- 1.78 (m, 2H), 1.47-1.37 (m, 1H), 1.37- 1.29 (m, 1H), 0.84-0.76 (m, 1H), 0.44- 0.36 (m, 2H), 0.06-0.04 (m, 2H). 31 of 32H reported (—COOH not observed) | Method B-2: Rt = 2.55 min; [M + H]⁺ = 417.5 |
| Ex-17 | 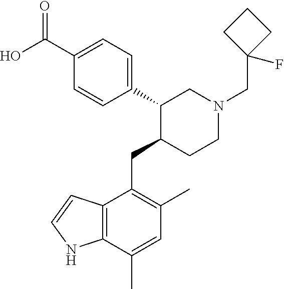 [SMILES: Cc1cc(C)c2[nH]ccc2c1C[C@@H]1CCN(CC2(F)CCC2)C[C@H]1c1ccc(C(=O)O)cc1] | 600 MHZ, DMSO-d₆ δ [ppm] 10.78 (s, 1H), 7.94 (d, J = 7.8 Hz, 2H), 7.48 (d, J = 7.8 Hz, 2H), 7.19-7.16 (m, 1H), 6.60 (s, 1H), 6.17-6.14 (m, 1H), 4.60-4.49 (m, 4H), 2.88-2.77 (m, 4H), 2.77-2.70 (m, 1H), 2.49-2.39 (m, 2H), 2.37-2.30 (m, 4H), 2.10 (s, 3H), 2.06- 1.97 (m, 1H), 1.88-1.78 (m, 1H), 1.45- 1.36 (m, 1H), 1.34-1.27 (m, 1H). 30 of 31H reported (—COOH not observed) | Method B-2: Rt = 2.86 min; [M + H]⁺ = 451.2 |
| Ex-18 | 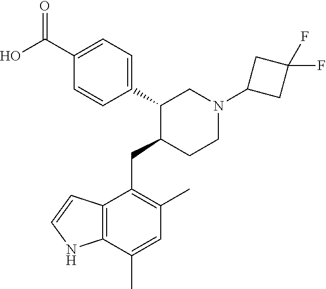 [SMILES: Cc1cc(C)c2[nH]ccc2c1C[C@@H]1CCN(C2CC(F)(F)C2)C[C@H]1c1ccc(C(=O)O)cc1] | 400 MHZ, DMSO-d₆ δ [ppm] 12.88 (br s, 1H), 10.79 (s, 1H), 7.96 (d, J = 8.0 Hz, 2H), 7.53 (d, J = 8.0 Hz, 2H), 7.20-7.15 (m, 1H), 6.61 (s, 1H), 6.15 (dd, J = 3.1, 1.9 Hz, 1H), 2.83-2.70 (m, 3H), 2.70-2.55 (m, 3H), 2.48-2.28 (m, 7H), 2.09 (s, 3H), 2.04-1.96 (m, 1H), 1.95-1.80 (m, 1H), 1.73-1.60 (m, 1H), 1.45-1.29 (m, 2H). 30 of 30H reported | Method B-2: Rt = 3.53 min; [M + H]⁺ = 453.2 |
| Ex-19 | 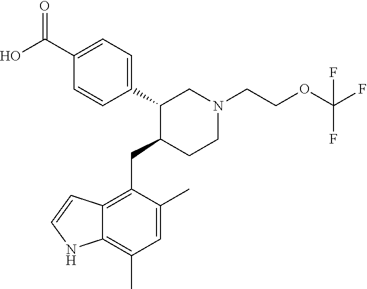 [SMILES: Cc1cc(C)c2[nH]ccc2c1C[C@@H]1CCN(CCOC(F)(F)F)C[C@H]1c1ccc(C(=O)O)cc1] | 600 MHZ, DMSO-d₆ δ [ppm] 12.95 (br s, 1H), 10.81 (s, 1H), 7.96 (d, J = 7.9 Hz, 2H), 7.53 (d, J = 7.9 Hz, 2H), 7.20-7.17 (m, 1H), 6.61 (s, 1H), 6.18- 6.14 (m, 1H), 4.11 (t, J = 5.6 Hz, 2H), 2.89-2.85 (m, 1H), 2.85-2.80 (m, 1H), 2.80-2.73 (m, 1H), 2.65-2.55 (m, 2H), 2.48-2.40 (m, 2H), 2.34 (s, 3H), 2.26- 2.17 (m, 1H), 2.10 (s, 3H), 1.92-1.81 (m, 2H), 1.44-1.36 (m, 1H), 1.35-1.28 (m, 1H). 29 of 29H reported | Method B-2: Rt 3.52 min; [M + H]⁺ = 475.3 |
| Ex-20 | 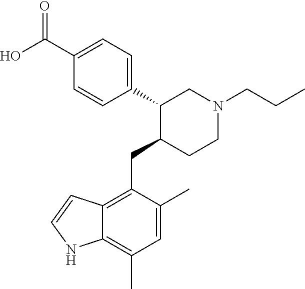 [SMILES: CCCN1CC[C@@H](Cc2c(C)cc(C)c3[nH]ccc23)[C@H](c2ccc(C(=O)O)cc2)C1] | 600 MHZ, DMSO-d₆ δ [ppm] 10.81 (s, 1H), 7.96 (d, J = 7.9 Hz, 2H), 7.52 (d, J = 7.8 Hz, 2H), 7.21-7.14 (m, 1H), 6.60 (s, 1H), 6.18-6.10 (m, 1H), 2.89-2.80 (m, 2H), 2.80-2.73 (m, 1H), 2.48-2.38 (m, 2H), 2.34 (s, 3H), 2.28- 2.18 (m, 2H), 2.16-2.06 (m, 4H), 1.90- 1.82 (m, 1H), 1.80-1.73 (m, 1H), 1.45- 1.36 (m, 3H), 1.36-1.30 (m, 1H), 0.82 (t, J = 7.4 Hz, 3H). 31 of 32H reported (—COOH not observed) | Method B-2: Rt 2.71 min; [M + H]⁺ = 405.5 |
| Ex-21 | 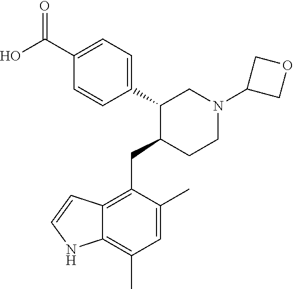 [SMILES: Cc1cc(C)c2[nH]ccc2c1C[C@@H]1CCN(C2COC2)C[C@H]1c1ccc(C(=O)O)cc1] | 600 MHZ, DMSO-d₆ (with 1 drop of TFA) δ [ppm] 13.05 (br s, 1H), 11.09 (br s, 1H), 10.91 (s, 1H), 8.03 (d, J = 7.9 Hz, 2H), 7.62 (d, J = 7.9 Hz, 2H), 7.24-7.21 (m, 1H), 6.63 (s, 1H), 6.12-6.07 (m, 1H), 4.85- 4.77 (m, 2H, partially overlapping with water peak), 4.72-4.60 (m, 2H, partially overlapping with water peak), 4.32- 4.22 (m, 1H), 3.41-3.36 (m, 1H), 3.34- 3.29 (m, 1H), 3.24-3.16 (m, 1H), 3.12- 3.05 (m, 1H), 2.80-2.72 (m, 1H), 2.50- 2.43 (m, 2H, partially overlapping with DMSO peak), 2.34 (s, 3H), 2.25-2.15 (m, 1H), 2.08 (s, 3H), 1.76-1.66 (m, 1H), 1.56-1.50 (m, 1H). TFA-salt; 31 of 31H reported | Method B-2: Rt 2.65 min; [M + H]⁺ = 419.5 |
| Ex-22 | 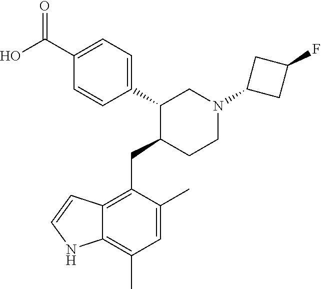 [SMILES: Cc1cc(C)c2[nH]ccc2c1C[C@@H]1CCN([C@H]2C[C@H](F)C2)C[C@H]1c1ccc(C(=O)O)cc1] | 600 MHZ, DMSO-d₆ δ [ppm] 10.81 (s, 1H), 7.96 (d, J = 7.9 Hz, 2H), 7.53 (d, J = 7.9 Hz, 2H), 7.20-7.17 (m, 1H), 6.60 (s, 1H), 6.15 (dd, J = 3.1, 1.9 Hz, 1H), 4.85-4.70 (m, 1H), 2.78-2.70 (m, 3H), 2.49-2.39 (m, 4H), 2.33 (s, 3H), 2.22-2.16 (m, 1H), 2.09 (s, 3H), 1.96-1.91 (m, 1H), 1.91-1.82 (m, 3H), 1.63-1.57 (m, 1H), 1.42-1.30 (m, 2H). 30 of 31H reported (—COOH not observed) | Method B-2: Rt 2.52 min; [M + H]⁺ = 435.3 |
| Ex-23 | 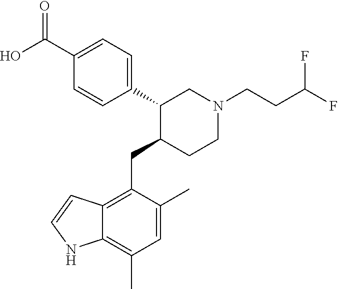 [SMILES: Cc1cc(C)c2[nH]ccc2c1C[C@@H]1CCN(CCC(F)F)C[C@H]1c1ccc(C(=O)O)cc1] | 600 MHz, DMSO-d₆ δ [ppm] 10.81 (s, 1H), 7.96 (d, J = 7.9 Hz, 2H), 7.53 (d, J = 7.9 Hz, 2H), 7.21-7.16 (m, 1H), 6.61 (s, 1H), 6.18-5.95 (m, 2H), 2.87-2.79 (m, 2H), 2.78-2.71 (m, 1H), 2.48-2.36 (m, 4H), 2.34 (s, 3H), 2.14- 2.08 (m, 4H), 2.02-1.91 (m, 2H), 1.89- 1.81 (m, 1H), 1.80-1.74 (m, 1H), 1.43- 1.35 (m, 1H), 1.35-1.29 (m, 1H). 29 of 30H reported (—COOH not observed) | Method B-2: Rt 2.64 min; [M + H]⁺ = 441.2 |
| Ex-24 | 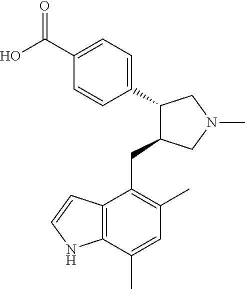 [SMILES: Cc1cc(C)c2[nH]ccc2c1C[C@@H]1CN(C)C[C@H]1c1ccc(C(=O)O)cc1] | 400 MHZ, DMSO-d₆ δ [ppm] 10.78 (s, 1H), 7.79 (d, J = 8.1 Hz, 2H), 7.26 (d, J = 8.2 Hz, 2H), 7.20-7.02 (m, 1H), 6.56 (s, 1H), 6.23-5.91 (m, 1H), 3.13-3.05 (m, 2H), 2.89-2.83 (m, 6H), 2.31 (s, 3H), 2.22 (s, 3H), 2.06 (s, 3H). 25 of 26H reported (—COOH not observed) | Method C-3: Rt = 1.12 min; [M + H]⁺ = 363.4 |
| Ex-25 | 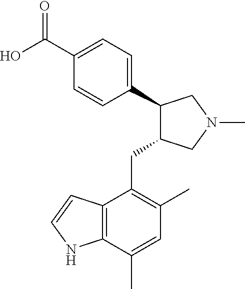 [SMILES: Cc1cc(C)c2[nH]ccc2c1C[C@H]1CN(C)C[C@@H]1c1ccc(C(=O)O)cc1] | 400 MHz, methanol-d4 δ [ppm] 7.95 (d, J = 8.1 Hz, 2H), 7.33 (d, J = 7.9 Hz, 2H), 7.20-7.07 (m, 1H), 6.63 (s, 1H), 6.32-6.14 (m, 1H), 3.74-3.58 (m, 1H), 3.48-3.39 (m, 2H), 3.35-3.30 (m, 2H, partially overlapping with solvent peak), 3.06-2.93 (m, 2H), 2.91-2.81 (m, 4H), 2.37 (s, 3H), 2.14 (s, 3H). 24 of 26H reported (—COOH, NH not observed) | Method C-3: Rt = 1.22 min; [M + H]⁺ = 363.2 |
| Ex-26 | 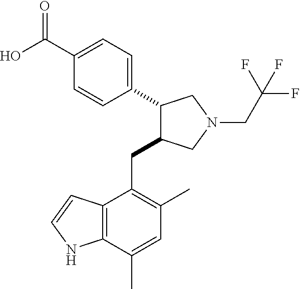 [SMILES: Cc1cc(C)c2[nH]ccc2c1C[C@@H]1CN(CC(F)(F)F)C[C@H]1c1ccc(C(=O)O)cc1] | Racemic mixture 400 MHz, methanol-d4 δ [ppm] 7.86 (d, J = 8.2 Hz, 2H), 7.22 (d, J = 8.3 Hz, 2H), 7.14-7.05 (m, 1H), 6.61 (S, 1H), 6.32-6.18 (m, 1H), 3.25-3.06 (m, 4H), 3.00-2.92 (m, 3H), 2.89-2.78 (m, 2H), 2.72-2.60 (m, 1H), 2.38 (s, 3H), 2.16 (s, 3H). 23 of 25H reported (—COOH, NH not observed) | Method C-3: Rt = 2.38 min; [M + H]⁺ = 431.3 |
| Ex-27 | 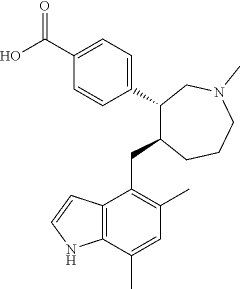 [SMILES: Cc1cc(C)c2[nH]ccc2c1C[C@@H]1CCCN(C)C[C@H]1c1ccc(C(=O)O)cc1] | 400 MHZ, DMSO-d₆ δ [ppm] 10.77 (s, 1H), 7.94 (d, J = 7.8 Hz, 2H), 7.51 (d, J = 8.0 Hz, 2H), 7.14-7.09 (m, 1H), 6.58 (s, 1H), 5.89-5.82 (m, 1H), 2.89-2.74 (m, 2H), 2.68-2.50 (m, 4H, partially overlapping with DMSO peak), 2.45-2.37 (m, 1H), 2.32 (s, 3H), 2.26 (s, 3H), 2.08-2.00 (m, 1H), 1.97 (s, 3H), 1.79-1.65 (m, 1H), 1.56-1.45 (m, 2H), 1.45-1.31 (m, 1H). 29 of 30H reported (—COOH not observed) | Method B-2: Rt = 2.64 min; [M + H]⁺ = 391.2 |
| Ex-28 | 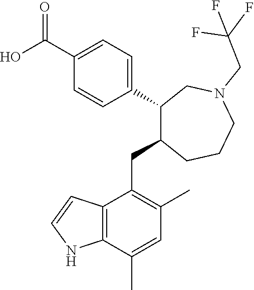 [SMILES: Cc1cc(C)c2[nH]ccc2c1C[C@@H]1CCCN(CC(F)(F)F)C[C@H]1c1ccc(C(=O)O)cc1] | 600 MHz, DMSO-d₆ δ [ppm] 10.80 (t, J = 2.3 Hz, 1H), 7.97-7.91 (m, 2H), 7.49 (d, J = 8.2 Hz, 2H), 7.15-7.12 (m, 1H), 6.59 (s, 1H), 5.90 (dd, J = 3.1, 2.0 Hz, 1H), 3.45-3.28 (m, 2H, partially overlapping with water peak), 3.21 (dd, J = 14.8, 9.7 Hz, 1H), 2.89 (dd, J = 11.4, 5.4 Hz, 1H), 2.78-2.68 (m, 3H), 2.50- 2.46 (m, 2H, partially overlapping with DMSO peak), 2.33 (s, 3H), 2.03-1.95 (m, 4H), 1.74-1.67 (m, 1H), 1.55-1.49 (m, 1H), 1.49-1.36 (m, 2H). 28 of 29H reported (—COOH not observed) | Method B-2: Rt = 7.03 min; [M + H]⁺ = 459.2 |
| Ex-29 | 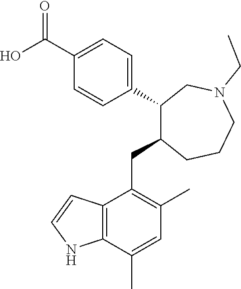 [SMILES: CCN1CCC[C@@H](Cc2c(C)cc(C)c3[nH]ccc23)[C@H](c2ccc(C(=O)O)cc2)C1] | 600 MHZ, DMSO-d₆ δ [ppm] 10.79 (s, 1H), 7.94 (d, J = 8.2 Hz, 2H), 7.52 (d, J = 8.0 Hz, 2H), 7.14-7.11 (m, 1H), 6.58 (s, 1H), 5.90-5.86 (m, 1H), 2.85 (dd, J = 13.9, 7.5 Hz, 1H), 2.74- 2.69 (m, 1H), 2.64-2.57 (m, 2H), 2.56- 2.45 (m, 5H, partially overlapping with DMSO peak), 2.32 (s, 3H), 2.06-1.96 (m, 4H), 1.76-1.67 (m, 1H), 1.54-1.46 (m, 2H), 1.42-1.33 (m, 1H), 0.90 (t, J = 7.0 Hz, 3H). 31 of 32H reported (—COOH not observed) | Method B-2: Rt = 2.68 min; [M + H]⁺ = 405.5 |
| Ex-30 | 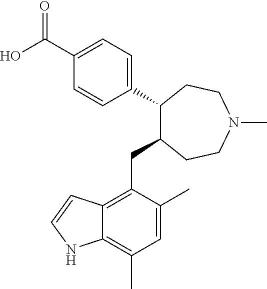 [SMILES: Cc1cc(C)c2[nH]ccc2c1C[C@@H]1CCN(C)CC[C@H]1c1ccc(C(=O)O)cc1] | 600 MHZ, DMSO-d₆ δ [ppm] 10.81 (s, 1H), 7.93 (d, J = 8.0 Hz, 2H), 7.45 (d, J = 8.0 Hz, 2H), 7.15-7.12 (m, 1H), 6.58 (s, 1H), 5.88-5.84 (m, 1H), 3.00-2.93 (m, 1H), 2.78-2.68 (m, 2H), 2.67-2.62 (m, 1H), 2.60-2.55 (m, 2H), 2.54-2.49 (m, 1H, partially overlapping with DMSO peak), 2.36-2.28 (m, 6H), 2.22-2.14 (m, 1H), 2.14-2.07 (m, 1H), 1.97 (s, 3H), 1.72-1.61 (m, 2H), 1.60- 1.52 (m, 1H). 29 of 30H reported (—COOH not observed) | Method B-2: Rt 2.52 min; [M + H]⁺ = 391.3 |
| Ex-31 | 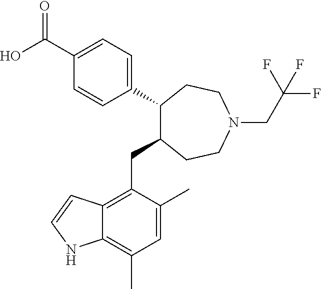 [SMILES: Cc1cc(C)c2[nH]ccc2c1C[C@@H]1CCN(CC(F)(F)F)CC[C@H]1c1ccc(C(=O)O)cc1] | 600 MHZ, DMSO-d₆ δ [ppm] 10.81 (s, 1H), 7.94 (d, J = 8.0 Hz, 2H), 7.48 (d, J = 8.0 Hz, 2H), 7.17-7.12 (m, 1H), 6.58 (s, 1H), 5.94-5.88 (m, 1H), 3.36-3.27 (m, 1H, partially overlapping with water peak), 3.25-3.16 (m, 1H), 3.04-2.98 (m, 1H), 2.94-2.87 (m, 2H), 2.72-2.65 (m, 2H), 2.54-2.49 (m, 1H, partially overlapping with DMSO peak), 2.46 (dd, J = 13.6, 3.4 Hz, 1H), 2.32 (s, 3H), 2.13-2.01 (m, 2H), 1.99 (s, 3H), 1.71-1.63 (m, 1H), 1.60-1.54 (m, 1H), 1.54-1.46 (m, 1H). 28 of 29H reported (—COOH not observed) | Method B-2: Rt 6.25 min; [M + H]⁺ = 459.1 |
| Ex-32 | 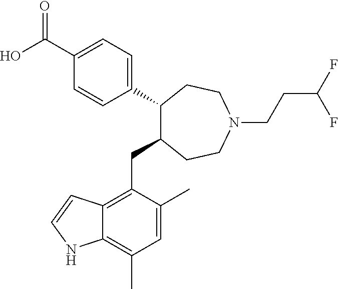 [SMILES: Cc1cc(C)c2[nH]ccc2c1C[C@@H]1CCN(CCC(F)F)CC[C@H]1c1ccc(C(=O)O)cc1] | 600 MHZ, DMSO-d₆ δ [ppm] 10.79 (s, 1H), 7.94 (d, J = 8.0 Hz, 2H), 7.49 (d, J = 8.0 Hz, 2H), 7.15-7.11 (m, 1H), 6.58 (s, 1H), 6.18-5.95 (m, 1H), 5.92-5.86 (m, 1H), 2.93-2.86 (m, 1H), 2.74-2.66 (m, 2H), 2.63-2.57 (m, 1H), 2.57-2.45 (m, 5H, partially overlapping with DMSO peak), 2.32 (s, 3H), 2.13- 2.04 (m, 2H), 2.03-1.90 (m, 5H), 1.71- 1.65 (m, 1H), 1.62-1.55 (m, 1H), 1.55- 1.48 (m, 1H). 31 of 32H reported (COOH not observed) | Method B-2: Rt 2.73 min; [M + H]⁺ = 455.4 |
| Ex-33 | 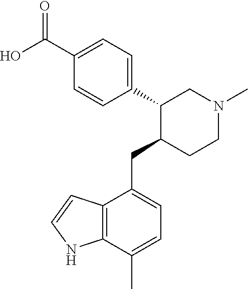 [SMILES: Cc1ccc(C[C@@H]2CCN(C)C[C@H]2c2ccc(C(=O)O)cc2)c2cc[nH]c12] | 600 MHZ, DMSO-d₆ δ [ppm] 10.97 (s, 1H), 7.97 (d, J = 7.9 Hz, 2H), 7.50 (d, J = 7.8 Hz, 2H), 7.27-7.20 (m, 1H), 6.73 (d, J = 7.1 Hz, 1H), 6.56 (d, J = 7.1 Hz, 1H), 6.13-6.08 (m, 1H), 2.81- 2.68 (m, 3H), 2.62 (d, J = 12.9 Hz, 1H), 2.38 (s, 3H), 2.23-2.11 (m, 4H), 2.09- 1.98 (m, 1H), 1.89-1.73 (m, 2H), 1.48- 1.38 (m, 1H), 1.35-1.24 (m, 1H) 25 of 26H reported (—COOH not observed) | Method B-2: Rt = 2.11 min; [M + H]⁺ = 363.3 |
| Ex-34 | 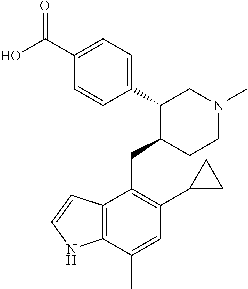 [SMILES: Cc1cc(C2CC2)c(C[C@@H]2CCN(C)C[C@H]2c2ccc(C(=O)O)cc2)c2cc[nH]c12] | 600 MHZ, DMSO-d₆ δ [ppm] 10.81 (s, 1H), 7.94 (d, J = 7.8 Hz, 2H), 7.51 (d, J = 7.8 Hz, 2H), 7.22-7.18 (m, 1H), 6.41 (s, 1H), 6.21-6.17 (m, 1H), 2.83-2.71 (m, 3H), 2.71-2.64 (m, 1H), 2.61-2.54 (m, 1H), 2.33 (s, 3H), 2.17 (s, 3H), 2.14-2.08 (m, 1H), 1.94-1.84 (m, 1H), 1.82-1.68 (m, 2H), 1.51-1.41 (m, 1H), 1.40-1.32 (m, 1H), 0.86-0.77 (m, 1H), 0.56-0.43 (m, 2H), 0.34-0.26 (m, 1H). 29 of 30H reported (—COOH not observed) | Method B-2: Rt = 2.64 min; [M + H]⁺ = 403.4 |
| Ex-35 | 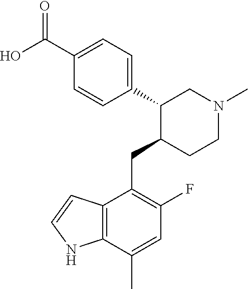 [SMILES: Cc1cc(F)c(C[C@@H]2CCN(C)C[C@H]2c2ccc(C(=O)O)cc2)c2cc[nH]c12] | 600 MHZ, DMSO-d₆ δ [ppm] 11.09 (br s, 1H), 7.96 (d, J = 7.9 Hz, 2H), 7.50 (d, J = 7.9 Hz, 2H), 7.33-7.30 (m, 1H), 6.66 (d, J = 10.8 Hz, 1H), 6.12 (dd, J = 3.1, 1.9 Hz, 1H), 2.82-2.72 (m, 3H), 2.48-2.37 (m, 5H), 2.16 (s, 3H), 2.09- 2.03 (m, 1H), 1.89-1.78 (m, 2H), 1.44- 1.33 (m, 2H). 24 of 25H reported (—COOH not observed) | Method B-2: Rt = 2.34 min; [M + H]⁺ = 381.2 |
| Ex-36 | 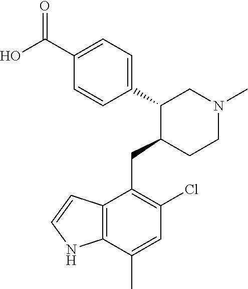 [SMILES: Cc1cc(Cl)c(C[C@@H]2CCN(C)C[C@H]2c2ccc(C(=O)O)cc2)c2cc[nH]c12] | 600 MHz, DMSO-d₆ δ [ppm] 11.20 (s, 1H), 7.94 (d, J = 7.9 Hz, 2H), 7.50 (d, J = 7.9 Hz, 2H), 7.34-7.31 (m, 1H), 6.84 (s, 1H), 6.24 (dd, J = 3.1, 1.9 Hz, 1H), 2.81-2.71 (m, 3H), 2.57 (d, J = 7.4 Hz, 2H), 2.38 (s, 3H), 2.16 (s, 3H), 2.10-2.02 (m, 1H), 2.02-1.92 (m, 1H), 1.81-1.74 (m, 1H), 1.52-1.43 (m, 1H), 1.33-1.26 (m, 1H). 24 of 25H reported (—COOH not observed) | Method B-2: Rt = 2.74 min; [M + H]⁺ = 397.2/399.2 |
| Ex-37 | 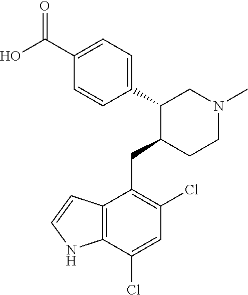 [SMILES: CN1CC[C@@H](Cc2c(Cl)cc(Cl)c3[nH]ccc23)[C@H](c2ccc(C(=O)O)cc2)C1] | 600 MHZ, DMSO-d₆ δ [ppm] 11.63 (s, 1H), 7.94 (d, J = 8.1 Hz, 2H), 7.50 (d, J = 7.9 Hz, 2H), 7.45-7.42 (m, 1H), 7.16 (s, 1H), 6.38 (dd, J = 3.2, 1.9 Hz, 1H), 2.83-2.76 (m, 2H), 2.76-2.70 (m, 1H), 2.66-2.56 (m, 2H), 2.15 (s, 3H), 2.09-2.03 (m, 1H), 2.02-1.93 (m, 1H), 1.82-1.75 (m, 1H), 1.53-1.44 (m, 1H), 1.30-1.24 (m, 1H). 21 of 22H reported (—COOH not observed) | Method B-2: Rt = 3.13 min; [M + H]⁺ = 417.2/419.2/ 421.1 |
| Ex-38 | 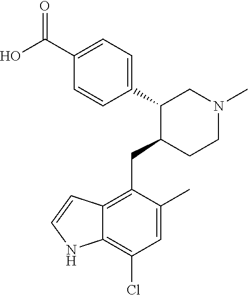 [SMILES: Cc1cc(Cl)c2[nH]ccc2c1C[C@@H]1CCN(C)C[C@H]1c1ccc(C(=O)O)cc1] | 600 MHZ, DMSO-d₆ δ [ppm] 11.22 (s, 1H), 7.95 (d, J = 7.9 Hz, 2H), 7.52 (d, J = 7.9 Hz, 2H), 7.30-7.26 (m, 1H), 6.91 (s, 1H), 6.26 (dd, J = 3.1, 1.9 Hz, 1H), 2.81-2.75 (m, 2H), 2.75-2.70 (m, 1H), 2.48-2.44 (m, 2H), 2.15 (s, 3H), 2.13 (s, 3H), 2.11-2.06 (m, 1H), 1.88-1.80 (m, 1H), 1.80-1.74 (m, 1H), 1.47-1.39 (m, 1H), 1.31-1.25 (m, 1H). 24 of 25H reported (—COOH not observed) | Method B-2: Rt 2.64 min; [M + H]⁺ = 397.2/399.2 |
| Ex-39 | 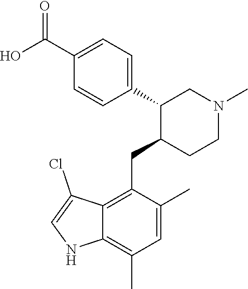 [SMILES: Cc1cc(C)c2[nH]cc(Cl)c2c1C[C@@H]1CCN(C)C[C@H]1c1ccc(C(=O)O)cc1] | 600 MHZ, DMSO-d₆ δ [ppm] 11.13 (d, J = 2.9 Hz, 1H), 7.88 (d, J = 7.9 Hz, 2H), 7.46 (d, J = 8.0 Hz, 2H), 7.33 (d, J = 2.7 Hz, 1H), 6.69 (s, 1H), 3.13-2.97 (m, 1H), 2.79-2.68 (m, 3H), 2.58-2.50 (m, 1H, partially overlapping with DMSO peak)), 2.31 (s, 3H), 2.15 (s, 3H), 2.12- 2.04 (m, 4H), 1.96-1.84 (m, 1H), 1.82- 1.73 (m, 1H), 1.64-1.52 (m, 1H), 1.15- 1.07 (m, 1H). 26 of 27H reported (—COOH not observed) | Method B-2: Rt 2.85 min; [M + H]⁺ = 411.2/413.2 |
| Ex-40 | 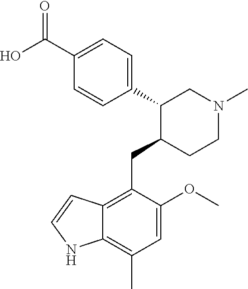 [SMILES: COc1cc(C)c2[nH]ccc2c1C[C@@H]1CCN(C)C[C@H]1c1ccc(C(=O)O)cc1] | 600 MHZ, DMSO-d₆ δ [ppm] 10.68 (s, 1H), 7.94 (d, J = 8.0 Hz, 2H), 7.48 (d, J = 7.9 Hz, 2H), 7.18-7.15 (m, 1H), 6.82 (s, 1H), 6.29-6.24 (m, 1H), 3.69 (s, 3H), 2.80-2.67 (m, 3H), 2.40- 2.30 (m, 2H), 2.23 (s, 3H), 2.15 (s, 3H), 2.03-1.95 (m, 1H), 1.86-1.73 (m, 2H), 1.46-1.31 (m, 2H). 27 of 28H reported (—COOH not observed) | Method B-2: Rt 2.42 min; [M + H]⁺ = 393.2 |
| Ex-41 | 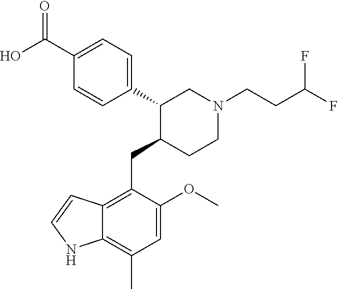 [SMILES: COc1cc(C)c2[nH]ccc2c1C[C@@H]1CCN(CCC(F)F)C[C@H]1c1ccc(C(=O)O)cc1] | 600 MHZ, DMSO-d₆ δ [ppm] 10.70 (s, 1H), 7.94 (d, J = 8.0 Hz, 2H), 7.50 (d, J = 7.9 Hz, 2H), 7.19-7.14 (m, 1H), 6.82 (s, 1H), 6.28-6.24 (m, 1H), 6.18-5.95 (m, 1H), 3.68 (s, 3H), 2.86- 2.79 (m, 2H), 2.72-2.64 (m, 1H), 2.44- 2.31 (m, 4H), 2.22 (s, 3H), 2.06-1.90 (m, 3H), 1.88-1.76 (m, 2H), 1.44-1.33 (m, 2H). 29 of 30H reported (—COOH not observed) | Method B-2: Rt 2.50 min; [M + H]⁺ = 457.3 |
| Ex-42 | 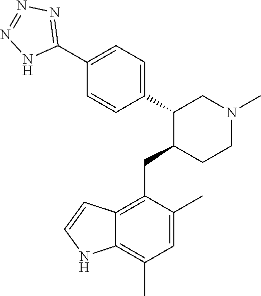 [SMILES: Cc1cc(C)c2[nH]ccc2c1C[C@@H]1CCN(C)C[C@H]1c1ccc(-c2nnn[nH]2)cc1] | 600 MHZ, DMSO-d₆ δ [ppm] 10.87 (s, 1H), 8.05 (d, J = 7.9 Hz, 2H), 7.50 (d, J = 7.8 Hz, 2H), 7.23-7.19 (m, 1H), 6.63 (s, 1H), 6.17-6.12 (m, 1H), 3.38-3.33 (m, 1H, partially overlapping with water peak), 3.25-3.18 (m, 1H, partially overlapping with water peak), 3.12-3.03 (m, 1H), 2.90-2.83 (m, 1H), 2.71-2.63 (m, 4H), 2.62-2.56 (m, 1H), 2.48-2.41 (m, 1H), 2.34 (s, 3H), 2.12 (s, 3H), 2.09-2.01 (m, 1H), 1.59-1.51 (m, 1H), 1.51-1.46 (m, 1H). 27 of 28H reported (tetrazole-NH not observed) | Method B-2: Rt = 2.40 min; [M + H]⁺ = 401.4 |
| Ex-43 | 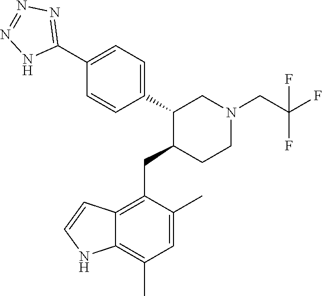 [SMILES: Cc1cc(C)c2[nH]ccc2c1C[C@@H]1CCN(CC(F)(F)F)C[C@H]1c1ccc(-c2nnn[nH]2)cc1] | 600 MHZ, DMSO-d₆ δ [ppm] 10.87 (s, 1H), 8.05 (d, J = 7.9 Hz, 2H), 7.50 (d, J = 7.8 Hz, 2H), 7.23-7.19 (m, 1H), 6.63 (s, 1H), 6.17-6.12 (m, 1H), 3.38-3.33 (m, 1H, partially overlapping with water peak), 3.25-3.18 (m, 1H, partially overlapping with water peak), 3.12-3.03 (m, 1H), 2.90-2.83 (m, 1H), 2.71-2.63 (m, 4H), 2.62-2.56 (m, 1H), 2.48-2.41 (m, 1H), 2.34 (s, 3H), 2.12 (s, 3H), 2.09-2.01 (m, 1H), 1.59-1.51 (m, 1H), 1.51-1.46 (m, 1H). 27 of 28H reported (tetrazole-NH not observed) | Method B-2: Rt = 5.94 min; [M + H]⁺ = 469.4 |
| Ex-44 | 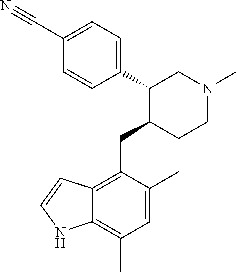 [SMILES: Cc1cc(C)c2[nH]ccc2c1C[C@@H]1CCN(C)C[C@H]1c1ccc(C#N)cc1] | 600 MHZ, DMSO-d₆ δ [ppm] 10.82 (s, 1H), 7.84 (d, J = 8.2 Hz, 2H), 7.61 (d, J = 7.9 Hz, 2H), 7.20-7.17 (m, 1H), 6.60 (s, 1H), 6.15-6.12 (m, 1H), 2.82-2.76 (m, 1H), 2.75-2.71 (m, 1H), 2.70-2.66 (m, 1H), 2.46-2.38 (m, 2H), 2.34 (s, 3H), 2.12 (s, 3H), 2.09 (s, 3H), 2.05-1.99 (m, 1H), 1.86-1.78 (m, 1H), 1.76-1.68 (m, 1H), 1.43-1.35 (m, 1H), 1.34-1.27 (m, 1H). 27 of 27H reported | Method B-2: Rt = 2.94 min; [M + H]⁺ = 358.4 |
| Ex-45 | 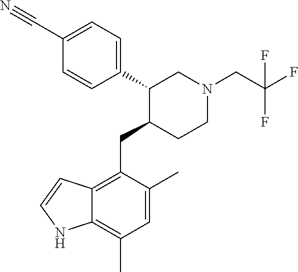 [SMILES: Cc1cc(C)c2[nH]ccc2c1C[C@@H]1CCN(CC(F)(F)F)C[C@H]1c1ccc(C#N)cc1] | 600 MHZ, DMSO-d₆ δ [ppm] 10.82 (s, 1H), 7.85 (d, J = 7.9 Hz, 2H), 7.64 (d, J = 7.9 Hz, 2H), 7.20-7.15 (m, 1H), 6.60 (s, 1H), 6.20-6.15 (m, 1H), 3.24-3.09 (m, 2H), 2.89-2.80 (m, 3H), 2.57-2.51 (m, 1H), 2.48-2.43 (m, 1H), 2.42-2.37 (m, 1H), 2.34 (s, 3H), 2.27- 2.21 (m, 1H), 2.09 (s, 3H), 1.93-1.84 (m, 1H), 1.46-1.36 (m, 1H), 1.32-1.26 (m, 1H). 26 of 26H reported | Method B-2: Rt = 6.77 min; [M + H]⁺ = 426.3 |
| Ex-46 | 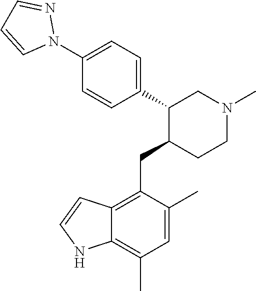 [SMILES: Cc1cc(C)c2[nH]ccc2c1C[C@@H]1CCN(C)C[C@H]1c1ccc(-n2cccn2)cc1] | 600 MHZ, DMSO-d₆ δ [ppm] 10.82 (s, 1H), 8.52 (d, J = 2.5 Hz, 1H), 7.85 (d, J = 8.2 Hz, 2H), 7.74 (d, J = 1.7 Hz, 1H), 7.51 (d, J = 8.2 Hz, 2H), 7.20- 7.16 (m, 1H), 6.61 (s, 1H), 6.55-6.53 (m, 1H), 6.19-6.16 (m, 1H), 2.79-2.75 (m, 1H), 2.73-2.67 (m, 2H), 2.58-2.53 (m, 1H), 2.44-2.38 (m, 1H), 2.34 (s, 3H), 2.13 (s, 3H), 2.12 (s, 3H), 2.06- 2.00 (m, 1H), 1.84-1.75 (m, 1H), 1.75- 1.67 (m, 1H), 1.44-1.35 (m, 1H), 1.35- 1.28 (m, 1H). 30 of 30H reported | Method B-2: Rt = 3.32 min; [M + H]⁺ = 399.0 |
| Ex-47 | 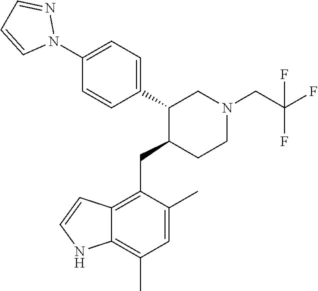 [SMILES: Cc1cc(C)c2[nH]ccc2c1C[C@@H]1CCN(CC(F)(F)F)C[C@H]1c1ccc(-n2cccn2)cc1] | 600 MHZ, DMSO-d₆ δ [ppm] 10.82 (s, 1H), 8.52 (d, J = 2.5 Hz, 1H), 7.86 (d, J = 8.1 Hz, 2H), 7.74 (d, J = 1.7 Hz, 1H), 7.53 (d, J = 8.2 Hz, 2H), 7.19- 7.17 (m, 1H), 6.61 (s, 1H), 6.56-6.53 (m, 1H), 6.22-6.19 (m, 1H), 3.23-3.09 (m, 2H), 2.94-2.89 (m, 1H), 2.88-2.82 (m, 1H), 2.78-2.72 (m, 1H), 2.57-2.51 (m, 2H), 2.47-2.41 (m, 1H), 2.34 (s, 3H), 2.25-2.18 (m, 1H), 2.12 (s, 3H), 1.90-1.81 (m, 1H), 1.47-1.38 (m, 1H), 1.34-1.25 (m, 1H). 29 of 29H reported | Method B-2: Rt = 6.98 min; [M + H]⁺ = 467.4 |
| Ex-48 | 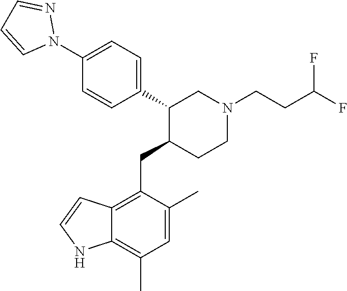 [SMILES: Cc1cc(C)c2[nH]ccc2c1C[C@@H]1CCN(CCC(F)F)C[C@H]1c1ccc(-n2cccn2)cc1] | 600 MHZ, DMSO-d₆ δ [ppm] 10.81 (s, 1H), 8.52 (d, J = 2.5 Hz, 1H), 7.86 (d, J = 8.5 Hz, 2H), 7.74 (d, J = 1.7 Hz, 1H), 7.53 (d, J = 8.2 Hz, 2H), 7.19- 7.17 (m, 1H), 6.61 (s, 1H), 6.55-6.53 (m, 1H), 6.19-5.96 (m, 2H), 2.89-2.84 (m, 1H), 2.83-2.78 (m, 1H), 2.74-2.67 (m, 1H), 2.58-2.53 (m, 1H), 2.46-2.36 (m, 3H), 2.36 (s, 3H), 2.15-2.09 (m, 4H), 2.03-1.92 (m, 2H), 1.88-1.81 (m, 1H), 1.80-1.73 (m, 1H), 1.44-1.29 (m, 2H). 32 of 32H reported | Method B-2: Rt 3.42 min; [M + H]⁺ = 463.3 |
| Ex-49 | 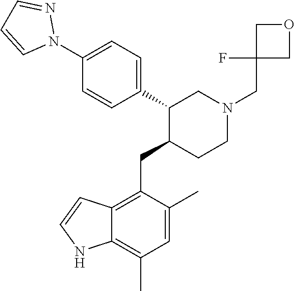 [SMILES: Cc1cc(C)c2[nH]ccc2c1C[C@@H]1CCN(CC2(F)COC2)C[C@H]1c1ccc(-n2cccn2)cc1] | 600 MHZ, DMSO-d₆ δ [ppm] 10.80 (s, 1H), 8.51 (d, J = 2.5 Hz, 1H), 7.85 (d, J = 8.1 Hz, 2H), 7.74 (d, J = 1.7 Hz, 1H), 7.52 (d, J = 8.1 Hz, 2H), 7.19- 7.15 (m, 1H), 6.61 (s, 1H), 6.54 (t, J = 2.1 Hz, 1H), 6.19 (t, J = 2.5 Hz, 1H), 4.62 -4.51 (m, 4H), 2.89-2.83 (m, 2H), 2.83 -2.77 (m, 2H), 2.75-2.69 (m, 1H), 2.56 -2.49 (m, 1H, partially overlapping with DMSO peak), 2.46-2.39 (m, 1H), 2.37 -2.30 (m, 4H), 2.12 (s, 3H), 2.06-1.96 (m, 1H), 1.87-1.78 (m, 1H), 1.45-1.36 (m, 1H), 1.33-1.27 (m, 1H). 33 of 33H reported | Method B-2: Rt 3.63 min; [M + H]⁺ = 473.3 |
| Ex-50 | 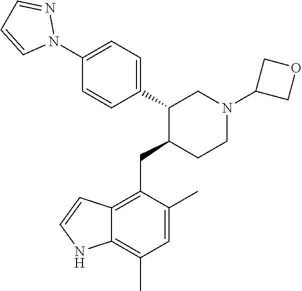 [SMILES: Cc1cc(C)c2[nH]ccc2c1C[C@@H]1CCN(C2COC2)C[C@H]1c1ccc(-n2cccn2)cc1] | 600 MHz, DMSO-d₆ δ [ppm] 10.81 (s, 1H), 8.51 (d, J = 2.5 Hz, 1H), 7.85 (d, J = 8.2 Hz, 2H), 7.74 (d, J = 1.7 Hz, 1H), 7.52 (d, J = 8.2 Hz, 2H), 7.21- 7.16 (m, 1H), 6.61 (s, 1H), 6.55-6.51 (m, 1H), 6.19 (t, J = 2.5 Hz, 1H), 4.50- 4.44 (m, 2H), 4.43-4.37 (m, 2H), 3.41- 3.32 (m, 1H, partially overlapping with water peak), 2.77-2.71 (m, 1H), 2.71- 2.66 (m, 1H), 2.66-2.61 (m, 1H), 2.58- 2.53 (m, 1H), 2.48-2.42 (m, 1H), 2.34 (s, 3H), 2.12 (s, 3H), 2.01-1.94 (m, 1H), 1.91-1.81 (m, 1H), 1.67-1.60 (m, 1H), 1.46-1.37 (m, 1H), 1.37-1.31 (m, 1H). 32 of 32H reported | Method B-2: Rt 3.43 min; [M + H]⁺ = 441.3 |
| Ex-51 | 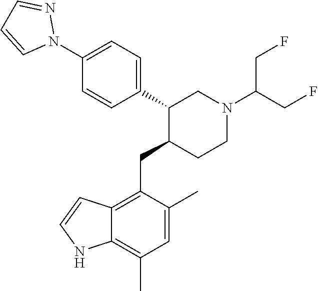 [SMILES: Cc1cc(C)c2[nH]ccc2c1C[C@@H]1CCN(C(CF)CF)C[C@H]1c1ccc(-n2cccn2)cc1] | 600 MHZ, DMSO-d₆ δ [ppm] 10.80 (s, 1H), 8.51 (d, J = 2.5 Hz, 1H), 7.86 (d, J = 8.5 Hz, 2H), 7.78-7.72 (m, 1H), 7.53 (d, J = 8.1 Hz, 2H), 7.21-7.15 (m, 1H), 6.61 (s, 1H), 6.56-6.52 (m, 1H), 6.22-6.17 (m, 1H), 4.61 (d, J = 5.3 Hz, 2H), 4.53 (d, J = 5.0 Hz, 2H), 3.11- 2.99 (m, 1H), 2.90-2.85 (m, 1H), 2.83- 2.77 (m, 1H), 2.70-2.61 (m, 2H), 2.56- 2.52 (m, 1H), 2.46-2.39 (m, 1H), 2.38- 2.27 (m, 4H), 2.13 (s, 3H), 1.91-1.83 (m, 1H), 1.40-1.29 (m, 2H). 32 of 32H reported | Method B-2: Rt 4.22 min; [M + H]⁺ = 463.5 |
| Ex-52 | 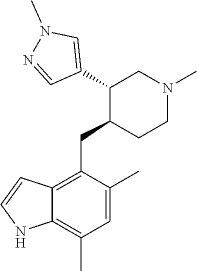 [SMILES: Cc1cc(C)c2[nH]ccc2c1C[C@@H]1CCN(C)C[C@H]1c1cnn(C)c1] | 600 MHZ, DMSO-d₆ δ [ppm] 10.80 (s, 1H), 7.63 (s, 1H), 7.41 (s, 1H), 7.19-7.16 (m, 1H), 6.62 (s, 1H), 6.16- 6.12 (m, 1H), 3.83 (s, 3H), 2.79-2.75 (m, 1H), 2.75-2.71 (m, 1H), 2.68-2.62 (m, 1H), 2.58-2.51 (m, 1H), 2.39-2.31 (m, 4H), 2.13 (s, 3H), 2.10 (s, 3H), 1.93- 1.87 (m, 1H), 1.66-1.59 (m, 1H), 1.56- 1.48 (m, 1H), 1.35-1.22 (m, 2H) 28 of 28H reported | Method B-2: Rt 2.08 min; [M + H]⁺ = 337.3 |
| Ex-53 | 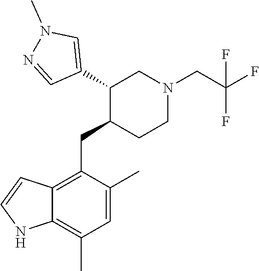 [SMILES: Cc1cc(C)c2[nH]ccc2c1C[C@@H]1CCN(CC(F)(F)F)C[C@H]1c1cnn(C)c1] | 600 MHZ, DMSO-d₆ δ [ppm] 10.80 (s, 1H), 7.65 (s, 1H), 7.42 (s, 1H), 7.22-7.16 (m, 1H), 6.62 (s, 1H), 6.25- 6.11 (m, 1H), 3.83 (s, 3H), 3.18-3.05 (m, 2H), 2.90-2.84 (m, 1H), 2.84-2.79 (m, 1H), 2.79-2.73 (m, 1H), 2.63-2.55 (m, 1H), 2.41-2.36 (m, 2H), 2.35 (s, 3H), 2.16-2.10 (m, 4H), 1.63-1.53 (m, 1H), 1.38-1.29 (m, 1H), 1.29-1.22 (m, 1H). 27 of 27H reported | Method B-2: Rt 5.73 min; [M + H]⁺ = 405.3 |
| Ex-54 | 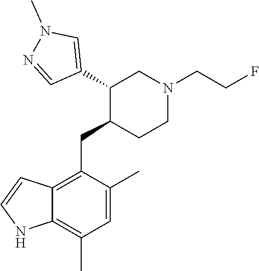 [SMILES: Cc1cc(C)c2[nH]ccc2c1C[C@@H]1CCN(CCF)C[C@H]1c1cnn(C)c1] | 600 MHz, DMSO-d₆ δ [ppm] 10.80 (s, 1H), 7.64 (s, 1H), 7.42 (s, 1H), 7.20-7.16 (m, 1H), 6.62 (s, 1H), 6.19- 6.14 (m, 1H), 4.54-4.42 (m, 2H), 3.83 (s, 3H), 2.89-2.83 (m, 1H), 2.81-2.73 (m, 2H), 2.59-2.51 (m, 3H), 2.41-2.34 (m, 4H), 2.13 (s, 3H), 2.09-2.02 (m, 1H), 1.84-1.76 (m, 1H), 1.62-1.52 (m, 1H), 1.37-1.21 (m, 2H). 29 of 29H reported | Method B-2: Rt 2.14 min; [M + H]⁺ = 369.3 |
| Ex-55 | 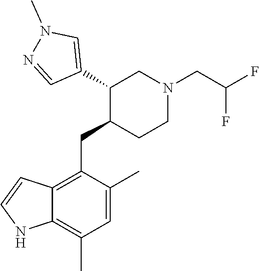 [SMILES: Cc1cc(C)c2[nH]ccc2c1C[C@@H]1CCN(CC(F)F)C[C@H]1c1cnn(C)c1] | 600 MHZ, DMSO-d₆ δ [ppm] 10.78 (s, 1H), 7.64 (s, 1H), 7.42 (s, 1H), 7.20-7.15 (m, 1H), 6.62 (s, 1H), 6.20- 5.98 (m, 2H), 3.83 (s, 3H), 2.90-2.84 (m, 1H), 2.82-2.74 (m, 2H), 2.73-2.60 (m, 2H), 2.59-2.53 (m, 1H), 2.41-2.33 (m, 4H), 2.25-2.17 (m, 1H), 2.13 (s, 3H), 1.98-1.92 (m, 1H), 1.62-1.53 (m, 1H), 1.37-1.29 (m, 1H), 1.29-1.22 (m, 1H). 28 of 28H reported | Method B-2: Rt 2.95 min; [M + H]⁺ = 387.3 |
| Ex-56 | 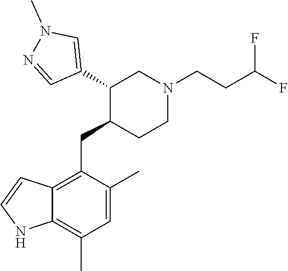 [SMILES: Cc1cc(C)c2[nH]ccc2c1C[C@@H]1CCN(CCC(F)F)C[C@H]1c1cnn(C)c1] | 600 MHZ, DMSO-d₆ δ [ppm] 10.80 (s, 1H), 7.64 (s, 1H), 7.42 (s, 1H), 7.19-7.16 (m, 1H), 6.62 (s, 1H), 6.17- 5.94 (m, 2H), 3.83 (s, 3H), 2.84-2.80 (m, 1H), 2.79-2.73 (m, 2H), 2.57-2.51 (m, 1H), 2.42-2.29 (m, 6H), 2.13 (s, 3H), 2.02-1.89 (m, 3H), 1.73-1.66 (m, 1H), 1.61-1.53 (m, 1H), 1.35-1.22 (m, 2H). 30 of 30H reported | Method B-2: Rt 2.21 min; [M + H]⁺ = 401.3 |
| Ex-57 | 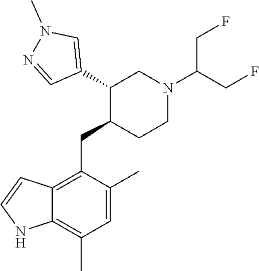 [SMILES: Cc1cc(C)c2[nH]ccc2c1C[C@@H]1CCN(C(CF)CF)C[C@H]1c1cnn(C)c1] | 400 MHZ, DMSO-d₆ δ [ppm] 10.78 (s, 1H), 7.64 (s, 1H), 7.42 (s, 1H), 7.21-7.15 (m, 1H), 6.62 (s, 1H), 6.18 (dd, J = 3.1, 1.9 Hz, 1H), 4.61 (d, J = 5.2 Hz, 2H), 4.49 (d, J = 5.2 Hz, 2H), 3.83 (s, 3H), 3.10-2.92 (m, 1H), 2.89-2.80 (m, 1H), 2.80-2.71 (m, 2H), 2.55-2.43 (m, 2H, partially overlapping with DMSO peak), 2.42-2.31 (m, 4H), 2.29-2.19 (m, 1H), 2.14 (s, 3H), 1.66-1.54 (m, 1H), 1.32-1.21 (m, 2H). 30 of 30H reported | Method B-2: Rt 2.67 min; [M + H]⁺ = 401.4 |
| Ex-58 | 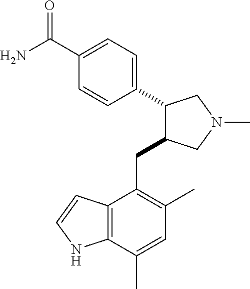 [SMILES: Cc1cc(C)c2[nH]ccc2c1C[C@@H]1CN(C)C[C@H]1c1ccc(C(N)=O)cc1] | Racemic mixture 400 MHz, methanol-d4 δ [ppm] 7.80- 7.72 (m, 2H), 7.29-7.24 (m, 2H), 7.11 (d, J = 3.2 Hz, 1H), 6.59 (s, 1H), 6.26 (d, J = 3.3 Hz, 1H), 4.58 (s, 1H), 3.23-3.12 (m, 2H, partially overlapping with solvent peak), 3.08 (t, J = 9.4 Hz, 1H), 3.02- 2.93 (m, 2H), 2.83-2.77 (m, 1H), 2.74- 2.67 (m, 1H), 2.42 (s, 3H), 2.37 (s, 3H), 2.14 (s, 3H). 24 of 27H reported (—CONH₂, tetrazole- NH not observed) | Method C-3: Rt = 0.92 min; [M + H]⁺ = 362.4 |
| Ex-59 | 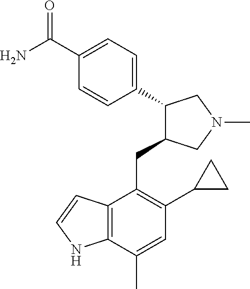 [SMILES: Cc1cc(C2CC2)c(C[C@@H]2CN(C)C[C@H]2c2ccc(C(N)=O)cc2)c2cc[nH]c12] | 400 MHZ, DMSO-d₆ δ [ppm] 10.76 (s, 1H), 7.74 (d, J = 7.9 Hz, 2H), 7.21 (d, J = 8.1 Hz, 2H), 7.13 (d, J = 2.8 Hz, 1H), 6.33 (s, 1H), 6.11 (d, J = 2.6 Hz, 1H), 3.12-3.05 (m, 1H), 3.03-2.98 (m, 2H), 2.83 (t, J = 9.0 Hz, 1H), 2.64-2.57 (m, 1H), 2.54-2.47 (m, 2H), 2.40-2.37 (m, 1H, partially overlapping with DMSO peak), 2.27 (s, 3H), 2.20 (s, 3H), 1.69- 1.62 (m, 1H), 0.58-0.50 (m, 2H), 0.42- 0.29 (m, 2H). 27 of 28H reported (—COOH not observed) | Method C-3: Rt = 1.44 min; [M + H]⁺ = 389.3 |
| Ex-60 | 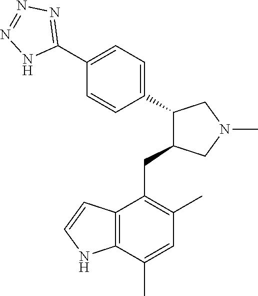 [SMILES: Cc1cc(C)c2[nH]ccc2c1C[C@@H]1CN(C)C[C@H]1c1ccc(-c2nnn[nH]2)cc1] | 400 MHZ, methanol-d4 δ [ppm] 8.01 (d, J = 8.1 Hz, 2H), 7.37 (d, J = 8.2 Hz, 2H), 7.14 (d, J = 3.0 Hz, 1H), 6.63 (s, 1H), 6.26 (d, J = 3.1 Hz, 1H), 3.56-3.50 (m, 1H), 3.44-3.35 (m, 2H), 3.18-3.14 (m, 2H), 3.07-2.94 (m, 2H), 2.89-2.80 (m, 1H), 2.76 (s, 3H), 2.36 (s, 3H), 2.16 (s, 3H). 24 of 26H reported (tetrazole and indole NH not observed) | Method C-3: Rt = 1.15 min; [M + H]⁺ = 387.5 |
| Ex-61 | 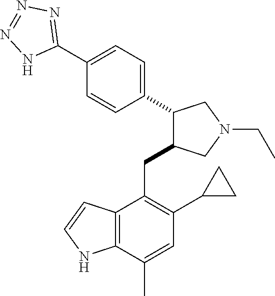 [SMILES: CCN1C[C@@H](Cc2c(C3CC3)cc(C)c3[nH]ccc23)[C@H](c2ccc(-c3nnn[nH]3)cc2)C1] | Racemic mixture 400 MHZ, methanol-d4 δ [ppm] 7.94 (d, J = 8.2 Hz, 2H), 7.33 (d, J = 8.3 Hz, 2H), 7.12 (d, J = 3.3 Hz, 1H), 6.43 (s, 1H), 6.32 (d, J = 3.0 Hz, 1H), 3.25-3.17 (m, 2H), 3.15-3.04 (m, 2H), 2.80 (dd, J = 9.5, 7.2 Hz, 3H), 2.73-2.46 (m, 3H), 2.36 (s, 3H), 1.79-1.73 (m, 1H), 1.11 (t, J = 7.2 Hz, 3H), 0.76-0.67 (m, 1H), 0.65-0.58 (m, 1H), 0.57-0.46 (m, 1H), 0.43-0.38 (m, 1H). 28 of 30H reported (tetrazole and indole NH not observed) | Method C-4: Rt = 1.34 min; [M + H]⁺ = 427.4 |

### LC-MS Methods

### Method A-1:

Column: Acquity UPLC BEH C18, 2.1×50 mm Column, 1.7 μm
Column temperature: 40° C.
Eluents: A: water+FA (0.1%)
   B: ACN
Flow rate: 0.8 mL/min
Gradient: Time/% B: 0.01/5; 0.3/5; 0.5/100; 1.8/100; 2.0/5; 3.0/5 (3 min method)

### Method A-2:

Column: Waters XBridge C18, 3.0×50 mm Column, 3.5 μm
Column temperature: 40° C.
Eluents: A: water+FA (0.1%)
   B: ACN
Flow rate: 1.0 mL/min
Gradient: Time/% B: 0.01/10; 0.2/10 4.5/95; 8.5/95; 8.5/10; 10.0/10; (10 min method)

### Method B-1:

Column: CORTECS C18+, 2.1×50 mm Column, 2.7 μm
Column temperature: 80° C.
Eluents: A: water+0.05% FA+3.75 mM ammonium acetate NH₄ (CH₃CO₂)
   B: IPA+0.05% FA
Flow rate: 1.0 mL/min
Gradient: initial 5% B; 5% to 50% B in 1.4 min, 50% to 98% B in 0.30 min; 0.10 min 98% B.

### Method B-2:

Column: Acquity UPLC BEH C18, 2.1×100 mm Column, 1.7 μm
Column temperature: 80° C.
Eluents: A: water+0.05% FA+3.75 mM ammonium acetate
   B: IPA+0.05% FA
Flow rate: 0.4 mL/min
Gradient: Pre-run with 5% B for 0.50 min; 5% to 60% B in 8.4 min, 60% to 98% B in 1.00 min; 0.40 min 98% B.

### Method C-1:

Column: Acquity UPLC BEH C18, 2.1×30 mm Column, 1.7 μm
Column temperature: 50° C.
Eluents: A: water+0.1% FA
   B: ACN+0.1% FA
Flow rate: 1.0 mL/min
Gradient: 2% B until 0.1 min; 2% to 98% B until 1.5 min, 98% B until 1.8 min.

### Method C-2:

Column: Acquity UPLC BEH C18, 2.1×30 mm Column, 1.7 μm
Column temperature: 50° C.
Eluents: A: 5 mM NH OH in water
   B: 5 mM NH₄OH in ACN
Flow rate: 1.0 mL/min
Gradient: 2% B until 0.1 min; 2% to 98% B until 1.5 min, 98% B until 1.8 min.

### Method C-3:

Column: Acquity UPLC BEH C18, 2.1×30 mm Column, 1.7 μm
Column temperature: 50° C.
Eluents: A: 0.1% FA in water
   B: 0.1% FA in ACN
Flow rate: 1.0 mL/min
Gradient: 2% to 98% B until 4.40 min, 98% B until 5.15 min.

### Method C-4:

Column: Acquity UPLC BEH C18, 2.1×30 mm Column, 1.7 μm
Column temperature: 50° C.
Eluents: A: 5 mM NH₄OH in water
   B: 5 mM NH₄OH in ACN
Flow rate: 1.0 mL/min
Gradient: 2% to 98% B until 4.40 min, 98% B until 5.15 min.

### Chiral HPLC Methods

### Method SFC-1:

### Preparative Chiral HPLC

Instrument: Sepiatec prep SFC-100
Column: Chiralpak AD, 250 mm×30 mm 5 μm
Eluent: A: 15% 0.05% NH₃ in IPA, B: 85% scCO₂ (isocratic)
Flow rate: 110.0 mL/min
Detection: UV 230 nm
Injection volume: 3×1.30 mL
Oven temperature: 40° C.
Pressure: 120 bar

### Analytical Chiral HPLC

Instrument: Analytical SFC-MS Waters UPC2
Injection: 5 μL
Mobile phase: A: 12% 0.05% NH₃ in IPA, B: 88% scCO₂ (isocratic)
Flow rate: 3 mL/min
Column: Chiralpak AD (4.6 mm×100 mm 5 μm)
Detection UV: DAD
Oven temperature: 40° C.
Pressure: 1800 psi

### Method SFC-2:

### Preparative Chiral HPLC

Instrument: Sepiatec prep SFC-100
Column: Chiralpak IH, 250×30 mm 5 μm
Eluent: A: 11% 0.05% NH₃ in IPA, B: 89% scCO₂ (isocratic)
Flow rate: 90.0 mL/min
Detection: UV 239 nm
Injection volume: 55×1.00 mL
Oven temperature: 40° C.
Pressure: 130 bar

### Analytical Chiral HPLC

Instrument: Analytical SFC-MS Waters UPC2
Injection: 5 μL
Mobile phase: A: 10% 0.05% NH₃ in IPA, B: 90% scCO₂ (isocratic)
Flow rate: 3 mL/min
Column: Chiralpak IH (4.6 mm×100 mm 5 μm)
Detection UV: DAD
Oven temperature: 40° C.
Pressure: 1800 psi

### Method SFC-3:

### Preparative Chiral HPLC

Instrument: Sepiatec prep SFC-250
Column: Chiralpak IG, 250×30 mm 5 μm
Eluent: A: 55% 0.05% NH₃ in MeOH/IPA 50/50, B: 45% scCO₂ (isocratic)
Flow rate: 80 mL/min
Detection: UV 240 nm
Injection volume: 38×2.0 mL
Oven temperature: 40° C.
Pressure: 120 bar

### Analytical Chiral HPLC

Instrument: Analytical SFC-MS Waters UPC2
Injection: 5 μL
Mobile phase: A: 50% 0.05% NH₃ in MeOH/IPA 50/50, B: 50% scCO₂ (isocratic)
Flow rate: 3.0 mL/min
Column: Chiralpak IG (4.6×100 mm 5 μm)
Detection UV: DAD
Oven temperature: 40° C.
Pressure: 1800 psi

### Method SFC-4:

### Preparative Chiral HPLC

Instrument: Waters THAR80 systems
Column: Chiralpak IG, 21×250 mm 5 μm
Eluent: A: 45% MeOH w/10 mM NH₃, B: 55% scCO₂ (isocratic)
Flow rate: 80 g/min
Detection: UV 270 nm
Injection volume: 4×3.0 mL
Oven temperature: RT
Pressure: 125 bar

### Analytical Chiral HPLC

Instrument: Analytical SFC-MS Waters UPC2
Injection: 3 μL
Mobile phase: A: CO₂, B: 5 to 55% MeOH with 0.1% NH₃
Flow rate: 2.5 mL/min
Column: Chiralpak IG-3 (3×100 mm 3 μm)
Detection UV: DAD
Oven temperature: 40° C.
Pressure: 1800 psi

### Method SFC-5:

### Preparative Chiral SFC

Instrument: Sepiatec SFC 100
Column: 2× Chiralcel oz in a raw, 250×30 mm 5 μm
Eluent: A: 22% 0.05% NH₃ in MeOH, B: 78% scCO₂ (isocratic)
Flow rate: 105 mL/min
Detection: UV 235 nm
Injection volume: 3.5 mL
Oven temperature: 40° C.
Pressure: 100 bar

### Analytical Chiral SFC

Instrument: Analytical SFC-MS Waters UPC2
Injection: 5 μL
Mobile phase: A: 12% 0.05% NH₃ in MeOH, B: 88% scCO₂ (isocratic)
Flow rate: 3 mL/min
Column: Chiralcel Oz (4.6×100 mm 5 μm)
Detection UV: DAD 235 nm
Oven temperature: 40° C.
Pressure: 1800 psi

### Method SFC-6:

### Preparative Chiral SFC

Instrument: Sepiatec SFC 100
Column: Chiralpak IG, 250×50 mm 5 μm
Eluent: A: 55% 0.05% NH₃ in IPA, B: 45% scCO₂ (isocratic)
Flow rate: 120 mL/min
Detection: UV 235 nm
Injection volume: 1.2 mL
Oven temperature: 40° C.
Pressure: 120 bar

### Analytical Chiral SFC

Instrument: Analytical SFC-MS Waters UPC2
Injection: 5 μL
Mobile phase: A: 40% 0.05% NH₃ in IPA, B: 60% scCO₂ (isocratic)
Flow rate: 3 mL/min
Column: Chiralpak AD (4.6×100 mm 5 μm)
Detection UV: DAD 235 nm
Oven temperature: 40° C.
Pressure: 1800 psi

### Method SFC-7:

### Preparative Chiral SFC

Instrument: Sepiatec SFC 100
Column: Chiralpak AD-H, 250×30 mm 5 μm
Eluent: A: 42% 0.05% NH₃ in IPA, B: 58% scCO₂ (isocratic)
Flow rate: 80 mL/min
Detection: UV 220 nm
Injection volume: 3.5 mL
Oven temperature: 40° C.
Pressure: 120 bar

### Analytical Chiral SFC

Instrument: Analytical SFC-MS Waters UPC2
Injection: 5 μL
Mobile phase: A: 40% 0.05% NH₃ in IPA, B: 60% scCO₂ (isocratic)
Flow rate: 3 mL/min
Column: Chiralpak AD (4.6×100 mm 5 μm)
Detection UV: DAD 220 nm
Oven temperature: 40° C.
Pressure: 1800 psi

### Method SFC-8:

### Preparative Chiral HPLC

Instrument: Sepiatec prep SFC-100
Column: Chiralpak AD, 250×30 mm 5 μm
Eluent: A: 20% 0.05% NH₃ in IPA, B: 80% scCO₂ (isocratic)
Flow rate: 80 mL/min
Detection: UV 235 nm
Injection volume: 23×1.10 mL
Oven temperature: 40° C.
Pressure: 120 bar

### Analytical Chiral HPLC

Instrument: Analytical SFC-MS Waters UPC2
Injection: 5 μL
Mobile phase: A: 20% 0.05% NH₃ in IPA, B: 80% scCO₂ (isocratic)
Flow rate: 3 mL/min
Column: Chiralpak AD (4.6×100 mm 5 μm)
Detection UV: DAD
Oven temperature: 40° C.
Pressure: 1800 psi

### Method SFC-9:

Preparative chiral HPLC
Instrument: Sepiatec prep SFC-100
Column: Chiralpak IC, 250×30 mm 5 μm
Eluent: A: 15% 0.05% NH₃ in MeOH, B: 85% scCO₂ (isocratic)
Flow rate: 80 mL/min
Detection: UV 240 nm
Injection volume: 28×0.80 mL
Oven temperature: 40° C.
Pressure: 130 bar

### Analytical Chiral HPLC

Instrument: Analytical SFC-MS Waters UPC2
Injection: 5 μL
Mobile phase: A: 10% 0.05% NH₃ in MeOH, B: 90% scCO₂ (isocratic)
Flow rate: 3 mL/min
Column: Chiralpak IK (4.6 mm×100 mm 5 μm)
Detection UV: DAD
Oven temperature: 40° C.
Pressure: 1800 psi

### Method C-10:

### Preparative Chiral HPLC

Instrument: Agilent 1260 Infinity
Column: Chiralpak IG, 250×21 mm, 5 μm
Eluent: A: 40% n-hexane, B: 60% EtOH/MeOH=1/1 (isocratic)
Flow rate: 15.0 mL/min
Detection: 210 nm
Oven/column temperature: 23° C.
Pressure: 80 bar

### Analytical Chiral HPLC

Instrument: Agilent, 1260 infinity II
Injection: 10 μL
Mobile phase: A: 50% n-hexane, B: 50% 0.1% DEA in EtOH//MeOH=70/30 (isocratic)
Flow rate: 1 mL/min
Column: Chiralpak IG (4.6×150 mm 5 μm)
Detection UV: DAD
Oven/column temperature: 25° C.
Pressure: 400 bar

### Method SFC-11:

### Preparative Chiral HPLC

Instrument: Waters SFC-100 (equipped with ABsys GmbH upgrade)
Column: Chiralpak IG, 250×30 mm 5 μm
Eluent: A: 35-45% 0.05% NH₃ in MeOH, B: 65-55% scCO₂
Flow rate: 110.0 mL/min
Detection: DAD
Injection volume: 3×1.30 mL
Oven temperature: 50° C.
Pressure: 120 bar

### Analytical Chiral HPLC

Instrument: Analytical SFC-MS Waters UPC2
Injection: 5 μL
Mobile phase: A: 38% 0.05% NH₃ in MeOH, B: 62% scCO₂ (isocratic)
Flow rate: 3 mL/min
Column: Chiralpak IG (4.6×100 mm 5 μm)
Detection UV: DAD
Oven temperature: 40° C.
Pressure: 1800 psi

### Method SFC-12:

### Preparative Chiral HPLC

Instrument: Sepiatec prep SFC-100
Column: Chiralpak IC, 250×21 mm 5 μm
Eluent: A: 40% 10 mM NH₃ in 4:1 MeOH/ACN, B: 60% scCO₂ (isocratic)
Flow rate: 80 g/min
Detection: UV 272 nm
Injection volume: 13×2 mL
Oven temperature: 40° C.
Pressure: 100 bar

### Analytical Chiral HPLC

Instrument: Analytical SFC-MS Waters UPC2
Injection: 5 μL
Mobile phase: A: 0.1% TEA in 4:1 MeOH/ACN, B: 0.1% TEA in scCO₂; 5-55% A to B gradient over 5 min
Flow rate: 2.5 mL/min
Column: Chiralpak IC (3×100 mm 3 μm)
Detection UV: DAD
Oven temperature: 40° C.
Pressure: 1800 psi

### Method SFC-13:

### Preparative Chiral HPLC

Instrument: Sepiatec prep SFC-100
Column: Chiralpak IE, 250×21 mm 5 μm
Eluent: A: 45% 10 mM NH₃ in MeOH, B: 55% scCO₂ (isocratic)
Flow rate: 80 g/min
Detection: UV 248 nm
Injection volume: 5×1 mL
Oven temperature: 40° C.
Pressure: 125 bar

### Analytical Chiral HPLC

Instrument: Analytical SFC-MS Waters UPC2
Injection: 5 μL
Mobile phase: A: 0.1% NH₃ in MeOH, B: scCO₂; 5-55% A to B gradient over 5 min
Flow rate: 2.5 mL/min
Column: Chiralpak IE (3×100 mm 3 μm)
Detection UV: DAD
Oven temperature: 40° C.
Pressure: 1800 psi

### Biological Data

### Determination of Complement Factor B Inhibition

### Biological Example 1: Human Complement Factor B ELISA Assay

CVF-Bb complex prepared from purified cobra venom factor (1 μM), recombinant human complement factor B (expressed in drosophila cells and purified using standard methods) and human complement factor D (expressed in E. Coli, refolded and purified using standard methods). CVF-Bb complex at 3 nM concentration was incubated with test compound at various concentrations for 1 h at RT in PBS pH 7.4 containing 10 mM MgCl₂ and 0.05% (w/v) CHAPS. Human complement C3 substrate purified from plasma was added to a final concentration of 1 μM. After 1 h incubation at RT, the enzyme reaction was stopped by addition of a cocktail of concentrated pan-protease inhibitors. The product of the reaction, C3a, was quantified by means of an enzyme-linked-immunosorbent assay. IC₅₀ values were calculated from percentage of inhibition of CVF-Bb activity as a function of test compound concentration.

### Biological Example 2: Human Complement Factor B TR-FRET Assay

Recombinant human factor B (expressed in drosophila cells and purified using standard methods) labeled with biotin (10 nM), europium-labeled streptavidin (5 nM) and (+) or (−)-2-(1E,3E,5E)-5-(1-(6-(2-(3-(4-((R)-3-amino-3-phenylpropanoyl)-1-(4-amino-6,7-dimethoxyquinazolin-2-yl)piperazin-2-yl)phenoxy)ethyl)amino)-6-oxohexyl)-3,3-dimethyl-5-sulfoindolin-2-ylidene) penta-1,3-dien-1-yl)-1-ethyl-3,3-dimethyl-5-sulfo-3H-indol-1-ium (prepared as described in WO2015/009616, 240 nM activity agaist factor B when tested using the assay of Biological Example 1) (75 nM) were incubated with test compound at various concentrations up to 2 hours at RT in 20 mM Tris/HCl, PH 7.4, 0.005% (v/v) Tween20.

The time-gated decrease in fluorescence intensity related to the competition between labeled and unlabeled factor B ligands was recorded at both 620 nm and 665 nm, 70 us after excitation at 337 nm using a microplate spectrofluorimeter. IC₅₀ values were calculated from percentage of inhibition of complement factor B-(+) or (−)-2-((1E,3E,5E)-5-(1-(6-((2-(3-(4-((R)-3-amino-3-phenylpropanoyl)-1-(4-amino-6,7-dimethoxyquinazolin-2-yl)piperazin-2-yl)phenoxy)ethyl)amino)-6-oxohexyl)-3,3-dimethyl-5-sulfoindolin-2-ylidene) penta-1,3-dien-1-yl)-1-ethyl-3,3-dimethyl-5-sulfo-3H-indol-1-ium (240 nM activity agaist factor B when tested using the assay of Biological Example 1) displacement as a function of test compound concentration.

Compounds of disclosure are active on factor B inhibition. Data on Table 1 collected using the assay of Biological Example 2.

TABLE 1

| | hFactor-B TR-FRET Qualified IC₅₀ |
|---|---|
| Example | A (≤0.1 μM), B (≤1 μM), C (≤30 μM) |
| Ex-1 | A |
| Ex-2 | C |
| Ex-3 | A |
| Ex-4 | C |
| Ex-5 | B |
| Ex-6 | C |
| Ex-7 | B |
| Ex-8 | A |
| Ex-9 | C |
| Ex-10 | A |
| Ex-11 | A |
| Ex-12 | A |
| Ex-13 | A |
| Ex-14 | A |
| Ex-15 | B |
| Ex-16 | A |
| Ex-17 | A |
| Ex-18 | A |
| Ex-19 | A |
| Ex-20 | A |
| Ex-21 | B |
| Ex-22 | A |
| Ex-23 | A |
| Ex-24 | A |
| Ex-25 | C |
| Ex-26 | C |
| Ex-27 | A |
| Ex-28 | A |
| Ex-29 | A |
| Ex-30 | A |
| Ex-31 | C |
| Ex-32 | B |
| Ex-33 | A |
| Ex-34 | A |
| Ex-35 | A |
| Ex-36 | A |
| Ex-37 | A |
| Ex-38 | A |
| Ex-39 | A |
| Ex-40 | A |
| Ex-41 | A |
| Ex-42 | A |
| Ex-43 | A |
| Ex-44 | B |
| Ex-45 | C |
| Ex-46 | A |
| Ex-47 | B |
| Ex-48 | A |
| Ex-49 | A |
| Ex-50 | B |
| Ex-51 | B |
| Ex-52 | B |
| Ex-53 | B |
| Ex-54 | B |
| Ex-55 | B |
| Ex-56 | B |
| Ex-57 | C |
| Ex-58 | C |
| Ex-59 | A |
| Ex-60 | A |
| Ex-61 | A |

The compounds of the disclosure inhibit complement factor B activity at micromolar IC₅₀ values. As such, the compounds of the disclosure may be useful in treating the diseases and/or disorders described herein, e.g., a disease/disorder mediated by complement factor B.

All publications and patent documents cited herein are incorporated herein by reference as if each such publication or document was specifically and individually indicated to be incorporated herein by reference. The present disclosure and its embodiments have been described in detail. However, the scope of the present disclosure is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present disclosure. Accordingly, one of ordinary skill in the art will readily appreciate from the disclosure that later modifications, substitutions, and/or variations performing substantially the same function or achieving substantially the same result as embodiments described herein may be utilized according to such related embodiments of the present disclosure. Thus, the following claims are intended to encompass within their scope modifications, substitutions, and variations to processes, manufactures, compositions of matter, compounds, means, methods, and/or steps disclosed herein. The claims should not be read as limited to the described order or elements unless stated to that effect. It should be understood that various changes in form and detail may be made without departing from the scope of the appended claims.

1. A compound of formula (I) or a pharmaceutically acceptable salt thereof, 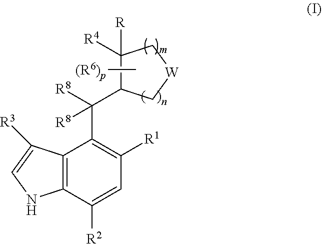
wherein: m is 0, 1, 2, or 3; n is 0, 1, 2, or 3, with the proviso that both m and n are not 0;
R is hydrogen, halogen, C₁-C₄alkyl, haloC₁-C₄alkyl, or hydroxyC₁-C₄alkyl;
R¹ is hydrogen, halogen, hydroxy, C₁-C₆alkyl, C₂-C₆alkenyl, C₃-C₆cycloalkyl, C₁-C₆alkoxy, haloC₁-C₆alkyl, haloC₃-C₆cycloalkyl, haloC₁-C₆alkoxy, hydroxyC₁-C₆alkyl, hydroxyC₃-C₆cycloalkyl aminoC₁-C₆alkyl, C₁-C₆alkoxyC₁-C₆alkyl, C₁-C₆alkoxyC₁-C₆alkoxy, C₃-C₆cycloalkylC₁-C₆alkoxy, haloC₁-C₆alkoxy, —S—C₁-C₆alkyl, —S(O) C₁-C₆alkyl, —S(O)₂C₁-C₆alkyl, CH₂NHC(O)C₁-C₄alkyl or —OCH₂C(O)R^{1a};
R^{1a} is hydroxy, C₁-C₆alkoxy, amino or mono- and di-C₁-C₆alkylamino;
R² is C₁-C₆alkyl, C₅cycloalkyl, C₁-C₆alkoxy, hydroxyC₁-C₆alkyl or halogen, wherein the C₁-C₆alkyl, C₅cycloalkyl, or C₁-C₆alkoxy are unsubstituted or substituted with 1, 2, or 3 halogen substituents;
p is 0, 1, 2, or 3;
R³ is hydrogen, halogen, or cyano;
R⁴ is phenyl, naphthyl or heteroaryl, where the heteroaryl is a five to 10 member heteroaryl having 1, 2 or 3 ring heteroatoms independently selected from N, O, or S, and where the phenyl or heteroaryl is unsubstituted or substituted with 1 R⁵ and substituted with 0, 1, or 2 R^{5′}; wherein
R⁵ is selected from —CO₂R^{5b}, —CON(R^{5c})₂, C₁-C₆alkyl, C₁-C₆alkoxyC₁-C₆alkyl, —CH₂CO₂R^{5b}, —CH₂CON(R^{5c})₂, —C(O)NHSO₂C₁-C₄alkyl, —SO₂NHC(O)C₁-C₄alkyl, —SO₂N(H)ₘ(C₁-C₄alkyl)₂₋ₘ, —SO₂C₁-C₄alkyl, cyano, hydroxy, halogen, 5- to 6-membered heteroaryl having 1-4 heteroatoms independently selected from N, O, and S, and 4- to 6-membered heterocyclyl having 1-2 heteroatoms independently selected from N, O, and S(O)_{q}, wherein the 5- to 6-membered heteroaryl and 4- to 6-membered heterocyclyl are unsubstituted or substituted with 1, 2, or 3 R^{5a};
each R^{5a} is independently selected from fluoro, hydroxyl and C₁-C₆alkyl that is unsubstituted or substituted with —COOH or 1, 2, or 3 fluoro;
wherein when R⁵ is a 4- to 6-membered heterocyclyl, two R^{5a} are not fluoro and hydroxyl substituted on the same position;
each R^{5b} is independently selected from hydrogen or C₁-C₅alkyl;
each R^{5c} is independently selected from hydrogen, C₁-C₄alkyl unsubstituted or substituted with halogen, hydroxy or C₁-C₄alkyl;
each R^{5′} is independently selected from the group consisting of haloC₁-C₄alkyl, C₁-C₄alkoxy, haloC₁-C₄alkoxy, halogen, cyanomethyl, hydroxyC₁-C₆alkyl, and C₃-C₅cycloalkyl;
W is O, C(R⁶)₂, or NR⁷;
R⁶ is independently selected at each occurrence from the group consisting of hydrogen, halogen, hydroxy, amino, mono- and di-C₁-C₄alkylamino, C₁-C₆alkyl, haloC₁-C₆alkyl, hydroxyC₁-C₆alkyl, cyanoC₁-C₆alkyl, C₁-C₆alkoxy, or haloC₁-C₆alkoxy; or two R⁶ in combination with the carbon atom to which they are attached form a spirocyclic carbocycle having 3 to 6 ring atoms, wherein the spirocyclic carbocycle is unsubstituted or substituted with 1 or 2 substituents selected from halogen, C₁-C₆alkyl, or C₁-C₆alkoxy; or two R⁶ in combination with the carbon atom to which they are attached form a spirocyclic heterocycle having 1 or 2 ring heteroatoms independently selected from N, O, or S(O)_{q}, wherein the spirocyclic heterocycle is unsubstituted or substituted with 1 or 2 halogen substituents, C₁-C₆alkyl, haloC₁-C₆alkyl, C₃-C₆cycloalkyl, haloC₃-C₆cycloalkyl;
q is 0, 1, or 2;
R⁷ is hydrogen, C₁-C₆alkyl, haloC₁-C₆alkyl, hydroxyC₂-C₆alkyl, C₃-C₆cycloalkyl, C₃-C₆cycloalkylC₁-C₆alkyl, haloC₃-C₆cycloalkyl, C₁-C₆alkoxyC₁-C₆alkyl, haloC₁-C₆alkoxyC₁-C₆alkyl, aryl, 4- to 6-membered heterocyclyl having 1-2 heteroatoms independently selected from N, O, and S(O)_{q}, or 5- to 6-membered heteroaryl having 1-4 heteroatoms independently selected from N, O, and S, wherein C₁-C₆alkyl is unsubstituted or substituted with 4- to 6-membered heterocyclyl having 1-2 heteroatoms independently selected from N, O, and S(O)_{q}, wherein the 4- to 6-membered heterocyclyl is unsubstituted or substituted with 1, 2, or 3 halogen, further wherein the aryl or heteroaryl is unsubstituted or substituted with halo, C₁-C₆alkyl, haloC₁-C₆alkyl, C₃-C₆cycloalkyl, haloC₃-C₆cycloalkyl;
each R⁸ is independently hydrogen, halogen, C₁-C₄alkyl, haloC₁-C₄alkyl, or two R⁸ together with the carbon atom to which they are attached form a C₃-C₆cycloalkyl or haloC₃-C₆cycloalkyl.

2-27. (canceled)

28. The compound of formula (I) according to claim 1, or a pharmaceutically acceptable salt thereof, selected from:
4-((3R,4R)-4-((5,7-dimethyl-1H-indol-4-yl)methyl)-1-(2,2,2-trifluoroethyl)piperidin-3-yl)benzoic acid;
4-(4-((5,7-dimethyl-1H-indol-4-yl)methyl)-1-(2,2,2-trifluoroethyl)piperidin-3-yl)benzoic acid;
4-((3R,4R)-4-((5-methoxy-7-methyl-1H-indol-4-yl)methyl)-1-(2,2,2-trifluoroethyl)piperidin-3-yl)benzoic acid;
4-(4-((5-methoxy-7-methyl-1H-indol-4-yl)methyl)-1-(2,2,2-trifluoroethyl)piperidin-3-yl)benzoic acid;
4-((3R,4R)-4-((5,7-dimethyl-1H-indol-4-yl)methyl)-1-methylpiperidin-3-yl)benzoic acid;
4-(4-((5,7-dimethyl-1H-indol-4-yl)methyl)-1-methylpiperidin-3-yl)benzoic acid;
4-((3R,4R)-4-((5-methoxy-7-methyl-1H-indol-4-yl)methyl)-1-methylpiperidin-3-yl)benzoic acid;
4-(4-((5-methoxy-7-methyl-1H-indol-4-yl)methyl)-1-methylpiperidin-3-yl)benzoic acid;
4-((3R,4R)-1-(2,2-difluoroethyl)-4-((5,7-dimethyl-1H-indol-4-yl)methyl)piperidin-3-yl)benzoic acid;
4-(1-(2,2-difluoroethyl)-4-((5,7-dimethyl-1H-indol-4-yl)methyl)piperidin-3-yl)benzoic acid;
4-((3R,4R)-4-((5-cyclopropyl-7-methyl-1H-indol-4-yl)methyl)-1-(2,2,2-trifluoroethyl)piperidin-3-yl)benzoic acid;
4-(4-((5-cyclopropyl-7-methyl-1H-indol-4-yl)methyl)-1-(2,2,2-trifluoroethyl)piperidin-3-yl)benzoic acid;
4-((3R,4R)-4-((5,7-dimethyl-1H-indol-4-yl)methyl)-1-(2-fluoroethyl)piperidin-3-yl)benzoic acid;
4-(4-((5,7-dimethyl-1H-indol-4-yl)methyl)-1-(2-fluoroethyl)piperidin-3-yl)benzoic acid;
4-((3R,4R)-4-((5-cyclopropyl-7-methyl-1H-indol-4-yl)methyl)-1-methylpiperidin-3-yl)benzoic acid;
4-(4-((5-cyclopropyl-7-methyl-1H-indol-4-yl)methyl)-1-methylpiperidin-3-yl)benzoic acid;
4-((3R,4R)-4-((5,7-dimethyl-1H-indol-4-yl)methyl)-1-isobutylpiperidin-3-yl)benzoic acid;
4-(4-((5,7-dimethyl-1H-indol-4-yl)methyl)-1-isobutylpiperidin-3-yl)benzoic acid;
4-((3R,4R)-1-(cyclopropylmethyl)-4-((5,7-dimethyl-1H-indol-4-yl)methyl)piperidin-3-yl)benzoic acid;
4-(1-(cyclopropylmethyl)-4-((5,7-dimethyl-1H-indol-4-yl)methyl)piperidin-3-yl)benzoic acid;
4-((3R,4R)-4-((5,7-dimethyl-1H-indol-4-yl)methyl)-1-(2-methoxyethyl)piperidin-3-yl)benzoic acid;
4-(4-((5,7-dimethyl-1H-indol-4-yl)methyl)-1-(2-methoxyethyl)piperidin-3-yl)benzoic acid;
4-(((3R,4R)-3-(4-(1H-tetrazol-5-yl)phenyl)-1-(2,2,2-trifluoroethyl)piperidin-4-yl)methyl)-5,7-dimethyl-1H-indole;
4-((3-(4-(1H-tetrazol-5-yl)phenyl)-1-(2,2,2-trifluoroethyl)piperidin-4-yl)methyl)-5,7-dimethyl-1H-indole;
4-((1R,2S)-2-((5,7-dimethyl-1H-indol-4-yl)methyl)-5,5-difluorocyclohexyl)benzoic acid;
4-(2-((5,7-dimethyl-1H-indol-4-yl)methyl)-5,5-difluorocyclohexyl)benzoic acid;
4-((6R,7S)-7-((5,7-dimethyl-1H-indol-4-yl)methyl)spiro[3.5]nonan-6-yl)benzoic acid;
4-(7-((5,7-dimethyl-1H-indol-4-yl)methyl)spiro[3.5]nonan-6-yl)benzoic acid;
4-((6R,7S)-7-((5,7-dimethyl-1H-indol-4-yl)methyl)-2,2-difluorospiro[3.5]nonan-6-yl)benzoic acid;
4-(7-((5,7-dimethyl-1H-indol-4-yl)methyl)-2,2-difluorospiro[3.5]nonan-6-yl)benzoic acid;
4-((1R,2S,5S)-2-((5,7-dimethyl-1H-indol-4-yl)methyl)-5-ethoxycyclohexyl)benzoic acid;
4-(2-((5,7-dimethyl-1H-indol-4-yl)methyl)-5-ethoxycyclohexyl)benzoic acid;
4-((1R,2S,5S)-5-(2,2-difluoroethoxy)-2-((5,7-dimethyl-1H-indol-4-yl)methyl)cyclohexyl)benzoic acid;
4-(5-(2,2-difluoroethoxy)-2-((5,7-dimethyl-1H-indol-4-yl)methyl)cyclohexyl)benzoic acid;
4-((1R,2S,5S)-5-(cyclopropylmethoxy)-2-((5,7-dimethyl-1H-indol-4-yl)methyl)cyclohexyl)benzoic acid;
4-(5-(cyclopropylmethoxy)-2-((5,7-dimethyl-1H-indol-4-yl)methyl)cyclohexyl)benzoic acid;
4-((3R,4S)-4-((5,7-dimethyl-1H-indol-4-yl)methyl)-1-(2,2,2-trifluoroethyl)pyrrolidin-3-yl)benzoic acid;
4-(4-((5,7-dimethyl-1H-indol-4-yl)methyl)-1-(2,2,2-trifluoroethyl)pyrrolidin-3-yl)benzoic acid;
4-((3R,4S)-4-((5,7-dimethyl-1H-indol-4-yl)methyl)-1-(2,2,2-trifluoroethyl)azepan-3-yl)benzoic acid;
4-(4-((5,7-dimethyl-1H-indol-4-yl)methyl)-1-(2,2,2-trifluoroethyl)azepan-3-yl)benzoic acid;
2-(4-((3R,4R)-4-((5,7-dimethyl-1H-indol-4-yl)methyl)-1-(2,2,2-trifluoroethyl)piperidin-3-yl)-1H-pyrazol-1-yl) acetic acid;
2-(4-(4-((5,7-dimethyl-1H-indol-4-yl)methyl)-1-(2,2,2-trifluoroethyl)piperidin-3-yl)-1H-pyrazol-1-yl) acetic acid;
4-(((3R,4R)-3-(1-(2-methoxyethyl)-1H-pyrazol-4-yl)-1-(2,2,2-trifluoroethyl)piperidin-4-yl)methyl)-5,7-dimethyl-1H-indole;
4-((3-(1-(2-methoxyethyl)-1H-pyrazol-4-yl)-1-(2,2,2-trifluoroethyl)piperidin-4-yl)methyl)-5,7-dimethyl-1H-indole;
4-((3R,4R)-4-((5,7-dimethyl-1H-indol-4-yl)methyl)piperidin-3-yl)benzoic acid;
4-(4-((5,7-dimethyl-1H-indol-4-yl)methyl)piperidin-3-yl)benzoic acid;
4-((3R,4R)-4-((5,7-dimethyl-1H-indol-4-yl)methyl)-1-ethylpiperidin-3-yl)benzoic acid;
4-(-((5,7-dimethyl-1H-indol-4-yl)methyl)-1-ethylpiperidin-3-yl)benzoic acid;
4-((3R,4R)-4-((5,7-dimethyl-1H-indol-4-yl)methyl)-1-isopropylpiperidin-3-yl)benzoic acid;
4-(4-((5,7-dimethyl-1H-indol-4-yl)methyl)-1-isopropylpiperidin-3-yl)benzoic acid;
4-((3R,4R)-4-((5,7-dimethyl-1H-indol-4-yl)methyl)-1-(2-hydroxy-2-methylpropyl)piperidin-3-yl)benzoic acid;
4-(4-((5,7-dimethyl-1H-indol-4-yl)methyl)-1-(2-hydroxy-2-methylpropyl)piperidin-3-yl)benzoic acid;
4-((3R,4R)-4-((5,7-dimethyl-1H-indol-4-yl)methyl)-1-((3-fluorooxetan-3-yl)methyl)piperidin-3-yl)benzoic acid;
4-(4-((5,7-dimethyl-1H-indol-4-yl)methyl)-1-((3-fluorooxetan-3-yl)methyl)piperidin-3-yl)benzoic acid;
4-((3R,4R)-1-(3,3-difluorocyclobutyl)-4-((5,7-dimethyl-1H-indol-4-yl)methyl)piperidin-3-yl)benzoic acid;
4-(1-(3,3-difluorocyclobutyl)-4-((5,7-dimethyl-1H-indol-4-yl)methyl)piperidin-3-yl)benzoic acid;
4-((3R,4R)-4-((5,7-dimethyl-1H-indol-4-yl)methyl)-1-(2-(trifluoromethoxy)ethyl)piperidin-3-yl)benzoic acid;
4-(4-((5,7-dimethyl-1H-indol-4-yl)methyl)-1-(2-(trifluoromethoxy)ethyl)piperidin-3-yl)benzoic acid;
4-((3R,4R)-4-((5,7-dimethyl-1H-indol-4-yl)methyl)-1-propylpiperidin-3-yl)benzoic acid;
4-(4-((5,7-dimethyl-1H-indol-4-yl)methyl)-1-propylpiperidin-3-yl)benzoic acid;
4-((3R,4R)-4-((5,7-dimethyl-1H-indol-4-yl)methyl)-1-(oxetan-3-yl)piperidin-3-yl)benzoic acid;
4-(4-((5,7-dimethyl-1H-indol-4-yl)methyl)-1-(oxetan-3-yl)piperidin-3-yl)benzoic acid;
4-((3R,4R)-4-((5,7-dimethyl-1H-indol-4-yl)methyl)-1-((1r,3R)-3-fluorocyclobutyl)piperidin-3-yl)benzoic acid;
4-(4-((5,7-dimethyl-1H-indol-4-yl)methyl)-1-(3-fluorocyclobutyl)piperidin-3-yl)benzoic acid;
4-((3R,4R)-1-(3,3-difluoropropyl)-4-((5,7-dimethyl-1H-indol-4-yl)methyl)piperidin-3-yl)benzoic acid;
4-(1-(3,3-difluoropropyl)-4-((5,7-dimethyl-1H-indol-4-yl)methyl)piperidin-3-yl)benzoic acid;
4-((3R,4S)-4-((5,7-dimethyl-1H-indol-4-yl)methyl)-1-methylpyrrolidin-3-yl)benzoic acid;
4-(4-((5,7-dimethyl-1H-indol-4-yl)methyl)-1-methylpyrrolidin-3-yl)benzoic acid;
4-((3R,4R)-4-((5,7-dimethyl-1H-indol-4-yl)methyl)-1-methylpiperidin-3-yl)benzonitrile;
4-(4-((5,7-dimethyl-1H-indol-4-yl)methyl)-1-methylpiperidin-3-yl)benzonitrile;
4-((3R,4S)-4-((5,7-dimethyl-1H-indol-4-yl)methyl)-1-methylazepan-3-yl)benzoic acid;
4-(4-((5,7-dimethyl-1H-indol-4-yl)methyl)-1-methylazepan-3-yl)benzoic acid;
4-(4-((5,7-dimethyl-1H-indol-4-yl)methyl)-1-(2,2,2-trifluoroethyl)azepan-3-yl)benzoic acid;
4-((3R,4S)-4-((5,7-dimethyl-1H-indol-4-yl)methyl)-1-ethylazepan-3-yl)benzoic acid;
4-(4-((5,7-dimethyl-1H-indol-4-yl)methyl)-1-ethylazepan-3-yl)benzoic acid;
4-((4R,5R)-5-((5,7-dimethyl-1H-indol-4-yl)methyl)-1-methylazepan-4-yl)benzoic acid;
4-(5-((5,7-dimethyl-1H-indol-4-yl)methyl)-1-methylazepan-4-yl)benzoic acid;
4-((3R,4R)-4-((5-fluoro-7-methyl-1H-indol-4-yl)methyl)-1-methylpiperidin-3-yl)benzoic acid;
4-(4-((5-fluoro-7-methyl-1H-indol-4-yl)methyl)-1-methylpiperidin-3-yl)benzoic acid;
4-((4R,5R)-1-(3,3-difluoropropyl)-5-((5,7-dimethyl-1H-indol-4-yl)methyl)azepan-4-yl)benzoic acid;
4-(1-(3,3-difluoropropyl)-5-((5,7-dimethyl-1H-indol-4-yl)methyl)azepan-4-yl)benzoic acid;
4-((3R,4R)-1-methyl-4-((7-methyl-1H-indol-4-yl)methyl)piperidin-3-yl)benzoic acid;
4-(1-methyl-4-((7-methyl-1H-indol-4-yl)methyl)piperidin-3-yl)benzoic acid;
4-((3R,4R)-4-((5-chloro-7-methyl-1H-indol-4-yl)methyl)-1-methylpiperidin-3-yl)benzoic acid;
4-(4-((5-chloro-7-methyl-1H-indol-4-yl)methyl)-1-methylpiperidin-3-yl)benzoic acid;
4-((3R,4R)-4-((5,7-dichloro-1H-indol-4-yl)methyl)-1-methylpiperidin-3-yl)benzoic acid;
4-(4-((5,7-dichloro-1H-indol-4-yl)methyl)-1-methylpiperidin-3-yl)benzoic acid;
4-((3R,4R)-4-((7-chloro-5-methyl-1H-indol-4-yl)methyl)-1-methylpiperidin-3-yl)benzoic acid;
4-(4-((7-chloro-5-methyl-1H-indol-4-yl)methyl)-1-methylpiperidin-3-yl)benzoic acid;
4-((3R,4R)-4-((3-chloro-5,7-dimethyl-1H-indol-4-yl)methyl)-1-methylpiperidin-3-yl)benzoic acid;
4-(4-((3-chloro-5,7-dimethyl-1H-indol-4-yl)methyl)-1-methylpiperidin-3-yl)benzoic acid;
4-((3R,4R)-1-(3,3-difluoropropyl)-4-((5-methoxy-7-methyl-1H-indol-4-yl)methyl)piperidin-3-yl)benzoic acid;
4-(1-(3,3-difluoropropyl)-4-((5-methoxy-7-methyl-1H-indol-4-yl)methyl)piperidin-3-yl)benzoic acid;
4-(((3R,4R)-3-(4-(1H-tetrazol-5-yl)phenyl)-1-methylpiperidin-4-yl)methyl)-5,7-dimethyl-1H-indole;
4-((3-(4-(1H-tetrazol-5-yl)phenyl)-1-methylpiperidin-4-yl)methyl)-5,7-dimethyl-1H-indole;
4-((3R,4R)-4-((5,7-dimethyl-1H-indol-4-yl)methyl)-1-(2,2,2-trifluoroethyl)piperidin-3-yl)benzonitrile;
4-(4-((5,7-dimethyl-1H-indol-4-yl)methyl)-1-(2,2,2-trifluoroethyl)piperidin-3-yl)benzonitrile;
4-(((3R,4R)-3-(4-(1H-pyrazol-1-yl)phenyl)-1-methylpiperidin-4-yl)methyl)-5,7-dimethyl-1H-indole;
4-((3-(4-(1H-pyrazol-1-yl)phenyl)-1-methylpiperidin-4-yl)methyl)-5,7-dimethyl-1H-indole;
4-(((3R,4R)-3-(4-(1H-pyrazol-1-yl)phenyl)-1-(2,2,2-trifluoroethyl)piperidin-4-yl)methyl)-5,7-dimethyl-1H-indole;
4-((3-(4-(1H-pyrazol-1-yl)phenyl)-1-(2,2,2-trifluoroethyl)piperidin-4-yl)methyl)-5,7-dimethyl-1H-indole;
4-(((3R,4R)-3-(4-(1H-pyrazol-1-yl)phenyl)-1-(3,3-difluoropropyl)piperidin-4-yl)methyl)-5,7-dimethyl-1H-indole;
4-((3-(4-(1H-pyrazol-1-yl)phenyl)-1-(3,3-difluoropropyl)piperidin-4-yl)methyl)-5,7-dimethyl-1H-indole;
4-(((3R,4R)-3-(4-(1H-pyrazol-1-yl)phenyl)-1-((3-fluorooxetan-3-yl)methyl)piperidin-4-yl)methyl)-5,7-dimethyl-1H-indole;
4-((3-(4-(1H-pyrazol-1-yl)phenyl)-1-((3-fluorooxetan-3-yl)methyl)piperidin-4-yl)methyl)-5,7-dimethyl-1H-indole;
4-(((3R,4R)-3-(4-(1H-pyrazol-1-yl)phenyl)-1-(oxetan-3-yl)piperidin-4-yl)methyl)-5,7-dimethyl-1H-indole;
4-((3-(4-(1H-pyrazol-1-yl)phenyl)-1-(oxetan-3-yl)piperidin-4-yl)methyl)-5,7-dimethyl-1H-indole;
4-(((3R,4R)-3-(4-(1H-pyrazol-1-yl)phenyl)-1-(1,3-difluoropropan-2-yl)piperidin-4-yl)methyl)-5,7-dimethyl-1H-indole;
4-((3-(4-(1H-pyrazol-1-yl)phenyl)-1-(1,3-difluoropropan-2-yl)piperidin-4-yl)methyl)-5,7-dimethyl-1H-indole;
5,7-dimethyl-4-(((3R,4R)-1-methyl-3-(1-methyl-1H-pyrazol-4-yl)piperidin-4-yl)methyl)-1H-indole;
5,7-dimethyl-4-((1-methyl-3-(1-methyl-1H-pyrazol-4-yl)piperidin-4-yl)methyl)-1H-indole;
5,7-dimethyl-4-(((3R,4R)-3-(1-methyl-1H-pyrazol-4-yl)-1-(2,2,2-trifluoroethyl)piperidin-4-yl)methyl)-1H-indole;
5,7-dimethyl-4-((3-(1-methyl-1H-pyrazol-4-yl)-1-(2,2,2-trifluoroethyl)piperidin-4-yl)methyl)-1H-indole;
4-(((3R,4R)-1-(2-fluoroethyl)-3-(1-methyl-1H-pyrazol-4-yl)piperidin-4-yl)methyl)-5,7-dimethyl-1H-indole;
4-((1-(2-fluoroethyl)-3-(1-methyl-1H-pyrazol-4-yl)piperidin-4-yl)methyl)-5,7-dimethyl-1H-indole;
4-(((3R,4R)-1-(2,2-difluoroethyl)-3-(1-methyl-1H-pyrazol-4-yl)piperidin-4-yl)methyl)-5,7-dimethyl-1H-indole;
4-((1-(2,2-difluoroethyl)-3-(1-methyl-1H-pyrazol-4-yl)piperidin-4-yl)methyl)-5,7-dimethyl-1H-indole;
4-(((3R,4R)-1-(3,3-difluoropropyl)-3-(1-methyl-1H-pyrazol-4-yl)piperidin-4-yl)methyl)-5,7-dimethyl-1H-indole;
4-((1-(3,3-difluoropropyl)-3-(1-methyl-1H-pyrazol-4-yl)piperidin-4-yl)methyl)-5,7-dimethyl-1H-indole;
4-(((3R,4R)-1-(1,3-difluoropropan-2-yl)-3-(1-methyl-1H-pyrazol-4-yl)piperidin-4-yl)methyl)-5,7-dimethyl-1H-indole;
4-((1-(1,3-difluoropropan-2-yl)-3-(1-methyl-1H-pyrazol-4-yl)piperidin-4-yl)methyl)-5,7-dimethyl-1H-indole;
4-((3R,4S)-4-((5,7-dimethyl-1H-indol-4-yl)methyl)-1-methylpyrrolidin-3-yl)benzamide;
4-(4-((5,7-dimethyl-1H-indol-4-yl)methyl)-1-methylpyrrolidin-3-yl)benzamide;
4-((3R,4S)-4-((5-cyclopropyl-7-methyl-1H-indol-4-yl)methyl)-1-methylpyrrolidin-3-yl)benzoic acid;
4-(4-((5-cyclopropyl-7-methyl-1H-indol-4-yl)methyl)-1-methylpyrrolidin-3-yl)benzoic acid;
4-(((3S,4R)-4-(4-(1H-tetrazol-5-yl)phenyl)-1-methylpyrrolidin-3-yl)methyl)-5,7-dimethyl-1H-indole;
4-((4-(4-(1H-tetrazol-5-yl)phenyl)-1-methylpyrrolidin-3-yl)methyl)-5,7-dimethyl-1H-indole;
4-(((3S,4R)-4-(4-(1H-tetrazol-5-yl)phenyl)-1-ethylpyrrolidin-3-yl)methyl)-5-cyclopropyl-7-methyl-1H-indole; and
4-((4-(4-(1H-tetrazol-5-yl)phenyl)-1-ethylpyrrolidin-3-yl)methyl)-5-cyclopropyl-7-methyl-1H-indole.

29. The compound of formula (I) according to claim 1, or a pharmaceutically acceptable salt thereof, selected from:

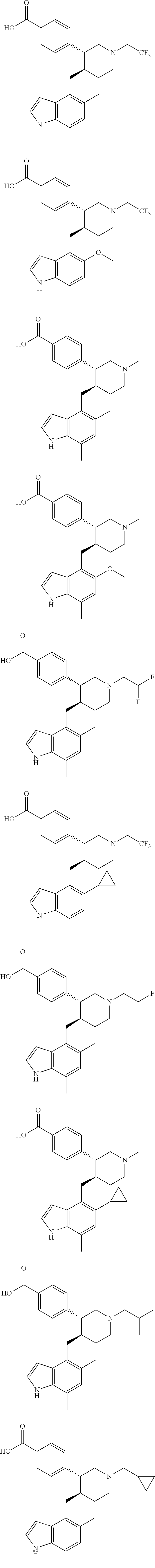

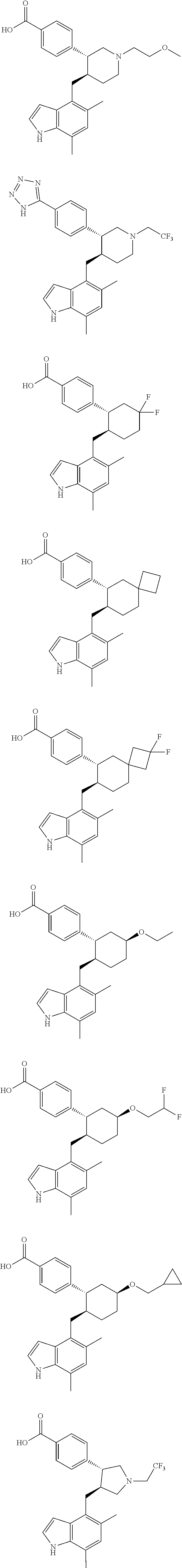

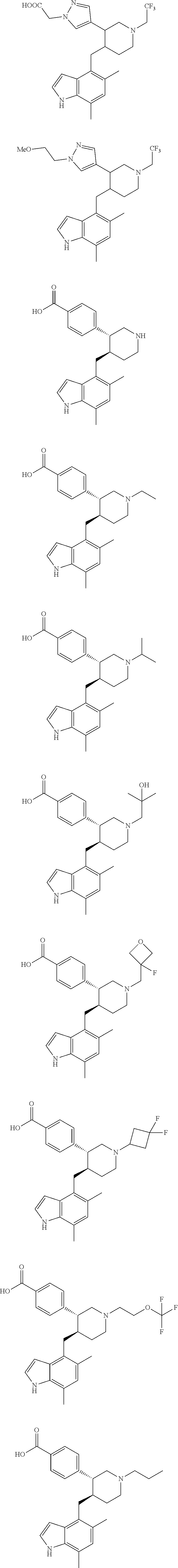

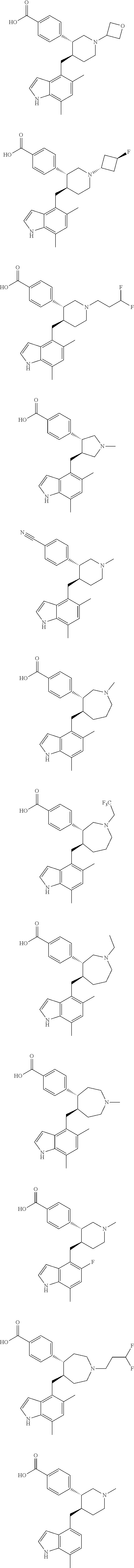

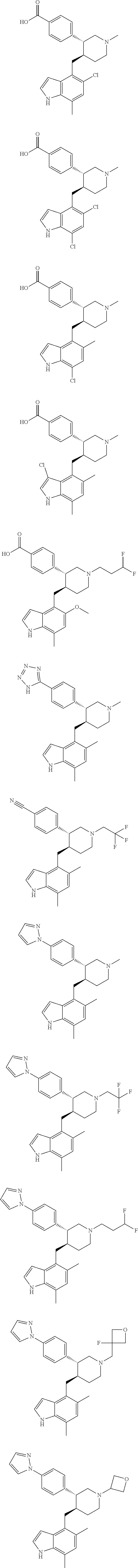

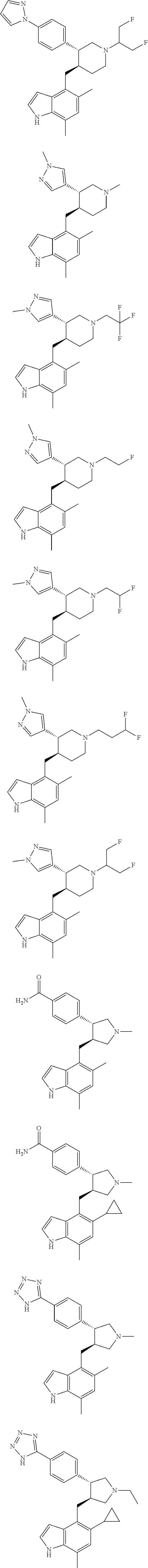

30. A pharmaceutical composition comprising a therapeutically effective amount of a compound according to claim 1, or a pharmaceutically acceptable salt thereof, and a pharmaceutically acceptable carrier or excipient.

31. A method of treating or preventing a disease or disorder in a subject in need thereof, the method comprising administering to the subject a therapeutically effective amount of a compound according to claim 1, or a pharmaceutically acceptable salt thereof.

32-36. (canceled)

37. The method according to claim 31, wherein the disease or disorder is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North carolina macular dystrophy, Stargardt disease, corneal inflammation, neurological disorders such as multiple sclerosis, stroke, Guillain Barre Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity; disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), immune complex membranoproliferative glomerulonephritis (IC-MPGN), IgA nephropathy, membranous nephropathy including idiopathic membranous nephropathy, diabetic nephropathy, atypical hemolytic uremic syndrome (aHUS), Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, neuromyelitis optica (NMO), IgA vasculitis (formerly known as Henoch-Schonlein purpura or HSP), CHAPLE syndrome, hematopoietic stem cell transplantation-associated thrombotic microangiopathy (HSCT-TMA), adult respiratory distress syndrome (ARDS,), myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury mesenteric artery reperfusion after aortic reconstruction, infectious disease or sepsis; COVID-19, immune complex disorders and autoimmune diseases, rheumatoid arthritis, osteoarthritis, Spondyloarthropathies, psoriatic arthritis, systemic lupus erythematosus (SLE), lupus nephritis, SLE nephritis, proliferative nephritis, myasthenia gravis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, antineutrophil cytoplasmic antibody (ANCA)-associated vasculitides (AAV), Buerger's vasculitis, cryoglobulinemia, Kawasaki disease, Takayasu arteritis, cryoglobulinemia, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopeni purpura (TTP), and abdominal aortic aneurisms, and Grave's disease.

38-48. (canceled)

49. A pharmaceutical combination comprising a compound according to claim 1, or a pharmaceutically acceptable salt thereof, and one or more additional therapeutic agent(s).
